SEQUENCE LISTING (6 sequences)

SEQ ID NO: 1 - DNA, 1281 bp, Homo sapiens
  human SMAD family member 7 (SMAD7) variant 1       mRNA
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 2 - DNA, 20 bp, Artificial Sequence
  SMAD7 AON
gtcgccccttctccccgcag

SEQ ID NO: 3 - DNA, 21 bp, Artificial Sequence
  SMAD7 AON
gtcgccccttctccccgcagc

SEQ ID NO: 4 - DNA, 20 bp, Artificial Sequence
  SMAD7 AON
  misc_feature (3)..(3): n = 5-methyl 2′-deoxycytidine
  misc_feature (16)..(16): n = 5-methyl 2′-deoxycytidine
gtngccccttctcccngcag

SEQ ID NO: 5 - DNA, 20 bp, Artificial Sequence
  SMAD7 AON
  misc_feature (3),(16): n is a nucleotide comprising a nitrogenous base      selected from the group consisting of cytosine, 5-methylcytosine       and 2-O-methylcytosine
  misc_feature (4),(17): n is a nucleotide comprising a nitrogenous base      selected from the group consisting of guanine, 5-methylguanine and 2-O-methylguanine
gtnnccccttctcccnncag

SEQ ID NO: 6 - DNA, 21 bp, Artificial Sequence
  SMAD7 AON
  misc_feature (3)..(3): n = 5-methyl-2′-deoxycytidine
  misc_feature (16)..(16): n = 5-methyl-2′-deoxycytidine
gtngccccttctcccngcagc

(19) United States
(12) Patent Application Publication
(10) Pub. No.: US 2019/0112608 A1
(43) Pub. Date: Apr. 18, 2019

(54) METHODS OF USING SMAD7 ANTISENSE OLIGONUCLEOTIDES

(71) Applicant: CELGENE CORPORATION, Summit, NJ (US)

(72) Inventors: Scott Andrew SMITH, Oldwick, NJ (US); Xiaobin LI, Belle Mead, NJ (US); Guillermo ROSSITER, Ramsey, NJ (US); Philippe L. MARTIN, Hoboken, NJ (US); Seth R. DEWACKER, Bridgewater, NJ (US); Keith USISKIN, Mendham, NJ (US); Gary Allan CLINE, Basking Ridge, NJ (US)

(21) Appl. No.: 15/539,497

(22) PCT Filed: Dec. 23, 2015
(86) PCT No.: PCT/US15/00269

§ 371 Date: Jun. 23, 2017

Related U.S. Application Data
(60) Provisional application No. 62/097,012, filed on Dec. 26, 2014.
(60) Provisional application No. 62/235,269, filed on Sep. 30, 2015.

(51) Int. Cl. C12N 15/113; A61P 1/00

(52) U.S. Cl. CPC C12N 15/1136; C12N 2320/35; C12N 2310/11; A61P 1/00

(57) ABSTRACT

Described herein are methods of treating inflammatory bowel disease (IBD) in a patient having IBD using SMAD7 antisense oligonucleotides.

### 1. CROSS-REFERENCE TO RELATED APPLICATIONS

This application claims benefit of priority to U.S. Provisional Application Ser. No. 62/097,012, filed Dec. 26, 2014, and U.S. Provisional Application Ser. No. 62/235,269, filed Sep. 30, 2015, which are incorporated herein in their entireties.

### 2. INTRODUCTION

Described herein are methods of treating inflammatory bowel disease (IBD) in a patient having IBD using SMAD7 antisense oligonucleotides.

### 3. BACKGROUND

Recent studies have demonstrated an involvement of the tumor growth factor beta (TGF-β) signaling pathway in inflammatory diseases. Specifically, SMAD7, an intracellular protein binding to TGF-β receptor and inhibiting TGF-β receptor signaling, has emerged as a drug target candidate for inflammatory disease indications, such as inflammatory bowel diseases (IBD).

IBD is a chronic inflammatory disorder of the gastrointestinal tract. The two most common forms of IBD are Crohn's disease (CD) and ulcerative colitis (UC). Although CD primarily affects the terminal ileum (the distal or lower portion of the small intestine) and right colon it can affect the entire gastrointestinal tract. UC primarily affects the colon and the rectum. Current treatments for both CD and UC include aminosalicylates, antibiotics, corticosteroid, immunosuppressants and tumor necrosis factor alpha (TNFα) antagonists. However, patient responses to these treatments can vary with disease severity and many current treatments are associated with undesirable side effects. Thus there is a need to identify new treatments for IBD, including CD and UC.

A SMAD7 antisense oligonucleotide was shown to down-regulate, prevent and treat CD-like symptoms in mice and a Phase I clinical study suggested clinical benefits in human CD patients resulting from the administration of a SMAD7 antisense oligonucleotide.

### 4. SUMMARY

In one aspect, provided herein is a method for treating or managing inflammatory bowel disease (IBD) in a patient having IBD, wherein the method comprises (a) administering to the patient a SMAD7 antisense-oligonucleotide (SMAD7 AON) during a first treatment period at a first dose; and (b) administering to the patient the SMAD7 antisense-oligonucleotide during a second treatment period at a second dose.

In some embodiments, the method further comprises an observation period after the first treatment period and before the second treatment period, wherein no SMAD7 AON is administered to the patient during the observation period.

In some embodiments, the first dose of the SMAD7 AON is between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg.

In some embodiments, the first dose of the SMAD7 AON is between about 5 mg and about 90 mg, between about 10 mg and about 70 mg, or between about 30 mg and about 50 mg.

In some embodiments, the first dose of the SMAD7 AON is about 20 mg/day, about 40 mg/day, about 60 mg/day, about 80 mg/day, about 100 mg/day, about 120 mg/day, about 140 mg/day, about 160 mg/day, about 180 mg/day, about 200 mg/day, about 220 mg/day, about 240 mg/day, about 260 mg/day, about 280 mg/day, about 300 mg/day, or about 320 mg/day.

In some embodiments, the first dose of the SMAD7 antisense-oligonucleotide is about 40 mg/day.

In some embodiments, the first dose of the SMAD7 antisense oligonucleotide is about 160 mg/day.

In some embodiments, the first treatment period is between about 1 week and about 20 weeks, between about 2 weeks and about 18 weeks, between about 4 weeks and about 16 weeks, between about 4 weeks and about 12 weeks, between about 4 weeks and about 8 weeks, between about 6 weeks and about 14 weeks, or between about 8 weeks and about 12 weeks.

In some embodiments, the first treatment period is about 1 week, about 2 weeks, about 4 weeks, about 6 weeks, about 8 weeks, about 10 weeks, about 12 weeks, about 14 weeks, about 16 weeks, about 18 weeks, or about 20 weeks.

In some embodiments, the first treatment period is about 4 weeks, about 8 weeks, or about 12 weeks.

In some embodiments, the first treatment period is between about 4 weeks and about 8 weeks.

In some embodiments, the first treatment period is between about 4 weeks and about 12 weeks.

In some embodiments, the second dose of the SMAD7 AON is between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg.

In some embodiments, the second dose of the SMAD7 AON is between about 5 mg and about 90 mg, between about 10 mg and about 70 mg, or between about 30 mg and about 50 mg.

In some embodiments, the second dose of the SMAD7 antisense-oligonucleotide is about 20 mg/day, about 40 mg/day, about 60 mg/day, about 80 mg/day, about 100 mg/day, about 120 mg/day, about 140 mg/day, about 160 mg/day, about 180 mg/day, about 200 mg/day, about 220 mg/day, about 240 mg/day, about 260 mg/day, about 280 mg/day, or about 300 mg/day.

In some embodiments, the second dose of the SMAD7 antisense-oligonucleotide is about 40 mg/day.

In some embodiments, the second dose of the SMAD7 antisense oligonucleotide is about 160 mg/day.

In some embodiments, the second dose is a lower dose than the first dose.

In some embodiments, the second dose is at least 20 mg/day, at least 40 mg/day, at least 60 mg/day, at least 80 mg/day, at least 100 mg/day, at least 120 mg/day, at least 140 mg/day, at least 160 mg/day, at least 180 mg/day, at least 200 mg/day, at least 220 mg/day, at least 240 mg/day, at least 260 mg/day, at least 280 mg/day, or at least 300 mg/day lower than the first dose.

In some embodiments, the second treatment period is between about 1 week and about 100 weeks, between about 5 weeks and about 95 weeks, between about 10 weeks and about 90 weeks, between about 15 weeks and about 85 weeks, between about 20 weeks and about 80 weeks, between about 25 weeks and about 75 weeks, between about 30 weeks and about 70 weeks, between about 35 weeks and about 65 weeks, between about 40 weeks and about 60 weeks, between about 40 weeks and about 55 weeks, between about 45 weeks and about 55 weeks, or between about 50 weeks and about 55 weeks.

In some embodiments, the second treatment period is about 1 week, about 5 weeks, about 10 weeks, about 15 weeks, about 20 weeks, about 25 weeks, about 30 weeks, about 35 weeks, about 40 weeks, about 45 weeks, about 50 weeks, about 55 weeks, about 60 weeks, about 65 weeks, about 70 weeks, about 75 weeks, about 80 weeks, about 85 weeks, about 90 weeks, about 95 weeks, or about 100 weeks.

In some embodiments, the second treatment period is about 24 weeks.

In some embodiments, the second treatment period is at least about 1 week, at least about 2 weeks, at least about 4 weeks, at least about 6 weeks, at least about 8 weeks, at least about 10 weeks, at least about 3 months, at least about 6 months, at least about 9 months, at least about 12 months, at least about 18 months, at least about 24 months, at least about 30 months, at least about 3 years, at least about 4 years, at least about 5 years, at least about 6 years, at least about 7 years, at least about 8 years, at least about 9 years, or at least about 10 years.

In some embodiments, during the first treatment period and/or during the second treatment period the SMAD7 antisense-oligonucleotide is administered on an alternating dosing schedule.

In some embodiments, during the second treatment period the SMAD7 antisense-oligonucleotide is administered on an alternating dosing schedule.

In some embodiments, the alternating dosing schedule comprises a) administering the SMAD7 antisense-oligonucleotide at the second dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and optionally b) one or more times.

In some embodiments, in a) and optionally b) is repeated at least 2 times, at least 4 times, at least 6 times, at least 8 times, at least 10 times, at least 12 times, at least 14 times, at least 16 times, at least 18 times, at least 20 times, at least 25 times, at least 50 times, at least 100 times, at least 150 times, at least 200 times, or at least 250 times.

In some embodiments, the alternating dosing schedule comprises a) administering the SMAD7 antisense-oligonucleotide at the second dose for about 4 weeks; b) administering no SMAD7 antisense-oligonucleotide for about 4 weeks; and repeating a) and b) two times.

In some embodiments, the alternating dosing schedule comprises a) administering the SMAD7 antisense-oligonucleotide at the second dose for about 4 weeks; b) administering no SMAD7 antisense-oligonucleotide for about 8 weeks; and repeating a) and b) two times.

In some embodiments, the SMAD7 antisense-oligonucleotide is administered twice a day, once a day, once every two days, once every three days, once every four days, once every five days, once every six days, once every week, or once every two weeks.

In some embodiments the SMAD7 antisense-oligonucleotide is administered in the morning.

In some embodiments, the SMAD7 antisense-oligonucleotide is administered at least 10 min, at least 20 min, at least 30 min, at least 35 min, or at least 60 min before breakfast.

In some embodiments, the SMAD7 antisense-oligonucleotide is administered with water.

In some embodiments, the SMAD7 antisense-oligonucleotide is administered orally.

In some embodiments, the SMAD7 antisense-oligonucleotide is administered once a day in the morning, at least 30 min before breakfast with water.

In some embodiments, the SMAD7 antisense-oligonucleotide is administered orally, once a day in the morning, at least 30 min before breakfast with water.

In some embodiments, the method further comprises if the patient received an IBD treatment before the first treatment period, then tapering off the IBD treatment at the end of the first treatment period.

In some embodiments, the IBD treatment is tapered off at least during the last 1 week, the last 2 weeks, the last 3 weeks, the last 4 weeks, the last 5 weeks, the last 6 weeks, the last 7 weeks, the last 8 weeks, the last 9 weeks, or the last 10 weeks of the first treatment period.

In some embodiments, the IBD treatment is tapered off before the second treatment period.

In some embodiments, the IBD treatment is selected from the group consisting of a corticosteroid, an aminosalicylate, a budesonide, an immunosuppressant.

In some embodiments, the IBD treatment comprises a corticosteroid.

In some embodiments, the method further comprises analyzing the clinical response in the patient at one or more time-points during the first treatment period and/or the second treatment period.

In some embodiments, the method further comprises, if the patient does not show a clinical response at the end of the first treatment period, then terminating the treatment or increasing the first dose and repeating the first treatment period.

In some embodiments, the treatment is terminated if the first dose exceeds the maximum tolerated dose.

In some embodiments, the clinical response in the patient is analyzed using the Simple Endoscopic Score for Crohn's Disease (SES-CD), the Crohn's Disease Activity Index (CDAI), a two-item patient reported outcome (PRO-2) test, an intestinal mucosal biopsy.

In some embodiments, the PRO-2 test comprises analyzing average daily liquid stool, average daily soft stool, or an average daily abdominal pain score.

In some embodiments, the patient shows a clinical response if the patient's CDAI score decreases ≥20 points, ≥30 points, ≥40 points, ≥50 points, ≥60 points, ≥70 points, ≥80 points, ≥90 points, ≥100 points, ≥110 points, ≥120 points, ≥130 points, ≥140 points, or ≥150 points from baseline during the first treatment period.

In some embodiments, the patient shows a clinical response if the patient's CDAI score decreases ≥100 points from baseline during the first treatment period.

In some embodiments, the patient shows a clinical response if the patient's CDAI score is <200, <190, <180, <170, <160, <150, <140, <130, <120, <110, or <100 at the end of the first treatment period.

In some embodiments, the patient shows a clinical response if the patient's CDAI score is <150 at the end of the first treatment period.

In some embodiments, the patient shows a clinical response if the patient's SES-CD score at the end of the first treatment period is <80%, <75%, <70%, <65%, <60%, <55%, <50%, <45%, <40%, <35%, <30%, <25%, or <20% of the patient's SES-CD score at the beginning of the first treatment period.

In some embodiments, the patient shows a clinical response if the patient's SES-CD score at the end of the first treatment period is <75% or <50% compared to the patient's SES-CD score at the beginning of the first treatment period.

In some embodiments, the patient shows a clinical response if the patient's SES-CD score is ≤5, ≤4, ≤3, ≤2, or ≤1 at the end of the first treatment period.

In some embodiments, the patient shows a clinical response if the patient's SES-CD score is ≤2 at the end of the first treatment period.

In some embodiments, the patient shows a clinical response if intestinal mucosal ulcerations are absent in the patient at the end of the first treatment period.

In some embodiments, the patient shows a clinical response if the patient's PRO-2 score at the end of the first treatment period is ≥2, ≥3, ≥4, ≥5, ≥6, ≥7, ≥8, ≥9, ≥10, ≥12, or ≥14 points lower than at the beginning of the first treatment period.

In some embodiments, the patient shows a clinical response if the patient's PRO-2 score at the end of the first treatment period is <14, <12, <10, <8, <6, <4, or <2.

In some embodiments, the patient shows a clinical response if the patient's average daily liquid or soft stool frequency score at the end of the first treatment period is reduced by ≥20%, ≥30%, ≥40%, ≥50%, ≥60%, ≥70%, ≥80%, or ≥90% compared to the patient's average daily liquid or soft stool frequency score at the beginning of the first treatment period.

In some embodiments, the patient shows a clinical response if the patient's average daily abdominal pain score at the end of the first treatment period is reduced by ≥20%, ≥30%, ≥40%, ≥50%, ≥60%, ≥70%, ≥80%, or ≥90% compared to the patient's average daily abdominal pain score at the beginning of the first treatment period.

In some embodiments, the patient shows a clinical response if the patient's abdominal pain score is ≤2.0, ≤1.5, or ≤1.0.

In some embodiments, the patient shows a clinical response if the patient's average daily liquid stool frequency score or average daily soft stool frequency score is ≤4.0, ≤3.5, ≤3.0, ≤2.5, or ≤2.0.

In some embodiments, the patient shows a clinical response if the patient's abdominal pain score is ≤2.0, ≤1.5, or ≤1.0 and if the patient's average daily liquid stool frequency score or average daily soft stool frequency score is ≤4.0, ≤3.5, ≤3.0, ≤2.5, or ≤2.0. In some embodiments, the patient's abdominal pain score is ≤1.0 and the average daily liquid or soft stool frequency is ≤3.0. In some embodiments, the patient's abdominal pain score is ≤1.0 and the average daily liquid or soft stool frequency is ≤1.5.

In some embodiments, the patient does not experience a fibrotic event during the first treatment period.

In some embodiments, the patient does not experience a fibrotic event during the first treatment period and the second treatment period.

In some embodiments, the patient does not experience a fibrotic event during the first treatment period, the second treatment period and for at least 1 week, at least 2 weeks, at least 3 weeks, at least 6 weeks, at least 9 weeks, or at least 12 weeks, at least 6 months, at least 9 months, at least 12 months, at least 18 months, at least two years, at least three years, at least four years, or at least five years following the second treatment period.

In some embodiments, the method further comprises analyzing SMAD7 antisense oligonucleotide levels in a patient sample.

In some embodiments, the patient sample is a serum sample or an intestinal mucosal biopsy sample.

In some embodiments, the patient was diagnosed with ileitis, or ileocilitis.

In some embodiments, the IBD is Crohn's Disease (CD) or ulcerative colitis (UC).

In some embodiments, the patient having IBD is a steroid-dependent patient with active CD.

In some embodiments, the patient having IBD is a steroid-resistant patient with active CD.

In some embodiments, the patient having IBD has a CDAI score ≥220 and ≤450 and a SES-CD score 27 at the beginning of the first treatment period.

In some embodiments, the patient having IBD has experienced treatment failure with or intolerance to an aminosalicylate, a budesonide, a systemic corticosteroid or an immunosuppressant.

In some embodiments, the immunosuppressant is 6-mercaptopurine (6-MP), azathiopine (AZ), or methotrexate (MTX).

In some embodiments, the patient's disease is restricted to the terminal ileum and/or the mid transverse colon.

In some embodiments, the patient does not experience a fibrotic event during the first or second treatment period.

In some embodiments, the SMAD7 antisense-oligonucleotide is administered orally to the patient having IBD.

In some embodiments, the SMAD7 antisense oligonucleotide targets region 108-128 of human SMAD7 (SEQ ID NO:1).

In some embodiments, the SMAD7 antisense oligonucleotide targets nucleotides 403, 233, 294, 295, 296, 298, 299 or 533 of human SMAD7 (SEQ ID NO:1).

In some embodiments, the SMAD7 antisense oligonucleotide comprises the nucleotide sequence of SEQ ID NO:2 (5′-GTCGCCCCTTCTCCCCGCAG-3′).

In some embodiments, the SMAD7 AON is COMPOUND (I).

In another aspect, provided herein is a method for treating or managing IBD in a patient having IBD, wherein the method comprises (a) administering to a CD patient a SMAD7 AON for a period of about 4 weeks, about 8 weeks, or about 12 weeks at a once-daily dose of about 160 mg; and (b) administering to the CD patient the SMAD7 AON for about 24 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; d) administering a placebo or no SMAD7 AON for about 4 weeks; and repeating (c) and d) for a total of 24 weeks.

In another aspect, provided herein is a method for treating or managing IBD in a patient having IBD, wherein the method comprises (a) administering to a CD patient a SMAD7 AON for a period of about 12 weeks at a once-daily dose of about 160 mg; and (b) administering to the CD patient the SMAD7 AON for about 24 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) administering a placebo or no SMAD7 AON for about 4 weeks; and repeating (c) and (d) for a total of 24 weeks.

In another aspect, provided herein is a method for treating or managing IBD in a patient having IBD, wherein the method comprises (a) administering to a CD patient a SMAD7 AON for a period of between about 4 weeks and about 8 weeks at a once-daily dose of about 40 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) administering a placebo or no SMAD7 AON for about 4 weeks; and repeating (c) and (d) 6 for a total of 52 weeks.

In another aspect, provided herein is a method for treating or managing IBD in a patient having IBD, wherein the method comprises (a) administering to a CD patient a SMAD7 AON for a period of between about 4 weeks and about 8 weeks at a once-daily dose of about 40 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) optionally administering a placebo or no SMAD7 AON for about 8 weeks; and repeating c) and d) for a total of 52 weeks.

In another aspect, provided herein is a method for treating or managing IBD in a patient having IBD, wherein the method comprises (a) administering to a CD patient a SMAD7 AON for a period of between about 4 weeks and about 8 weeks at a once-daily dose of about 160 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) optionally administering a placebo or no SMAD7 AON for about 4 weeks; and repeating (c) and (d) for a total of 52 weeks.

In another aspect, provided herein is a method for treating or managing IBD in a patient having IBD, wherein the method comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 160 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) optionally administering a placebo or no SMAD7 AON for about 8 weeks; and repeating (c) and (d) for a total of 52 weeks.

In another aspect, provided herein is a method for treating or managing IBD in a patient having IBD, wherein the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) optionally administering a placebo or no SMAD7 AON for about 4 weeks; and repeating (c) and (d) for a total of 52 weeks.

In another aspect, provided herein is a method for treating or managing IBD in a patient having IBD, wherein the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 40 mg.

In another aspect, provided herein is a method for treating or managing IBD in a patient having IBD, wherein the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 160 mg on an alternating dosing schedule, wherein the alternating dosing schedule comprises (c) administering the SMAD7 antisense oligonucleotide at a once-daily dose of about 160 mg for about 4 weeks; (d) optionally administering a placebo or no SMAD7 AON for about 4 weeks; and repeating (c) and (d) for a total of 52 weeks.

In another aspect, provide herein is a method for treating or managing IBD in a patient having IBD, wherein the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 40 mg on an alternating dosing schedule, wherein the alternating dosing schedule comprises (c) administering the SMAD7 antisense oligonucleotide at a once-daily dose of about 40 mg for about 4 weeks; (d) optionally administering a placebo or no SMAD7 AON for about 8 weeks; and repeating (c) and (d) for a total of 52 weeks.

In some embodiments, in an alternating dosing schedule the SMAD7 AON is administered first and the placebo or no SMAD7 AON is administered second.

In some embodiments, in an alternating dosing schedule the placebo or no SMAD7 AON is administered first and the SMAD7 AON is administered second

In another aspect, provided herein is a method for treating or managing IBD in a patient having IBD, wherein the method comprises (a) administering to an IBD patient a SMAD7 AON for a first period at a once-daily dose of about 160 mg; and (b) administering to the IBD patient the SMAD7 AON for a second period at a once-daily dose of about 40 mg or 160 mg using an alternating dosing schedule, wherein the alternating dosing schedule comprises c) administering to the IBD patient a placebo or no SMAD7 AON for a first alternating period; d) administering to the IBD patient the SMAD7 AON at a once-daily dose of about 40 mg or about 160 mg for a second alternating period; and repeating c) and d) until the end of the second period. In some embodiments, the first period is about 12 weeks, the second period is up to about 40 weeks, and the first and second alternating periods each are about 4 weeks. In some embodiments, the second period is not predetermined, but depends on the patient's response to treatment, e.g., as determined by results from colonoscopy or ileocolonoscopy tests, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5 and/or abdominal pain score ≤1; TMS ≤2, MMS ≤2, ES=1 or 0, and the like). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 160 mg; and (b) administering to the IBD patient the SMAD7 AON for a second period at a once-daily dose of about 40 mg. In some embodiments, the first period is about 12 weeks and the second period is up to about 40 weeks. In some embodiments, the second period is not predetermined, but depends on the patient's response to treatment, e.g., as determined by results from colonoscopy or ileocolonoscopy results, biomarker levels or others (e.g., achievement or maintenance for a specific time of CDAI <150, SES-CD ≤2, PRO-2 score <8, or CRP levels <1.0 mg/L, average daily liquid or soft stool frequency score ≤3 and/or ≤1.5, abdominal pain score ≤1; TMS ≤2, MMS ≤2, ES=1 or 0, and the like). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 160 mg; and (b) administering to the IBD patient the SMAD7 AON for a second period at a once-daily dose of about 160 mg using an alternating dosing schedule, wherein the alternating dosing schedule comprises c) administering to the IBD patient a placebo or no SMAD7 AON for a first alternating period; d) administering to the IBD patient the SMAD7 AON at a once-daily dose of about 160 mg for a second alternating period; and repeating c) and d) until the end of the second period. In some embodiments, the first period is about 12 weeks, the second period is up to about 196 weeks, and the first and second alternating periods each are about 4 weeks. In some embodiments, the second period is not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5, and/or abdominal pain score ≤1; TMS ≤2, MMS ≤2, ES=1 or 0). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 160 mg using an alternating dosing schedule, wherein the alternating dosing schedule comprises b) administering to the IBD patient the SMAD7 AON at a once-daily dose of about 160 mg for a first alternating period; c) administering to the IBD patient a placebo or no SMAD7 AON for a second alternating period; and repeating b) until the end of the first period; d) administering to the IBD patient the SMAD7 AON for a second period at a once-daily dose of up to about 160 mg using an alternating dosing schedule, wherein the alternating dosing schedule comprises e) administering to the IBD patient a placebo or no SMAD7 AON for a third alternating period; f) administering to the IBD patient the SMAD7 AON at a once-daily dose of about 160 mg for a fourth alternating period; and repeating e) and f) until the end of the second period. In some embodiments, the first period is about 12 weeks, the second period is up to about 196 weeks, and the first, second and third alternating periods each are about 4 weeks. In some embodiments, the second period not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, or CRP levels <1.0 mg/L, average daily liquid or soft stool frequency score ≤3 or ≤1.5 and/or abdominal pain score ≤1; TMS ≤2, MMS ≤2, or ES=1 or 0). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 40 mg; and (b) administering to the IBD patient the SMAD7 AON for a second period a once-daily dose of about 40 mg. In some embodiments, the first period is about 12 weeks and the second period is up to about 196 weeks. In some embodiments, the second period is not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5 and/or abdominal pain score ≤1; TMS ≤2, MMS ≤2, or ES=1 or 0). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 40 mg using an alternating dosing schedule, wherein the alternating dosing schedule comprises b) administering to the IBD patient a placebo or no SMAD7 AON for a first alternating period; c) administering to the IBD patient the SMAD7 AON at a once-daily dose of about 40 mg for a second alternating period; and repeating b) until the end of the first period; d) administering to the IBD patient the SMAD7 AON for a second period at a once-daily dose of up to about 40 mg using an alternating dosing schedule, wherein the alternating dosing schedule comprises e) administering to the IBD patient the SMAD7 AON at a once-daily dose of about 40 mg for a third alternating period; f) administering to the IBD patient a placebo or no SMAD7 AON for a fourth alternating period; and repeating e) and f) until the end of the second period. In some embodiments, the first period is about 12 weeks, the second period is up to about 196 weeks, and the first, second and third alternating periods each are about 4 weeks. In some embodiments, the second period is not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5 and/or abdominal pain score ≤1; TMS ≤2, MMS ≤2, or ES=1 or 0). In some embodiments, the IBD can be CD.

In another aspect, provided herein is a method for treating or managing IBD in a patient having IBD, wherein the method comprises (a) administering to an IBD patient a SMAD7 AON for a first period at a once-daily dose of about 160 mg; and (b) administering to the IBD patient the SMAD7 AON for a second period at a once-daily dose of about 160 mg using an alternating dosing schedule, wherein the alternating dosing schedule comprises c) administering to the IBD patient the SMAD7 AON at a once-daily dose of about 160 mg for a first alternating period; d) administering a placebo or no SMAD7 AON for a second alternating period; and repeating c) and d) until the end of the second period. In some embodiments, the first period is about 8 weeks, the second period is up to about 44 weeks, and the first, second and third alternating periods each are about 4 weeks. In some embodiments, the second period is not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5 and/or abdominal pain score ≤1; TMS ≤2, MMS ≤2, or ES=1 or 0). In some embodiments, the IBD can be UC.

In another aspect provided herein is a method for treating or managing IBD in a patient having IBD, wherein the method comprises (a) administering to an IBD patient a SMAD7 AON for a first period at a once-daily dose of up to 320 mg; and (b) administering to the IBD patient the SMAD7 AON for a second period at a once-daily dose of about 160 mg using an alternating dosing schedule, wherein the alternating dosing schedule comprises c) administering to the IBD patient the SMAD7 AON at a once-daily dose of about 160 mg for a first alternating period; d) administering to the IBD patient a placebo or no SMAD7 AON for a second alternating period; and repeating c) and d) until the end of the second period. In some embodiments, the first period is about 8 weeks, the second period is up to about 44 weeks, and the first, second and third alternating periods each are about 4 weeks. In some embodiments, the period of time is not predetermined, but depends on the patient's response to treatment, e.g., as determined by results from colonoscopy, ileocolonoscopy, biomarker levels or others (e.g., achievement or maintenance for a specific time of CDAI <150, SES-CD ≤2, PRO-2 score <8, CRP levels <1.0 mg/L, average daily liquid or soft stool frequency score ≤3 or ≤1.5, and/or abdominal pain score ≤1; TMS ≤2, MMS ≤2, or ES=1 or 0). In some embodiments, the IBD can be UC.

In any embodiments comprising an alternating dosing schedule, the alternating dosing schedule can start with either a drug administration (e.g., SMAD7 AON administration) or with the administration of a placebo or no treatment.

In some embodiments, in one or more alternating dosing schedules, the SMAD7 AON is administered first and the and the placebo or no treatment is administered second.

In some embodiments, in one or more alternating dosing schedules the placebo or no treatment is administered first and the SMAD7 AON is administered second.

Any administration schedule described herein can be preceeded by the same or by any other administration schedule described herein.

In another aspect, provided herein is a SMAD7 antisense oligonucleotide for use in a method for treating or managing IBD described herein.

### 5. BRIEF DESCRIPTION OF THE DRAWINGS

FIG. 1 shows a graphic illustrating an exemplary method provided herein. See also, Example 1. The asterix (*) indicates randomization stratified by distal colon involvement yes/no; the solid arrow indicates treatments with Compound (1) 160 mg/day; the open dotted arrow indicates treatments with Compound (1) 40 mg/day; the solid dotted arrow indicates placebo treatments. BSL=baseline; CDAI=Crohn's Disease Activity Index; IP=investigational product; C=Ileocolonoscopy. The observation phase is up to 52 weeks until subject experiences partial loss of response. No IP is dispensed during the Observation Phase.

FIG. 2 illustrates the nucleotide sequence of COMPOUND (I), an exemplary SMAD7 antisense-oligonucleotide (SEQ ID NO:6).

FIG. 3 shows a graphic illustrating an exemplary method provided herein. See also Example 2. The solid line indicates continuous or alternating treatments with COMPOUND (I) 160 mg/day or 40 mg/day; the broken line indicates placebo treatments. QD=once daily; PBO=placebo. The Follow-up Period is up to 4 weeks. No IP is dispensed during the Follow-up Period.

FIG. 4 shows a graphic illustrating an exemplary method provided herein. See also Example 3. The solid arrow indicates continuous or alternating treatments with COMPOUND (1) 160 mg/day or 40 mg/day; the broken arrow indicates placebo treatments. The Follow-up Period is up to 4 weeks after the last dose of IP. No IP is dispensed during the Follow-up Period.

FIG. 5 shows a graphic illustrating an exemplary method provided herein. See also Example 4. The solid arrow indicates continuous or alternating treatments with COMPOUND (1) 160 mg/day during and Induction Period and a Maintenance Period. The Follow-up Period is up to 4 weeks after the last dose of IP. No IP is dispensed during the Follow-up Period. B=Time point for biomarker specimen collection from subjects; C=Time point for ileocolonoscopy procedures and biopsy specimen collections from subjects.

FIG. 6 shows a graphic illustrating an exemplary method provided herein. See also Example 5. The solid line indicates continuous or alternating treatments with Compound (1) 160 mg/day or 320 mg/day. BSL=baseline; Flex-sig=Flexible rectosigmoidoscopy; TNF-α=tumor necrosis factor alpha; Wk=week. The Observation Follow-up Period is up to 4 weeks after the last dose of IP. No IP is dispensed during the Follow-up Period.

### 6. ABBREVIATIONS AND CONVENTIONS

The abbreviation “AZA,” as used herein, means “azathioprine.”

The abbreviation “BSL,” as used herein, means “baseline.”

The abbreviation “CD,” as used herein means “Cluster of Differentiation,” e.g., Cluster of Differentiation 4 (CD4).

The abbreviation “CDAI,” as used herein, means “Crohn's Disease Activity Index.”

The abbreviation “CDEIS,” as used herein, means “Crohn's Disease Endoscopic Index of Severity.”

The abbreviation “hsCRP,” as used herein, means “high-sensitivity CRP” and refers to CRP levels determined by a test that can analyze low levels of CRP. For example, hsCRP can be analyzed with a high-sensitivity test using laser nephelometry. Some hsCRP tests can analyze hsCRP with a sensitivity down to 0.04 mg/ml.

The abbreviation “ES,” as used herein, means “Endoscopy Score.”

The abbreviation “FCP,” as used herein, means “Fecal Calprotectin,” a protein also known as S100 Calcium Binding Protein A9 (S100A9)).

The abbreviation “Flex-sig,” as used herein, means “flexible rectosigmoidoscopy.”

The abbreviation “HLA,” as used herein, means Human Leukocyte Antigen.

The abbreviation “IFN,” as used herein, means interferon, e.g., IFNg.

The abbreviation “IL,” as used herein, means “interleukin,” e.g., interleukin 6 (IL6).

The abbreviation “IP,” as used herein, means “investigational product.” IP can refer, for example to a pharmaceutical composition comprising a SMAD7 AON, such as COMPOUND (I).

The abbreviation “IVRS,” as used herein, means “Interactive Voice Response System.”

The abbreviation “IWRS,” as used herein, means “Interactive Web Response System.”

The abbreviation “LLN,” as used herein means “Lower Limit of Normal.”

The abbreviation “6-MP,” as used herein, means “6-mercaptopurine.”

The abbreviation “MMS,” as used herein, means “Modified Mayo Score.”

The abbreviation “MTX,” as used herein, means “methotrexate.”

The abbreviation “PBO,” as used herein, means “placebo.”

The abbreviation “PBO QD,” as used herein means “placebo daily dose.”

The abbreviation “PD,” as used herein, means “pharmacodynamic.”

The abbreviation “PGA,” as used herein, means “Physician's Global Assessment Subscore.”

The abbreviation “PMS,” as used herein, means “Partial Mayo Score.”

The abbreviation “PRO-2,” as used herein, stands for “two-item Patient Reported Outcome (PRO-2).”

The abbreviation “PT,” as used herein stands for “Prothrombine Time.”

The abbreviation “PTT,” as used herein stands for “Partial Thromboplastin Time.”

The abbreviation “QD,” as used herein, refers to a “once daily” (quaque die) dose, e.g., of a SMAD7 AON, such as COMPOUND (I).

The abbreviation “QOL” or “QoL,” as used herein, means “quality of life.”

The abbreviation “RBS,” as used herein, means “Rectal Bleeding Subscore.”

The abbreviation “SES-CD,” as used herein, means “Simple Endoscopic Score for Crohn's Disease.”

The abbreviation “SFS,” as used herein, means “Stool Frequency Subscore.”

The abbreviation “SMAD7 AON” as used herein, means SMAD7 antisense oligonucleotide.

The abbreviation “TMS,” as used herein, means “Total Mayo Score.”

The abbreviation “UCDAI,” as used herein, means “Ulcerative Colitis Disease Activity Index.”

The abbreviation “ULN,” as used herein, means “Upper Limit of Normal.”

The abbreviation “Wk,” as used herein, means “week.”

### 7. DETAILED DESCRIPTION

“Inflammatory bowel disease” or “IBD,” as used herein, can refer to a number of chronic inflammatory diseases including Crohn's disease (CD), gastroduodenal Crohn's disease, Crohn's (granulomatous) colitis, ulcerative colitis (UC), collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis. CD and UC are the two most common forms of IBD. IBD is an autoimmune disease of the digestive system. CD can be localized to any portion of the gastrointestinal tract, including the terminal ileum, and can impact all cell types of the gastrointestinal tract. UC is localized to the colon and rectum, and affects cells of the mucosa only.

Both environmental and genetic factors are believed to play a role in IBD, although the identity of such factors is not well-defined. Environmental components can comprise alterations in flora of the gut which are affected by exposure to ingested foods and medications.

IBD is associated with symptoms including abdominal pain, vomiting, diarrhea, rectal bleeding, severe cramps, muscle spasms, weight loss, malnutrition, fever, anemia, skin lesions, joint pain, eye inflammation, liver disorders, arthritis, pyoderma gangrenosum, primary sclerosing cholangitis, and non-thyroidal illness syndrome. Children suffering from UC can suffer from growth defects.

Forms of CD comprise steroid-dependent and steroid-resistant forms of CD, including active CD. Patients with IBD who suffer from a steroid-dependent form of CD are responsive to treatment with steroid therapy, but cannot terminate or curtail steroid therapy without suffering from an increase in occurrence of symptoms associated with CD. Patients with IBD who suffer from a steroid-resistant form of CD are not responsive to treatment with steroid therapy. Steroid therapeutics commonly prescribed and/or administered to patients with IBD comprise: corticosteroids, for example, prednisone, dexamethasone, hydrocortisone, methylprednisolone, prednisone, and budesonide. A human patient suffering from active CD is a patient actively suffering from symptoms of CD, for example, but not limited to, bloody stool, weight loss, and/or abdominal cramps.

Ulcerative colitis is one of the most common forms of IBD. UC typically involves dysregulation or overstimulation of the mucosal immune system. Clinical characteristics can include rectal bleeding, diarrhea, and abdominal pain, as well as extraintestinal manifestations involving the skin, liver, and other sites. Patients with UC often have a poor quality of life (QoL) and are at risk for disease flares leading to hospitalizations and/or surgeries.

The objectives in the treatment of UC patients include inducement and maintenance of remission of symptoms, as well as, healing of mucosal inflammation in order to improve patients' QoL. Treatment of UC can involve pharmacological treatment and surgery. Treatment often takes into consideration the level of clinical activity combined with the extent of disease (proctitis, left-sided disease, extensive disease, or pancolitis). Pharmacological treatment usually involves aminosalicylates and glucocorticoids as an initial approach. Various immunosuppressants, as well as biologic TNF blockers, are used in refractory or severe disease. Although these drugs can provide clinical benefit, they have important limitations. Aminosalicylates are only modestly effective. Glucocorticoids can cause unacceptable adverse events (AEs) and often do not provide a benefit as maintenance therapy. Additionally, use of immunosuppressants, such as azathioprine and 6-mercaptopurine has been restricted to maintenance therapy and is also associated with significant potential toxicities. TNF blockers, although efficacious, can predispose patients to serious infections (including opportunistic infections) and possibly malignancies. Surgery is typically indicated when pharmacological treatment fails or when an emergency requires surgical intervention.

A “patient” or “subject” as described herein, refers to any animal at risk for, suffering from or diagnosed for IBD, including, but not limited to, mammals, primates, and humans. In certain embodiments, the subject may be a non-human mammal such as, e.g., a cat, a dog, or a horse. In a preferred embodiment, the subject is a human subject. A subject may be an individual diagnosed with a high risk of developing IBD, someone who has been diagnosed with IBD, someone who previously suffered from IBD, or an individual evaluated for symptoms or indications of IBD, for example, a high CDAI index score.

“A patient with IBD,” as used herein, refers to a patient suffering from any of the symptoms or manifestations of IBD, a patient who may suffer from any of the symptoms or manifestations of IBD, or any patient who might benefit from a method of the invention for treating or evaluating treatment for IBD. A patient in need can comprise a patient who is diagnosed with a risk of developing IBD, a patient who has suffered from IBD in the past, or a patient who has previously been treated for IBD. In some embodiments, the patient with IBD is a Crohn's disease (CD) patient. In some embodiments, the patient with IBD is an ulcerative colitis (UC) patient.

As used herein, “Crohn's Disease Activity Index” or “CDAI” refers to a measurement or index used to assess the progress of patients suffering from CD as described by Best et al., Gastroenterology, 70:439-44 (1976). CDAI scores of 150 or below are generally associated with inactive disease and are indicative of better prognosis than higher scores. Values above 150 are generally associated with active disease and values above 450 are associated with extremely severe disease. CDAI scores may be used to determine how well a patient is responding to therapy and may be used to identify patients in remission. In certain embodiments, a benchmark clinical response means that the subject displays a decrease in CDAI score by at least 100 points. In a clinical trial, a CDAI score of 150 or below is generally associated with remission.

As used herein, “Ulcerative Colitis Disease Activity Index” or “UCDAI” refers to a measurement or index used to assess the progress of patients suffering from UC as described by Sutherland et al., Gastroenterology, 92:1894-98 (1987). The UCDAI is a series of qualifiers about the symptoms of UC including stool frequency, rectal bleeding, the appearance of the colon lining, and a physician's rating of disease activity. Each of these qualifiers is given a number from 0 to 3, with 3 being the highest disease activity. In a clinical trial, remission is often defined as a UCDAI score of 1 or less, and improvement is a reduction of 3 or more points from the score at the beginning of the trial. UCDAI may be used in clinical trials to determine how well a patient is responding to therapy and may be used to identify patients in remission. Other commonly used indices for measuring disease severity in UC patients comprise the Truelove and Witts Index, the St. Mark's Index, the Simple Clinical Colitis Activity Index (SCCAI), the Lichtiger Index, the Ulcerative Colitis Symptom Score (UCSS), and the Mayo Clinic Score.

As used herein, “SMAD7” (also known as CRCS3, FLJ16482, MADH7, MADH8, MAD (mothers against decapentaplegic, Drosophila) homolog 7, MAD homolog 8, SMAD, mothers against DPP homolog 7, mothers against DPP homolog 8) means the human protein or any of the mRNA transcripts encoded by the gene identified by Entrez GeneID No. 4092 and allelic variants thereof.

As used herein, “CRP” (also known as C-reactive protein, pentraxin-related; Pentraxin; and PTX 1) means the human protein or any of the mRNA transcripts encoded by the gene identified by Entrez GeneID No. 1401 and allelic variants thereof.

As used herein, “CD4” (also known as Cluster of Differentiation 4), means the human protein or any of the mRNA transcripts encoded by the gene identified by Entrez GeneID No. 920 and allelic variants thereof.

As used herein, “CD8” (also known as Cluster of Differentiation 8), means the human protein or any of the mRNA transcripts encoded by the gene identified by Entrez GeneID No. 920A or 920B and allelic variants thereof.

As used herein, “IL6” (also known as Interleukin-6; B-Cell Stimulatory Factor 2 (BSF2), Hybridoma Growth Factor (HGF), Hepatocyte Stimulating Factor (HSF), Interferon Beta-2 (IFNB2)) means the human protein or any of the mRNA transcripts encoded by the gene identified by Entrez GeneID No. 3569 and allelic variants thereof.

As used herein, “IL8” (also known as Interleukin-8 (IL-8); Tumor Necrosis Factor-Induced Gene 1; NAF; Granulocyte Chemotactic Protein 1 (GCP1); LECT; LUCT; Protein 3-10C; Beta-Thromboglobulin-Like Protein; Neutrophil-Activating Peptide 1; Neutrophil-Activating Protein 1 (NAP1; NAP-1); Emoctakin; GCP-1; LYNAP; Lymphocyte Derived Neutrophil Activating Peptide; Lung Giant Cell Carcinoma-Derived Chemotactic Protein; Small Inducible Cytokine Subfamily B, Member 8; Beta Endothelial Cell-Derived Neutrophil Activating Peptide; Monocyte-Derived Neutrophil Chemotactic Factor (MDNCF); Monocyte-Derived Neutrophil-Activating Peptide (MONAP); Alveolar Macrophage Chemotactic Factor I; C—X—C Motif Chemokine 8; and Chemokine (C—X—C Motif) Ligand 8 (CXCL8)) means the human protein or any of the mRNA transcripts encoded by the gene identified by Entrez GeneID No. 3576 and allelic variants thereof.

As used herein, “IL12” (also known as Interleukin-12 (IL-12); Natural Killer Cell Stimulatory Factor (NKSF1), or Cytotoxic Lymphocyte Maturation Factor 1 (p35, 35 kDA Subunit), means the human protein or any of the mRNA transcripts encoded by the gene identified by Entrez GeneID No. 3592 and allelic variants thereof.

As used herein, “IL17” (also known as Interleukin-17A (IL-17A); Cytotoxic T-Lymphocyte-Associated Serine Esterase 8 or Cytotoxic T-Lymphocyte-Associated Antigen 8 (CTLA8)), means the human protein or any of the mRNA transcripts encoded by the gene identified by Entrez GeneID No. 3605 (IL17A).

As used herein, “IFNγ” (also known as Interferon gamma) means the human protein or any of the mRNA transcripts encoded by the IFNγ gene identified by Entrez GeneID Nos. 3458 and allelic variants thereof.

As used herein “HLA-DR” (also known as Human Leukocyte Antigen DR, a MHC class II cell surface receptor), means a human protein or any of the mRNA transcripts encoded by any member of the HLA-DR gene family, including HLA-DRA, HLA-DRB1, HLA-DRB3, HLA-DRB4, and HLA-DRB5, which are identified by Entrez GeneID Nos. 3122, 3123, 3125, 3126, and 3127 and allelic variants thereof.

As used herein, “TNFα” (also known as Tumor Necrosis Factor, DIF, Tumor Necrosis Factor Ligand Superfamily Member 2 (TNFSF2), APC1 Protein, cachectin, Tumor Necrosis Factor A (TNFA), Tumor Necrosis Factor-α (TNF-α), and Tumor Necrosis Factor-alpha (TNF-alpha)) means the human protein or any of the mRNA transcripts encoded by the gene identified by Entrez GeneID No. 7124 and allelic variants thereof.

As used herein, “FCP” (also known as Fecal Calprotectin or S100 Calcium Binding Protein A9 (S100A9)) means the human protein or any of the mRNA transcripts encoded by the gene identified by Entrez GeneID No. 6280 and allelic variants thereof.

7.1 Treatment Regimen

The methods provided herein are based, in part, on the recognition that inflammatory bowel disease (IBD), such as Crohn's disease (CD) or ulcerative colitic (US), can be treated or managed in a patient having IBD by administering an anti-SMAD7 therapy, e.g., a SMAD7 antisense oligonucleotide (AON), to the patient using an administration regime comprising administering the anti-SMAD7 therapy at a first dose during a first treatment period and administering the anti-SMAD7 therapy at a second dose during a second treatment period. See, e.g., Section 7.1.1. In some embodiments, the dose of the anti-SMAD7 therapy administered during the first treatment period is higher than the dose administered during the second treatment period.

Antisense oligonucleotides can be short synthetic oligonucleotide sequences complementary to the messenger RNA (mRNA), which encodes the target protein (e.g., SMAD7). Without being bound by theory, antisense oligonucleotide sequences are thought to hybridize to the mRNA resulting in a double-strand hybrid that can lead to the activation of ubiquitary catalytic enzymes, such as RNase H, which degrades DNA/RNA hybrid strands, thus preventing protein translation. Without being bound by theory, an antisense oligonucleotide described in this section and useful in the methods provided herein can hybridize to its target sequence as RNA or DNA. Thus, even if a DNA sequence is provided as target, the corresponding RNA sequence (including uracil instead of thymine) is included. The antisense oligonucleotide can be either RNA or DNA. Exemplary antisense oligonucleotides that can be used in connection with the methods described herein are described, e.g., in Section 7.10.

In some embodiments of the methods provided herein, the IBD patient is a CD patient. In some embodiments, the IBD patient is a UC patient.

In some embodiments, the treatment regimen further comprises administering the anti-SMAD7 therapy at a third dose during a third treatment period. In some embodiments, the dose of the anti-SMAD7 therapy administered during the first and/or second treatment period is higher than the dose administered during the third treatment period. In some embodiments, the dose of the anti-SMAD7 therapy administered during the first and/or second treatment period is lower than the dose administered during the third treatment period. In some embodiments, the dose of the anti-SMAD7 therapy administered during the first and/or second treatment period is the same as the dose administered during the third treatment period.

In some embodiments, the second and/or third treatment periods are optional. In some embodiments, the second treatment period is optional. In some embodiments, the third treatment period is optional. In some embodiments, the second and third treatment periods are optional.

In the administration regimes provided herein, the anti-SMAD7 therapy can be administered to the patient using different administration schedules (e.g., a continuous administration schedule or an alternating administration schedule). See, e.g., Section 7.1.2. In some embodiments, the anti-SMAD7 therapy is administered following a continuous administration schedule during the first treatment period (e.g., once daily) and following an alternating treatment schedule during the second treatment period (e.g., 4 weeks of treatment alternating with 4 weeks of no treatment or placebo treatment).

In some embodiments, the anti-SMAD7 therapy is administered following a continuous administration schedule during the first treatment period (e.g., once daily) and following a continuous administration schedule during the second treatment period (e.g., once daily).

In some embodiments, the anti-SMAD7 therapy is administered following a continuous administration schedule during the first treatment period (e.g., once daily), following an alternating treatment schedule during the second treatment period (e.g., 4 weeks of treatment alternating with 4 weeks of no treatment or placebo treatment), and following an alternating treatment schedule during the third treatment period (e.g., 4 weeks of treatment alternating with 4 weeks of no treatment or placebo treatment). In some embodiments, the anti-SMAD7 therapy is administered following a continuous administration schedule during the first treatment period (e.g., once daily), following a continuous administration schedule during the second treatment period (e.g., once daily), and following an alternating treatment schedule during the third treatment period (e.g., 4 weeks of treatment alternating with 4 weeks of no treatment or placebo treatment). In some embodiments, the anti-SMAD7 therapy is administered following a continuous administration schedule during the first treatment period (e.g., once daily), following an alternating treatment schedule during the second treatment period (e.g., 4 weeks of treatment alternating with 4 weeks of no treatment or placebo treatment), and following a continuous administration schedule during the third treatment period (e.g., once daily).

A patient's response to the anti-SMAD7 therapies can be monitored, e.g., during the first and/or second and/or third treatment period and the administration regimes provided herein can be adjusted, depending on the IBD patient's clinical response. See, e.g., Section 7.2, Section 7.1.1.3 and Section 7.1.1.6. For example, if an IBD patient is found to respond to the anti-SMAD7 therapy during the first and/or second treatment period the first and/or second treatment period can be shortened or ended, and the IBD patient can enter the second and/or third treatment period. In some embodiments, the dose of the anti-SMAD7 therapy can be adjusted depending on the IBD patient's clinical response during the first, second and/or third treatment period. An IBD patient's response to the anti-SMAD7 therapy can be analyzed using a number of clinical parameters, such as endoscopic outcomes (e.g., Simple Endoscopic Score for Crohn's disease; SES-CD), patient reported outcomes (Crohn's Disease Activity Index, CDAI; Two-Item Patient Reported Outcome; PRO-2 score), or biomarker levels (e.g., C-reactive protein, CRP; fecal calprotectin, FCP). See, e.g., Section 7.2.

In some embodiments, if an IBD patient is found to respond to the anti-SMAD7 therapy during the first treatment period and the IBD patient loses some or all of the response during an observation period without treatment, the patient can enter the second treatment period. In some embodiments, if an IBD patient having responded to the anti-SMAD7 therapy during the first treatment period, is found to lose some or all of the response to the anti-SMAD7 therapy during the second treatment period, the second treatment period can be shortened or ended, and the IBD patient can enter the third treatment period.

In some embodiments, if an IBD patient is found not to respond to the anti-SMAD7 therapy during the first and/or second treatment period, the dosage of the anti-SMAD7 therapy can be increased (e.g., by 50%, 2-fold, 4-fold, 6-fold, 8-fold or more) and/or the first and/or second treatment period can be repeated.

7.1.1 Administration Regimen

In one aspect, provided herein is a method for treating or managing inflammatory bowel disease (IBD) in a patient having IBD, wherein the method comprises (a) administering to the patient a SMAD7 antisense-oligonucleotide (SMAD7 AON) during a first treatment period at a first dose; and (b) administering to the patient the SMAD7 AON during a second treatment period at a second dose.

In another aspect, provided herein is a method for treating or managing inflammatory bowel disease (IBD) in a patient having IBD, wherein the method comprises (a) administering to the patient a SMAD7 antisense-oligonucleotide (SMAD7 AON) during a first treatment period at a first dose; (b) administering to the patient the SMAD7 AON during a second treatment period at a second dose; and (c) administering to the patient the SMAD7 AON during a third treatment period at a third dose.

The first and/or second and/or third treatment periods each can have a duration of weeks, months, or years. The length of the first and/or second and/or third treatment period can be adjusted depending, e.g., on whether an IBD patient responds to the anti-SMAD7 therapy, on how strongly the patient responds (e.g., the degree of the clinical response or the occurrence of remission), or on whether a patient, who has previously responded to the anti-SMAD7 therapy, relapses.

In one aspect, provided herein is a method for preventing inflammatory bowel disease (IBD) in a patient at risk of developing IBD, wherein the method comprises (a) administering to the patient a SMAD7 AON during a first treatment period at a first dose; and (b) administering to the patient the SMAD7 AON during a second treatment period at a second dose.

In another aspect, provided herein is a method for preventing inflammatory bowel disease (IBD) in a patient at risk of developing IBD, wherein the method comprises (a) administering to the patient a SMAD7 AON during a first treatment period at a first dose; (b) administering to the patient the SMAD7 AON during a second treatment period at a second dose; and (c) administering to the patient the SMAD7 AON during a third treatment period at a third dose.

7.1.1.1 First Treatment Period

In some embodiments, the first treatment period is between about 1 week and about 20 weeks, between about 2 weeks and about 18 weeks, between about 4 weeks and about 16 weeks, between about 6 weeks and about 14 weeks, or between about 8 weeks and about 12 weeks.

In some embodiments, the first treatment period is about 1 week, about 2 weeks, about 4 weeks, about 6 weeks, about 8 weeks, about 10 weeks, about 12 weeks, about 14 weeks, about 16 weeks, about 18 weeks, or about 20 weeks.

In some embodiments, the first treatment period is about 4 weeks, about 8 weeks, or about 12 weeks.

In some embodiments, the first treatment period is at least about 1 week, at least about 2 weeks, at least about 4 weeks, at least about 6 weeks, at least about 8 weeks, at least about 10 weeks, at least about 52 weeks, at least about 56 weeks, at least about 60 weeks, at least about 3 months, at least about 6 months, at least about 9 months, at least about 12 months, at least about 18 months, at least about 24 months.

In some embodiments, the first treatment period is about 12 weeks.

In some embodiments, the first treatment period is about 8 weeks.

In some embodiments, the first treatment period is between about 1 week and about 100 weeks, between about 10 weeks and about 90 weeks, between about 20 weeks and about 80 weeks, between about 30 weeks and about 70 weeks and between about 40 weeks and about 60 weeks.

In some embodiments, the first treatment period is between about 4 weeks and about 8 weeks.

In some embodiments, the first treatment period is between about 4 weeks and about 12 weeks.

In some embodiments, the first treatment period lasts until the IBD patient shows a response to a SMAD7 AON (e.g., decrease of SES-CD score from baseline ≥25% or ≥50%; decrease of CDAI score from baseline ≥100 points; decrease of PRO-2 score from baseline ≥8 points, decrease of average daily liquid or soft stool frequency score ≤1 and/or decrease of abdominal pain score ≤1; decrease of TMS score from baseline ≥30% and ≥3 points; decrease of ES from baseline ≥1; decrease of PMS score from baseline ≥25% and ≥2 points; decrease of MMS score from baseline ≥25% and ≥2 points) or until the IBD patient having IBD experiences remission (e.g., SES-CD score 52; CDAI score ≤150; PRO-2 score ≤8; average daily liquid or soft stool frequency score ≤1.5 and/or abdominal pain score ≤1; TMS score ≤2 points; ES=0; PMS score ≤2 or MMS score ≤2).

In some embodiments, the first treatment period lasts until the IBD patient shows a response to a SMAD7 AON (e.g., decrease of TMS score from baseline ≥30% and ≥3 points, along with decrease of RBS score ≥1 or absolute RBS ≤1; decrease of endoscopic subscore from baseline ≥1; decrease of PMS score from baseline ≥25% and ≥2 points, along with decrease of RBS score ≥1 or an absolute RBS ≤1; decrease of MMS score from baseline ≥25% and ≥2 points, along with a reduction in RBS score of ≥1 or an absolute RBS ≤1) or until the IBD patient having IBD experiences remission (e.g., TMS score ≤2 points with no individual subscore >1; endoscopic subscore=0; PMS score ≤2 points with no individual subscore >1; MMS score ≤2 points with no individual subscore >1).

In some embodiments, the first treatment period lasts until the patient shows dose-limiting toxicity or experiences an adverse event.

7.1.1.2 Dose During First Treatment Period

In the methods provided herein, during the first treatment period, a first dose of an anti-SMAD7 therapy (e.g., a SMAD7 AON) is administered to the IBD patient.

In some embodiments, the first dose of the SMAD7 AON is between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg.

In some embodiments, the first dose of the SMAD7 AON is between about 30 mg and about 620 mg, between about 60 mg and about 580 mg, between about 100 mg and about 540 mg, between about 140 mg and about 500 mg, between about 180 mg and about 460 mg, between about 220 mg and about 420 mg, between about 260 mg and about 380 mg, or between about 300 mg and about 340 mg.

In some embodiments, the first dose of the SMAD7 AON is between about 5 mg and about 90 mg, between about 10 mg and about 70 mg, or between about 30 mg and about 50 mg.

In some embodiments, the first dose of the SMAD7 AON is about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, or about 320 mg/day.

In some embodiments, the first dose of the SMAD7 AON is about 40 mg, about 80 mg, about 120 mg, about 160 mg, about 200 mg, about 240 mg, about 280 mg, about 320 mg, about 360 mg, about 400 mg, about 440 mg, about 480 mg, about 520 mg, about 560 mg, about 600 mg, or about 640 mg.

In some embodiments, the first dose of the SMAD7 AON is about 40 mg.

In some embodiments, the first dose of the SMAD7 AON is about 160 mg.

In some embodiments, the first dose of the SMAD7 AON is about 320 mg.

In some embodiments, the first dose of the SMAD7 AON is between about 30 mg/day and about 310 mg/day, between about 50 mg/day and about 290 mg/day, between about 70 mg/day and about 270 mg/day, between about 90 mg/day and about 250 mg/day, between about 110 mg/day and about 230 mg/day, between about 130 mg/day and about 190 mg/day, or between about 150 mg/day and about 170 mg/day.

In some embodiments, the first dose of the SMAD7 AON is between about 30 mg/day and about 620 mg/day, between about 60 mg/day and about 580 mg/day, between about 100 mg/day and about 540 mg/day, between about 140 mg/day and about 500 mg/day, between about 180 mg/day and about 460 mg/day, between about 220 mg/day and about 420 mg/day, between about 260 mg/day and about 380 mg/day, or between about 300 mg/day and about 340 mg/day.

In some embodiments, the first dose of the SMAD7 AON is between about 5 mg/day and about 90 mg/day, between about 10 mg/day and about 70 mg/day, or between about 30 mg/day and about 50 mg/day.

In some embodiments, the first dose of the SMAD7 AON is about 20 mg/day, about 40 mg/day, about 60 mg/day, about 80 mg/day, about 100 mg/day, about 120 mg/day, about 140 mg/day, about 160 mg/day, about 180 mg/day, about 200 mg/day, about 220 mg/day, about 240 mg/day, about 260 mg/day, about 280 mg/day, about 300 mg/day, or about 320 mg/day.

In some embodiments, the first dose of the SMAD7 AON is about 40 mg/day, about 80 mg/day, about 120 mg/day, about 160 mg/day, about 200 mg/day, about 240 mg/day, about 280 mg/day, about 320 mg/day, about 360 mg/day, about 400 mg/day, about 440 mg/day, about 480 mg/day, about 520 mg/day, about 560 mg/day, about 600 mg/day, or about 640 mg/day.

In some embodiments, the first dose of the SMAD7 AON is about 40 mg/day.

In some embodiments, the first dose of the SMAD7 AON is about 160 mg/day.

In some embodiments, the first dose of the SMAD7 AON is about 320 mg/day.

7.1.1.3 Transition from First to Second Treatment Period

In the methods for treating IBD provided herein, an IBD patient can transition from a first treatment period to a second treatment period.

In some embodiments, the IBD patient transitions directly from the first treatment period to the second treatment period, i.e., without an intermediate period, such as an observation period. In some embodiments, the IBD patient transitions from the first to the second treatment period through an intermediate period, such as an observation period.

In some embodiments, the transition can occur based on a time dependent schedule, e.g., without considering the IBD patients response to the anti-SMAD7 therapy. For example, in some embodiments, the first treatment period has a predetermined length (e.g., 4 weeks, 8 weeks, or 12 weeks) and at the end of the first treatment period the IBD patient transitions from the first treatment period to the second treatment period, regardless of any results from monitoring the activity of the anti-SMAD7 therapy during the first treatment period (e.g., regardless of the observation of any response to the anti-SMAD7 therapy in the IBD patient).

In some embodiments, an IBD patient transitions from the first treatment period to the second treatment period if the IBD patient, at one or more timepoints during the first treatment period or at the end of the first treatment period, shows a clinical response to the anti-SMAD7 therapy (e.g., a SMAD7 AON), or if the IBD patient goes into remission. See, e.g., Section 7.2.1, Section 7.2.2, Section 7.6 and Section 7.7. The clinical response or remission of the IBD patient can be analyzed, e.g., based on an endoscopic outcome, a clinical activity parameter, a safety or tolerability parameter, a biomarker of intestinal inflammation or tissue damage, a histological score, expression of a biomarker in an intestinal mucosal biopsy. See, e.g., Section 7.2.

The treatment regimen, e.g., during the first and/or second treatment period can be adjusted depending on the strength of the clinical response in the IBD patient (e.g., depending on the decrease in CDAI from baseline), or depending on the timepoint of at which the patient shows a clinical response. For example, the stronger an IBD patient's clinical response is at the end of the first treatment period, the more the dose of the anti-SMAD7 therapy can be reduced during the second treatment period. If the IBD patient shows a response earlier during the first treatment period, the patient can transition into the second treatment period earlier. See, e.g., Section 7.7.

In some embodiments, the IBD patient transitions from the first treatment period to the second treatment period, if the patient shows a decrease of SES-CD score from baseline ≥25% or ≥50%, a decrease of CDAI score from baseline ≥100 points, a decrease of PRO-2 score from baseline ≥8 points, a decrease of average daily liquid or soft stool frequency scores ≥1 and/or a decrease of an abdominal pain score ≥1; TMS ≤2, PMS ≤2, MMS ≤2, or ES=1 or 0.

In some embodiments, baseline is the SES-CD score at a timepoint during week 0 of the first treatment period. In some embodiments, baseline is the SES-CD score at a timepoint immediately prior to the first administration of the anti-SMAD7 therapy.

In some embodiments, baseline is the CDAI score at a timepoint during week 0 of the first treatment period. In some embodiments, baseline is the CDAI score at a timepoint immediately prior to the first administration of the anti-SMAD7 therapy.

In some embodiments, baseline is the PRO-2 score at a timepoint during week 0 of the first treatment period. In some embodiments, baseline is the PRO-2 score at a timepoint immediately prior to the first administration of the anti-SMAD7 therapy.

In some embodiments, the IBD patient transitions from the first treatment period to the second treatment period, if the patient shows a SES-CD score ≤2, a CDAI score <150, a PRO-2 score ≤8, an average daily liquid or soft stool frequency score ≤3 or ≤1.5 and/or an abdominal pain score ≤1; a TMS ≤2, a PMS ≤2, an MMS ≤2, or an ES=1 or 0.

In some embodiments, the IBD patient transitions from the first treatment period to the second treatment period, if the patient shows a decrease of TMS score from baseline ≥30% and ≥3 points, along with a decrease of RBS score ≥1 or absolute RBS ≤1; a decrease of endoscopic subscore from baseline ≥1; a decrease of PMS score from baseline ≥25% and ≥2 points, along with a decrease of RBS score ≥1 or an absolute RBS ≤1; a decrease of MMS score from baseline ≥25% and ≥2 points, along with a reduction in RBS score of ≥1 or an absolute RBS ≤1.

In some embodiments, the IBD patient transitions from the first treatment period to the second treatment period, if the patient shows a TMS score ≤2 points with no individual subscore >1; an endoscopic subscore (ES)=0; a PMS score ≤2 points with no individual subscore >1; an MMS score ≤2 points with no individual subscore >1.

In some embodiments, baseline is the TMS score at a timepoint during week 0 of the first treatment period. In some embodiments, baseline is the TMS score at a timepoint immediately prior to the first administration of the anti-SMAD7 therapy.

In some embodiments, baseline is the endoscopic subscore at a timepoint during week 0 of the first treatment period. In some embodiments, baseline is the endoscopic subscore at a timepoint immediately prior to the first administration of the anti-SMAD7 therapy.

In some embodiments, baseline is the PMS score at a timepoint during week 0 of the first treatment period. In some embodiments, baseline is the PMS score at a timepoint immediately prior to the first administration of the anti-SMAD7 therapy.

In some embodiments, baseline is the MMS score at a timepoint during week 0 of the first treatment period. In some embodiments, baseline is the MMS score at a timepoint immediately prior to the first administration of the anti-SMAD7 therapy.

In some embodiments, the IBD patient transitions from the first treatment period to the second treatment period if the level of a biomarker, such as, e.g., SMAD7 (e.g., SMAD7 protein or SMAD7 mRNA), SMAD3 phosphorylation, HLA-DR, IL6, IL8, IL12, IL17A, CD4, CD8, IFN-γ, CRP, FCP, TNFα, or the like, in a sample from the patient having IBD is at least 20%, at least 30%, at least 40%, at least 50%, at least 60%, at least 70%, at least 80%, or at least 90% decreased from baseline (e.g., respective biomarker level at timepoint during week 0 of first treatment period).

In some embodiments, the IBD patient transitions from the first treatment period to the second treatment period if the level of a biomarker, such as, e.g., SMAD7, SMAD3 phosphorylation, HLA-DR, IL6, IL8, IL12, IL17A, IFN-γ, CD4, CD8, CRP, FCP, TNFα, or the like, in a sample from the patient having IBD is within a standard deviation (SD) range of 2σ, 3σ, 5σ, 6σ, or 10σ of the average, median, or mean level of the biomarker in a healthy control group.

In some embodiments, the IBD patient transitions from the first treatment period to the second treatment period if the patient shows mucosal healing, as indicated, e.g., by the absence of an intestinal mucosal ulceration.

In some embodiments, if the IBD patient at the end of the first treatment period (e.g., during week 4, week 8, or week 12) does not show a response to the anti-SMAD7 therapy the IBD patient does not transition from the first treatment period to the second treatment period. In some embodiments, the non-responding IBD patient repeats the first treatment period. In some embodiments, the non-responding IBD patient repeats the first treatment period and is administered with an increased first dose of the anti-SMAD7 therapy. In some embodiments, the treatment of the IBD patient is terminated (e.g., if the IBD patient was already treated with the maximum tolerated dose of the anti-SMAD7 therapy, or if the IBD patient experienced an adverse effect). In some embodiments, the increased first dose is about 1.5-fold, about 2-fold, about 4-fold, about 8-fold, about 16-fold of the initial first dose.

In some embodiments, if the IBD patient shows a response to the anti-SMAD7 treatment or if the IBD is in remission at the end of the first treatment period, the anti-SMAD7 treatment is terminated and the IBD patient does not transition to the second treatment period.

In some embodiments, if an IBD patient shows a response to the anti-SMAD7 treatment or if the IBD patient is in remission at the end of the first treatment period, and if, during a subsequent observation period without treatment, the patient experiences loss of remission or loss of some or all of the response observed at the end of the first treatment period, the patient transitions to the second treatment period. In some embodiments, if an IBD patient shows a response to the anti-SMAD7 treatment or if the IBD patient is in remission at the end of the first treatment period, and if, during a subsequent observation period without treatment, the patient experiences loss of partial response (e.g., at 2 consecutive visits the patient has a CADI score >150 and an increase of CDAI score >50 points from the CDAI score when the patient was first a responder during the first treatment period), the patient transitions to the second treatment period. In some embodiments, if an IBD patient receives a corticosteroid treatment prior to or during the first treatment period and the patient cannot taper the corticosteroid during the subsequent observation period, the patient transitions to the second treatment period.

7.1.1.4 Second Treatment Period

In some embodiments, the second treatment period is between about 1 week and about 50 weeks, between about 2 weeks and about 48 weeks, between about 4 weeks and about 46 weeks, between about 6 weeks and about 44 weeks, between about 8 weeks and about 42 weeks, between about 10 weeks and about 40 weeks, between about 12 weeks and about 38 weeks, between about 14 weeks and about 36 weeks, between about 16 weeks and about 34 weeks, between about 18 weeks and about 32 weeks, between about 20 weeks and about 30 weeks, between about 22 week and about 28 weeks, between about 22 weeks and about 26 weeks, or between about 24 weeks and about 26 weeks.

In some embodiments, the second treatment period is about 1 week, about 2 weeks, about 4 weeks, about 6 weeks, about 8 weeks, about 10 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, or about 50 weeks.

In some embodiments, the second treatment period is about 24 weeks.

In some embodiments, the second treatment period is between about 1 week and about 100 weeks, between about 5 weeks and about 95 weeks, between about 10 weeks and about 90 weeks, between about 15 weeks and about 85 weeks, between about 20 weeks and about 80 weeks, between about 25 weeks and about 75 weeks, between about 30 weeks and about 70 weeks, between about 35 weeks and about 65 weeks, between about 40 weeks and about 60 weeks, between about 40 weeks and about 55 weeks, between about 45 weeks and about 55 weeks, or between about 50 weeks and about 55 weeks.

In some embodiments, the second treatment period is about 1 week, about 5 weeks, about 10 weeks, about 15 weeks, about 20 weeks, about 25 weeks, about 30 weeks, about 35 weeks, about 40 weeks, about 45 weeks, about 50 weeks, about 55 weeks, about 60 weeks, about 65 weeks, about 70 weeks, about 75 weeks, about 80 weeks, about 85 weeks, about 90 weeks, about 95 weeks, or about 100 weeks.

In some embodiments, the second treatment period is about 52 weeks.

In some embodiments, the second treatment period is about 40 weeks.

In some embodiments, the second treatment period is about 44 weeks.

In some embodiments, the second treatment period is at least about 1 week, at least about 2 weeks, at least about 4 weeks, at least about 6 weeks, at least about 8 weeks, at least about 10 weeks, at least about 3 months, at least about 6 months, at least about 9 months, at least about 12 months, at least about 18 months, at least about 24 months, at least about 30 months, at least about 3 years, at least about 4 years, at least about 5 years, at least about 6 years, at least about 7 years, at least about 8 years, at least about 9 years, or at least about 10 years.

In some embodiments, the second treatment period lasts until the patient shows dose-limiting toxicity or experiences an adverse event.

In some embodiments, the second treatment period lasts for the duration of a patient's remaining life span.

In some embodiments, the second treatment period lasts for an indefinite period of time, i.e., a period of time that is not predetermined. In some embodiments, the second treatment period lasts until the patient shows a certain response to the treatment or meets a specified, predetermined clinical milestone, e.g., as determined by results from colonoscopy or ileocolonoscopy tests, biomarker levels or other tests, such as patient reported outcomes, or quality of life measurements (e.g., achievement or maintenance for a specific time of CDAI <150, SES-CD ≤2, PRO-2 score <8, or CRP levels <1.0 mg/L, average daily liquid or soft stool frequency score ≤3 or ≤1.5 and/or an abdominal pain score ≤1; TMS ≤2, PMS ≤2, MMS ≤2, ES=1 or 0, and the like).

In some embodiments, the second treatment period is between about 1 week and about 400 weeks, between about 40 weeks and about 340 weeks, between about 80 weeks and about 320 weeks, between about 120 weeks and about 280 weeks, between about 160 weeks and about 240 weeks, or between about 180 weeks and about 200 weeks.

In some embodiments, the second treatment period is about 196 weeks.

In some embodiments, the second treatment period lasts until the patient having IBD shows a loss of response to the SMAD7 AON (e.g., increase of SES-CD score ≥50%, compared to SES-CD score at time of first response; increase of CDAI score ≥50 points compared to CDAI score at time of first response; increase of PRO-2 score of ≥8 points compared to PRO-2 score at time of first response; increase of daily liquid or soft stool frequency score of ≥1 point and/or of an abdominal pain score of ≥1 points compared to liquid or soft stool frequency and/or abdominal pain scores at time of first response).

7.1.1.5 Dosing During Second Treatment Period

In the methods provided herein, during the second treatment period, the IBD patient is administered with a second dose of an anti-SMAD7 therapy (e.g., a SMAD7 AON).

In some embodiments, the second dose of the SMAD7 AON is between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg.

In some embodiments, the second dose of the SMAD7 AON is between about 30 mg and about 620 mg, between about 60 mg and about 580 mg, between about 100 mg and about 540 mg, between about 140 mg and about 500 mg, between about 180 mg and about 460 mg, between about 220 mg and about 420 mg, between about 260 mg and about 380 mg, between about 300 mg and about 340 mg.

In some embodiments, the second dose of the SMAD7 AON is between about 5 mg and about 90 mg, between about 10 mg and about 70 mg, or between about 30 mg and about 50 mg.

In some embodiments, the second dose of the SMAD7 AON is about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg.

In some embodiments, the second dose of the SMAD7 AON is about 40 mg, about 80 mg, about 120 mg, about 160 mg, about 200 mg, about 240 mg, about 280 mg, about 320 mg, about 360 mg, about 400 mg, about 440 mg, about 480 mg, about 520 mg, about 560 mg, about 600 mg or about 640 mg.

In some embodiments, the second dose of the SMAD7 AON is about 40 mg.

In some embodiments, the second dose of the SMAD7 AON is about 160 mg.

In some embodiments, the second dose of the SMAD7 AON is about 320 mg.

In some embodiments, the second dose of the SMAD7 AON is between about 30 mg/day and about 310 mg/day, between about 50 mg/day and about 290 mg/day, between about 70 mg/day and about 270 mg/day, between about 90 mg/day and about 250 mg/day, between about 110 mg/day and about 230 mg/day, between about 130 mg/day and about 190 mg/day, or between about 150 mg/day and about 170 mg/day.

In some embodiments, the second dose of the SMAD7 AON is between about 30 mg/day and about 620 mg/day, between about 60 mg/day and about 580 mg/day, between about 100 mg/day and about 540 mg/day, between about 140 mg/day and about 500 mg/day, between about 180 mg/day and about 480 mg/day, between about 220 mg/day and about 420 mg/day, between about 260 mg/day and about 380 mg/day or between about 300 mg/day and about 340 mg/day.

In some embodiments, the second dose of the SMAD7 AON is between about 5 mg/day and about 90 mg/day, between about 10 mg/day and about 70 mg/day, or between about 30 mg/day and about 50 mg/day.

In some embodiments, the second dose of the SMAD7 AON is about 20 mg/day, about 40 mg/day, about 60 mg/day, about 80 mg/day, about 100 mg/day, about 120 mg/day, about 140 mg/day, about 160 mg/day, about 180 mg/day, about 200 mg/day, about 220 mg/day, about 240 mg/day, about 260 mg/day, about 280 mg/day, about 300 mg/day, or about 320 mg/day.

In some embodiments, the second dose of the SMAD7 AON is about 40 mg/day, about 80 mg/day, about 120 mg/day, about 160 mg/day, about 200 mg/day, about 240 mg/day, about 280 mg/day, about 320 mg/day, about 360 mg/day, about 400 mg/day, about 440 mg/day, about 480 mg/day, about 520 mg/day, about 560 mg/day, about 600 mg/day, or about 640 mg/day.

In some embodiments, the second dose of the SMAD7 AON is about 40 mg/day.

In some embodiments, the second dose of the SMAD7 AON is about 160 mg/day.

In some embodiments, the second dose of the SMAD7 AON is about 320 mg/day.

In some embodiments, the first and second dose of the SMAD7 AON are the same dose. In some embodiments, the first and the second dose of the SMAD7 AON are different doses.

In some embodiments, the second dose of the SMAD7 AON is lower than the first dose of the SMAD7 AON. In some embodiments, the second dose is at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, or at least about 300 mg lower than the first dose.

In some embodiments, the second dose of the SMAD7 AON is lower than the first dose of the SMAD7 AON. In some embodiments, the second dose is at least about 20 mg/day, at least about 40 mg/day, at least about 60 mg/day, at least about 80 mg/day, at least about 100 mg/day, at least about 120 mg/day, at least about 140 mg/day, at least about 160 mg/day, at least about 180 mg/day, at least about 200 mg/day, at least about 220 mg/day, at least about 240 mg/day, at least about 260 mg/day, at least about 280 mg/day, or at least about 300 mg/day lower than the first dose.

In some embodiments, the second dose of the SMAD7 AON is higher than the first dose of the SMAD7 AON. In some embodiments, the second dose is at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, or at least about 300 mg higher than the first dose.

In some embodiments, the second dose of the SMAD7 AON is higher than the first dose of the SMAD7 AON. In some embodiments, the second dose is at least about 20 mg/day, at least about 40 mg/day, at least about 60 mg/day, at least about 80 mg/day, at least about 100 mg/day, at least about 120 mg/day, at least about 140 mg/day, at least about 160 mg/day, at least about 180 mg/day, at least about 200 mg/day, at least about 220 mg/day, at least about 240 mg/day, at least about 260 mg/day, at least about 280 mg/day, or at least about 300 mg/day higher than the first dose.

In some embodiments, the first and second dose of the SMAD7 AON are the same dose. In some embodiments, the first and second dose are at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, or at least about 320 mg.

In some embodiments, the first and second dose of the SMAD7 AON are the same dose. In some embodiments, the first and second dose are at least about 20 mg/day, at least about 40 mg/day, at least about 60 mg/day, at least about 80 mg/day, at least about 100 mg/day, at least about 120 mg/day, at least about 140 mg/day, at least about 160 mg/day, at least about 180 mg/day, at least about 200 mg/day, at least about 220 mg/day, at least about 240 mg/day, at least about 260 mg/day, at least about 280 mg/day, at least about 300 mg/day, or at least about 320 mg/day.

7.1.1.6 End of Second Treatment Period

The second treatment period can end on a time dependent schedule, or on a schedule that is dependent on the clinical response of an IBD patient to an anti-SMAD7 therapy. For example, the length of the second treatment period can be predetermined. A second treatment period of predetermined length can end at the predetermined time, regardless of whether the IBD patient, at that predetermined timepoint or at any timepoint during the second treatment period, responds to the anti-SMAD7 therapy or whether the IBD patient shows a partial or complete loss of response.

In some embodiments, if the IBD patient shows a partial or total loss of response to the anti-SMAD7 therapy (e.g., SMAD7 AON) during the second treatment period, the patient exits the second treatment period and reenters the first treatment period. See, e.g., Section 7.2.3. A patient reentering the first treatment period can be administered with a previously used first dose of the anti-SMAD7 therapy, or with an increased dose of the anti-SMAD7 therapy.

In some embodiments, the IBD patient exits the second treatment period if the patient shows an increase of SES-CD ≥50% or ≥75%, compared to the IBD patient's SES-CD at time of first response; if the patient shows an increase of CDAI score ≥50 points compared to CDAI score at time of first response, if the patient shows an increase of PRO-2 score of ≥8 points compared to PRO-2 score at time of first response, if the patient shows an increase of daily liquid or soft stool frequency score of ≥1 point and/or of an abdominal pain score of ≥1 point compared to the liquid or soft stool frequency and/or abdominal pain scores at time of first response; if the patient shows ≥30% or ≥3 points increase of TMS from baseline; if the patient shows a ≥25% or ≥2 points increase of PMS from baseline; if the patient shows a ≥25% or ≥2 points increase of MMS from baseline, or if the patient shows a ≥1 point increase in ES from baseline.

In some embodiments, if the IBD patient shows a response to the anti-SMAD7 treatment or if the IBD is in remission at the end of the second treatment period, the anti-SMAD7 treatment is terminated.

7.1.1.7 Transition to Third Treatment Period

In the methods for treating IBD provided herein, an IBD patient can optionally transition to a third treatment period. In some embodiments, the IBD patient transitions to the third treatment period at the end of a first treatment period. In some embodiments, the IBD patient transitions to the third treatment period at the end of a second treatment period. In some embodiments, the IBD patient transitions to the third treatment period at a time point during a first and/or a second treatment. In some embodiments, the IBD patient transitions to the third treatment period at the end of or at a time point during an observation period. In some embodiments, the observation period occurs between a first and a second time period. In some embodiments, the observation period follows a second time period. In some embodiments, the IBD patient does not transition to a third treatment period.

In some embodiments, the IBD patient transitions directly from a first or second treatment period to the third treatment period, i.e., without an intermediate period, such as an observation period. In some embodiments, the IBD patient transitions from the first or second treatment period to the third treatment period through an intermediate period, such as an observation period.

In some embodiments, the transition to the third time period can occur based on a time dependent schedule, e.g., without considering the IBD patient's response to the anti-SMAD7 therapy. For example, in some embodiments, the second treatment period can have a predetermined length (e.g., 12 weeks, 24 weeks, 36 weeks, 48 weeks, or 52 weeks) and at the end of the second treatment period the IBD patient transitions from the second treatment period to the third treatment period, regardless of any results from monitoring the activity of the anti-SMAD7 therapy during the second treatment period (e.g., regardless of the observation of any response to the anti-SMAD7 therapy in the IBD patient).

In some embodiments, an IBD patient transitions from the second treatment period to the third treatment period if the IBD patient, at one or more timepoints during the second treatment period or at the end of the second treatment period, shows a clinical response to the anti-SMAD7 therapy (e.g., a SMAD7 AON), or if the IBD patient goes into remission. See, e.g., Section 7.2.1, Section 7.2.2, Section 7.6 and Section 7.7. The remission or clinical response of the IBD patient can be analyzed, e.g., based on an endoscopic outcome, a clinical activity parameter, a safety or tolerability parameter, a biomarker of intestinal inflammation or tissue damage, a histological score, expression of a biomarker in an intestinal mucosal biopsy. See, e.g., Section 7.2.

The treatment regimen, e.g., during the first, second and/or third treatment period can be adjusted depending on the strength of the clinical response in the IBD patient (e.g., depending on the decrease in the IBD patient's CDAI from baseline), or depending on the timepoint at which the patient shows a clinical response. For example, the stronger an IBD patient's clinical response is at the end of the prior (e.g., the first or second) treatment period, the further the dose of the anti-SMAD7 therapy can be reduced during the third treatment period. In some embodiments, if the IBD patient shows a response to the anti-SMAD7 therapy before the end of the prior (first or second) treatment period, the patient can transition into the third treatment as soon as the IBD patient shows the response. See, e.g., Section 7.7.

In some embodiments, the IBD patient transitions to the third treatment period, if the patient shows a decrease of SES-CD score from baseline ≥25% or ≥50%, a decrease of CDAI score from baseline ≥100 points, a decrease of PRO-2 score from baseline ≥8 points, a decrease of daily liquid or soft stool frequency score of ≥1 point and/or of an abdominal pain score of ≥1 point compared to the liquid or soft stool frequency and/or abdominal pain scores at baseline, a ≥3 points or ≥30% increase in TMS from baseline, a ≥2 points or ≥25% increase in PMS from baseline, a ≥2 or ≥25% points increase in MMS from baseline, or a ≥1 point increase in ES from baseline.

In some embodiments, SES-CD, CDAI, PRO-2, daily liquid or soft stool frequency score, abdominal pain score, MMS, PMS, TMS or ES baseline is the corresponding SES-CD, CDAI, PRO-2 score, daily liquid or soft stool frequency score, abdominal pain score, MMS, PMS, TMS or ES at a timepoint during week 0 of the first treatment period. In some embodiments, SES-CD, CDAI, PRO-2, daily liquid or soft stool frequency score, abdominal pain score, MMS, PMS, TMS or ES baseline is the corresponding SES-CD, CDAI, PRO-2, daily liquid or soft stool frequency score, abdominal pain score, MMS, PMS, TMS or ES score at a timepoint immediately prior to the first administration of the anti-SMAD7 therapy.

In some embodiments, the IBD patient transitions to the third treatment period, if the patient shows a SES-CD score ≤2, a CDAI score <150, a PRO-2 score ≤8, a daily liquid or soft stool frequency ≤3 or ≤1.5 and/or an abdominal pain score ≤1; TMS ≤2, PMS ≤2, MMS ≤2, or ES=1 or 0.

In some embodiments, the IBD patient transitions from the first treatment period to the second treatment period, if the patient shows a decrease of TMS score from baseline ≥30% and ≥3 points, along with a decrease of RBS score ≥1 or absolute RBS ≤1; a decrease of endoscopic subscore from baseline ≥1; a decrease of PMS score from baseline ≥25% and ≥2 points, along with a decrease of RBS score ≥1 or an absolute RBS ≤1; a decrease of MMS score from baseline ≥25% and ≥2 points, along with a reduction in RBS score of ≥1 or an absolute RBS ≤1.

In some embodiments, the IBD patient transitions from the first treatment period to the second treatment period, if the patient shows a TMS score ≤2 points with no individual subscore >1; an endoscopic subscore=0; a PMS score ≤2 points with no individual subscore ≥i; an MMS score ≤2 points with no individual subscore >1.

In some embodiments, TMS score, PMS score, MMS score, RBS score, or endoscopic score baseline is the corresponding TMS score, PMS score, MMS score, RBS score, or endoscopic score at a timepoint during week 0 of the first treatment period. In some embodiments, TMS score, PMS score, MMS score, RBS score, or endoscopic score baseline is the corresponding TMS score, PMS score, MMS score, RBS score, or endoscopic score at a timepoint immediately prior to the first administration of the anti-SMAD7 therapy.

In some embodiments, the IBD patient transitions to the third treatment period if the level of a biomarker, such as, e.g., SMAD7, SMAD3 phosphorylation, HLA-DR, CD4, CD8, CRP, FCP, TNFα, IFN-γ, IL8, IL-12, IL17A or IL6 or the like, in a sample from the patient having IBD is at least 20%, at least 30%, at least 40%, at least 50%, at least 60%, at least 70%, at least 80%, or at least 90% decreased from baseline (e.g., respective biomarker level at timepoint during week 0 of first treatment period).

In some embodiments, the IBD patient transitions to the third treatment period if the level of a biomarker, such as, e.g., SMAD7, SMAD3 phosphorylation, HLA-DR, CD4, CD8, CRP, FCP, TNFα, IFN-γ, IL8, IL-12, IL17A or IL6 level, or the like, in a sample from the patient having IBD is within a standard deviation (SD) range of 2σ, 3σ, 5σ, 6σ, or 10σ of the average, median, or mean level of the biomarker in a healthy control group.

In some embodiments, the IBD patient transitions to the third treatment period if the patient shows mucosal healing, as indicated, e.g., by the absence of an intestinal mucosal ulceration.

In some embodiments, if the IBD patient at the end of the second treatment period (e.g., during week 12, week 24, week 36, week 48, or week 52) does not show a response to the anti-SMAD7 therapy the IBD patient does not transition from the second treatment period to the third treatment period. In some embodiments, the non-responding IBD patient repeats the second treatment period. In some embodiments, the non-responding IBD patient repeats the second treatment period and is administered with an increased second dose of the anti-SMAD7 therapy. In some embodiments, the treatment of the IBD patient is terminated (e.g., if the IBD patient was already treated with the maximum tolerated dose of the anti-SMAD7 therapy, or if the IBD patient experienced an adverse effect).

In some embodiments, if the IBD patient shows a response to the anti-SMAD7 treatment or if the IBD is in remission at the end of the first or second treatment period, the anti-SMAD7 treatment is terminated and the IBD patient does not transition to the third treatment period.

In some embodiments, if an IBD patient does not show a clinical improvement (e.g., the patient does not have a CDAI <180 and a reduction of ≥70 points in the CDAI score as compared to the baseline) at any time point during the second treatment period, the IBD patient transitions from the second treatment period to the third treatment period.

7.1.1.8 Third Treatment Period

In some embodiments, the third treatment period is between about 1 week and about 8 years, between about 12 weeks and about 7 years, between about 24 weeks and about 6 years, between about 36 weeks and about 5 years, or between about 52 weeks and about 4 years (i.e., about 208 weeks).

In some embodiments, the third treatment period is about 1 week, about 12 weeks, about 24 weeks, about 36 weeks, about 52 weeks, about 1.5 years, about 2 years, about 2.5 years, about 3 years, about 3.5 years, about 4 years (i.e., about 208 weeks), about 5 years, about 6 years, about 7 year weeks, or about 8 years.

In some embodiments, the third treatment period is about 26 weeks, about 52 weeks, about 78 weeks, about 104 weeks, about 130 weeks, about 156 weeks, about 182 weeks, about 196 weeks, about 208 weeks, or about 312 weeks.

In some embodiments, the third treatment period is at least about 1 week, at least about 2 weeks, at least about 4 weeks, at least about 6 weeks, at least about 8 weeks, at least about 10 weeks, at least about 52 weeks, at least about 56 weeks, at least about 60 weeks, at least about 3 months, at least about 6 months, at least about 9 months, at least about 12 months, at least about 18 months, at least about 24 months, at least about 36 months, at least 48 months, at least 60 months, or at least 72 months.

In some embodiments, the third treatment period is about 208 weeks.

In some embodiments, the third treatment period is about 196 weeks.

In some embodiments, the third treatment period is between about 1 week and about 460 week, between about 40 weeks and about 420 weeks, between about 80 weeks and about 380 weeks, between about 120 weeks and about 340 weeks, between about 160 weeks and about 300 weeks, and between about 180 weeks and about 260 weeks.

In some embodiments, the third treatment period lasts until the IBD patient shows a response to a SMAD7 AON (e.g., decrease of SES-CD score from baseline ≥25% or ≥50%; decrease of CDAI score from baseline ≥100 points; decrease of PRO-2 score from baseline ≥8 points; decrease of average daily liquid or soft stool frequency score from baseline of 1 point and/or decrease of abdominal pain score ≥1 point) or until the IBD patient having IBD experiences remission (e.g., SES-CD score ≤2; CDAI score ≤150; PRO-2 score ≤8; average daily liquid or soft stool frequency score ≤1.5 and/or abdominal pain score ≤1. TMS score ≤2; ES=0; PMS ≤2 points; MMS ≤2).

In some embodiments, the third treatment period lasts until the patient shows dose-limiting toxicity or experiences an adverse event.

In some embodiments, the third treatment period lasts until the end of the patient's remaining life span.

In some embodiments, the third treatment period lasts for an indefinite period of time, i.e., a period of time that is not predetermined. In some embodiments, the third treatment period lasts until the patient shows a certain response to the treatment or meets a specified, predetermined clinical milestone, e.g., as determined by results from colonoscopy or ileocolonoscopy tests, biomarker levels or other tests, such as patient reported outcomes, or quality of life measurements (e.g., achievement or maintenance for a specific time of CDAI <150, SES-CD ≤2, PRO-2 score ≤8, or CRP levels <1.0 mg/L, average daily liquid or soft stool frequency of ≤3.0 or ≤1.5 points, and/or decrease of abdominal pain score ≤1 point, TMS score ≤2; ES=0 or 1; PMS ≤2 points; MMS ≤2, and the like).

7.1.1.9 Dose During Third Treatment Period

In the methods provided herein, during the third treatment period, a third dose of an anti-SMAD7 therapy (e.g., a SMAD7 AON) is administered to the IBD patient.

In some embodiments, the third dose of the SMAD7 AON is between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg.

In some embodiments, the third dose of the SMAD7 AON is between about 30 mg and about 620 mg, between about 60 mg and about 580 mg, between about 100 mg and about 540 mg, between about 140 mg and about 500 mg, between about 180 mg and about 460 mg, between about 220 mg and about 420 mg, or between 260 mg and about 380 mg, or between 300 mg and about 340 mg.

In some embodiments, the third dose of the SMAD7 AON is between about 5 mg and about 90 mg, between about 10 mg and about 70 mg, or between about 30 mg and about 50 mg.

In some embodiments, the third dose of the SMAD7 AON is about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg.

In some embodiments, the third dose of the SMAD7 AON is about 40 mg, about 80 mg, about 120 mg, about 160 mg, about 200 mg, about 240 mg, about 280 mg, about 320 mg, about 360 mg, about 400 mg, about 440 mg, about 480 mg, about 520 mg, about 560 mg, about 600 mg or about 640 mg.

In some embodiments, the third dose of the SMAD7 AON is about 40 mg.

In some embodiments, the third dose of the SMAD7 AON is about 80 mg.

In some embodiments, the third dose of the SMAD7 AON is about 160 mg.

In some embodiments, the third dose of the SMAD7 AON is about 320 mg.

In some embodiments, the third dose of the SMAD7 AON is between about 30 mg/day and about 310 mg/day, between about 50 mg/day and about 290 mg/day, between about 70 mg/day and about 270 mg/day, between about 90 mg/day and about 250 mg/day, between about 110 mg/day and about 230 mg/day, between about 130 mg/day and about 190 mg/day, or between about 150 mg/day and about 170 mg/day.

In some embodiments, the third dose of the SMAD7 AON is between about 30 mg/day and about 620 mg/day, between about 60 mg/day and about 580 mg/day, between about 100 mg/day and about 540 mg/day, between about 140 mg/day and about 500 mg/day, between about 180 mg/day and about 460 mg/day, between about 220 mg/day and about 420 mg/day or between about 260 mg/day and about 380 mg/day, or between about 300 mg/day and about 340 mg/day.

In some embodiments, the third dose of the SMAD7 AON is between about 5 mg/day and about 90 mg/day, between about 10 mg/day and about 70 mg/day, or between about 30 mg/day and about 50 mg/day.

In some embodiments, the third dose of the SMAD7 AON is about 20 mg/day, about 40 mg/day, about 60 mg/day, about 80 mg/day, about 100 mg/day, about 120 mg/day, about 140 mg/day, about 160 mg/day, about 180 mg/day, about 200 mg/day, about 220 mg/day, about 240 mg/day, about 260 mg/day, about 280 mg/day, about 300 mg/day, or about 320 mg/day.

In some embodiments, the third dose of the SMAD7 AON is about 40 mg/day, about 80 mg/day, about 120 mg/day, about 160 mg/day, about 200 mg/day, about 240 mg/day, about 280 mg/day, about 320 mg/day, about 360 mg/day, about 400 mg/day, about 440 mg/day, about 480 mg/day, about 520 mg/day, about 560 mg/day, about 600 mg/day, or about 640 mg/day.

In some embodiments, the third dose of the SMAD7 AON is about 40 mg/day.

In some embodiments, the third dose of the SMAD7 AON is about 80 mg/day.

In some embodiments, the third dose of the SMAD7 AON is about 160 mg/day.

In some embodiments, the third dose of the SMAD7 AON is about 320 mg/day.

In some embodiments, the first, second and third (optional) doses are the same doses (e.g., the first dose is the same as the second dose.). In some embodiments, one of the first, second and third doses is different from at least one of the two other doses (e.g., the first dose is different from the second dose). In some embodiments, each of the first, second and third doses is different from each of the other two doses.

7.1.1.10 Additional Time Periods

In some embodiments, the methods provided herein further comprise an initial screening period prior to the first treatment period, e.g., to assess or monitor the IBD disease severity or to taper or discontinue an additional IBD treatment prior to the first administration of the anti-SMAD7 therapy (e.g., a SMAD7 AON)

In some embodiments, no SMAD7 AON is administered during the screening period.

In some embodiments, the screening period is between about 1 week and about 10 weeks, between about 2 weeks and about 9 weeks, between about 3 weeks and about 8 weeks, between about 4 weeks and about 7 weeks, or between weeks and about 6 weeks. In some embodiments, the screening period is about 1 week, about 2 weeks, about 3 weeks, about 4 weeks, about 5 weeks, about 6 weeks, about 7 weeks, about 8 weeks, about 9 weeks or about 10 weeks. In some embodiments, the screening period is about 5 weeks.

In some embodiments, the methods provided herein further comprise an observation period between the first and the second treatment period, e.g., to monitor the IBD patient's response to the anti-SMAD7 therapy (e.g., SMAD7 AON) after the first treatment period.

In some embodiments, the methods provided herein further comprise an observation period between the second and the third treatment period, e.g., to monitor the IBD patient's response to the anti-SMAD7 therapy (e.g., SMAD7 AON) after the second treatment period. In some embodiments, no SMAD7 AON is administered to the patient having IBD during the observation period.

In some embodiments, the observation period is between about 1 week and about 100 weeks, between about 10 weeks and about 90 weeks, between about 20 weeks and about 80 weeks, between about 30 weeks and about 70 weeks, between about 40 weeks and about 60 weeks. In some embodiments, the observation period is up to about 10 weeks, up to about 20 weeks, up to about 30 weeks, up to about 40 weeks, up to about 50 weeks, up to about 60 weeks, up to about 70 weeks, up to about 80 weeks, up to about 90 weeks or up to about 100 weeks. In some embodiments, the observation period is up to about 52 weeks.

In some embodiment, tapering of an additional IBD treatment (i.e., an IBD treatment that is already in place prior to the start of the first and/or second treatment period) occurs during at least the first week, at least the second week, at least the third week, at least the fourth week, at least the fourth week, at least the fifth week, at least the sixth week, at least the seventh week, at least the eighth week, at least the ninth week, or at least the tenth week of the observation period.

In some embodiments, the methods provided herein further comprise a follow up period after the second treatment period, e.g., to monitor the IBD patient's response to the anti-SMAD7 therapy (e.g., SMAD7 AON) after the end of the second treatment period.

In some embodiments, the methods provided herein further comprise a follow up period after the third treatment period, e.g., to monitor the IBD patient's response to the anti-SMAD7 therapy (e.g., SMAD7 AON) after the end of the third treatment period.

In some embodiments, no SMAD7 AON is administered to the patient having IBD during the follow up period.

In some embodiments, the follow up period is between about 1 week and about 10 weeks, between about 2 weeks and about 9 weeks, between about 3 weeks and about 8 weeks, or between about 4 weeks and about 7 weeks. In some embodiments, the screening period is up to about 1 week, up to about 2 weeks, up to about 3 weeks, up to about 4 weeks, up to about 5 weeks, up to about 6 weeks, up to about 7 weeks, up to about 8 weeks, up to about 9 weeks, up to about 10 weeks, up to about 3 months, up to about 6 months, up to about 9 months, up to about 12 months, up to about 18 months, up to about 24 months, up to about 30 months, up to about 36 months, up to about 42 months, up to about 48 months, up to about 54 months, or up to about 60 months. In some embodiments, the follow up period is up to about 4 weeks.

In some embodiments, the methods provided herein do not comprise and additional time period. In some embodiments, the methods provided herein are consisting of a first, second and, optionally, a third treatment period.

7.1.2 Alternating Dosing Schedules

The SMAD7 AON can be administered continuously (e.g., once daily for 12 weeks) or on an alternating dosing schedule (e.g., once daily during week 0-4, no treatment during week 5-8, once daily during week 9-12) during the first, second and/or third treatment period. Continuous administrations can be at the same dose or at different doses (e.g., increasing or decreasing doses over time). In alternating dosing schedules, drug treatment periods can alternate with no drug treatment or placebo treatment periods (drug holiday periods), or treatment periods with two or more different doses can alternate.

In the alternating dosing schedules described herein two or more periods can be alternating. In some embodiments, the alternating dosing schedule can have a first alternating period and a second alternating period. In some embodiments, the first alternating period is a drug (e.g., SMAD7 AON) treatment period and the second alternating period is a no-treatment or placebo treatment period. In some embodiments, the first alternating period is a no-treatment or placebo treatment period and the second alternating period is a drug (e.g., SMAD7 AON) treatment period. The two or more alternating periods in an alternating dosing schedule can have the same length, or the alternating periods can each individually differ in length. For example, a first alternating period can be longer or shorter than a second alternating period.

Alternating periods can have a length ranging from days, to weeks, to months, to years. In some embodiments, the alternating periods can each individually be between 1 week and 7 weeks, between 2 weeks and 6 weeks, or between 3 weeks and 5 weeks. In some embodiments, the alternating periods can each individually be between 1 week and 15 weeks, between 2 weeks and 14 weeks, between 3 weeks and 13 weeks, between 4 weeks and 12 weeks, between 5 weeks and 11 weeks, between 6 weeks and 10 weeks, or between 7 weeks and 9 weeks. In some embodiments, an alternating period can be 4 weeks. In some embodiments, an alternating period can be 8 weeks. In some embodiments, an alternating period can be at least one month, at least 2 months, at least 3 months, at least 4 months, at least 5 months, at least 6 months, at least 7 months, at least 8 months, at least 9 months, at least 10 months, at least 11 months, or at least 12 months.

In some embodiments, the alternating dosing schedule starts with a drug treatment period that is then followed by a no-treatment or placebo treatment period. In some embodiments, the alternating dosing schedule, e.g., during a second or third treatment period, starts with a no-treatment or placebo treatment period that is then followed by a drug treatment period.

In some embodiments, in an alternating dosing schedule, the SMAD7 AON treatment period occurs first (e.g., during a first alternating period) and the no-treatment or placebo treatment period occurs second (e.g., during a second alternating period). In some embodiments, in the alternating dosing schedule, the no-treatment or placebo treatment period occurs first (e.g., during a first alternating period) and the SMAD7 AON treatment period occurs second (e.g., during a second alternating period).

In some embodiments, the drug treatment period and the no-treatment or placebotreatment periods are of the same length (e.g., 4 weeks each). In some embodiments, the drug treatment period and the no-treatment period are of different lengths (e.g., drug treatment period of 2 weeks, followed by a no-treatment period of 4 weeks). In some embodiments, the drug treatment period is longer than the no-treatment or placebo treatment period. In some embodiments, the no-treatment or placebo treatment period is longer than the drug treatment period.

In some embodiments, the SMAD7 AON is administered continuously during the first treatment period and during the second treatment period. In some embodiments, the SMAD7 AON is administered continuously during the first treatment period and is administered on an alternating dosing schedule during the second treatment period. In some embodiments, the SMAD7 AON is administered on an alternating dosing schedule during the first treatment period and is administered continuously during the second treatment period. In some embodiments, the SMAD7 AON is administered on an alternating dosing schedule during the first treatment period and during the second treatment period.

In some embodiments, the SMAD7 AON is administered continuously during the first, second and (optional) third treatment periods. In some embodiments, the SMAD7 AON is administered continuously during the first and (optional) third treatment periods and is administered on an alternating dosing schedule during the second treatment period. In some embodiments, the SMAD7 AON is administered continuously during the first and second treatment periods and is administered on an alternating dosing schedule during the (optional) third treatment period. In some embodiments, the SMAD7 AON is administered continuously during the second and (optional) third treatment periods and is administered on an alternating dosing schedule during the first treatment period. In some embodiments, the SMAD7 AON is administered continuously during the first treatment period and is administered on an alternating dosing schedule during the second and (optional) third treatment periods. In some embodiments, the SMAD7 AON is administered continuously during the second treatment period and is administered on an alternating dosing schedule during the first and (optional) third treatment periods. In some embodiments, the SMAD7 AON is administered continuously during the (optional) third treatment period and is administered on an alternating dosing schedule during the first and second treatment periods. In some embodiments, the SMAD7 AON is administered on an alternating dosing schedule during the first, second and (optional) third treatment periods.

In some embodiments, continuously administering the SMAD7 AON comprises administering the SMAD7 AON daily (e.g., once daily, twice daily, and the like), weekly, biweekly or monthly, e.g., during the first, second and/or third treatment period.

In some embodiments, the alternating dosing schedule comprises a) administering the SMAD7 AON for a drug administration period; b) administering no SMAD7 AON or administering a placebo during a drug holiday period; and repeating a) and, optionally, b) one or more times.

In some embodiments, the alternating dosing schedule, e.g., during a second or third treatment period, comprises a) administering no SMAD7 AON or administering a placebo during a drug holiday period; b) administering the SMAD7 AON for a drug administration period; and repeating a) and, optionally, b) one or more times.

In some embodiments, a drug administration period for use with the treatment regimen provided herein can be between about 1 week and about 7 weeks, between about 2 weeks and about 6 weeks, or between about 3 weeks and about 5 weeks. In some embodiments, the drug treatment period for use with the treatment regimen provided herein can be between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks. In some embodiments, the drug treatment period for use with the treatment regimen provided herein can be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, or about 15 weeks.

In some embodiments, the drug administration period is up to 1 month, up to 2 months, up to 3 months, up to 4 months, up to 5 months, up to 6 months, up to 7 months, up to 8 months, up to 9 months, up to 10 months, up to 11 months, or up to 12 months.

In some embodiments, the drug administration period is about 1 month, about 2 months, about 3 months, about 4 months, about 5 months, about 6 months, about 7 months, about 8 months, about 9 months, about 10 months, about 11 months, or about 12 months.

In some embodiments, the drug holiday period for use with the treatment regimen provided herein can be between about 1 week and about 7 weeks, between about 2 weeks and about 6 weeks, or between about 3 weeks and about 5 weeks. In some embodiments, the drug holiday period for use with the treatment regimen provided herein can be between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks. In some embodiments, the drug holiday period for use with the treatment regimen provided herein can be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, or about 15 weeks.

In some embodiments, a drug administration period for use with the treatment regimen provided herein is about 4 weeks. In some embodiments, a drug holiday period for use with the treatment regimen provided herein is about 4 weeks.

In some embodiments, the alternating dosing schedule comprises drug holiday periods of between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks, which are alternating with drug administration periods of between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks.

In some embodiments, the alternating dosing schedule comprises drug holiday periods of between about 1 week and about 7 weeks, between about 2 weeks and about 6 weeks, or between about 3 weeks and about 5 weeks, which are alternating with drug administration periods of between about 1 week and about 7 weeks, between about 2 weeks and about 6 weeks, or between about 3 weeks and about 5 weeks.

In some embodiments, the alternating dosing schedule comprises drug holiday periods of up to 1 month, up to 2 months, up to 3 months, up to 4 months, up to 5 months, up to 6 months, up to 7 months, up to 8 months, up to 9 months, up to 10 months, up to 11 months, or up to 12 months, which are alternating with drug administration periods of up to 1 month, up to 2 months, up to 3 months, up to 4 months, up to 5 months, up to 6 months, up to 7 months, up to 8 months, up to 9 months, up to 10 months, up to 11 months, or up to 12 months.

In some embodiments, the alternating dosing schedule comprises drug holiday periods of about 1 month, about 2 months, about 3 months, about 4 months, about 5 months, about 6 months, about 7 months, about 8 months, about 9 months, about 10 months, about 11 months, or about 12 months, which are alternating with drug administration periods of about 1 month, about 2 months, about 3 months, about 4 months, about 5 months, about 6 months, about 7 months, about 8 months, about 9 months, about 10 months, about 11 months, or about 12 months.

In some embodiments, an alternating dosing schedule is applied during the first, second, and/or third treatment period and comprises a) administering the SMAD7 antisense-oligonucleotide at the first, second, and/or third dose for between about 1 week and about 7 weeks, between about 2 weeks and about 6 weeks, or between about 3 weeks and about 5 weeks; b) administering a placebo or no SMAD7 antisense-oligonucleotide for between about 1 week and about 7 weeks, between about 2 weeks and about 6 weeks, or between about 3 weeks and about 5 weeks; and repeating a) and, optionally, b) one or more times.

In some embodiments, an alternating dosing schedule is applied during the first, second, or third treatment period and comprises a) administering a placebo or no SMAD7 antisense-oligonucleotide for between about 1 week and about 7 weeks, between about 2 weeks and about 6 weeks, or between about 3 weeks and about 5 weeks; b) administering the SMAD7 antisense-oligonucleotide at the first, second, or third dose for between about 1 week and about 7 weeks, between about 2 weeks and about 6 weeks, or between about 3 weeks and about 5 weeks; and repeating a) and, optionally, b) one or more times.

As discussed in Section 7.1.1.4, the total length of the second and/or third treatment periods can be at least about 1 week, at least about 2 weeks, at least about 4 weeks, at least about 6 weeks, at least about 8 weeks, at least about 10 weeks, at least about 3 months, at least about 6 months, at least about 9 months, at least about 12 months, at least about 18 months, at least about 24 months, at least about 30 months, at least about 3 years, at least about 4 years, at least about 5 years, at least about 6 years, at least about 7 years, at least about 8 years, at least about 9 years, or at least about 10 years.

In some embodiments, the total length of the second/and or third treatment period can be the length of the patient's remaining life span.

In some embodiments, the alternating dosing schedule is applied during the first, second or third treatment period and comprises a) administering the SMAD7 antisense-oligonucleotide at the first, second, or third dose for between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks; b) administering a placebo or no SMAD7 antisense-oligonucleotide for between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks; and repeating a) and optionally b) one or more times.

In some embodiments, the alternating dosing schedule is applied during the first, second, and/or third treatment period and comprises a) administering a placebo or no SMAD7 antisense-oligonucleotide for between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks; b) administering the SMAD7 antisense-oligonucleotide at the first, second and/or third dose for between about 1 week and about 15 weeks, between about 2 weeks and about 14 weeks, between about 3 weeks and about 13 weeks, between about 4 weeks and about 12 weeks, between about 5 weeks and about 11 weeks, between about 6 weeks and about 10 weeks, or between about 7 weeks and about 9 weeks; and repeating a) and optionally b) one or more times.

In some embodiments, the alternating dosing schedule is applied during the first, second, and/or third treatment period and comprises a) administering the SMAD7 antisense-oligonucleotide at the first, second and/or third dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and, optionally, b) one or more times.

In some embodiments, the alternating dosing schedule is applied during the first, second, and/or third treatment period and comprises a) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering the SMAD7 antisense-oligonucleotide at the first, second, and/or third dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and, optionally, b) one or more times.

In some embodiments, the alternating dosing schedule is applied during the first, second, and/or third treatment period and comprises a) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering the SMAD7 antisense-oligonucleotide at the first, second, and/or third dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and, optionally, b) one or more times.

In some embodiments, the alternating dosing schedule is applied during the first, second, and/or third treatment period and comprises a) administering the SMAD7 antisense-oligonucleotide at the first, second and/or third dose for at least about month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months; b) administering a placebo or no SMAD7 antisense-oligonucleotide for at least about month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months; and repeating a) and, optionally, b) one or more times.

In some embodiments, the alternating dosing schedule is applied during the first, second, or third treatment period and comprises a) administering the SMAD7 antisense-oligonucleotide at the first, second, or third dose for about 2 weeks; b) administering a placebo or no SMAD7 antisense-oligonucleotide for about 2 weeks; and repeating a) and, optionally, b) 5 more times.

In some embodiments, the alternating dosing schedule is applied during the first, second, or third treatment period and comprises a) administering the SMAD7 antisense-oligonucleotide at the first, second, or third dose for about 4 weeks; b) administering a placebo or no SMAD7 antisense-oligonucleotide for about 4 weeks; and repeating a) and, optionally, b) 2 more times.

In some embodiments, the alternating dosing schedule is applied during the first, second, or third treatment period and comprises a) administering the SMAD7 antisense-oligonucleotide at the first, second, or third dose for about 4 weeks; b) administering a placebo or no SMAD7 antisense-oligonucleotide for about 4 weeks; and repeating a) and, optionally, b) 6 more times.

In some embodiments, the alternating dosing schedule is applied during the first, second, and/or third treatment period and comprises a) administering a placebo or no SMAD7 antisense-oligonucleotide for about 4 weeks; b) administering the SMAD7 antisense-oligonucleotide at the first, second, and/or third dose for about 4 weeks; and repeating a) and, optionally b) 2 more times.

In some embodiments, the alternating dosing schedule is applied during the first, second, and/or third treatment period and comprises a) administering a placebo or no SMAD7 antisense-oligonucleotide for about 4 weeks; b) administering the SMAD7 antisense-oligonucleotide at the first, second, and/or third dose for about 4 weeks; and repeating a) and, optionally, b) 6 more times.

In some embodiments, the alternating dosing schedule is applied during the first, second, or third treatment period and comprises a) administering the SMAD7 antisense-oligonucleotide at the first, second, or third dose for about 4 weeks; b) administering a placebo or no SMAD7 antisense-oligonucleotide for about 8 weeks; and repeating a) and, optionally, b) 4 more times.

In some embodiments, the alternating dosing schedule is applied during the first, second, or third treatment period and comprises a) administering a placebo or no SMAD7 antisense-oligonucleotide for about 8 weeks; b) administering the SMAD7 antisense-oligonucleotide at the first, second, or third dose for about 4 weeks; and repeating a) and, optionally b) 4 more times.

In some embodiments, the alternating dosing schedule is applied during the third treatment period and comprises a) administering the SMAD7 antisense-oligonucleotide at the second dose for about 4 weeks; b) administering a placebo or no SMAD7 antisense-oligonucleotide for about 4 weeks; and repeating a) and optionally b) 25 more times.

In some embodiments, the alternating dosing schedule is applied during the third treatment period and comprises a) administering a placebo or no SMAD7 antisense-oligonucleotide for about 4 weeks; b) administering the SMAD7 antisense-oligonucleotide at the second dose for about 4 weeks; and repeating a) and, optionally b) 25 more times.

In some embodiments, a) and, optionally, b) are repeated at least 1 time, at least 2 times, at least 4 times, at least 6 times, at least 8 times, at least 10 times, at least 12 times, at least 14 times, at least 16 times, at least 18 times, at least 20 times, at least 25 times, at least 50 times, at least 100 times, at least 150 times, at least 200 times, or at least 250 times.

In some embodiments, a) and, optionally, b) are repeated at least 1 more time, at least 2 more times, at least 3 more times, at least 4 more times, at least 5 more times, at least 6 more times, at least 7 more times, at least 8 more times, at least 9 more times, at least 10 more times, at least 11 more times, at least 12 more times, at least 13 more times, at least 14 more times, at least 15 more times, at least 16 more times, at least 17 more times, at least 18 more times, at least 19 more times, at least 20 more times, at least 21 more times, at least 22 more times, at least 23 more times, at least 24 more times, at least 25 more times, at least 26 more times, at least 27 more times, at least 28 more times, at least 29 more times, at least 30 more times, at least 31 more times, at least 32 more times, at least 33 more times, at least 34 more times, at least 35, at least 40, at least 50, at least 60, at least 70, at least 80, at least 90, or at least 100 more times.

In some embodiments, a) and, optionally, b) are repeated up to 5 more times, up to 10 more times, up to 15 more times, up to 20 more times, up to 25 more times, up to 30 more times, up to 35 more times, up to 40 more times, up to 45 more times, up to 50, up to 60, up to 70, up to 80, up to 90, or up to 100 more times.

In some embodiments, the alternating dosing schedule comprises a) administering the SMAD7 antisense-oligonucleotide at the second dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and optionally b) one or more times.

In some embodiments, a) and optionally b) is repeated at least 2 times, at least 4 times, at least 6 times, at least 8 times, at least 10 times, at least 12 times, at least 14 times, at least 16 times, at least 18 times, at least 20 times, at least 25 times, at least 50 times, at least 100 times, at least 150 times, at least 200 times, or at least 250 times.

In some embodiments, the alternating dosing schedule comprises a) administering the SMAD7 antisense-oligonucleotide at the second dose for about 4 weeks; b) administering no SMAD7 antisense-oligonucleotide for about 4 weeks; and repeating a) and b) two times.

In some embodiments, the alternating dosing schedule comprises a) administering the SMAD7 antisense-oligonucleotide at the second dose for about 4 weeks; b) administering no SMAD7 antisense-oligonucleotide for about 8 weeks; and repeating a) and b) two times.

7.1.3 Illustrative Treatment Regimens

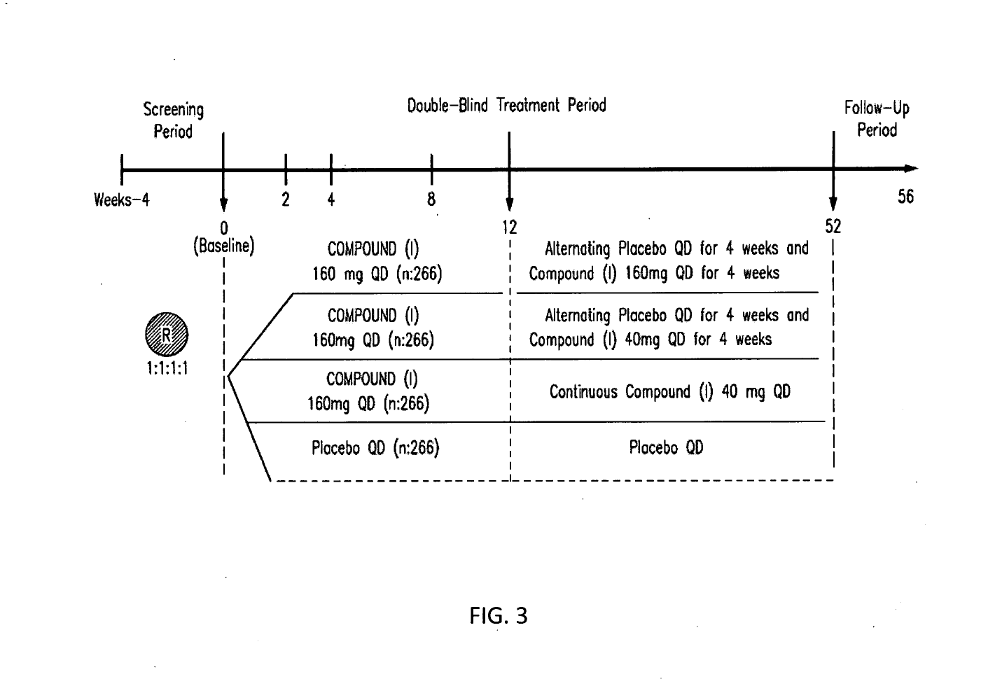

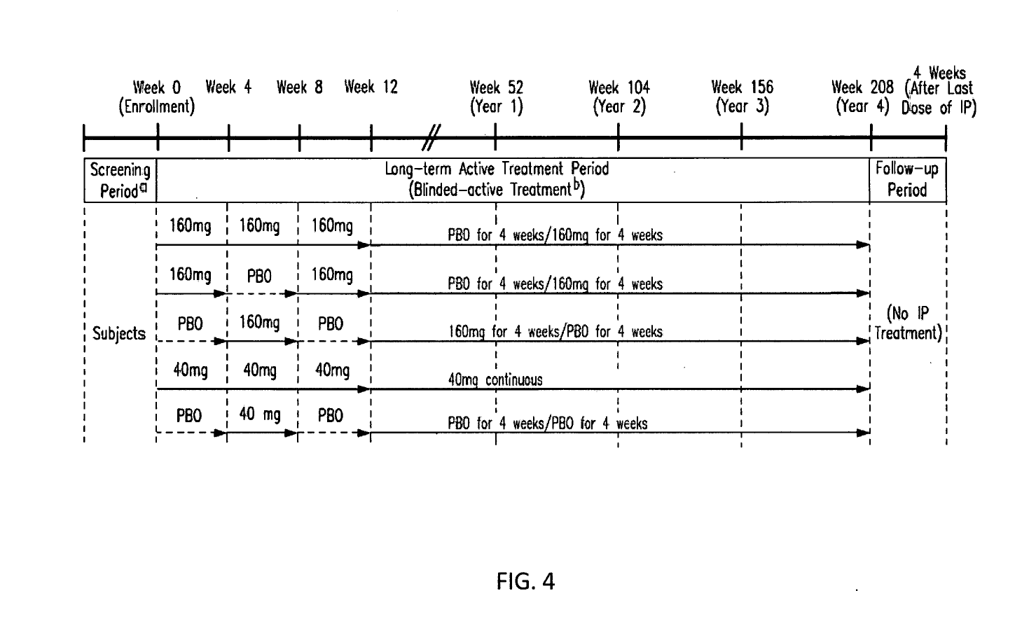

In some embodiments, a method for use with the treatment regimens provided herein comprises (a) continuously administering to an IBD patient a SMAD7 AON for a first treatment period at a first once-daily dose; (b) continuously administering to the IBD patient the SMAD7 AON for a second treatment period at a second once-daily dose; and, optionally, (c) administering to the IBD patient the SMAD7AON for a third treatment period at a third dose. See, e.g., FIG. 3 and FIG. 4; Example 2.

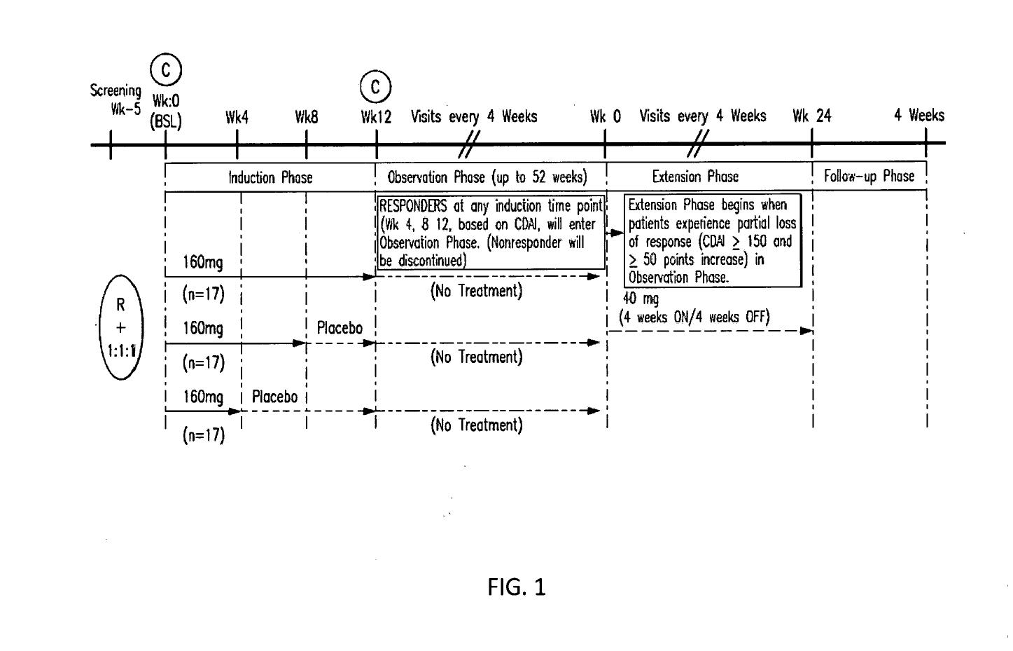

In some embodiments, a method for use with the treatment regimens provided herein comprises (a) continuously administering to an IBD patient a SMAD7 AON for a first treatment period at a first once-daily dose; (b) administering to the IBD patient the SMAD7 AON for a second treatment period at a second once-daily dose using an alternating dosing schedule, wherein the alternating dosing schedule comprises c) administering to the IBD patient a placebo or no SMAD7 AON for a first alternating period; d) administering to the IBD patient the SMAD7 AON at the second once-daily dose for a second alternating period, and (e) repeating (c) and (d) one or more times, and, optionally, f) administering to the IBD patient the SMAD7 AON for a third treatment period at a third dose (e.g., continuously, or using an alternating dosing schedule). See, e.g., FIG. 1 and FIGS. 3-6; Examples 1-5. In some embodiments, in the alternating dosing schedule, the SMAD7 AON treatment period occurs first (during the first alternating period) and the no-treatment or placebo treatment period occurs second (during the second alternating period). In some embodiments, in the alternating dosing schedule, the no-treatment or placebo treatment period occurs first (during the first alternating period) and the SMAD7 AON treatment period occurs second (during the second alternating period).

In some embodiments, a method for use with the treatment regimens provided herein comprises (a) administering to an IBD patient the SMAD7 AON for a first treatment period at a first once-daily dose using an alternating dosing schedule, wherein the alternating dosing schedule comprises (b) administering to the IBD patient the SMAD7 AON at the first once-daily dose for a first alternating period, (c) administering to the IBD patient a placebo or no SMAD7 AON for a second alternating period, and (d) repeating (b) and (c) one or more times; (e) administering to the IBD patient the SMAD7 AON for a second treatment period at a second once-daily dose using an alternating dosing schedule, wherein the alternating dosing schedule comprises (f) administering to the IBD patient the SMAD7 AON at the second once-daily dose for a third alternating period; (g) administering to the IBD patient a placebo or no SMAD7 AON for a fourth alternating period, and (h) repeating (f) and (g) one or more times, and, optionally, (i) administering to the IBD patient the SMAD7 AON for a third treatment period at a third dose (e.g., continuously, or using an alternating dosing schedule). See, e.g., FIG. 4; Example 3. In some embodiments, independently in each individual alternating dosing schedule, the SMAD7 AON treatment period occurs first (e.g., during the first alternating period) and the no-treatment or placebo treatment period occurs second (e.g., during the second alternating period). In some embodiments, independently in each alternating dosing schedule, the no-treatment or placebo treatment period occurs first (e.g., during the first alternating period) and the SMAD7 AON treatment period occurs second (e.g, during the second alternating period).

In some embodiments, administering to the IBD patient the SMAD7AON for a third treatment period at a third dose comprises continuously administering to the IBD patient the SMAD7 AON at a third once-daily dose.

In some embodiments, administering to the IBD patient the SMAD7AON for a third treatment period at a third dose comprises using an alternating dosing schedule, wherein the alternating dosing schedule comprises (a) administering to the IBD patient a placebo or no SMAD7 AON for a first alternating period; (b) administering to the IBD patient the SMAD7 AON at the third once-daily dose for a second alternating period, and (c) repeating (a) and (b) one or more times. In some embodiments, in the alternating dosing schedule, the SMAD7 AON treatment period occurs first (during the first alternating period) and the no-treatment or placebo treatment period occurs second (during the second alternating period). In some embodiments, in the alternating dosing schedule, the no-treatment or placebo treatment period occurs first (during the first alternating period) and the SMAD7 AON treatment period occurs second (during the second alternating period).

In some embodiments, the patient transitions directly from the first to the second treatment period, from the second to the third treatment period, and/or from the first to the optional third treatment period.

In some embodiments, the patient transitions through an intermediate period, such as an observation period from the first to the second treatment period, from the second to the third treatment period, and/or from the first to the optional third treatment period.

In some embodiments, the durations of the first, second and/or third treatment periods are not predetermined, but depends on the patient's response to treatment, e.g., as determined by results from colonoscopy, ilonooscopy, biomarker levels or others (e.g., achievement or maintenance for a specific time of CDAI <150, SES-CD ≤2, PRO-2 score ≤8, CRP levels <1.0 mg/L, average daily liquid or soft stool frequency ≤3.0 or ≤1.5 points and/or decrease of abdominal pain score ≤1 point; TMS score ≤2; ES=0 or 1; PMS ≤2 points; MMS ≤2). In some embodiments, the transition of a patient from one to another treatment period, e.g., the transition of the patient from the first to the second treatment period, is triggered by the patient's response to treatment.

In some embodiments, in an alternative dosing schedule, the SMAD7 AON treatment period is of the same length as the alternating no-treatment or placebo treatment period. In some embodiments, the SMAD7 AON treatment period and the no-treatment or placebo treatment periods are of different lengths. In some embodiments, the SMAD7 AON treatment period is longer than the no-treatment period or the placebo treatment period. In some embodiments, the no-treatment or placebo treatment period is longer than the SMAD7 AON treatment period.

As described in Section 7.1.2, the alternating periods described herein periods can have a length ranging from days, to weeks, to months, to years. In some embodiments, the alternating periods can each individually be between 1 week and 7 weeks, between 2 weeks and 6 weeks, or between 3 weeks and 5 weeks. In some embodiments, the alternating periods can each individually be between 1 week and 15 weeks, between 2 weeks and 14 weeks, between 3 weeks and 13 weeks, between 4 weeks and 12 weeks, between 5 weeks and 11 weeks, between 6 weeks and 10 weeks, or between 7 weeks and 9 weeks. In some embodiments, an alternating period can be 4 weeks. In some embodiments, an alternating period can be 8 weeks. In some embodiments, an alternating period can be at least one month, at least 2 months, at least 3 months, at least 4 months, at least 5 months, at least 6 months, at least 7 months, at least 8 months, at least 9 months, at least 10 months, at least 11 months, or at least 12 months.

In one embodiment, a method for use with the treatment regimens provided herein comprises (a) administering to a CD patient a SMAD7 AON for a period of about 4 weeks, about 8 weeks, or about 12 weeks at a once-daily dose of about 160 mg; and (b) administering to the CD patient the SMAD7 AON for about 24 weeks at a once-daily dose of about 40 mg using an alternating dosing schedule. See, e.g., FIG. 1. In some embodiments, the alternating dosing schedule comprises three drug treatment periods of 4-weeks each (weeks 0-3, weeks 8-11, and weeks 16-19) that are alternating with three drug holiday periods of 4 weeks each (weeks 4-7, weeks 12-15, and weeks 20-23). See, e.g., Example 1, Table 3.

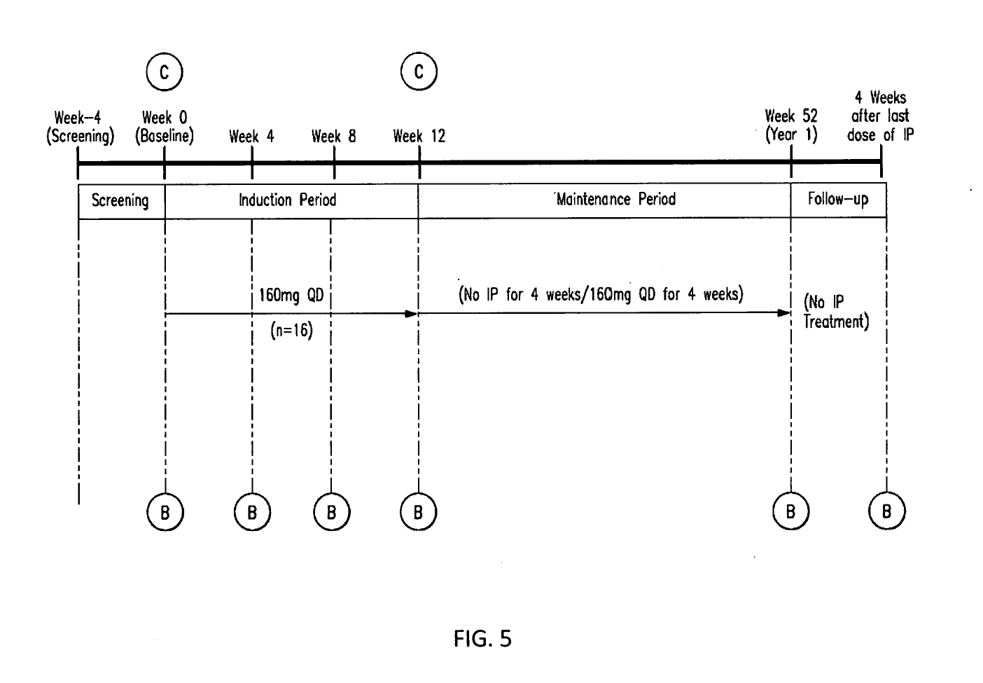

In one embodiment, a method for use with the treatment regimens provided herein comprises (a) administering to an IBD patient a SMAD7 AON for a period of about 12 weeks at a once-daily dose of about 160 mg; and (b) administering to the IBD patient the SMAD7 AON for about 40 weeks at a once-daily dose of about 40 mg or 160 mg using an alternating dosing schedule, wherein the alternating dosing schedule comprises c) administering to the IBD patient a placebo or no SMAD7 AON for about 4 weeks; d) administering to the IBD patient the SMAD7 AON at a once-daily dose of about 40 mg or about 160 mg; and repeating c) and d) for a period of time. See, e.g., FIG. 3 and FIG. 5. In some embodiments, the period of time is up to about 40 weeks. In some embodiments, the period of time is longer than about 40 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In some embodiments, the alternating dosing schedule comprises five SMAD7 AON treatment periods of 4-weeks each (weeks 16-19, weeks 24-27, weeks 32-35, weeks 40-43, and weeks 48-51) that are alternating with five no-treatment or placebo-treatment periods of 4 weeks each (weeks 12-15, weeks 20-23, weeks 28-31, weeks 36-39, and weeks 44-47). See, e.g., Example 2, Table 4.

In one embodiment, a method for use with the treatment regimens provided herein comprises (a) administering to an IBD patient a SMAD7 AON for a period of about 12 weeks at a once-daily dose of about 160 mg; and (b) administering to the IBD patient the SMAD7 AON for a period of time at a once-daily dose of about 40 mg. See, e.g., FIG. 3 and Example 2, Table 4. In some embodiments, the period of time is up to about 40 weeks. In some embodiments, the period of time is longer than about 40 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In one embodiment, a method for use with the treatment regimens provided herein comprises (a) administering to an IBD patient a SMAD7 AON for a period of about 12 weeks at a once-daily dose of about 160 mg; and (b) administering to the IBD patient the SMAD7 AON for up to about 196 weeks at a once-daily dose of about 160 mg using an alternating dosing schedule, wherein the alternating dosing schedule comprises c) administering to the IBD patient a placebo or no SMAD7 AON for about 4 weeks; d) administering to the IBD patient the SMAD7 AON at a once-daily dose of about 160 mg for about 4 weeks; and repeating c) and d) for a period of time. See, e.g., FIG. 4. In some embodiments, the period of time is up to about 196 weeks. In some embodiments, the period of time is longer than about 196 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In some embodiments, the alternating dosing schedule comprises up to twenty-four SMAD7 AON treatment periods of 4-weeks each (weeks 16-19, weeks 24-27, weeks 32-35, weeks 40-43, weeks 48-51, weeks 56-59, weeks 64-67, weeks 72-75, weeks 80-83, weeks 88-91, weeks 96-99, weeks 104-107, weeks 112-115, weeks 120-123, weeks 128-131, weeks 136-139, weeks 144-147, weeks 152-155, weeks 160-163, weeks 168-171, weeks, 176-179, weeks 184-187, weeks 192-195, and weeks 200-203) that are alternating with up to twenty-five no-treatment or placebo treatment periods of 4 weeks each (weeks 12-15, weeks 20-23, weeks 28-31, weeks 36-39, weeks 44-47, weeks 52-55, weeks 60-63, weeks 68-71, weeks 76-79, weeks 84-87, weeks 92-95, weeks 100-103, weeks 108-111, weeks 116-119, weeks 124-127, weeks 132-135, weeks 140-143, weeks 148-151, weeks 156-159, weeks 164-167, weeks 172-175, weeks 180-183, weeks 188-191, weeks 196-199, and weeks 204-207). See, e.g., Example 3, Tables 7-10.

In one embodiment, a method for use with the treatment regimens provided herein comprises (a) administering to an IBD patient a SMAD7 AON for a period of about 12 weeks at a once-daily dose of about 160 mg using an alternating dosing schedule, wherein the alternating dosing schedule comprises b) administering to the IBD patient the SMAD7 AON at a once-daily dose of about 160 mg for about 4 weeks; c) administering to the IBD patient a placebo or no SMAD7 AON for a period of about 4 weeks; and repeating b) once; d) administering to the IBD patient the SMAD7 AON for up to about 196 weeks at a once-daily dose of up to about 160 mg using an alternating dosing schedule, wherein the alternating dosing schedule comprises e) administering to the IBD patient a placebo or no SMAD7 AON for about 4 weeks; f) administering to the IBD patient the SMAD7 AON at a once-daily dose of about 160 mg for about 4 weeks; and repeating e) and f) for a period of time. See, e.g., FIG. 4. In some embodiments, the period of time is up to about 196 weeks. In some embodiments, the period of time is longer than about 196 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In some embodiments, the alternating dosing schedule comprises up to twenty-six SMAD7 AON treatment periods of 4-weeks each (weeks 0-3, weeks 8-11, weeks 16-19, weeks 24-27, weeks 32-35, weeks 40-43, weeks 48-51, weeks 56-59, weeks 64-67, weeks 72-75, weeks 80-83, weeks 88-91, weeks 96-99, weeks 104-107, weeks 112-115, weeks 120-123, weeks 128-131, weeks 136-139, weeks 144-147, weeks 152-155, weeks 160-163, weeks 168-171, weeks, 176-179, weeks 184-187, weeks 192-195, and weeks 200-203) that are alternating with up to twenty-six no-treatment or placebo treatment periods of 4 weeks each (weeks, 4-7, weeks 12-15, weeks 20-23, weeks 28-31, weeks 36-39, weeks 44-47, weeks 52-55, weeks 60-63, weeks 68-71, weeks 76-79, weeks 84-87, weeks 92-95, weeks 100-103, weeks 108-111, weeks 116-119, weeks 124-127, weeks 132-135, weeks 140-143, weeks 148-151, weeks 156-159, weeks 164-167, weeks 172-175, weeks 180-183, weeks 188-191, weeks 196-199, and weeks 204-207). See, e.g., Example 3, Tables 7-10, FIG. 4.

In some embodiments, the alternating dosing schedule comprises up to twenty-six SMAD7 no-treatment or placebo treatment periods of 4-weeks each (weeks 0-3, weeks 8-11, weeks 16-19, weeks 24-27, weeks 32-35, weeks 40-43, weeks 48-51, weeks 56-59, weeks 64-67, weeks 72-75, weeks 80-83, weeks 88-91, weeks 96-99, weeks 104-107, weeks 112-115, weeks 120-123, weeks 128-131, weeks 136-139, weeks 144-147, weeks 152-155, weeks 160-163, weeks 168-171, weeks, 176-179, weeks 184-187, weeks 192-195, and weeks 200-203) that are alternating with up to twenty-six SMAD7 AON treatment periods of 4 weeks each (weeks, 4-7, weeks 12-15, weeks 20-23, weeks 28-31, weeks 36-39, weeks 44-47, weeks 52-55, weeks 60-63, weeks 68-71, weeks 76-79, weeks 84-87, weeks 92-95, weeks 100-103, weeks 108-111, weeks 116-119, weeks 124-127, weeks 132-135, weeks 140-143, weeks 148-151, weeks 156-159, weeks 164-167, weeks 172-175, weeks 180-183, weeks 188-191, weeks 196-199, and weeks 204-207). See, e.g., Example 3, Tables 7-10, FIG. 4.

In one embodiment, a method for use with the treatment regimens provided herein comprises (a) administering to an IBD patient a SMAD7 AON for a period of about 12 weeks at a once-daily dose of about 40 mg; and (b) administering to the IBD patient the SMAD7 AON for a period of time at a once-daily dose of about 40 mg. See, e.g., FIG. 4 and Example 3, Tables 7-10. In some embodiments, the period of time is up to about 196 weeks. In some embodiments, the period of time is longer than about 196 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In one embodiment, a method for use with the treatment regimens provided herein comprises (a) administering to an IBD patient a SMAD7 AON for a period of about 12 weeks at a once-daily dose of about 40 mg using an alternating dosing schedule, wherein the alternating dosing schedule comprises b) administering to the IBD patient a placebo or no SMAD7 AON for a period of about 4 weeks; c) administering to the IBD patient the SMAD7 AON at a once-daily dose of about 40 mg for about 4 weeks; and repeating b) once; d) administering to the IBD patient the SMAD7 AON for up to about 196 weeks at a once-daily dose of up to about 40 mg using an alternating dosing schedule, wherein the alternating dosing schedule comprises e) administering to the IBD patient the SMAD7 AON at a once-daily dose of about 40 mg for about 4 weeks; f) administering to the IBD patient a placebo or no SMAD7 AON for about 4 weeks; and repeating e) and f) for a total of up to about 196 weeks. See, e.g., FIG. 4. In some embodiments, the period of time is up to about 196 weeks. In some embodiments, the period of time is longer than about 196 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is CD.

In some embodiments, the alternating dosing schedule comprises up to twenty-six drug treatment periods of 4-weeks each (weeks 4-7, weeks 12-15, weeks 20-23, weeks 28-31, weeks 36-39, weeks 44-47, weeks 52-55, weeks 60-63, weeks 68-71, weeks 76-79, weeks 84-87, weeks 92-95, weeks 100-103, weeks 108-111, weeks 116-119, weeks 124-127, weeks 132-135, weeks 140-143, weeks 148-151, weeks 156-159, weeks 164-167, weeks 172-175, weeks 180-183, weeks 188-191, weeks 196-199, and weeks 204-207) that are alternating with up to twenty-six drug holiday periods of 4 weeks each (weeks 0-3, weeks 8-11, weeks 16-19, weeks 24-27, weeks 32-35, weeks 40-43, weeks 48-51, weeks 56-59, weeks 64-67, weeks 72-75, weeks 80-83, weeks 88-91, weeks 96-99, weeks 104-107, weeks 112-115, weeks 120-123, weeks 128-131, weeks 136-139, weeks 144-147, weeks 152-155, weeks 160-163, weeks 168-171, weeks, 176-179, weeks 184-187, weeks 192-195, and weeks 200-203). See, e.g., Example 3, Tables 7-10.

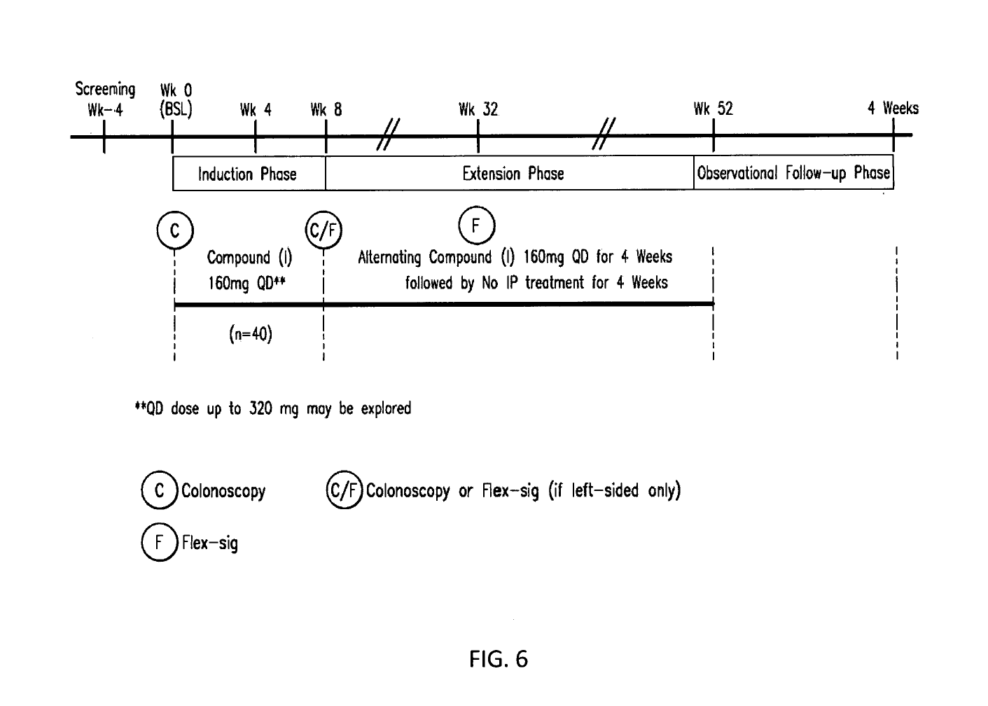

In one embodiment, a method for use with the treatment regimens provided herein comprises (a) administering to an IBD patient a SMAD7 AON for a period of about 8 weeks at a once-daily dose of about 160 mg; and (b) administering to the IBD patient the SMAD7 AON for up to about 44 weeks at a once-daily dose of about 160 mg using an alternating dosing schedule, wherein the alternating dosing schedule comprises c) administering to the IBD patient the SMAD7 AON at a once-daily dose of about 160 mg for about 4 weeks; d) administering a placebo or no SMAD7 AON for about 4 weeks; and repeating c) and d) for a period of time. See, e.g., FIG. 6. In some embodiments, the period of time is up to about 44 weeks. In some embodiments, the period of time is longer than about 44 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is UC.

In some embodiments, the alternating dosing schedule comprises up to five SMAD7 AON treatment periods of 4-weeks each (weeks 12-15, weeks 20-23, weeks 28-31, weeks 36-39, and weeks 44-47) that are alternating with up to six no treatment or placebo treatment periods of 4 weeks each (weeks 8-11, weeks 16-19, weeks 24-27, weeks 32-35, weeks 40-43, and weeks 48-51). See, e.g., FIG. 4.

In one embodiment, a method for use with the treatment regimens provided herein comprises (a) administering to an IBD patient a SMAD7 AON for a period of about 8 weeks at a once-daily dose of about 320 mg; and (b) administering to the IBD patient the SMAD7 AON for up to about 44 weeks at a once-daily dose of about 320 mg using an alternating dosing schedule, wherein the alternating dosing schedule comprises c) administering to the IBD patient the SMAD7 AON at a once-daily dose of about 320 mg for about 4 weeks; d) administering to the IBD patient a placebo or no SMAD7 AON for about 4 weeks; and repeating c) and d) for a period of time. See, e.g., FIG. 6. In some embodiments, the period of time is up to about 44 weeks. In some embodiments, the period of time is longer than about 44 weeks. In some embodiments, the period of time is not predetermined, but based on a patient's clinical response to treatment, as, e.g., determined by colonoscopy, or iliocolonoscopy tests, biomarker levels, patient reported outcomes, or other tests described herein. In some embodiments, the IBD is UC.

In some embodiments, the alternating dosing schedule comprises up to five SMAD7 AON treatment periods of 4-weeks each (weeks 12-15, weeks 20-23, weeks 28-31, weeks 36-39, and weeks 44-47) that are alternating with up to six placebo or no-treatment periods of 4 weeks each (weeks 8-11, weeks 16-19, weeks 24-27, weeks 32-35, weeks 40-43, and weeks 48-51). See, e.g., FIG. 6.

In any embodiments comprising an alternating dosing schedule, the alternating dosing schedule can start with either a drug administration (e.g., SMAD7 AON administration) or with the administration of a placebo or no treatment.

In some embodiments, in one or more alternating dosing schedules, the SMAD7 AON is administered first and the and the placebo or no treatment is administered second.

In some embodiments, in one or more alternating dosing schedules the placebo or no treatment is administered first and the SMAD7 AON is administered second.

Any administration schedule described herein can be preceeded by the same or by any other administration schedule described herein.

In some embodiments, the IBD patient is a CD patient. In some embodiments, the IBD patient is a UC patient

As discussed in Sections 7.1.1.4. and 7.1.1.8, in some embodiments, the total length of the second and/or third treatment period can be at least about 1 week, at least about 2 weeks, at least about 4 weeks, at least about 6 weeks, at least about 8 weeks, at least about 10 weeks, at least about 3 months, at least about 6 months, at least about 9 months, at least about 12 months, at least about 18 months, at least about 24 months, at least about 30 months, at least about 3 years, at least about 4 years, at least about 5 years, at least about 6 years, at least about 7 years, at least about 8 years, at least about 9 years, or at least about 10 years.

In some embodiments, a method for use with the treatment regimens provided herein comprises (a) administering to a CD patient a SMAD7 AON for a period of about 12 weeks at a once-daily dose of about 160 mg; and (b) administering to the CD patient the SMAD7 AON for about 24 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) 2 more times. See e.g., Example 1, Table 3.

In some embodiments, a method for use with the treatment regimens provided herein comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 40 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 52 weeks.

In some embodiments, a method for use with the treatment regimens provided herein comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 40 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 8 weeks; and repeating c) and d) for a total of 52 weeks.

In some embodiments, a method for use with the treatment regimens provided herein comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 52 weeks.

In some embodiments, a method for use with the treatment regimens provided herein comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 8 weeks; and repeating c) and d) for a total of 52 weeks.

In some embodiments, a method for use with the treatment regimens provided herein method comprises (a) administering to a CD patient a SMAD7 AON for a period of between about 4 weeks and about 12 weeks (e.g., for about 4 weeks, about 5 weeks, about 6 weeks, about 7 weeks, about 8 weeks, about 9 weeks, about 10 weeks, about 11 weeks, or about 12 weeks) at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 52 weeks.

In some embodiments, a method for use with the treatment regimens provided herein comprises (a) administering to a CD patient a SMAD7 AON for a period of between about 4 weeks and about 12 weeks (e.g., for about 4 weeks, about 5 weeks, about 6 weeks, about 7 weeks, about 8 weeks, about 9 weeks, about 10 weeks, about 11 weeks, or about 12 weeks) at a once-daily dose of about 160 mg; and (b) administering to the CD patient the SMAD7 AON for about 52 weeks a once-daily dose of about 40 mg.

In some embodiments, a method for use with the treatment regimens provided herein comprises (a) administering to a CD patient a SMAD7 AON for a period of between about 4 weeks and about 12 weeks (e.g., for about 4 weeks, about 5 weeks, about 6 weeks, about 7 weeks, about 8 weeks, about 9 weeks, about 10 weeks, about 11 weeks, or about 12 weeks) at a once-daily dose of about 160 mg; and (b) administering to the CD patient the SMAD7 AON for about 52 weeks a once-daily dose of about 160 mg on an alternating dosing schedule, wherein the alternating dosing schedule comprises c) administering the SMAD7 antisense oligonucleotide at a once-daily dose of about 160 mg for about 4 weeks; d) administering a placebo or no SMAD7 AON for about 4 weeks; and repeating c) and d) for a total of 52 weeks.

In some embodiments, a method for use with the treatment regimens provided herein comprises (a) administering to a CD patient a SMAD7 AON for a period of between about 4 weeks and about 12 weeks (e.g., for about 4 weeks, about 5 weeks, about 6 weeks, about 7 weeks, about 8 weeks, about 9 weeks, about 10 weeks, about 11 weeks, or about 12 weeks) at a once-daily dose of about 160 mg; and (b) administering to the CD patient the SMAD7 AON for a bout 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 8 weeks; and repeating c) and d) for a total of 52 weeks. s.

The SMAD7 AON can be administered at any time during the day, including at night time. In some embodiments, the SMAD7 AON is administered in the morning (e.g., between about 5 am and about 11 am, e.g., at about 5 am, about 6 am, about 7 am, about 8 am, about 9 am, about 10 am, or about 11 am). In some embodiments, the SMAD7 AON is administered around noon (e.g., between about 11 am and about 1 pm, e.g., at about 12 am or about 1 pm). In some embodiments, the SMAD7 AON is administered in the afternoon (e.g., between about 1 pm and about 5 pm, e.g., at about 2 pm, about 3 pm, about 4 pm, or about 5 pm). In some embodiments, the SMAD7 AON is administered in the evening (e.g., between about 5 pm and about 10 pm, e.g., at about 6 pm, about 7 pm, about 8 pm, about 9 pm or about 10 pm). In some embodiments, the SMAD7 AON is administered at night (e.g., between about 10 pm and about 4 am, e.g., at about 11 pm, about 12 pm, about 1 am, about 2 am, about 3 am, or about 4 am).

In some embodiments, the method for treating or managing IBD in a patient having IBD comprises (a) administering to a CD patient a SMAD7 AON for a period of about 4 weeks, about 8 weeks, or about 12 weeks at a once-daily dose of about 160 mg; and (b) administering to the CD patient the SMAD7 AON for about 24 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 24 weeks.

In some embodiments, the method comprises (a) administering to a CD patient a SMAD7 AON for a period of about 12 weeks at a once-daily dose of about 160 mg; and (b) administering to the CD patient the SMAD7 AON for about 24 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 24 weeks.

In some embodiments, the method comprises (a) administering to a CD patient a SMAD7 AON for a period of between about 4 weeks and about 8 weeks at a once-daily dose of about 40 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) administering a placebo or no SMAD7 AON for about 4 weeks; and repeating c) and d) for a total of 52 weeks.

In some embodiments, the method comprises (a) administering to a CD patient a SMAD7 AON for a period of between about 4 weeks and about 8 weeks at a once-daily dose of about 40 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) optionally administering a placebo or no SMAD7 AON for about 8 weeks; and repeating c) and d) for a total of 52 weeks.

In some embodiments, the method comprises (a) administering to a CD patient a SMAD7 AON for a period of between about 4 weeks and about 8 weeks at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) optionally administering a placebo or no SMAD7 AON for about 4 weeks; and repeating c) and d) for a total of 52 weeks.

In some embodiments, the method comprises (a) administering to a CD patient a SMAD7 AON for a period of between about 4 weeks and about 8 weeks (e.g., for about 4 weeks, about 5 weeks, about 6 weeks, about 7 weeks, or about 8 weeks) at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) optionally administering a placebo or no SMAD7 AON for about 8 weeks; and repeating c) and d) for a total of 52 weeks.

In some embodiments, the method comprises (a) administering to a CD patient a SMAD7 AON for a period of between about 4 weeks and about 12 weeks at a once-daily dose of about 160 mg; and (b) administering to the CD patient the SMAD7 AON forabout 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) optionally administering a placebo or no SMAD7 AON for about 4 weeks; and repeating c) and d) for a total of 52 weeks.

In some embodiments, the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 40 mg.

In some embodiments, the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 160 mg on an alternating dosing schedule, wherein the alternating dosing schedule comprises c) administering the SMAD7 antisense oligonucleotide at a once-daily dose of about 160 mg for about 4 weeks; d) optionally administering a placebo or no SMAD7 AON for about 4 weeks; and repeating c) and d) for a total of 52 weeks.

In some embodiments, the method comprises (a) administering to a CD patient a SMAD7 AON for a period of between about 4 weeks and about 12 weeks at a once-daily dose of about 160 mg; and (b) administering to the CD patient the SMAD7 AON for about 52 weeks a once-daily dose of about 40 mg on an alternating dosing schedule, wherein the alternating dosing schedule comprises c) administering the SMAD7 antisense oligonucleotide at a once-daily dose of about 40 mg for about 4 weeks; d) optionally administering a placebo or no SMAD7 AON for about 8 weeks; and repeating c) and d) for a total of 52 weeks.

In some embodiments, in an alternating dosing schedule the SMAD7 AON is administered first and the placebo or no SMAD7 AON is administered second.

In some embodiments, in an alternating dosing schedule the placebo or no SMAD7 AON is administered first and the SMAD7 AON is administered second.

In some embodiments, the method comprises (a) administering to an IBD patient a SMAD7 AON for a first period at a once-daily dose of about 160 mg; and (b) administering to the IBD patient the SMAD7 AON for a second period at a once-daily dose of about 40 mg or 160 mg using an alternating dosing schedule, wherein the alternating dosing schedule comprises c) administering to the IBD patient a placebo or no SMAD7 AON for a first alternating period; d) administering to the IBD patient the SMAD7 AON at a once-daily dose of about 40 mg or about 160 mg for a second alternating period; and repeating c) and d) until the end of the second period.

In some embodiments, the first period is about 12 weeks, the second period is up to about 40 weeks, the first alternating period is about 4 weeks and the second alternating period is about 4 weeks.

In some embodiments, the method comprises (a) administering to an IBD patient a SMAD7 AON for a first period at a once-daily dose of about 160 mg; and (b) administering to the IBD patient the SMAD7 AON for a second period at a once-daily dose of about 40 mg.

In some embodiments, the first period is about 12 weeks and the second period is up to about 40 weeks.

In some embodiments, the method comprises (a) administering to an IBD patient a SMAD7 AON for a first period at a once-daily dose of about 160 mg; and (b) administering to the IBD patient the SMAD7 AON for a second period at a once-daily dose of about 160 mg using an alternating dosing schedule, wherein the alternating dosing schedule comprises c) administering to the IBD patient a placebo or no SMAD7 AON for a first alternating period; d) administering to the IBD patient the SMAD7 AON at a once-daily dose of about 160 mg for a second alternating period; and repeating c) and d) until the end of the second period.

In some embodiments, the second period is up to about 196 weeks, the first alternating period is about 4 weeks and the second alternating period is about 4 weeks.

In some embodiments, the method comprises (a) administering to an IBD patient a SMAD7 AON for a first period at a once-daily dose of about 160 mg using an alternating dosing schedule, wherein the alternating dosing schedule comprises b) administering to the IBD patient the SMAD7 AON at a once-daily dose of about 160 mg for a first alternating period; c) administering to the IBD patient a placebo or no SMAD7 AON for a second alternating period; and repeating b) until the end of the first period; d) administering to the IBD patient the SMAD7 AON for a second period at a once-daily dose of up to about 160 mg using an alternating dosing schedule, wherein the alternating dosing schedule comprises e) administering to the IBD patient a placebo or no SMAD7 AON for a third alternating period; f) administering to the IBD patient the SMAD7 AON at a once-daily dose of about 160 mg for a fourth alternating period; and repeating e) and f) until the end of the second period.

In some embodiments, the first period is about 12 weeks, the second period is up to about 196 weeks, and the first, second and third alternating periods each are about 4 weeks.

In some embodiments, the method comprises (a) administering to an IBD patient a SMAD7 AON for a first period at a once-daily dose of about 40 mg; and (b) administering to the IBD patient the SMAD7 AON for a second period a once-daily dose of about 40 mg.

In some embodiments, the first period is about 12 weeks and the second period is up to about 196 weeks.

In some embodiments, the method comprises (a) administering to an IBD patient a SMAD7 AON for a first period at a once-daily dose of about 40 mg using an alternating dosing schedule, wherein the alternating dosing schedule comprises b) administering to the IBD patient a placebo or no SMAD7 AON for a first alternating period; c) administering to the IBD patient the SMAD7 AON at a once-daily dose of about 40 mg for a second alternating period; and repeating b) until the end of the first period; d) administering to the IBD patient the SMAD7 AON for a second period at a once-daily dose of up to about 40 mg using an alternating dosing schedule, wherein the alternating dosing schedule comprises e) administering to the IBD patient the SMAD7 AON at a once-daily dose of about 40 mg for a third alternating period; f) administering to the IBD patient a placebo or no SMAD7 AON for a fourth alternating period; and repeating e) and f) until the end of the second period.

In some embodiments, the first period is about 12 weeks, the second period is up to about 196 weeks, and the first, second and third alternating periods each are about 4 weeks.

In some embodiments, the method comprises (a) administering to an IBD patient a SMAD7 AON for a first period at a once-daily dose of about 160 mg; and (b) administering to the IBD patient the SMAD7 AON for a second period at a once-daily dose of about 160 mg using an alternating dosing schedule, wherein the alternating dosing schedule comprises c) administering to the IBD patient the SMAD7 AON at a once-daily dose of about 160 mg for a first alternating period; d) administering a placebo or no SMAD7 AON for a second alternating period; and repeating c) and d) until the end of the second period.

In some embodiments, the first period is about 8 weeks, the second period is up to about 44 weeks, and the first, second and third alternating periods each are about 4 weeks.

In some embodiments, the method comprises (a) administering to an IBD patient a SMAD7 AON for a first period at a once-daily dose of up to 320 mg; and (b) administering to the IBD patient the SMAD7 AON for a second period at a once-daily dose of about 160 mg using an alternating dosing schedule, wherein the alternating dosing schedule comprises c) administering to the IBD patient the SMAD7 AON at a once-daily dose of about 160 mg for a first alternating period; d) administering to the IBD patient a placebo or no SMAD7 AON for a second alternating period; and repeating c) and d) until the end of the second period.

The method of claim 105, wherein the first period is about 8 weeks, wherein the second period is up to about 44 weeks, and wherein the first, second and third alternating periods each are about 4 weeks.

In some embodiments, in one or more alternating dosing schedules, the SMAD7 AON is administered first and the and the placebo or no SMAD7 AON is administered second.

In some embodiments, in one or more alternating dosing schedules the placebo or no SMAD7 AON is administered first and the SMAD7 AON is administered second.

7.1.4 Rates and Manner of Administration

In some embodiments, the SMAD7 AON is administered orally. In some embodiments, the SMAD7 AON is administered with food or drinks. In some embodiments, the SMAD7 AON is administered without food or drinks. In some embodiments, the SMAD7 AON is administered with a meal, such as breakfast, lunch, or dinner. The SMAD7 AON can be administered, e.g., shortly before, shortly after, or at the same time the meal is taken. In some embodiments, the SMAD7 AON is administered in the morning shortly before breakfast. In some embodiments, the SMAD7 AON is administered at least about 5 min, at least about 10 min, at least about 20 min, at least about 30 min, at least about 45 min, at least about 60 min, at least about 75 min, at least about 90 min, or at least about 120 min before a meal. In some embodiments, the SMAD7 AON is administered within about 5 min, within about 10 min, within about 20 min, within about 30 min, within about 45 min, within about 60 min, within about 75 min, within about 90 min, or within about 120 min after a meal.

In some embodiments, the SMAD7 AON is administered in the morning shortly before breakfast with water (e.g., a glass of water). In some embodiments, the SMAD7 AON is administered in the morning within about 30 min before breakfast.

In some embodiments, the SMAD7 AON is administered once a day, twice a day, or three times a day. In some embodiments, the SMAD7 AON is administered once a day. In some embodiments, the SMAD7 AON is administered once every 2 days, once every 3 days, once every 4 days, once every 5 days, once every 6 days, once every week, once every 10 days, once every two weeks, once every three weeks, once every month, once every 6 weeks, or once every two months.

7.1.5 Additional Treatments

In the methods provided herein, the SMAD7 AON can be administered alone, or in combination with one or more additional IBD treatments (e.g., anti-SMAD7 treatments that are not SMAD7 AON, or IBD treatments that are not anti-SMAD7 treatments).

An additional IBD treatment (e.g., a drug tablet) can be administered concurrently with the SMAD7 AON or the additional drug can be administered before or after the SMAD7 AON.

The additional IBD treatment can be administered via the same route as the SMAD7 AON (e.g., oral administration) or via a different route (e.g., per i.v.).

Additional IBD treatments that can be administered in the methods provided herein in combination with the SMAD7 AON include, without limitation, one or more of the following aminosalicylates, antibiotics, steroids, immunomodulators, or inflammatory cytokine antagonists, or combinations thereof:

Aminosalicylates

In some embodiments, the additional IBD treatment comprises an aminosalicylate.

In some embodiments, the additional IBD treatment comprises 5-aminosalicylic acid (5-ASA or mesalamine), sulfasalazine, balsalazide, or olsalazine.

In some embodiments, the additional IBD treatment comprises 2-hydroxy-4-(4-(5-(2-methyl-3-phenylprop-2-enylidene)-4-oxo-2-sulfanylidene-1,3-thiazolidin-3-yl)butanoylamino)benzoic acid, 2-methoxy-5-amino-N-hydroxybenzamide, 3-methoxysalicylamine, 4-(N-(4-cyclohexylbenzyl)-2-(N,2,4,6-tetramethylphenylsulfonamido)acetamido)-2-hydroxybenzoic acid, 5-(7-hydroxy-3-O-phosphonocholyl)aminosalicylic acid, 5-aminomethylsalicylic acid, 5-aminosalicyl-glycine, 5-aminosalicyltaurine, acetyl 4-aminosalicylic acid, acetyl-4-dimethylaminosalicylic acid, acetyl-5-aminosalicylic acid, aminosalicylic acid, dersalazine, dextran-5-aminosalicylic acid, Dolo-Menthoneurin, ipsalazide, 2-hydroxy-5-(N-((2,5-dihydroxyphenyl)methyl)amino)benzoic acid 3-phenylpropyl ester, methyl 5-aminosalicylate, N-acetyl-5-aminosalicylic acid, N-glucopyranosyl-5-aminosalicylic acid, N-methacryloyl-5-aminosalicylic acid, N,N′-bis(5-aminosalicyl)cysteine, NO-mesalamine, NSC 74859, olsalazine-O-sulfate, pasiniazide, phenyl 4-aminosalicylate, diethylamine salicylate, or UR 12746.

In some embodiments, the additional IBD treatment comprises a compound related to sulfasalazine, such as homosulfasalazine, methylsulfasalazine, salazodimethoxine, salazodin, salicylazoiminopyridine, susalimod, or TL-118.

Antibiotics

In some embodiments, the additional IBD treatment comprises an antibiotic. In some embodiments, the additional IBD treatment comprises a penicillin, a cephalosporin, a polymyxin, a rifampicin, a lipiarmycin (fidaxomicin), a quinolone, a sulfonamide, a macrolide, a lincosamide, a tetracycline, a aminoglycoside, a cyclic lipopeptide, a glycylcycline, or an oxaindole.

In some embodiments, the additional IBD treatment comprises a penicillin, such as benzylpenicillin, phenoxymethylpenicillin, benzathine benzylpenicillin, benzathine phenoxymethylpenicillin, penicillin G, penicillin G procaine, penicillin V, carfecillin, ampicillin, pivampicillin, carbenicillin, amoxicillin, carindacillin, bacampicillin, pivmecillinam, azlocillin, mezlocillin, piperacillin, ticarcillin, talampicillin, sulbenicillin, hetacillin, propicillin, pheneticillin, dicloxacillin, cloxacillin, meticillin, oxacillin, flucloxacillin, biapenem, apalcillin, aspoxicillin, ciclacillin, clemizole penicillin, imipenem, lenampicillin, nafcillin, or panipenem.

In some embodiments, the additional IBD treatment comprises a cephalosporin, such as cefatrizine, cefamandole, cefuzoname, cefpimizole, cephapirin, cephaloridine, cefsulodin, cefotiam, ceforanide, ceftexzole, cefoxitin, latamoxef, flomoxef, cefmetazole, cefotetan, cefpiramide, cephaloglycin, cephalexin, cefadroxil, cefroxadine, ceferadine, cefacloror, or cefoperazone.

In some embodiments, the additional IBD treatment comprises a polymyxin, such as polysporin, neosporin, polymyxin B, polymyxin E, polymyxin S, or polymyxin T.

In some embodiments, the additional IBD treatment comprises a rifampicin, such as 18,19-dihydrorifampicin, 21-(O-phosphoryl)rifampicin, 23-(O-(beta-glucopyranosyl))rifampicin, 23-(O-ribofuranosyl)rifampicin, 25-deacetylrifampicin, 25-desacetylrifapentine, 3-formyl-21-(O-phosphoryl)rifamycin SV, 3-formyl-23-(O-(beta-glucopyranosyl))rifamycin SV, 3-formyl-23-(O-ribofuranosyl)rifamycin SV, CGP 43371, CGS 24565, cotrifazid, dehydrorifampicin, DMB-rifampicin, Myrin P, rifamazid, rifampicin N-oxide, rifapentine, Rifaprim, rivicycline, or Sinerdol EH.

In some embodiments, the additional IBD treatment comprises a quinolones, such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, delafloxacin JNJ-Q2, or nemonoxacin.

In some embodiments, the additional IBD treatment comprises an antibacterial sulfonamide, such as sulfacetamide, sulfadiazine, sulfadimidine, sulfafurazole, sulfisomidine (sulfaisodimidine), sulfadoxine, sulfamethoxazole, sulfamoxole, sulfadimethoxine, sulfamethoxypyridazine, sulfametoxydiazine, sulfadoxine, or sulfametopyrazine.

In some embodiments, the additional IBD treatment comprises a macrolide, such as azithromycin, clarithromycin, erythromycin, telithromycin, carbomycin A, josamycin, kitasamycin, midecamycin/midecamycin acetate, oleandomycin, solithromycin, spiramycin, troleandomycin, or tylosin/tylocine.

In some embodiments, the additional IBD treatment comprises a lincosamide, such as 7-azido-7-deoxylincomycin, 7-deoxylincomycin, antibiotic Bu 2545, chloramlincomycin, Clindamycin, Linco-HAP, lincomycin sulfone, lincomycin sulfoxide, lincospectin, sparsolincomycin, Stomapin, dl-N-ethylclindamycin, mirincamycin, pirlimycin, or pirlimycin adenylate.

In some embodiments, the additional IBD treatment comprises a tetracyline antibiotic, such as tetracycline, chlortetracycline, oxytetracycline, demeclocycline, semi-synthetic, lymecycline, meclocycline, methacycline, minocycline, or rolitetracycline.

In some embodiments, the additional IBD treatment comprises an aminoglycoside antibiotic, such as genatmicin, kanamycin A, amikacin, tobramycin, dibekacin, gentamicin, sisomicin, netilmicin, neomycins B and C, neomycin E (paromomycin), or streptomycin.

In some embodiments, the additional IBD treatment comprises a cyclic lipopeptide antibiotic such as daptomycin and battacin.

In some embodiments, the additional IBD treatment comprises a glycylcycline such as tigecycline.

In some embodiments, the additional IBD treatment comprises an oxazolidinone, such as linezolid, posizolid, torezolid, tedizolid, radezolid, or cycloserine.

In some embodiments, the additional IBD treatment comprises a benzoyl peroxide, rifaximin, clofazimine, isoniazid, tinidazole, vancomycin, or metronidazole.

Steroids

In some embodiments, the additional IBD treatment comprises a steroid, e.g., a corticosteroid.

In some embodiments, the additional IBD treatment comprises a corticosteroid, such as budesonide, dexamethasone (e.g., 21-acetate), betamethasone (e.g., 17-valerate), tixocortol pivalate, triamcinolone, triamcinolone (e.g., acetonide, acetonide 21-palmitate, diacetate, or hexacetonide), mometasone, amcinonide, desonide, fluocinonide, halcinonide, fluocortolone, hydrocortisone, fluticasone propionate, mometasone furotate, prednisone, prednisolone, beclomethasone (e.g., dipropionate (e.g., monohydrate)), flunisolide, or methylprednisolone (e.g., acetate or sodium succinate).

In some embodiments, the additional IBD treatment comprises a corticosteroid, such as 6-hydroxydexamethasone, 9-fluorocortisone, a clobetasol (e.g., propionate), a clobetasone, a clocortolone (e.g., pivalate), a cortisone (e.g., acetate), a dichlorisone, a diflorasone (e.g., diacetate), diflucortolone, doxibetasol, flucmolone, a flumethasone (e.g., pivalate), a fluocinolone (e.g., acetonide), fluorohydroxyandrostenedione, a fluorometholone (e.g., acetate), fluoxymesterone, flupredidene, fluprednisolone, halometasone, halopredone, hydrocortisone, a isoflupredone (e.g., acetate), meclorisone, or a paramethasone (e.g., acetate).

Immunomodulators

In some embodiments, the additional IBD treatment comprises an immunomodulator, e.g., an immunosuppressant. In some embodiments, the additional IBD treatment comprises an immunomodulator, such as purine analog (e.g., azathioprine (AZA) and 6-mercaptopurine (6-MP)), a folic acid analogs (e.g., methotrexate (MTX)), a pyrimidine analogs (e.g., fluorouracil), or a cytotoxic antibiotic (e.g., dactinomycin, mitomycin C, bleomycin, mithramycin, anthracycline, and minocycline). In some embodiments, the additional IBD treatment comprises an immunomodulator, such as tacrolimus, mitoxantrone, cyclophosphamide, mycophenolate mofetil, or rapamycin.

Inflammatory Cytokine Antagonists

In some embodiments, the additional IBD treatment comprises an inflammatory cytokine antagonist, e.g., a tumor necrosis factor (TNF) antagonist or an IL-10 antagonist. In some embodiments, the additional IBD treatment comprises an inflammatory cytokine antagonist, such as infliximab, adalimumab, certolizumab pegol, vedolizumab, golimumab, etanercept, pentoxifylline, or bupropion.

In some embodiments of the methods provided herein, the additional IBD treatment has been administered to the patient prior to the first administration of the SMAD7 AON. In some embodiments, the patient has discontinued the additional IBD treatment prior to the first administration of the SMAD7 AON, e.g., more than 1 week, more than 2 weeks, more than 4 weeks, more than 6 weeks, more than 8 weeks, more than 3 months, more than 6 months, more than 9 months, more than 1 year, more than 1.5 years, more than 2 years, more than 3 years, more than 4 years, or more than 5 years prior. In some embodiments, the additional IBD treatment is administered to the patient during the first and/or second treatment period. In some embodiments, the additional IBD treatment is tapered during the first and/or second treatment period. In some embodiments, the additional IBD treatment is completely tapered at the end of the first treatment period. In some embodiments, the additional IBD treatment is a corticosteroid, the corticosteroid was administered to the patient prior to the SMAD7 AON and the corticosteroid is tapered completely at the end of the first treatment period.

In some embodiments, the additional IBD treatment is a corticosteroid, the corticosteroid was administered to the patient prior to the SMAD7 AON and the corticosteroid is tapered completely at the end of the observation period after the first and/or second treatment period.

7.1.6 Tapering

In some embodiments, the patient having IBD is tapering off one or more additional IBD treatments (other than the anti-SMAD7 therapy; e.g., a corticosteroid) during the first treatment period and/or the second treatment period. In some embodiments, the patient having IBD receives a corticosteroid at the beginning of the first treatment period and is partially or completely tapering off the corticosteroid during the first treatment period and/or the second treatment period. In some embodiments, the patient shows corticosteroid-free clinical remission at the end of the first or the second treatment period.

In some embodiments, the patients having IBD receives a corticosteroid at the beginning of the first treatment period and is partially or completely tapering off the corticosteroid during the observation period after the first and/or second treatment period.

In some embodiments, the patient having IBD is administered with one or more additional IBD treatments during some or all of the first treatment period. In some embodiments, the IBD patient is tapering off one or more additional IBD treatments during the first treatment period. In some embodiments, the IBD patient is tapering off a corticosteroid during the first treatment period (e.g., prednisone). In some embodiments, the IBD patient tapers off an additional IBD treatment comprising a corticosteroid, an aminosalicylate, a budesonide, or an immunosuppressant. In some embodiments, the IBD patient tapers off a corticosteroid. In some embodiments, the IBD patient tapers off the additional IBD treatment during the last 1 week, the last 2 weeks, the last 3 weeks, the last 4 weeks, the last 5 weeks, the last 6 weeks, the last 7 weeks, the last 8 weeks, the last 9 weeks, or the last 10 weeks of the first treatment period. In some embodiments, the IBD patient tapers off one or more additional IBD treatments completely during the first treatment period (the one or more additional IBD treatments are no longer administered to the IBD patient at the end of the first treatment period). In some embodiments, the IBD patient tapers off one or more additional IBD treatment partially during the first treatment period (the IBD patient is administered with one or more additional IBD treatments at a lower dose at the end of the first treatment period than at the beginning of the first treatment period).

In some embodiments, the IBD patient tapers off one or more additional treatments during some or all of the second treatment period. In some embodiments, the IBD patient tapers off one or more additional treatments at least during the first week, second week, third week, fourth week, fifth week, sixth week, seventh week, eighth week, ninth week, or tenth week of the second treatment period.

In some embodiments, tapering off comprises reducing the dose (e.g., daily, weekly, monthly dose) of an additional IBD treatment every 1 day, every 2 days, every 3 days, every 4 days, every 5 days, every 6 days, every 1 week, every 10 days, every 2 weeks, or every 4 weeks.

In some embodiments, tapering off comprises reducing the dose (e.g., daily, weekly, monthly dose) of an additional IBD treatment in increments of at least about 1%, at least about 3%, at least about 5%, at least about 10%, at least about 15%, at least about 20%, at least about 25%, or at least about 30%, at least about 40%, or at least about 50%.

In some embodiments, tapering off comprises reducing the dose (e.g., daily, weekly, monthly dose) of an additional IBD treatment in increments of at least 1 mg, at least 2 mg, at least 3 mg, at least 4 mg, at least 5 mg, at least 6 mg, at least 7 mg, at least 8 mg, at least 9 mg, or at least 10 mg.

In some embodiments, the additional IBD treatment is a corticosteroid (e.g., prednisone) administered to the patient having IBD at a daily dose of >10 mg and tapering off comprises reducing the daily dose once a week by about 5 mg until a dose of 10 mg/day is reached and then further reducing the daily dose once a week by about 2.5 mg until discontinuation.

In some embodiments, the additional IBD treatment is a corticosteroid (e.g., prednisone) administered to the patient having IBD at a daily dose of <10 mg and tapering off comprises reducing the daily dose once a week by about 2.5 mg until discontinuation.

In some embodiments, the additional IBD treatment is a corticosteroid (e.g., budesonide) administered to the patient having IBD and tapering off comprises reducing the daily dose once every 3 weeks by about 3 mg until discontinuation.

In some embodiments, the patient having IBD, who was administered with one or more additional treatments prior to the first treatment period, achieves remission without the one or more additional IBD treatments. In some embodiments, the patient having IBD achieves corticosteroid-free remission. In some embodiments, the patient having IBD achieves corticosteroid-free remission at week 24 of the second treatment period.

7.2 Monitoring Activities

In some embodiments, the methods provided herein further comprise monitoring the activity of the SMAD7 AON in the patient having IBD at one or more timepoints. The one or more timepoints can be, e.g., during the initial screening period, the first treatment period, the observation period, the second treatment period, the follow up period, the third treatment period, or combinations thereof. In some embodiments, the methods comprise monitoring the activity of the SMAD7 AON at one or more timepoint during the first, second and/or third treatment periods.

The one or more timepoints for monitoring can be during any week of any one or more of the initial screening period, the first treatment period, the observation period, the second treatment period, the follow up period, and the third treatment period. Each of the one or more timepoints can be at a set time before or after a given SMAD7 AON administration or it can coincide with the time of a SMAD7 AON administration.

In some embodiments, one timepoint of the one or more timepoints is at or around the beginning of the first treatment period (e.g., during first treatment period—week 0). In some embodiments, one timepoint of the two or more timepoints is at or around the end of the first treatment period (e.g., during first treatment period—week 4, week 8, or week 12). In some embodiments, one timepoint of the two or more timepoints is during week 12 of the first treatment period. In some embodiments, one timepoint of the two or more timepoints is at or around the beginning of the second treatment period (e.g., during second treatment period—week 0). In some embodiments, one timepoint is at or around the end of the second treatment period (e.g., during second treatment period—week 4, week 8, week 12, week 16, week 20, week 24, week 28, week 32, week 36, week 40, week 44, week 48, or week 52).

In some embodiments, one timepoint of the two or more timepoints is during week 24 of the second treatment period. In some embodiments, one timepoint of the two or more timepoints is during week 52 of the second treatment period. In some embodiments, one timepoint of the one or more timepoints is at or around the beginning of the third treatment period (e.g., during the third treatment period—week 0). In some embodiments, one timepoint of the two or more timepoints is at or around the end of the third treatment period (e.g., during the third treatment period—week 4, week 8, week 12, week 24, week 52, week 104, or week 208). In some embodiments, one timepoint of the two or more timepoints is during week 208 of the third treatment period.

In some embodiments, monitoring the activity of the SMAD7 AON in the patient having IBD comprises analyzing a baseline (e.g., a baseline score, level, or value). In some embodiments, the baseline is analyzed at a timepoint during the first week (e.g., week 0) of the first treatment period. In some embodiments, the baseline is analyzed prior to the first administration of the anti-SMAD7 therapy (e.g., at least 1 week, at least 2 weeks, at least 4 weeks, at least 6 weeks, at least 2 months, at least 3 months, at least 6 months, at least 9 months, at least 1 year, at least 3 years, or at least 5 years prior). In some embodiments, the baseline is analyzed during a chronic phase of IBD (flare up), e.g., prior to the first administration of an anti-SMAD7 therapy. In some embodiments, the baseline is analyzed when the patient is on remission (e.g., while receiving a previous IBD treatment, which can be an IBD treatment other than an anti-SMAD7 therapy). In some embodiments, the baseline is an average score from several timepoints prior to treatment.

In some embodiments, monitoring the activity of the SMAD7 AON in the patient having IBD comprises determining a timepoint, e.g., during the first, second and/or third treatment periods when the patient having IBD shows a response to the SMAD7 AON (e.g., decrease of SES-CD score from baseline ≥25% or ≥50%; decrease of CDAI score from baseline ≥100 points; decrease of PRO-2 score from baseline ≥8 points; decrease of average daily liquid or soft stool frequency score from baseline of ≥1 point, and/or decrease of abdominal pain score from baseline ≥1 point, decrease of TMS score from baseline ≥30% and ≥3 points; decrease of ES subscore from baseline ≥1; decrease of PMS score from baseline ≥25% and ≥2 points; or decrease of MMS score from baseline ≥25% and ≥2 points).

In some embodiments, monitoring the activity of the SMAD7 AON in the patient having IBD comprises determining a timepoint, e.g., during the first, second and/or third treatment periods when the patient having IBD experiences remission (e.g., SES-CD score ≤2; CDAI score ≤150; PRO-2 score ≤8; abdominal pain score ≤1 and/or average daily liquid or soft stool frequency ≤1.5; TMS score ≤2 points; ES subscore=0; PMS score ≤2 or MMS score ≤2).

In some embodiments, monitoring the activity of the SMAD7 AON in the patient having IBD comprises analyzing an endoscopic outcome, a clinical activity parameter, a safety or tolerability parameter, a biomarker of intestinal inflammation or tissue damage, a histological score, expression of a biomarker in an intestinal mucosal biopsy, or the systemic exposure of the SMAD7 AON in the patient having IBD.

In some embodiments, monitoring the activity of the SMAD7 AON in the patient having IBD comprises analyzing Quality Of Life (QOL) and Health Economics Assessments (HEA) (e.g., Medical Outcome Study Short Form 36-item Health Survey, Version 2 (SF-36 v2); IBD Questionnaire; Work Productivity and Activity impairment Questionnaire for CD (WPAI-CD); European Quality of Life-5 Dimensions Questionnaire (ED-5D); Harvey-Bradshaw Index (HBI)).

In some embodiments, monitoring the activity of the SMAD7 AON in the patient having IBD comprises analyzing endoscopy (e.g., colonoscopy, flexible rectosignoidoscopy) and intestinal mucosal biopsy, Total Mayo Score (TMS), Partial Mayo Score (PMS), Modified Mayo Score (MMS).

In some embodiments, analyzing the endoscopic outcome comprises analyzing the Simple Endoscopic Score for Crohn's disease (SES-CD). In some embodiments, the SES-CD is analyzed at a timepoint during week 4 or week 12 of the first treatment period. In some embodiments, analyzing the SES-CD comprises analyzing the presence and size of ulcers, the extent of ulcerated surface, the extent of affected surface, or the presence and type of narrowings in the ileum, the right colon, the transverse colon, the left colon, the rectum, or in a combination or all of the listed colon regions.

In some embodiments, the SES-CD is analyzed at a timepoint during week 4, week 8, week 12, week 16, week 20, week 24, week 28, week 32, week 36, week 40, week 44, week 48, or week 52 of the second treatment period. In some embodiments, the SES-CD is analyzed at a timepoint during week 4, week 8, week 12, week 24, week 36, week 52, week 104, week 156, week 208 of the third treatment period.

In some embodiments, analyzing the SES-CD comprises analyzing an absolute SES-CD. In some embodiments, analyzing the SES-CD comprises analyzing changes in SES-CD from baseline (e.g., SES-CD increases or decreases).

In some embodiments, SES-CD variables are defined according to Table 1.

TABLE 1
Exemplary Definition of SES-CD Variables

| | SES-CD Values | | | |
|---|---|---|---|---|
| Variable | 0 | 1 | 2 | 3 |
| Size of | None | Aphthous | Large ulcers | Very large |
| ulcers | | ulcers | (Diameter: | ulcers |
| | | (Diameter: | 0.5 to 2.0 cm) | (Diameter: |
| | | 0.1 to 0.5 cm) | | >2.0 cm) |
| Ulcerated | None | <10% | 10%-30% | >30% |
| surface | | | | |
| Affected | Unaffected | <50% | 50%-75% | >70% |
| surface | segment | | | |
| Presence of | None | Single, can | Multiple, can | Cannot be |
| narrowing | | be passed | be passed | passed |

In some embodiments, the patient having IBD shows a response to the SMAD7 AON if the SES-CD is reduced ≥25% or ≥50% from baseline. In some embodiments, the patient having IBD shows a response to the SMAD7 AON if the SES-CD is reduced by 4 points relative to baseline. In some embodiments, the baseline SES-CD is the SES-CD at or around the beginning of the first treatment period, e.g., at a timepoint during week 0.

In some embodiments, the patient having IBD experiences remission if the SES-CD ≤2.

In some embodiments, analyzing an endoscopic outcome comprises analyzing mucosal healing. In some embodiments, mucosal healing is analyzed at a timepoint during week 12 of the first treatment period.

In some embodiments, the patient having IBD experiences mucosal healing if ulceration is absent in the patient.

In some embodiments, the patient having IBD experiences mucosal healing if the SES-CD ≤2.

In some embodiments, analyzing a histological score comprises analyzing an absolute histological score from intestinal mucosa of the patient having IBD. In some embodiments, analyzing the histological score comprises analyzing changes in the histological score from intestinal mucosa of the patient having IBD from baseline. In some embodiments, analyzing the histological score comprises analyzing a change in the histological score from the intestinal mucosa of the patient having IBD from baseline at a timepoint during week 12 of the first treatment period.

In some embodiments, analyzing the histological score comprises analyzing a change in the histological score from the intestinal mucosa of the patient having IBD from baseline at a timepoint during week 12 or week 24 of the second treatment period. In some embodiments, analyzing the histological score comprises analyzing a change in the histological score from the intestinal mucosa of the patient having IBD from baseline at a timepoint during week 12, week 24, week 36, week 52, week 104, week 156, week 208 of the third treatment period.

In some embodiments, analyzing an endoscopic outcome comprises analyzing the Crohn's disease endoscopic index of severity (CDEIS).

In some embodiments, analyzing a clinical activity parameter comprises analyzing the Crohn's disease activity index (CDAI; range 0-600).

The CDAI is a useful measure in clinical studies evaluating the efficacy of new therapies in CD patients with predominantly inflammatory disease. This index is based, in part, on a self-assessment questionnaire completed by the subject. Self-assessment questionnaires are known in the art. The CDAI can assess how CD affects the subject's quality of life and the effect of treatment. CDAI determination can involve processing a self assessment questionnaire with patient responses scored numerically and weighted. Scores (range 0 to 600) are then ranked according to severity of the disease. Mild active disease can be defined by a score of ≥150 and ≤219, moderate active disease can be defined by a score of ≥220 and ≤450, whereas severe disease can be defined as a CDAI score >450. Remission can be defined as a CDAI score ≤150. CDAI determinations can consider a number of variables, including, e.g., number of liquid or soft stools per day (e.g., each day for 7 days), abdominal pain/cramps (e.g., each day for 7 days), general well-being (e.g., each day for 7 days), number of complications, such as, e.g., arthritis or arthralgias, iritis or uveitis, erythema nodosum, pyoderma gangrenosum, or aphthous ulcers, anal fissures, fistulae, or abscess, other fistula fever higher than 37.8° C. during previous week, taking loperamide, diphenoxylate, or opiates for diarrhea, abdominal mass, hematocrit of less than 0.47 in men and less than 0.42 in women, percentage of deviation above or below standard weight.

In some embodiments, analyzing the CDAI score comprises analyzing an absolute CDAI score. In some embodiments, analyzing the CDAI score comprises analyzing changes in CDAI score from baseline (e.g., CDAI score increases or decreases).

In some embodiments, the baseline CDAI is the CDAI at or around the beginning of the first treatment period, e.g., at a timepoint during week 0.

In some embodiments, the CDAI score is analyzed at one or more timepoints during the first, second and/or third treatment periods. In some embodiments, the CDAI is analyzed at the beginning of the first treatment period (e.g., at a timepoint during week 0 of the first treatment period) and at the end of the first treatment period (e.g., at a timepoint during week 4, week 8, or week 12 of the first treatment period). In some embodiments, the CDAI is analyzed at the beginning of the second treatment period (e.g., at a timepoint during week 0 of the second treatment period) and at the end of the second treatment period (e.g., at a timepoint during week 4, week 8, week 12, week 16, week 20, week 24, week 28, week 32, week 36, week 40, week 44, week 48, or week 52 of the second treatment period).

In some embodiments, the CDAI is analyzed at the beginning of the third treatment period (e.g., at a timepoint during week 0 of the third treatment period) and at the end of the third treatment period (e.g., at a timepoint during week 24, week 52, week 104, week 156, or week 208 of the third treatment period).

In some embodiments, analyzing the CDAI comprises analyzing the time to partial loss of response.

In some embodiments, the patient having IBD shows a clinical response to the SMAD7 AON if the CDAI is reduced ≥100 points from baseline. In some embodiments, the patient having IBD shows a clinical improvement if the absolute CDAI score is <180 and the CDAI score is reduced ≥70 points from baseline.

In some embodiments, the patient having IBD experiences remission if the CDAI <150.

In some embodiments, analyzing a clinical activity parameter comprises analyzing a Patient Reported Outcome (PRO). PROs analysis involves patients quantifying their own symptoms, which can be useful in assessing IBD severity. A two-item Patient Reported Outcome (PRO-2) for CD considers two CDAI variables, e.g., liquid or soft stool frequency and abdominal pain. Methods for determining PRO-2 scores are well known in the art. For example, a total PRO-2 score can be calculated based on information provided in a patient questionnaire or diary. Daily scores of liquid or soft stool frequency and abdominal pain, can be averaged over 7 days and weighted, e.g., using multiplication factors applied also during CDAI determinations. In some embodiments, PRO-2 values of 8, 14, and 34 points can correspond to CDAI scores of 150, 220, and 450 points and PRO-2 score changes from baseline of 2, 5, and 8 points can correspond to changes in CDAI scores of 50, 70, and 100 points.

In some embodiments, analyzing the clinical activity parameter comprises analyzing a two-item patient reported outcome (PRO-2) score.

In some embodiments, analyzing the PRO-2 score comprises analyzing an absolute PRO-2 score. In some embodiments, analyzing the PRO-2 score comprises analyzing changes in PRO-2 score from baseline (e.g., PRO-2 score increases or decreases).

In some embodiments, the baseline PRO-2 score is the PRO-2 score at or around the beginning of the first treatment period, e.g., at a timepoint during week 0.

In some embodiments, the PRO-2 score is analyzed at one or more timepoints during the first, second and/or third treatment periods. In some embodiments, PRO-2 is analyzed at a timepoint during week 2, week 4, week 8, or week 12 of the first treatment period. In some embodiments, PRO-2 is analyzed at a timepoint during week 4, week 8, week 12, week 16, week 20, week 24, week 28, week 32, week 36, week 40, week 44, week 48, or week 52 of the second treatment period.

In some embodiments, PRO-2 is analyzed at a timepoint during week 24, week 52, week 104, week 156, or week 208 of the third treatment period.

In some embodiments, analyzing PRO-2 comprises analyzing an average daily liquid stool, an average daily soft stool, or an average daily abdominal pain score.

In some embodiments, analyzing PRO-2 comprises analyzing the average daily liquid or soft stool frequency at a timepoint during the first, second and/or third treatment period. In some embodiments, analyzing PRO-2 comprises analyzing the average daily liquid or soft stool frequency at a timepoint during week 2, week 4, week 8, or week 12 of the first treatment period, during week 4, week 8, week 12, week 16, week 20, or week 24 of the second treatment period, or during week 24, week 52, week 104, week 156, or week 208 of the third treatment period.

In some embodiments, analyzing PRO-2 comprises analyzing the average daily abdominal pain score at a timepoint during the first, second and/or third treatment period. In some embodiments, analyzing PRO-2 comprises analyzing the average daily abdominal pain score at a timepoint during week 2, week 4, week 8, or week 12 of the first treatment period, during week 4, week 8, week 12, week 16, week 20, or week 24 of the second treatment period, or during week 24, week 52, week 104, week 156, or week 208 of the third treatment period.

In some embodiments, the patients having IBD achieve an average daily liquid or soft stool frequency ≤6, ≤5, ≤4, ≤3, ≤2, or ≤1 at a time point during week 2, week 4, week 8, or week 12 of the first treatment period, during week 4, week 8, week 12, week 16, week 20, week 24, week 28, week 32, week 36, week 40, week 44, week 48, or week 52 of the second treatment period, or during week 24, week 52, week 104, week 156, or week 208 of the third treatment period.

In some embodiments, the patients having IBD achieve an average daily abdominal pain score ≤3, ≤2, or ≤1 at a time point during week 2, week 4, week 8, or week 12 of the first treatment period, during week 4, week 8, week 12, week 16, week 20, week 24, week 28, week 32, week 36, week 40, week 44, week 48, or week 52 of the second treatment period, or during week 24, week 52, week 104, week 156, or week 208 of the third treatment period.

In some embodiments, the patients having IBD achieve an average daily liquid or soft stool frequency ≤4, ≤3.5, ≤3.0, ≤2.5, or ≤2.0 and an abdominal pain score ≤2.0, ≤1.5, or ≤1.0 at a time point at a time point during week 2, week 4, week 8, or week 12 of the first treatment period, during week 4, week 8, week 12, week 16, week 20, week 24, week 28, week 32, week 36, week 40, week 44, week 48, or week 52 of the second treatment period, or during week 24, week 52, week 104, week 156, or week 208 of the third treatment period.

In some embodiments, the patients having IBD achieve an average daily liquid or soft stool frequency ≤3 and abdominal pain score ≤1 at week 4, week 12, or week 52 of the dosing regimen.

In some embodiments, the patients having IBD achieve an average daily liquid or soft stool frequency ≤1.5 and abdominal pain score ≤1 at week 4, week 12, or week 52 of the dosing regimen.

In some embodiments, the patient having IBD shows a response to the SMAD7 AON if the PRO-2 score is reduced ≥8 points from baseline.

In some embodiments, the patient having IBD experiences remission if the PRO-2 score ≤8.

In some embodiments, the patient having IBD experiences remission (e.g., SES-CD ≤2, CDAI <150, PRO-2≤8, abdominal pain score ≤1 and/or average daily liquid or soft stool frequency score ≤1.5; TMS ≤2.0, MMS ≤2.0, PMS ≤2.0, or ES=0) without prior or concurrent corticorsteroid treatment.

It can be useful for the analysis of CDAI and PRO-2 scores to have the patient having IBD record information in a diary, such as number of liquid or soft stools per day, abdominal pain/cramps, general well-being, fever higher than 37.8° C. during previous week, administration of, e.g., diphenoxylate/atropine, loperamide, or opiates for diarrhea.

In some embodiments, the QOL and HEA questionnaires comprise the Medical Outcome Study Short Form 36-item Health Survey, version 2 (SF-36 v2). SF-36 v2 is a self-administered 36-item general health status instrument often used in clinical trials and health services research. SF-36 v2 typically includes 8 multi-item scales that assess 8 health domains, such as 1) limitations in physical activities because of health problems; 2) limitations in social activities because of physical or emotional problems; 3) limitations in usual role activities because of physical health problems; 4) bodily pain; 5) general mental health (psychological distress and well-being); 6) limitations in usual role activities because of emotional problems; 7) vitality (energy and fatigue); and 8) general health perceptions. The concepts measured by the SF-36 are not specific to any age, disease, or treatment group, allowing comparison of relative burden of different diseases and the relative benefit of different treatments.

In some embodiments, the QOL and HEA questionnaires comprise the Inflammatory Bowel Disease Questionnaire (IBDQ). The IBDQ is a responsive instrument for reflecting quick change in the quality of life of patients with CD within a 2-week period. The IBDQ has evolved into a standard for measuring disease-specific quality of life in patients with CD. The IBDQ is a self-administered, 32-item questionnaire concerning 4 dimensions of quality of life (Hlavaty, 2006), including bowel function dimension (BD), emotional status dimension (ED), symptoms dimension (SysD), and social functioning dimension (SocD). Each dimension is typically scored up to 7 points for each item. The total IBDQ score ranges from 32 to 224 points, with higher scores indicating a better quality of life.

In some embodiments, the QOL and HEA questionnaires comprise the Work Productivity and Activity Impairment Questionnaire for Crohn's Disease (WPAI-CD). The WPAI-CD assesses the impact of CD on a patient's work and activity during the past 7 days. The WPAI-CD comprises 6 questions that capture information, such as employment status, hours of work missed because of CD, hours missed because of other reasons, hours actually worked, the degree to which CD has affected productivity while working from 0 (no effect) to 10 (maximum impairment), the degree to which CD affected other (non-work) regular activities (0-10). The WPAI-CD questions are used to create 4 dimensions, with scores expressed in percentage of impairment; higher scores indicate greater impairment and reduced productivity: absenteeism (work time missed in employed subjects), presenteeism (reduced productivity while at work), overall work impairment (absenteeism plus presenteeism), activity impairment (reduced productivity in daily activities). The minimally important difference (MID), i.e., the change in WPAI-CD score deemed to be clinically meaningful, is approximately 7%.

In some embodiments, the QOL and HEA questionnaires comprise the European Quality of Life-5 Dimensions Questionnaire (EQ-5D). The EQ-5D (The EuroQol Group, 1990) is a validated, 6-item, self-administered instrument designed to measure generic health status. The EQ-5D typically has two components: 1) the EQ-5D descriptive system (five items; EQ-5D Index Score) and 2) the EQ Visual Analog Scale (EQ-5D VAS). The EQ-5D Index Score includes five dimensions of health (mobility, self-care, usual activities, pain/discomfort, and anxiety/depression). Each dimension can have three levels reflecting “no problems,” “some problems,” and “extreme problems.” A unique EQ-5D health state is defined by combining one level from each of the five dimensions. Health states are on a scale of 0.0 (death) to 1.0 (perfect health). Because the worst possible health state may be judged by respondents as worse than death, negative values are possible. Preference values for many countries have been established through a series of studies. The EQ-5D VAS is a vertical scale with endpoints labeled “best imaginable health” and “worst imaginable health” anchored at 100 and 0, respectively. Respondents are asked to indicate how they rate their own “health state today” by drawing a line from an anchor box to that point on the EQ-5D VAS which best represents their own health on that day. The EQ-5D has demonstrated good reproducibility (intra-class correlation [ICC]=0.77 for the EQ VAS score and ICC=0.89 for the EQ-5D Index score) and evidence of known-groups validity.

In some embodiments, the QOL and HEA questionnaires comprise the Harvey-Bradshaw Index (HBI). The HBI was devised in 1980 as a simpler version of the CDAI for data collection purposes. It consists of only clinical parameters, the first three items are scored by the subject for the previous day, and the remaining 2 items are scored by the Investigator or delegate at the scheduled visit. The HBI is far simpler to use than the CDAI and does not require biochemical tests. The HBI scores range from 0 to >18, the upper range may vary based on the number of liquid or soft stool per day, and are then ranked according to severity of the disease. Remission is defined by a score of <5; mild active disease is defined by a score of >5 and <7; moderate active disease is defined by a score of >8 and <16; severe disease is defined by a score of >16. The HBI consists of 8 variables: general well-being rating (from yesterday), abdominal pain rating (from yesterday), total number of liquid or soft stools (from yesterday), abdominal mass presence (on the day of the visit), complications (check any that apply, on the day of the visit, e.g., none, arthritis, uveitis, erythema nodosum, aphthous ulcers, pyoderma gangrenosum, anal fissures, new fistula, and abscess).

In some embodiments, the QOL and HEA questionnaires comprise Healthcare Resource Utilization (HRU) assessment. The HRU assessment will be evaluated in this study to assess the impact of CD and health-related outcomes (hospitalizations, emergency department or urgent care clinic visits, and physician visits).

In some embodiments, Physician's Global Assessment (PGA) is done as part of the Mayo score. The PGA acknowledges 3 criteria: the subject's daily recollection of abdominal discomfort, general sense of wellbeing, and other observations, such as physical findings and the subject's performance status.

In some embodiments, a colonoscopy is performed if a subject has extensive colitis; or a flexible rectosigmoidoscopy is performed if a subject only has left-sided colitis. The colonoscopy and/or flexible rectosigmoidoscopy is performed at about week 1, about week 2, about week 4, about week 8, about week 12, about week 24, about week 32, about week 40, about week 52, about year 2, about year 4.

In some embodiments, Total Mayo Score (TMS) is assessed. The TMS is an instrument designed to measure disease activity of UC. The TMS typically ranges from 0 to 12 points. It consists of 4 subscores, each graded from 0 to 3 with higher scores indicating more severe disease: Stool Frequency Subscore (SFS), Rectal Bleeding Subscore (RBS), Endoscopy Subscore, Physician's Global Assessment. Clinical response is defined as a decrease from baseline in TMS of at least 3 points, and at least a 30% decrease in the TMS, with an accompanying decrease in the RBS of at least 1 point or absolute RBS of 0 or 1. Clinical remission is generally defined as TMS ≤2, with no individual subscore >1. In some embodiments, clinical remission can occur without normalization of stool frequency and comprise a Stool Frequency Subscore of 1. Endoscopic response is defined as a 1 point or greater decrease from baseline in the endoscopy subscore. Endoscopic remission is defined as endoscopy subscore of 0. In some embodiments, endoscopic remission comprises an endoscopy subscore of 0 or 1, where a subscore of I does not include friability. In some embodiments, any indication of friability indicates an endoscopy subscore of at least 2. The TMS assessment is performed at about week 4, about week 8, about week 12, about week 24, about week 32, about week 40, about week 52, about year 2, about year 4.

In some embodiments, Partial Mayo Score (PMS) is assessed. The PMS is the sum of the RBS, SFS, and PGA, and ranges from 0 to 9 points. Clinical response is defined as a decrease from baseline in PMS of at least 2 points and at least 25%, with an accompanying decrease in the RBS of at least 1 point or an absolute RBS int or absolute RBS of 0 or 1. Clinical remission is defined as TMS ≤2,no individual subscore >1. In some embodiments, clinical remission comprises an RBS=0. The PMS assessment is performed at about week 4, about week 8, about week 12, about week 24, about week 32, about week 40, about week 52, about year 2, about year 4.

In some embodiments, Modified Mayo Score (MMS) is assessed. The MMS will be based on the stool frequency, rectal bleeding and endoscopy subscores of the total Mayo score, and will exclude the PGA subscore, since this is a global measure that is subjective in nature. The modified Mayo score ranges from 0 to 9 points. Clinical response is defined as a decrease from baseline in MMS of at least 2 points and at least 25%, with an accompanying decrease in the RBS of at least 1 point or an absolute RBS of 0 or 1. Based on the MMS, clinical remission is defined as a MMS of ≤2, with no individual subscore >1. The MMS assessment is performed at about week 4, about week 8, about week 12, about week 24, about week 32, about week 40, about week 52, about year 2, about year 4.

Rectal bleeding subscore (RBS) is one of the four measurements for Mayo Scoring System (MSS). RBS often ranges from 0 to 3, with 0 representing no blood seen, 1 representing streaks of blood with stool less than half the time, 2 representing obvious blood with stool most of the time, and 3 representing blood alone passed. The daily RBS represents the most severe bleeding of the day. In some embodiments, clinical remission comprises an RBS=0.

In some embodiments, the safety or tolerability parameter comprises an adverse event, a physical examination, vital signs, body weight, an electrocardiogram (EKG), a clinical laboratory safety evaluation, a stool culture, or a pregnancy test.

In some embodiments, analyzing a safety or tolerability parameter comprises assessing type, frequency or severity of an adverse event, a relationship of the adverse event to the administration of the SMAD7 AON, discontinuation of SMAD7 AON administration due to an adverse event, or clinically significant changes in vital signs, ECGs, or laboratory findings.

In some embodiments, the clinical laboratory safety evaluation comprises a hematology test, a coagulation test, a serum chemistry test, and/or a urinanalysis. In some embodiments, the hematology test comprises a red blood cell (RBC) count, a hemoglobin level, a hematocrit, mean corpuscular hemoglobin (MCH), mean corpuscular hemoglobin concentration (MCHC), mean corpuscular volume (MCV), a differential white blood cell (WBC) count, an absolute WBC count, or a platelet count. In some embodiments, the coagulation test comprises analyzing the prothrombin time (PT) or the activated partial thromboplastin time (APTT). In some embodiments, the serum chemistry test comprises analyzing total protein, albumin, calcium, phosphorous, glucose, total cholesterol, triglycerides, uric acid, total bilirubin, alkaline phosphatase, aspartate aminotransferase (AST)/serum glutamic-oxaloacetic transaminase (SGOT), alanine aminotransferase (ALT)/serum glutamic-pyruvic transaminase (SGPT), sodium, potassium, chloride, carbon dioxide, blood urea nitrogen (BUN), creatinine, lactic dehydrogenase (LDH), magnesium, or complement activation (e.g., Bb, C3a and C5a).

In some embodiments, the stool culture comprises a test for Clostridium difficile (C. difficile) toxin.

In some embodiments, the urinanalysis comprises dipstick urinanalysis or microscopic urinanalysis. In some embodiments, the dipstick urinanalysis comprises analyzing specific gravity, pH, glucose, ketones, total protein, bilirubin, leukocyte esterase, nitrite, or urobilinogen. In some embodiments, microscopic urinanalysis comprises analyzing epithelial cells, RBCs, or WBCs.

In some embodiments, analyzing a biomarker of intestinal inflammation or tissue damage comprises analyzing SMAD7, SMAD3 phosphorylation, HLA-DR, CD4, CD8, CRP (e.g., measured as hsCRP), FCP, TNFα, IL8, IFN-γ, IL-12, IL17A or IL6. Biomarker analysis can comprise the analysis of absolute biomarker levels or the analysis of changes in biomarker levels (e.g., decreases or increases from baseline, or from another reference value from the patient's medical history). Biomarker levels in the patient having IBD can be compared to corresponding biomarker levels in healthy control groups, or changes in biomarker levels can be followed in the patient having IBD over time. The biomarkers can be analyzed at any one or more timepoints during the initial screening period, the first treatment period, the second treatment period, the third treatment period, or the follow up period, or combinations thereof. In some embodiments, the CRP is analyzed in a blood sample obtained from the patient having IBD. In some embodiments, the FCP is analyzed in a fecal sample obtained from the patient having IBD.

In some embodiments, analyzing a biomarker of intestinal inflammation or tissue damage comprises analyzing FCP, CRP, CD4, CD8, HLA-DR, SMAD3 phosphorylation, SMAD7 mRNA or protein level, TNF-α, IL-8, IFN-γ, IL-12, IL-17, IL-6, Reg-3α, IL-10, IL-25, CCL20, IL-17A, Foxp3, CCR9, IL-5, IL-13, IL4 and TGF-β1.

Methods for analyzing biomarkers in a patient sample are known in the art and comprise, e.g., ELISA, Western Blot, RT-PCR, HPLC, LC-MS, fluorescence microscopy, immunocytochemistry, and the like.

In some embodiments, CRP levels (e.g., measured as hsCRP), e.g., in a blood, serum or plasma sample from a patient having IBD, indicates if the patient having IBD responds to the SMAD7 AON.

In some embodiments, other biomarkers (e.g., IL-10, CCL20, TNF-α) will be analyzed from serum blood samples and intestinal mucosal biopsies. In some embodiments, intestinal microbiome and FCP will be assessed from fecal samples. In some embodiments, whole blood sample will be collected at the specified time-points to isolate PBMC for evaluating the expression of immune biomarkers (e.g., IL-17A).

In some embodiments, analyzing a biomarker of intestinal inflammation or tissue damage comprises analyzing the change in CRP from baseline at a timepoint during week 4, week 8, or week 12, of the first treatment period, during week 0, week 4, week 8, week 12, week 16, week 20, or week 24 of the second treatment period, during week 24, week 52, week 104, week 156, or week 208 of the third treatment period, or during week 20 or 52 of the observation period.

In some embodiments, analyzing expression of a biomarker in an intestinal mucosal biopsy comprises analyzing biomarkers such as cluster of differentiation 4 (CD4), cluster of differentiation 8 (CD8), a major histocompatibility complex (MHC) class I or class II (e.g., HLA-DR), SMAD3 (e.g., SMAD3 phosphorylation) or SMAD7 (e.g., SMAD7 mRNA levels or protein levels).

7.2.1 Clinical Response

Clinical responses in an IBD patient to the treatment methods and/or administration regimes provided herein can inform adjustments to the treatment methods and/or administration regimes, e.g., during the transition from the first treatment period to the second treatment period, during the transition from the second treatment period to the third treatment period, or upon exit from the second or third treatment period. See, e.g., Section 7.1.1.3, Section 7.1.1.6, Section 7.6 and Section 7.7. For example, the administration regime of an IBD patient responding to an anti-SMAD7 therapy can be adjusted by, e.g., shortening the length of the first and/or second treatment period, by allowing the IBD patient to transition from the first to the second treatment period or from the second to the third treatment period, by lowering the dose of the anti-SMAD7 therapy, or by ending the anti-SMAD7 therapy. The administration regime of an IBD patient not responding to the anti-SMAD7 therapy can be adjusted, e.g., by increasing the dose of the anti-SMAD7 therapy.

In some embodiments, the patient having IBD shows a clinical response if the patient having IBD shows ≥50% reduction of SES-CD from baseline (e.g., a timepoint during week 0 of first treatment period), ≥100 points decrease from baseline in CDAI score, ≥8 point decrease from baseline PRO-2 score, ≥3.0 or ≥1.5 points decrease from baseline in average daily liquid or soft stool frequency and/or ≥1 point decrease from baseline in abdominal pain score, decrease of TMS score from baseline ≥30% and ≥3 points, decrease of ES from baseline ≥1; decrease of PMS score from baseline ≥25% and ≥2 points; decrease of MMS score from baseline ≥25% and ≥2 points (e.g., at a timepoint during the first, second and/or third treatment periods).

In some embodiments, the patient having IBD shows a clinical improvement if the patient having IBD shows an absolute CDAI score ≤180 and a CDAI score reduction >70 points from baseline (e.g., a timepoint during week 0 of first treatment period).

In some embodiments, the patient having IBD shows a clinical response if the patient having IBD shows a decrease of TMS score ≥30% and ≥3 points from baseline (e.g., a timepoint during week 0 of first treatment period), along with a decrease of RBS score ≥1 or absolute RBS ≤1; a decrease of endoscopic subscore from baseline ≥1; a decrease of PMS score from baseline ≥25% and ≥2 points, along with a decrease of RBS score ≥1 or an absolute RBS ≤1; a decrease of MMS score from baseline ≥25% and ≥2 points, along with a reduction in RBS score of ≥1 or an absolute RBS ≤1.

In some embodiments, the patient having IBD shows a clinical response if the levels of one or more biomarker of intestinal inflammation, such as an inflammatory cytokine (e.g., TNFα, IFN-γ, IL6, IL8, or IL12, or another biomarker, such as, e.g., SMAD7, SMAD3 phosphorylation, FCP, CRP, CD4, CD8, or HLA-DR), are decreased from baseline at least 10%, at least 20%, at least 30%, at least 40%, at least 50%, at least 60%, at least 70%, at least 80%, at least 90%, at least 95%, at least 98%, or at least 99% in a sample obtained from the patient having IBD at a timepoint before or during the first treatment period, during the second treatment period, and/or during the third treatment period.

In some embodiments, the patient having IBD shows a clinical response if the levels of one or more biomarkers of intestinal inflammation, such as an anti-inflammatory cytokine e.g., IL10, are decreased from baseline at least 2-fold, at least 3-fold, at least 5-fold, at least 10-fold, at least 15-fold, at least 20-fold, at least 25-fold, at least 50-fold, at least 100-fold, at least 250-fold, at least 500-fold or at least 1,000-fold in a sample obtained from the patient having IBD at a timepoint before or during the first treatment period, during the second treatment period, and/or during the third treatment period.

In some embodiments, the patient having IBD shows a clinical response the levels of one or more biomarker of intestinal inflammation, (e.g., SMAD3 phosphorylation), are increased from baseline at least 2-fold, at least 3-fold, at least 5-fold, at least 10-fold, at least 15-fold, at least 20-fold, at least 25-fold, at least 50-fold, at least 75-fold, or at least 100-fold in a sample obtained from the patient having IBD at a timepoint during the first treatment period and/or the second treatment period.

In some embodiments, the patient having IBD shows a clinical response if SMAD3 phosphorylation is increased from baseline (e.g., a timepoint during week 0 of first treatment period) at least 2-fold, at least 3-fold, at least 5-fold, at least 10-fold, at least 15-fold, at least 20-fold, at least 25-fold, at least 50-fold, at least 100-fold, at least 250-fold, at least 500-fold or at least 1,000-fold in a sample obtained from the patient having IBD.

In some embodiments, the patient having IBD shows a clinical response if SMAD7 mRNA levels and/or SMAD7 protein levels are decreased at least 2-fold, at least 3-fold, at least 5-fold, at least 10-fold, at least 15-fold, at least 20-fold, at least 25-fold, at least 50-fold, at least 100-fold, at least 250-fold, at least 500-fold or at least 1,000-fold in a sample obtained from the patient at the end of the first treatment period compared to a sample obtained from the patient at the beginning of the first treatment period.

In some embodiments, the IBD patient shows a clinical response if the CRP levels (e.g., measured at hsCRP) in a blood, serum or plasma sample from the patient having IBD are <3.0 mg/L, <2.5 mg/L, <2.0 mg/L, <1.5 mg/L, <1.0 mg/L, or <0.5 mg/L.

In some embodiments, the IBD patient shows a clinical response if the TNFα levels in a blood, serum or plasma sample from the patient having IBD are <30 pg/ml, <25 pg/ml, <20 pg/ml, or <15 pg/ml.

In some embodiments, the IBD patient shows a clinical response if the IL6 levels in a blood, serum or plasma sample from the patient having IBD are <600 pg/ml, <500 pg/ml, <400 pg/ml, or <300 pg/ml.

In some embodiments, the IBD patient shows a clinical response if the IL8 levels in a blood, serum, or plasma sample from the patient having IBD are <30 pg/ml, <25 pg/ml, <20 pg/ml, or <15 pg/ml.

In some embodiments, the IBD patient shows a clinical response if the IL12 levels in a blood, serum or plasma sample from the patient having IBD are <100 pg/ml, <75 pg/ml, <50 pg/ml, or <25 pg/ml.

In some embodiments, the IBD patient shows a clinical response if the IL17A levels in a blood, serum or plasma sample from the patient having IBD are <100 pg/ml, <75 pg/ml, <50 pg/ml, or <25 pg/ml.

In some embodiments, the IBD patient shows a clinical response if the FCP levels in a fecal sample from the patient having IBD are ≤25 μg/g stool, ≤50 μg/g stool, ≤75 μg/g stool, ≤100 μg/g stool, ≤150 μg/g stool, or ≤200 μg/g stool.

7.2.2 Clinical Remission

In some embodiments, the patient having IBD shows remission, if the patient shows a SES-CD score ≤2, a CDAI score ≤150, a PRO-2 score <8, abdominal pain score ≤1 and/or average daily liquid or soft stool frequency score ≤1.5; TMS score ≤2; ES=0; PMS score ≤2; MMS score ≤2 or mucosal healing, as indicated, e.g., by the absence of an intestinal mucosal ulceration, e.g., at a timepoint during the first, second and/or third treatment periods.

In some embodiments, the patient having IBD shows remission, if the patient shows a TMS score ≤2 points with no individual subscore >1; an endoscopic subscore=0; a PMS score ≤2 points with no individual subscore >1; an MMS score ≤2 points with no individual subscore >1, e.g., at a timepoint during the first, second and/or third treatment periods.

In some embodiments, the patient having IBD shows remission, if the level of one or more biomarkers of intestinal inflammation (e.g., TNFα, IFN-γ, IL6, IL8 or IL2, or another biomarker, such as, e.g., FCP, CRP, SMAD3 phosphorylation, IL-17A, CD4, CD8, or HLA-DR) in a sample from the patient are within a standard deviation (SD) range of 2σ, 3σ, 5σ, 7σ, or 10σ, from the average, median, or mean levels of the biomarker in a healthy control group (e.g., matched by medical history, age, gender, race, or other factors).

7.2.3 Loss of Response

In some embodiments, the patient experiences a partial loss of response, or a complete loss of response, if the CDAI score is increased by >50 points and if the CDAI score is >150 at 2 or more consecutive timepoints or after the patient first showed a response to the SMAD7 AON, e.g., during the first, second or third treatment period.

7.3 Fecal Calprotectin

In the methods provided herein, the biomarker FCP can be used to monitor the activities of an anti-SMAD7 treatment, as described in Section 7.2 (e.g., to analyze if a patient shows a clinical response to a SMAD7 AON, or if a patient experiences remission). In some embodiments, FCP levels in an IBD patient sample can inform a decision regarding whether an IBD patient is transitioning from the first to the second treatment phase (e.g., if the patient shows a clinical response to a SMAD7 AON or if the patient shows remission), e.g., as described in Section 7.1.

In other methods provided herein, FCP can be used as a biomarker for patient selection.

In another aspect, provided herein is a method for treating or managing IBD in a patient having IBD. In one embodiment, the method comprises the following steps: (a) of administering to the patient an initial dose of a SMAD7 AON; (b) analyzing the level of FCP in the patient; and (c) if the level of FCP is above normal levels of FCP, then administering to the patient a subsequent dose that is greater than or equal to the initial dose. Alternatively, if in step (c), the level of FCP is below normal levels of FCP as determined in step (b), then step (c) comprises administering to the patient a subsequent dose that is equal to or smaller than the initial dose.

In another aspect, provided herein is a method for treating or managing inflammatory bowel disease (IBD) in a patient having IBD, wherein the method comprises (a) establishing a control level of FCP for the patient; (b) administering to the patient an initial dose of a SMAD7 antisense-oligonucleotide; c) analyzing the level of FCP in the patient; and (d) if the level of FCP is lower than the control level, then administering to the patient a subsequent dose that is the same as the, initial dose or smaller than the initial dose, or, if the level of FCP is unchanged or increased compared to the control level, then administering to the patient a subsequent dose that is the same as the initial dose or greater than the initial dose or terminating the treatment.

In some embodiments, the control level for the IBD patient is the FCP level in a sample obtained from the IBD patient prior to administration of the first anti-SMAD7 treatment during a chronic disease period, e.g., when the patient was in remission. In some embodiments, the control level for the IBD patient is the FCP level in a sample obtained from the IBD patient prior to administration of the first anti-SMAD7 treatment during an acute disease period (e.g., CD patient: CDAI >150; CDAI ≥250 and ≤450; UC patient: MMS ≥4 and ≤9, and ES ≥2). In some embodiments, the control level for the IBD patient is the FCP level in a sample obtained from the IBD patient during a period when the patient is administered with an anti-SMAD7 treatment, or at the beginning of a treatment period (e.g., during week 0, baseline level). In some embodiments, the control level of the IBD patient is the FCP level in a sample obtained from the IBD patient at an earlier timepoint during an anti-SMAD7 treatment period.

In another aspect, provided herein is a method for treating or managing IBD in a patient having IBD with respect to administration of an initial dose of a SMAD7 AON. In one embodiment, the provided herein is a method for treating or managing IBD in a patient having IBD, where the method comprises the following steps: (a) analyzing the level of FCP in the patient; and (b) if the level of FCP is above normal levels of FCP, then administering to the patient an initial dose of a SMAD7 AON. Additionally, the method can further comprise the steps of: (c) analyzing the level of FCP in the patient after said administering step, i.e., step (b); and (d) if the level of FCP is above normal levels of FCP, then administering to the patient a subsequent dose that is greater than or equal to the initial dose. Alternatively, if in step (d), the level of FCP is below normal levels of FCP, as determined in step (c), then step (d) comprises administering to the patient a subsequent dose that is equal to or smaller than the initial dose. In some instances, if the subsequent dose administered in step (d) is equal to or greater than the maximum tolerated dose (MTD), then the method comprises the step of terminating the treatment.

The level of FCP can be analyzed at any timepoint during an administration schedule in a method for treating IBD provided herein. For example, the FCP level can be analyzed before or after administering an anti-SMAD7 therapy (e.g., at least 1 day, at least 3 days, at least 5 days, at least 1 week, at least 2 weeks, at least 3 weeks, at least 1 month, at least 2 months, at least 4 months, or at least 6 months), or concurrently with administering the anti-SMAD7 therapy.

The level of FCP can be analyzed at varying time points following an administering step (b). For instance, in some embodiments, following an administering step (b), the level of FCP is analyzed at least 1 day, at least 3 days, at least 5 days, at least 1 week, at least 2 weeks, at least 3 weeks, at least 1 month, at least 2 months, at least 4 months, or at least 6 months after said administration step. In some embodiments, the level of FCP is analyzed immediately after said administration step. In yet other embodiments, the level of FCP is analyzed about 7 days, about 10 days, about 15 days, about 20 days, about 25 days, or about 28 days after said administration step.

Normal levels of FCP can be determined based on numerical reference values or with respect to levels of FCP in a healthy control group. For instance, in some embodiments, normal levels of FCP are about ≤20 μg/g (feces), about ≤40 μg/g, about ≤60 μg/g, about ≤80 μg/g, about ≤100 μg/g, about ≤120 μg/g, about ≤140 μg/g, about ≤160 μg/g, about ≤180 μg/g, about ≤200 μg/g, about ≤220 μg/g, or about ≤240 μg/g. In some embodiments, normal levels of FCP in a healthy control aged 2-9 years are between about 100 μg/g and about 200 μg/g, between about 110 μg/g and about 190 μg/g, between about 120 μg/g and about 180 μg/g, between about 130 g/g and about 170 μg/g, or between about 140 μg/g and about 160 μg/g. In some embodiments, normal levels of FCP in a healthy control aged 10-59 years are about 166 μg/g. In some embodiments, normal levels of FCP in a healthy control aged 10-59 years is between about g/g and about 100 μg/g, between about 20 μg/g and about 90 μg/g, between about 30 μg/g and about 80 μg/g, between about 40 μg/g and about 70 μg/g, or between about 50 μg/g and about 60 μg/g. In some embodiments, normal levels of FCP in a healthy control aged 10-59 years are about 51 μg/g. In some embodiments, normal levels of FCP in a healthy control aged >60 years are between about 60 μg/g and about 160 μg/g, between about 70 μg/g and about 150 g/g, between about 80 μg/g and about 140 μg/g, between about 90 μg/g and about 130 μg/g, or between about 100 μg/g and about 120 μg/g. In some embodiments, normal levels of FCP in a healthy control aged >60 years are about 112 μg/g.

In other embodiments of the invention, normal levels of FCP are defined as median levels of FCP in a healthy control group. A healthy control group can be defined based on various criteria related to genetic background, habits, and physical attributes matched to the same set of criteria in the patient. For instance, in some embodiments, the healthy control group and the patient having IBD are matched with respect to age, gender, ethnic origin, smoking habits, dietary habits, body-mass index (BMI), and/or exercise habits.

In various embodiments of the invention, the initial dose of a SMAD7 AON administered to a patient having IBD can vary. For instance, in some embodiments, the initial dose of a SMAD7 AON administered to a patient having IBD is less than 500 mg/day, less than 400 mg/day, less than 300 mg/day, less than 200 mg/day, less than 100 mg/day, less than 90 mg/day, less than 80 mg/day, less than 70 mg/day, less than 60 mg/day, less than 50 mg/day, less than 40 mg/day, less than 30 mg/day, less than 20 mg/day, or less than 10 mg/day. Alternatively, in other embodiments, the initial dose is at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 200 mg/day, at least 300 mg/day, at least 400 mg/day, or at least 500 mg/day. In yet other embodiments, the initial dose is about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, or about 500 mg/day. In some embodiments, the initial dose is 5 mg/day, 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day.

In some embodiments of a method for treating or managing inflammatory bowel disease (IBD) provided in this section, after analyzing the level of FCP in the patient in a step (b) or (c), if the level of FCP is above normal levels of FCP, then the method can comprise the step of administering to the patient a subsequent dose that is greater than the initial dose. In some embodiments, after analyzing the level of FCP in the patient in a step (b) or (c), if the level of FCP is below normal levels of FCP, then the method can comprise the step of administering to the patient a subsequent dose that is smaller than the initial dose.

In another aspect, provided herein is a method for determining the level of a subsequent dose of SMAD7 AON with respect to an initial dose of SMAD7 AON based on levels of FCP in a patient having IBD. For instance, in embodiments of the invention described herein, if FCP levels in a patient having IBD are above normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day, at least about 170 mg/day, at least about 180 mg/day, at least about 190 mg/day, or at least about 200 mg/day greater than the initial dose. Alternatively, in some embodiments, if FCP levels in a patient having IBD are below normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day smaller than the initial dose. Furthermore, in some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

In another aspect, provided herein is a method for modulating treatment with a SMAD7 AON in a patient with IBD based on a comparison of relative levels of FCP in a patient before and after an initial administering step. The method comprises the following steps: (a) analyzing the level of FCP in the patient; and (b) if the level of FCP is above normal levels of FCP, then administering to the patient an initial dose of a SMAD7 AON; (c) analyzing the level of FCP in the patient after said administering step; and (d) if the level of FCP is lower after said administration step than the level of FCP before said administration step, then administering to the patient a subsequent dose that is the same as the initial dose or smaller than the initial dose. Alternatively, in step (d) if the level of FCP is unchanged or increased after said administration step (i.e., step (b)) compared to the level of FCP before said administration step, then step (d) comprises administering to the patient a subsequent dose that is greater than the initial dose or terminating the treatment. Alternatively, in step (d) if the patient is in clinical remission and the level of FCP is unchanged or increased after said administration step (i.e., step (b)) compared to the level of FCP before said administration step, then step (d) comprises terminating the treatment.

In some embodiments of a method provided in this section, a change in FCP levels observed after an initial administration step (of SMAD7 AON) compared to FCP levels prior to the administration step can be analyzed, for example, as a change in percent of FCP levels, to determine the amount of a subsequent dose of SMAD7 AON to be administered to a patient with IBD. For example, in some embodiments, if the level of FCP is at least 10%, at least 20%, at least 30%, at least 40%, at least 50%, at least 60%, at least 70%, at least 80%, at least 90%, or at least 95% decreased after said administration step (e.g., an administration step (b)) compared to the level of FCP before said administration step, then the method comprises a step (e.g., an administration step (d)) of administering to the patient a subsequent dose that is the same as the initial dose or smaller than the initial dose.

In another aspect, provided herein is a method for determining the probability that a patient having IBD will experience clinical remission following treatment with a SMAD7 AON based on a comparison of FCP levels, for example, based on a comparison of percent change in FCP levels before and after treatment with a SMAD7 AON. For example, in some embodiments, the methods described herein further comprise the step of determining that the patient having IBD has a greater than 20%, greater than 30%, greater than 40%, greater than 50%, greater than 60%, greater than 70%, greater than 80%, greater than 90% or greater than 100% chance of experiencing clinical remission of the IBD for a time period of at least 1 week, at least 2 weeks, at least 3 weeks, at least 4 weeks, at least 6 weeks or at least 8 weeks, if the level of FCP after an administering step (e.g., an administering step (b)) is at least 5%, at least 10%, at least 20%, at least 30%, at least 40%, at least 50%, at least 60%, at least 70%, at least 80%, at least 90%, or at least 95% decreased compared to the level of FCP before the administration step.

Clinical remission, as described herein, can be determined by comparison to a reference value, for example, a Crohn's Disease Activity Index (CDAI) or a Modified Mayo Score (MMS). In some embodiments of the invention, clinical remission in a patient having IBD is indicated by a CDAI score of less than 150 (CDAI <150), or a MMS ≤2. See, e.g., Section 7.2.2.

In some embodiments of a method provided in this section, a clinical response or clinical remission can be observed at a given time point or within a given time frame with respect to administration of the SMAD7 AON (e.g., using an analysis described in Section 7.2, including, e.g., CDAI score or MMS). For example, in some embodiments, clinical remission is observed about one day, about 3 days, about one week, about two weeks, about three weeks, about four weeks, about six weeks, about eight weeks, or about ten weeks after an administration step (for example, an administration step (b)) and maintained for a period of at least 3 days, at least 1 week, at least 2 weeks, at least 3 weeks, at least 4 weeks, at least 6 weeks at least 8 weeks, or at least 10 weeks. Similarly, some embodiments of the invention comprise a method of determining that the patient having IBD has a chance of experiencing clinical remission of IBD, where the patient having IBD had a CDAI of between about 220 and about 400, between about 150 and about 200, between about 200 and about 250, between about 250 and about 300, between about 300 and about 350, between about 350 and about 400, between about 400 and about 450, or greater than about 450 one week prior to an anti-SMAD7 therapy administration step (for example, an administration step (b)). Some embodiments of the invention comprise a method of determining that the patient having IBD has a chance of experiencing clinical remission of IBD, where the patient having IBD had a MMS of between about 4 and about 9, between about 2 and about 4, between about 4 and about 6, between about 6 and about 8, or greater than about 8 one week prior to an anti-SMAD7 therapy administration step (for example, an administration step (b)).

In some embodiments, a method of treating or managing IBD in a patient with above normal levels of FCP, comprises administering to the patient a dose of SMAD7 AON. Furthermore, in some embodiments, a methods for treating or managing IBD in a patient who has above normal FCP levels following administration of a dose of a SMAD7 AON, comprises administered a further dose of the SMAD7 AON that is greater than or equal to the prior dose. Similarly, in some embodiments of a method of treating or managing IBD, the patient having IBD has below normal FCP levels following administration of a dose of SMAD7 AON. In the latter case, the method will comprise administering to the patient a further dose of the SMAD7 AON that is less than or equal to the prior dose. In some embodiments, administration of the SMAD7 AON to the patient is repeated until the patient shows a clinical response or remission, e.g., based on monitoring a clinical parameter described in Section 7.2, e.g., until the levels of a biomarker, e.g., SMAD7, SMAD3-phosphorylation, HLA-DR, CD4, CD8, IFN-γ, IL-12, IL17A, IL6, IL8, CRP, TNFα, FCP reach normal levels or until the patient achieves a CDAI score of less than 150, or based on any other clinical parameter described in Section 7.2.

In some embodiments of a method of treating or managing IBD in a patient having above normal levels of FCP, the amount of a SMAD7 AON administered to the patient is increased until FCP levels in the patient decrease. In such embodiments, levels of SMAD7 AON administered to the patient can be increased until the level of FCP in the patient decreases to about a normal level of FCP or a below normal level of FCP.

In some embodiments of a method of treating or managing IBD comprises monitoring the treatment or management of IBD in a patient with IBD, that comprises analyzing FCP levels in the patient following each SMAD7 AON administration. Utilizing these methods, the absence of a decrease in FCP levels indicates that the treatment or management is not effective. In such embodiments, FCP levels can be analyzed one time or multiple times, for instance, two times, three times, four times, about five times, about 10 times, about 15 times, about 20 times, or about 30 times, after each administration of SMAD7 AON. Furthermore, the timing of the measurement of FCP levels can vary with respect to the time of SMAD7 AON administration such that FCP levels can be analyzed immediately after, about 1 hour after, about 3 hours after, about 6 hours after, about 12 hours after, about 1 day after, about 3 days after, about 1 week after, about 2 weeks after, and/or about 1 month after SMAD7 AON administration.

In order to determine levels of a biomarker or analyte, for example, FCP, in a patient having IBD using the methods described herein, a sample can be obtained from the patient. Therefore, in some embodiments of a method of treating or managing IBD provided in this section, the level of FCP in the patient having IBD is determined in a sample obtained from the patient having IBD. Analytes other than or in addition to FCP, for example, but not limited to Interleukin-6 (IL6), Interleukin-8 (IL8), Interleukin-12 (IL12), Interleukin-17A (IL17A), Interferon gamma (IFN-γ,) Tumor Necrosis Factor alpha (TNFα), Cluster of Differentiation 4 (CD4), Cluster of Differentiation 8 (CD8), Human Leukocyte Antigen-DR (HLA-DR), and C-Reactive Protein (CRP), can also be determined in methods of the invention. Thus, in some embodiments, the method comprises determining a level, or multiple levels, of one or more additional analytes in the patient having IBD. Analytes of TNFα comprise RNA, DNA, and protein products of or derived from the TNFα gene, described by NCBI Reference Sequence: NG_007462.1. Analytes of CRP comprise RNA, DNA, and protein products of or derived from the CRP gene, described by NCBI Reference Sequence: NG_013007.1. Analytes of IL8 comprise RNA, DNA, and protein products of or derived from the TNFα gene, described by NCBI Reference Sequence: NG_029889.1. Analytes of FCP comprise RNA, DNA, and protein products of or derived from the FCP gene, described by Entrez GeneID No. 6280. Analytes of IL6 comprise RNA, DNA, and protein products of or derived from the IL6 gene, described by Entrez GeneID No. 3569. Analytes of IL8 comprise RNA, DNA, and protein products of or derived from the IL8 gene, described by Entrez GeneID No. 3567. Analytes of IL12 comprise RNA, DNA, and protein products of or derived from the IL12 gene, described by Entrez GeneID No. 3593. Analytes of IL17A comprise RNA, DNA, and protein products of or derived from the IL17A gene described by Entrez GeneID No. 3605. Analytes of IFNγ comprise RNA, DNA, and protein products of or derived from the IFNγ gene, described by Entrez GeneID No. 3458. Analytes of CD4 comprise RNA, DNA, and protein products of or derived from the CD4 gene, described by Entrez GeneID No. 920. Analytes of CD8 comprise RNA, DNA, and protein products of or derived from the CD8 gene, described by Entrez GeneID No. 925. Analytes of HLA-DR comprise RNA, DNA, and protein products of or derived from the HLA-DR gene family (including, e.g., HLA-DRA, HLA-DRB1, HLA-DRB3, HLA-DRB4, and HLA-DRB5), described, e.g., by Entrez GeneID Nos. 3122, 3123, 3125, 3126, and 3127. Analytes of Foxp3 comprise RNA, DNA, and protein products of or derived from the Foxp3 gene, described by Entrez GeneID No. 50943.

Samples containing analytes of interest, for example, SMAD7, phosphor-SMAD3, HLA-DR, TNFα, CRP, IFN-γ, IL6, IL8, IL12, IL17A, CD4 and/or CD8, obtained from the patient having IBD, can comprise blood, serum, or plasma samples. Samples containing FCP can comprise stool samples. Stool samples can be wet stool samples or dry stool samples. Samples can also comprise tissue samples such as, but not limited to, tissue, gastrointestinal, mucosal, submucosal, intestinal, esophageal, ileal, rectal, or lymphatic samples. Levels of analytes of interest in a sample from a patient having IBD can be determined using various assays. For example, in methods of the invention, the level of FCP and/or another analyte can be determined by immunochemistry, for example, by an enzyme-linked immunosorbent assay (ELISA), or by nucleotide analysis.

Methods provided herein comprise methods for treating and managing various forms of IBD. For example, the invention comprises methods for treating and managing IBD, where the IBD is Crohn's Disease (CD) or ulcerative colitis (UC). The contemplated invention also provides methods for treating different types of patients with IBD, including, for example, but not limited to, IBD patients that are steroid-dependent patients with active CD; and steroid-resistant patients with active CD.

It will be appreciated that the SMAD7 AON administered to the patient having IBD in methods of the invention described herein, can be administered by various administration routes. In various embodiments, the SMAD7 AON can be administered by one or several routes, including orally, topically, parenterally, e.g., by subcutaneous injection, by inhalation spray, or rectally. The term parenteral as used herein comprises subcutaneous injections, intrapancreatic administration, intravenous, intramuscular, intraperitoneal, intrasternal injection or infusion techniques. In a preferred embodiment, the SMAD7 AON may be administered orally to the patient having IBD.

The SMAD7 AON described in Section 7.11 can, for example, be used in the methods of the invention described herein.

7.4 IL6, IL12, and HLA-DR

In the methods provided herein, the biomarkers IL6, IL12, or HLA-DR can be used to monitor the activities of an anti-SMAD7 treatment, as described in Section 7.2 (e.g., to analyze if a patient shows a clinical response to a SMAD7 AON, or if a patient experiences remission). In some embodiments, IL6, IL12 or HLA-DR levels in an IBD patient sample can inform a decision regarding whether an IBD patient is transitioning from the first to the second treatment phase (e.g., if the patient shows a clinical response to a SMAD7 AON or if the patient shows remission), e.g., as described in Section 7.1.

In other methods provided herein, IL6, IL12 or HLA-DR can be used as biomarkers for patient selection.

In another aspect, provided herein is a method for treating or managing IBD in a patient having IBD. In one embodiment, the method comprises the following steps: (a) of administering to the patient an initial dose of a SMAD7 AON; (b) analyzing the level of IL6, IL12 or HLA-DR in the patient; and (c) if the level of IL6, IL12 or HLA-DR is above normal levels of IL6, IL12 or HLA-DR then administering to the patient a subsequent dose that is greater than or equal to the initial dose. Alternatively, if in step (c), the level of IL6, IL12 or HLA-DR is below normal levels of IL6, IL12 or HLA-DR as determined in step (b), then step (c) comprises administering to the patient a subsequent dose that is equal to or smaller than the initial dose.

In another aspect, provided herein is a method for treating or managing inflammatory bowel disease (IBD) in a patient having IBD, wherein the method comprises (a) establishing a control level of IL6, IL12 or HLA-DR for the patient; (b) administering to the patient an initial dose of a SMAD7 antisense-oligonucleotide; c) analyzing the level of IL6, IL12 or HLA-DR in the patient; and (d) if the level of IL6, IL12 or HLA-DR is lower than the control level, then administering to the patient a subsequent dose that is the same as the initial dose or smaller than the initial dose, or, if the level of IL6, IL12 or HLA-DR is unchanged or increased compared to the control level, then administering to the patient a subsequent dose that is the same as the initial dose or greater than the initial dose or terminating the treatment.

In some embodiments, the control level for the IBD patient is the IL6, IL12 or HLA-DR level in a sample obtained from the IBD patient prior to administration of the first anti-SMAD7 treatment during a chronic disease period, e.g., when the patient was in remission. In some embodiments, the control level for the IBD patient is the IL6, IL12 or HLA-DR level in a sample obtained from the IBD patient prior to administration of the first anti-SMAD7 treatment during an acute disease period (e.g., CDAI >150; CDAI >250 and ≤450; or a Modified Mayo Score (MMS)). In some embodiments, the control level for the IBD patient is the IL6, IL10, IL12 or HLA-DR level in a sample obtained from the IBD patient during a period when the patient is administered with an anti-SMAD7 treatment, or at the beginning of a treatment period (e.g., during week 0, baseline level). In some embodiments, the control level of the IBD patient is the IL6, IL12 or HLA-DR in a sample obtained from the IBD patient at an earlier timepoint during an anti-SMAD7 treatment period.

In another aspect, provided herein is a method for treating or managing IBD in a patient having IBD with respect to administration of an initial dose of a SMAD7 AON. In one embodiment, the provided herein is a method for treating or managing IBD in a patient having IBD, where the method comprises the following steps: (a) analyzing the level of IL6, IL12 or HLA-DR in the patient; and (b) if the level of IL6, IL12 or HLA-DR is above normal levels of IL6, IL12 or HLA-DR then administering to the patient an initial dose of a SMAD7 AON. Additionally, the method can further comprise the steps of: (c) analyzing the level of IL6, IL12, or HLA-DR in the patient after said administering step, i.e., step (b); and (d) if the level of IL6, IL12, or HLA-DR is above normal levels of IL6, IL12 or HLA-DR then administering to the patient a subsequent dose that is greater than or equal to the initial dose. Alternatively, if in step (d), the level of IL6, IL12 or HLA-DR is below normal levels of IL6, L12 or HLA-DR, as determined in step (c), then step (d) comprises administering to the patient a subsequent dose that is equal to or smaller than the initial dose. In some instances, if the subsequent dose administered in step (d) is equal to or greater than the maximum tolerated dose (MTD), then the method comprises the step of terminating the treatment.

The level of IL6, IL12 or HLA-DR can be analyzed at any timepoint during an administration schedule in a method for treating IBD provided herein. For example, the IL6, IL12 or HLA-DR level can be analyzed before or after administering an anti-SMAD7 therapy (e.g., at least 1 day, at least 3 days, at least 5 days, at least 1 week, at least 2 weeks, at least 3 weeks, at least 1 month, at least 2 months, at least 4 months, or at least 6 months), or concurrently with administering the anti-SMAD7 therapy.

The level of IL6, IL12 or HLA-DR can be analyzed at varying time points following an administering step (b). For instance, in some embodiments, following an administering step (b), the level of IL6, IL12 or HLA-DR is analyzed at least 1 day, at least 3 days, at least 5 days, at least 1 week, at least 2 weeks, at least 3 weeks, at least 1 month, at least 2 months, at least 4 months, or at least 6 months after said administration step. In some embodiments, the level of IL6, IL12 or HLA-DR is analyzed immediately after said administration step. In yet other embodiments, the level of IL6, IL12 or HLA-DR is analyzed about 7 days, about 10 days, about 15 days, about 20 days, about 25 days, or about 28 days after said administration step.

Normal levels of IL6, IL12 or HLA-DR can be determined based on numerical reference values or with respect to levels of IL6, IL12 or HLA-DR in a healthy control group. For instance, in some embodiments, normal levels of IL6 levels in a blood, serum or plasma sample from the patient having IBD are <600 pg/ml, <500 pg/ml, <400 pg/ml, or <300 pg/ml. In some embodiments, normal levels of IL12 levels in a blood, serum or plasma sample from the patient having IBD are <100 pg/ml, <75 pg/ml, <50 pg/ml, or <25 pg/ml.

In other embodiments of the invention, normal levels of IL6, IL12 or HLA-DR are defined as median levels of IL6, IL12 or HLA-DR in a healthy control group. A healthy control group can be defined based on various criteria related to genetic background, habits, and physical attributes matched to the same set of criteria in the patient. For instance, in some embodiments, the healthy control group and the patient having IBD are matched with respect to age, gender, ethnic origin, smoking habits, dietary habits, body-mass index (BMI), and/or exercise habits.

In various embodiments of the invention, the initial dose of a SMAD7 AON administered to a patient having IBD can vary. For instance, in some embodiments, the initial dose of a SMAD7 AON administered to a patient having IBD is less than 500 mg/day, less than 400 mg/day, less than 300 mg/day, less than 200 mg/day, less than 100 mg/day, less than 90 mg/day, less than 80 mg/day, less than 70 mg/day, less than 60 mg/day, less than 50 mg/day, less than 40 mg/day, less than 30 mg/day, less than 20 mg/day, or less than 10 mg/day. Alternatively, in other embodiments, the initial dose is at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 200 mg/day, at least 300 mg/day, at least 400 mg/day, or at least 500 mg/day. In yet other embodiments, the initial dose is about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, or about 500 mg/day. In some embodiments, the initial dose is 5 mg/day, 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day.

In some embodiments of a method for treating or managing inflammatory bowel disease (IBD) provided in this section, after analyzing the level of IL6, IL12 or HLA-DR in the patient in a step (b) or (c), if the level of IL6, IL12 or HLA-DR is above normal levels of IL6, IL12 or HLA-DR then the method can comprise the step of administering to the patient a subsequent dose that is greater than the initial dose. In some embodiments, after analyzing the level of IL6, IL12 or HLA-DR in the patient in a step (b) or (c), if the level of IL6, IL12 or HLA-DR is below normal levels of IL6, IL12 or HLA-DR then the method can comprise the step of administering to the patient a subsequent dose that is smaller than the initial dose.

In another aspect, provided herein is a method for determining the level of a subsequent dose of SMAD7 AON with respect to an initial dose of SMAD7 AON based on levels of IL6, IL12 or HLA-DR in a patient having IBD. For instance, in embodiments of the invention described herein, if IL6, IL12 or HLA-DR levels in a patient having IBD are above normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day, at least about 170 mg/day, at least about 180 mg/day, at least about 190 mg/day, or at least about 200 mg/day greater than the initial dose. Alternatively, in some embodiments, if IL6, IL12 or HLA-DR levels in a patient having IBD are below normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day smaller than the initial dose. Furthermore, in some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

In another aspect, provided herein is a method for modulating treatment with a SMAD7 AON in a patient with IBD based on a comparison of relative levels of IL6, IL12 or HLA-DR in a patient before and after an initial administering step. The method comprises the following steps: (a) analyzing the level of IL6, IL12 or HLA-DR in the patient; and (b) if the level of IL6, IL12 or HLA-DR is above normal levels of IL6, IL12 or HLA-DR, then administering to the patient an initial dose of a SMAD7 AON; (c) analyzing the level of IL6, IL12 or HLA-DR in the patient after said administering step; and (d) if the level of IL6, IL12 or HLA-DR is lower after said administration step than the level of IL6, IL12, or HLA-DR before said administration step, then administering to the patient a subsequent dose that is the same as the initial dose or smaller than the initial dose. Alternatively, in step (d) if the level of IL6, IL12, or HLA-DR is unchanged or increased after said administration step (i.e., step (b)) compared to the level of IL6, IL12 or HLA-DR before said administration step, then step (d) comprises administering to the patient a subsequent dose that is greater than the initial dose or terminating the treatment. Alternatively, in step (d) if the patient is in clinical remission and the level of IL6, IL12 or HLA-DR is unchanged or increased after said administration step (i.e., step (b)) compared to the level of IL6, IL12 or HLA-DR before said administration step, then step (d) comprises terminating the treatment.

In some embodiments of a method provided in this section, a change in IL6, IL12 or HLA-DR levels observed after an initial administration step (of SMAD7 AON) compared to IL6, IL12 or HLA-DR levels prior to the administration step can be analyzed, for example, as a change in percent of IL6, IL12 or HLA-DR to determine the amount of a subsequent dose of SMAD7 AON to be administered to a patient with IBD. For example, in some embodiments, if the level of IL6, IL12 or HLA-DR is at least 10%, at least 20%, at least 30%, at least 40%, at least 50%, at least 60%, at least 70%, at least 80%, at least 90%, or at least 95% decreased after said administration step (e.g., an administration step (b)) compared to the level of IL6, 1L12 or HLA-DR before said administration step, then the method comprises a step (e.g., an administration step (d)) of administering to the patient a subsequent dose that is the same as the initial dose or smaller than the initial dose.

In another aspect, provided herein is a method for determining the probability that a patient having IBD will experience clinical remission following treatment with a SMAD7 AON based on a comparison of IL6, IL12 or HLA-DR, for example, based on a comparison of percent change in IL6, IL12, or HLA-DR levels before and after treatment with a SMAD7 AON. For example, in some embodiments, the methods described herein further comprise the step of determining that the patient having IBD has a greater than 20%, greater than 30%, greater than 40%, greater than 50%, greater than 60%, greater than 70%, greater than 80%, greater than 90% or greater than 100% chance of experiencing clinical remission of the IBD for a time period of at least 1 week, at least 2 weeks, at least 3 weeks, at least 4 weeks, at least 6 weeks or at least 8 weeks, if the level of IL6, IL12 or HLA-DR after an administering step (e.g., an administering step (b)) is at least 5%, at least 10%, at least 20%, at least 30%, at least 40%, at least 50%, at least 60%, at least 70%, at least 80%, at least 90%, or at least 95% decreased compared to the level of IL6, IL12 or HLA-DR before the administration step.

Clinical remission, as described herein, can be determined by comparison to a reference value, for example, a Crohn's Disease Activity Index (CDAI) or a Modified Mayo Score (MMS). In some embodiments of the invention, clinical remission in a patient having IBD is indicated by a CDAI score of less than 150 (CDAI <150) or by an MMS of 2 or less (MMS ≤2). See, e.g., Section 7.2.2.

In some embodiments of a method provided in this section, a clinical response or clinical remission can be observed at a given time point or within a given time frame with respect to administration of the SMAD7 AON (e.g., using an analysis described in Section 7.2, including, e.g., CDAI score). For example, in some embodiments, clinical remission is observed about one day, about 3 days, about one week, about two weeks, about three weeks, about four weeks, about six weeks, about eight weeks, or about ten weeks after an administration step (for example, an administration step (b)) and maintained for a period of at least 3 days, at least 1 week, at least 2 weeks, at least 3 weeks, at least 4 weeks, at least 6 weeks at least 8 weeks, or at least 10 weeks. Similarly, some embodiments of the invention comprise a method of determining that the patient having IBD has a chance of experiencing clinical remission of IBD, where the patient having IBD had a CDAI of between about 220 and about 400, about 150 and about 200, about 200 and about 250, about 250 and about 300, about 300 and about 350, about 350 and about 400, about 400 and about 450, or greater than about 450 one week prior to an anti-SMAD7 therapy administration step (for example, an administration step (b)). Some embodiments of the invention comprise a method of determining that the patient having IBD has a chance of experiencing clinical remission of IBD, where the patient having IBD had a MMS of between about 4 and about 9, between about 2 and about 4, between about 4 and about 6, between about 6 and about 8, or greater than about 8 one week prior to an anti-SMAD7 therapy administration step (for example, an administration step (b)).

In some embodiments, a method of treating or managing IBD in a patient with above normal levels of IL6, IL12 or HLA-DR, comprises administering to the patient a dose of SMAD7 AON. Furthermore, in some embodiments, a methods for treating or managing IBD in a patient who has above normal IL6, IL12 or HLA-DR levels following administration of a dose of a SMAD7 AON, comprises administered a further dose of the SMAD7 AON that is greater than or equal to the prior dose. Similarly, in some embodiments of a method of treating or managing IBD, the patient having IBD has below normal IL6, IL12 or HLA-DR levels following administration of a dose of SMAD7 AON. In the latter case, the method will comprise administering to the patient a further dose of the SMAD7 AON that is less than or equal to the prior dose. In some embodiments, administration of the SMAD7 AON to the patient is repeated until the patient shows a clinical response or remission, e.g., based on monitoring a clinical parameter described in Section 7.2, e.g., until the levels of a biomarker, e.g., SMAD7, Foxp3, CCR9, CCL20, IL10, CD4, CD8, IFN-γ, IL17, IL4, IL8, CRP, TNFα, FCP reach normal levels or until the patient achieves a CDAI score of less than 150, or based on any other clinical parameter described in Section 7.2.

In some embodiments of a method of treating or managing IBD in a patient having above normal levels of IL6, IL12 or HLA-DR, the amount of a SMAD7 AON administered to the patient is increased until IL6, IL12 or HLA-DR levels in the patient decrease. In such embodiments, levels of SMAD7 AON administered to the patient can be increased until the level of IL6, IL12 or HLA-DR in the patient decreases to about a normal level of IL6, IL12 or HLA-DR or a below normal level of IL6, IL2 or HLA-DR.

In some embodiments of a method of treating or managing IBD comprises monitoring the treatment or management of IBD in a patient with IBD, that comprises analyzing IL6, IL12 or HLA-DR levels in the patient following each SMAD7 AON administration. Utilizing these methods, the absence of a decrease in IL6, IL12 or HLA-DR levels indicates that the treatment or management is not effective. In such embodiments, IL6, IL12 or HLA-DR levels can be analyzed one time or multiple times, for instance, two times, three times, four times, about five times, about 10 times, about 15 times, about 20 times, or about 30 times, after each administration of SMAD7 AON. Furthermore, the timing of the measurement of IL6, IL12 or HLA-DR levels can vary with respect to the time of SMAD7 AON administration such that IL6, IL12 or HLA-DR levels can be analyzed immediately after, about 1 hour after, about 3 hours after, about 6 hours after, about 12 hours after, about 1 day after, about 3 days after, about 1 week after, about 2 weeks after, and/or about 1 month after SMAD7 AON administration.

In order to determine levels of a biomarker or analyte, for example, IL6, IL12 or HLA-DR, in a patient having IBD using the methods described herein, a sample can be obtained from the patient. Therefore, in some embodiments of a method of treating or managing IBD provided in this section, the level of IL6, IL12 or HLA-DR in the patient having IBD is determined in a sample obtained from the patient having IBD. Analytes other than or in addition to IL6, IL12 or HLA-DR, for example, but not limited to Fecal Calprotectin (FCP), Interleukin-8 (IL8), Interleukin-17 (IL7A), Interferon gamma (IFN-γ,) Tumor Necrosis Factor alpha (TNFα), Cluster of Differentiation 4 (CD4), Cluster of Differentiation 8 (CD8) and C-Reactive Protein (CRP), can also be determined in methods of the invention. Thus, in some embodiments, the method comprises determining a level, or multiple levels, of one or more additional analytes in the patient having IBD. Analytes of TNFα comprise RNA, DNA, and protein products of or derived from the TNFα gene, described by NCBI Reference Sequence: NG_007462.1. Analytes of CRP comprise RNA, DNA, and protein products of or derived from the CRP gene, described by NCBI Reference Sequence: NG_013007.1. Analytes of IL8 comprise RNA, DNA, and protein products of or derived from the TNFα gene, described by NCBI Reference Sequence: NG_029889.1. Analytes of FCP comprise RNA, DNA, and protein products of or derived from the FCP gene, described by Entrez GeneID No. 6280. Analytes of IL6 comprise RNA, DNA, and protein products of or derived from the IL6 gene, described by Entrez GeneID No. 3569. Analytes of IL8 comprise RNA, DNA, and protein products of or derived from the IL8 gene, described by Entrez GeneID No. 3567. Analytes of IL12 comprise RNA, DNA, and protein products of or derived from the IL12 gene, described by Entrez GeneID No. 3593. Analytes of IL17 comprise RNA, DNA, and protein products of or derived from the IL17A gene, described by Entrez GeneID No. 3605. Analytes of IFNγ comprise RNA, DNA, and protein products of or derived from the IFNγ gene, described by Entrez GeneID No. 3458. Analytes of CD4 comprise RNA, DNA, and protein products of or derived from the CD4 gene, described by Entrez GeneID No. 920. Analytes of CD8 comprise RNA, DNA, and protein products of or derived from the CD8 gene, described by Entrez GeneID No. 925. Analytes of HLA-DR comprise RNA, DNA, and protein products of or derived from the HLA-DR gene family (including, e.g., HLA-DRA, HLA-DRB1, HLA-DRB3, HLA-DRB4, and HLA-DRB5), described, e.g., by Entrez GeneID Nos. 3122, 3123, 3125, 3126, and 3127.

Samples containing analytes of interest, for example, SMAD7, phosphor-SMAD3, HLA-DR, TNFα, CRP, IFN-γ, IL6, IL8, IL12, IL17, CD4 and/or CD8, obtained from the patient having IBD, can comprise blood, serum, or plasma samples. Samples containing FCP can comprise stool samples. Stool samples can be wet stool samples or dry stool samples. Samples can also comprise tissue samples such as, but not limited to, tissue, gastrointestinal, mucosal, submucosal, intestinal, esophageal, ileal, rectal, or lymphatic samples. Levels of analytes of interest in a sample from a patient having IBD can be determined using various assays. For example, in methods of the invention, the level of FCP and/or another analyte can be determined by immunochemistry, for example, by an enzyme-linked immunosorbent assay (ELISA), or by nucleotide analysis.

Methods provided herein comprise methods for treating and managing various forms of IBD. For example, the invention comprises methods for treating and managing IBD, where the IBD is Crohn's Disease (CD) or ulcerative colitis (UC). The contemplated invention also provides methods for treating different types of patients with IBD, including, for example, but not limited to, IBD patients that are steroid-dependent patients with active CD; and steroid-resistant patients with active CD.

It will be appreciated that the SMAD7 AON administered to the patient having IBD in methods of the invention described herein, can be administered by various administration routes. In various embodiments, the SMAD7 AON can be administered by one or several routes, including orally, topically, parenterally, e.g., by subcutaneous injection, by inhalation spray, or rectally. The term parenteral as used herein comprises subcutaneous injections, intrapancreatic administration, intravenous, intramuscular, intraperitoneal, intrasternal injection or infusion techniques. In a preferred embodiment, the SMAD7 AON may be administered orally to the patient having IBD

The SMAD7 AONs described in Section 7.11 can, for example, be used in the methods of the invention described herein.

7.5 Phospho-SMAD3

In the methods provided herein, the biomarker phospho-SMAD3 can be used to monitor the activities of an anti-SMAD7 treatment, as described in Section 7.2 (e.g., to analyze if a patient shows a clinical response to a SMAD7 AON, or if a patient experiences remission). In some embodiments, phospho-SMAD3 levels in an IBD patient sample can inform a decision regarding whether an IBD patient is transitioning from the first to the second treatment phase (e.g., if the patient shows a clinical response to a SMAD7 AON or if the patient shows remission), e.g., as described in Section 7.1.

In other methods provided herein, phospho-SMAD3 can be used as biomarkers for patient selection.

In another aspect, provided herein is a method for treating or managing IBD in a patient having IBD. In one embodiment, the method comprises the following steps: (a) of administering to the patient an initial dose of a SMAD7 AON; (b) analyzing the level of phospho-SMAD3 in the patient; and (c) if the level of phospho-SMAD3 is below normal levels of phospho-SMAD3 then administering to the patient a subsequent dose that is greater than or equal to the initial dose. Alternatively, if in step (c), the level of phospho-SMAD3 is above normal levels of phospho-SMAD3 as determined in step (b), then step (c) comprises administering to the patient a subsequent dose that is equal to or smaller than the initial dose.

In another aspect, provided herein is a method for treating or managing inflammatory bowel disease (IBD) in a patient having IBD, wherein the method comprises (a) establishing a control level of phospho-SMAD3 for the patient; (b) administering to the patient an initial dose of a SMAD7 antisense-oligonucleotide; c) analyzing the level of phospho-SMAD3 in the patient; and (d) if the level of phospho-SMAD3 is higher than the control level, then administering to the patient a subsequent dose that is the same as the initial dose or smaller than the initial dose, or, if the level of phospho-SMAD3 is unchanged or lower compared to the control level, then administering to the patient a subsequent dose that is the same as the initial dose or greater than the initial dose or terminating the treatment.

In some embodiments, the control level for the IBD patient is the phospho-SMAD3 level in a sample obtained from the IBD patient prior to administration of the first anti-SMAD7 treatment during a chronic disease period, e.g., when the patient was in remission. In some embodiments, the control level for the IBD patient is the phospho-SMAD3 level in a sample obtained from the IBD patient prior to administration of the first anti-SMAD7 treatment during an acute disease period (e.g., CDAI >150; CDAI ≥250 and ≤450; MMS ≥4 and ≤9). In some embodiments, the control level for the IBD patient is the phospho-SMAD3 level in a sample obtained from the IBD patient during a period when the patient is administered with an anti-SMAD7 treatment, or at the beginning of a treatment period (e.g., during week 0, baseline level). In some embodiments, the control level of the IBD patient is the phospho-SMAD3 in a sample obtained from the IBD patient at an earlier timepoint during an anti-SMAD7 treatment period.

In another aspect, provided herein is a method for treating or managing IBD in a patient having IBD with respect to administration of an initial dose of a SMAD7 AON. In one embodiment, the provided herein is a method for treating or managing IBD in a patient having IBD, where the method comprises the following steps: (a) analyzing the level of phospho-SMAD3 in the patient; and (b) if the level of phospho-SMAD3 is below normal levels of phospho-SMAD3 then administering to the patient an initial dose of a SMAD7 AON. Additionally, the method can further comprise the steps of: (c) analyzing the level of phospho-SMAD3 in the patient after said administering step, i.e., step (b); and (d) if the level of phospho-SMAD3 is below normal levels of phospho-SMAD3 then administering to the patient a subsequent dose that is greater than or equal to the initial dose. Alternatively, if in step (d), the level of phospho-SMAD3 is above normal levels of phospho-SMAD3, as determined in step (c), then step (d) comprises administering to the patient a subsequent dose that is equal to or smaller than the initial dose. In some instances, if the subsequent dose administered in step (d) is equal to or greater than the maximum tolerated dose (MTD), then the method comprises the step of terminating the treatment.

The level of phospho-SMAD3 can be analyzed at any timepoint during an administration schedule in a method for treating IBD provided herein. For example, the phospho-SMAD3 can be analyzed before or after administering an anti-SMAD7 therapy (e.g., at least 1 day, at least 3 days, at least 5 days, at least 1 week, at least 2 weeks, at least 3 weeks, at least 1 month, at least 2 months, at least 4 months, or at least 6 months), or concurrently with administering the anti-SMAD7 therapy.

The level of phospho-SMAD3 can be analyzed at varying time points following an administering step (b). For instance, in some embodiments, following an administering step (b), the level of phospho-SMAD3 is analyzed at least 1 day, at least 3 days, at least 5 days, at least 1 week, at least 2 weeks, at least 3 weeks, at least 1 month, at least 2 months, at least 4 months, or at least 6 months after said administration step. In some embodiments, the level of phospho-SMAD3 is analyzed immediately after said administration step. In yet other embodiments, the level of phospho-SMAD3 is analyzed about 7 days, about 10 days, about 15 days, about 20 days, about 25 days, or about 28 days after said administration step.

Normal levels of phospho-SMAD3 can be determined by comparison with levels of phospho-SMAD3 in a healthy control group.

In other embodiments of the invention, normal levels of phospho-SMAD3 are defined as median levels of phospho-SMAD3 in a healthy control group. A healthy control group can be defined based on various criteria related to genetic background, habits, and physical attributes matched to the same set of criteria in the patient. For instance, in some embodiments, the healthy control group and the patient having IBD are matched with respect to age, gender, ethnic origin, smoking habits, dietary habits, body-mass index (BMI), and/or exercise habits.

In various embodiments of the invention, the initial dose of a SMAD7 AON administered to a patient having IBD can vary. For instance, in some embodiments, the initial dose of a SMAD7 AON administered to a patient having IBD is less than 500 mg/day, less than 400 mg/day, less than 300 mg/day, less than 200 mg/day, less than 100 mg/day, less than 90 mg/day, less than 80 mg/day, less than 70 mg/day, less than 60 mg/day, less than 50 mg/day, less than 40 mg/day, less than 30 mg/day, less than 20 mg/day, or less than 10 mg/day. Alternatively, in other embodiments, the initial dose is at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 200 mg/day, at least 300 mg/day, at least 400 mg/day, or at least 500 mg/day. In yet other embodiments, the initial dose is about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, or about 500 mg/day. In some embodiments, the initial dose is 5 mg/day, 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day.

In some embodiments of a method for treating or managing inflammatory bowel disease (IBD) provided in this section, after analyzing the level of phospho-SMAD3 in the patient in a step (b) or (c), if the level of phospho-SMAD3 is below normal levels of phospho-SMAD3 then the method can comprise the step of administering to the patient a subsequent dose that is greater than the initial dose. In some embodiments, after analyzing the level of phospho-SMAD3 in the patient in a step (b) or (c), if the level of phospho-SMAD3 is above normal levels of phospho-SMAD3 then the method can comprise the step of administering to the patient a subsequent dose that is smaller than the initial dose.

In another aspect, provided herein is a method for determining the level of a subsequent dose of SMAD7 AON with respect to an initial dose of SMAD7 AON based on levels of phospho-SMAD3 in a patient having IBD. For instance, in embodiments of the invention described herein, phospho-SMAD3 levels in a patient having IBD are below normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day, at least about 170 mg/day, at least about 180 mg/day, at least about 190 mg/day, or at least about 200 mg/day greater than the initial dose. Alternatively, in some embodiments, if phospho-SMAD3 levels in a patient having IBD are above normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day smaller than the initial dose. Furthermore, in some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

In another aspect, provided herein is a method for modulating treatment with a SMAD7 AON in a patient with IBD based on a comparison of relative levels of phospho-SMAD3 in a patient before and after an initial administering step. The method comprises the following steps: (a) analyzing the level of phospho-SMAD3 in the patient; and (b) if the level of phospho-SMAD3 is below normal levels of phospho-SMAD3, then administering to the patient an initial dose of a SMAD7 AON; (c) analyzing the level of phospho-SMAD3 in the patient after said administering step; and (d) if the level of phospho-SMAD3 is higher after said administration step than the level of phospho-SMAD3 before said administration step, then administering to the patient a subsequent dose that is the same as the initial dose or smaller than the initial dose. Alternatively, in step (d) if the level of phospho-SMAD3 is unchanged or lower after said administration step (i.e., step (b)) compared to the level of phospho-SMAD3 before said administration step, then step (d) comprises administering to the patient a subsequent dose that is greater than the initial dose or terminating the treatment. Alternatively, in step (d) if the patient is in clinical remission and the level of phospho-SMAD3 is unchanged or decreased after said administration step (i.e., step (b)) compared to the level of phospho-SMAD3 before said administration step, then step (d) comprises terminating the treatment.

In some embodiments of a method provided in this section, a change in phospho-SMAD3 levels observed after an initial administration step (of SMAD7 AON) compared to phospho-SMAD3 levels prior to the administration step can be analyzed, for example, as a change in percent of phospho-SMAD3 to determine the amount of a subsequent dose of SMAD7 AON to be administered to a patient with IBD. For example, in some embodiments, if the level of phospho-SMAD3 is at least 2-fold, at least 3-fold, at least 4-fold, at least 5-fold, at least 6-fold, at least 7-fold, at least 8-fold, at least 9-fold, or at least 10-fold increased after said administration step (e.g., an administration step (b)) compared to the level of phospho-SMAD3 before said administration step, then the method comprises a step (e.g., an administration step (d)) of administering to the patient a subsequent dose that is the same as the initial dose or smaller than the initial dose.

In another aspect, provided herein is a method for determining the probability that a patient having IBD will experience clinical remission following treatment with a SMAD7 AON based on a comparison of phospho-SMAD3, for example, based on a comparison of percent change in phospho-SMAD3 levels before and after treatment with a SMAD7 AON. For example, in some embodiments, the methods described herein further comprise the step of determining that the patient having IBD has a greater than 20%, greater than 30%, greater than 40%, greater than 50%, greater than 60%, greater than 70%, greater than 80%, greater than 90% or greater than 100% chance of experiencing clinical remission of the IBD for a time period of at least 1 week, at least 2 weeks, at least 3 weeks, at least 4 weeks, at least 6 weeks or at least 8 weeks, if the level of phospho-SMAD3 after an administering step (e.g., an administering step (b)) is at least 2-fold, at least 3-fold, at least 4-fold, at least 5-fold, at least 6-fold, at least 7-fold, at least 8-fold, at least 9-fold, or at least 10-fold increased compared to the level of phospho-SMAD3 before the administration step.

Clinical remission, as described herein, can be determined by comparison to a reference value, for example, a Crohn's Disease Activity Index (CDAI) or Modified Mayo Score (MMS). In some embodiments of the invention, clinical remission in a patient having IBD is indicated by a CDAI score of less than 150 (CDAI <150) or an MMS ≤2. See, e.g., Section 7.2.2.

In some embodiments of a method provided in this section, a clinical response or clinical remission can be observed at a given time point or within a given time frame with respect to administration of the SMAD7 AON (e.g., using an analysis described in Section 7.2, including, e.g., CDAI score or MMS). For example, in some embodiments, clinical remission is observed about one day, about 3 days, about one week, about two weeks, about three weeks, about four weeks, about six weeks, about eight weeks, or about ten weeks after an administration step (for example, an administration step (b)) and maintained for a period of at least 3 days, at least 1 week, at least 2 weeks, at least 3 weeks, at least 4 weeks, at least 6 weeks at least 8 weeks, or at least 10 weeks. Similarly, some embodiments of the invention comprise a method of determining that the patient having IBD has a chance of experiencing clinical remission of IBD, where the patient having IBD had a CDAI of between about 220 and about 400, between about 150 and about 200, between about 200 and about 250, between about 250 and about 300, between about 300 and about 350, between about 350 and about 400, between about 400 and about 450, or greater than about 450 one week prior to an anti-SMAD7 therapy administration step (for example, an administration step (b)). Some embodiments of the invention comprise a method of determining that the patient having IBD has a chance of experiencing clinical remission of IBD, where the patient having IBD had a MMS of between about 4 and about 9, between about 2 and about 4, between about 4 and about 6, between about 6 and about 8, or greater than about 8 one week prior to an anti-SMAD7 therapy administration step (for example, an administration step (b)).

In some embodiments, a method of treating or managing IBD in a patient with above normal levels of phospho-SMAD3, comprises administering to the patient a dose of SMAD7 AON. Furthermore, in some embodiments, a methods for treating or managing IBD in a patient who has above normal phospho-SMAD3 levels following administration of a dose of a SMAD7 AON, comprises administered a further dose of the SMAD7 AON that is greater than or equal to the prior dose. Similarly, in some embodiments of a method of treating or managing IBD, the patient having IBD has below normal phospho-SMAD3 levels following administration of a dose of SMAD7 AON. In the latter case, the method will comprise administering to the patient a further dose of the SMAD7 AON that is less than or equal to the prior dose. In some embodiments, administration of the SMAD7 AON to the patient is repeated until the patient shows a clinical response or remission, e.g., based on monitoring a clinical parameter described in Section 7.2, e.g., until the levels of a biomarker, e.g., SMAD7, Foxp3, CCR9, CCL20, IL4, IL10, CD4, CD8, IFN-γ, IL17, IL8, CRP, TNFα, FCP reach normal levels or until the patient achieves a CDAI score of less than 150, or based on any other clinical parameter described in Section 7.2.

In some embodiments of a method of treating or managing IBD in a patient having below normal levels of phospho-SMAD3, the amount of a SMAD7 AON administered to the patient is increased until phospho-SMAD3 levels in the patient increase. In such embodiments, levels of SMAD7 AON administered to the patient can be increased until the level of phospho-SMAD3 in the patient increases to about a normal level of phospho-SMAD3 or a above normal level of phospho-SMAD3.

In some embodiments of a method of treating or managing IBD comprises monitoring the treatment or management of IBD in a patient with IBD, that comprises analyzing phospho-SMAD3 levels in the patient following each SMAD7 AON administration. Utilizing these methods, the absence of an increase in phospho-SMAD3 levels indicates that the treatment or management is not effective. In such embodiments, phospho-SMAD3 levels can be analyzed one time or multiple times, for instance, two times, three times, four times, about five times, about 10 times, about 15 times, about 20 times, or about 30 times, after each administration of SMAD7 AON. Furthermore, the timing of the measurement of phospho-SMAD3 levels can vary with respect to the time of SMAD7 AON administration such that phospho-SMAD3 levels can be analyzed immediately after, about 1 hour after, about 3 hours after, about 6 hours after, about 12 hours after, about 1 day after, about 3 days after, about 1 week after, about 2 weeks after, and/or about 1 month after SMAD7 AON administration.

In order to determine levels of a biomarker or analyte, for example, phospho-SMAD3, in a patient having IBD using the methods described herein, a sample can be obtained from the patient. Therefore, in some embodiments of a method of treating or managing IBD provided in this section, the level of phospho-SMAD3 in the patient having IBD is determined in a sample obtained from the patient having IBD. Analytes other than or in addition to phospho-SMAD3, for example, but not limited to Fecal Calprotectin (FCP), Interleukin-8 (IL8), Interleukin-17 (IL17), Interferon gamma (IFN-γ,) Tumor Necrosis Factor alpha (TNFα), Cluster of Differentiation 4 (CD4), Cluster of Differentiation 8 (CD8) and C-Reactive Protein (CRP), can also be determined in methods of the invention. Thus, in some embodiments, the method comprises determining a level, or multiple levels, of one or more additional analytes in the patient having IBD. Analytes of TNFα comprise RNA, DNA, and protein products of or derived from the TNFα gene, described by NCBI Reference Sequence: NG_007462.1. Analytes of CRP comprise RNA, DNA, and protein products of or derived from the CRP gene, described by NCBI Reference Sequence: NG_013007.1. Analytes of IL8 comprise RNA, DNA, and protein products of or derived from the TNFα gene, described by NCBI Reference Sequence: NG_029889.1. Analytes of FCP comprise RNA, DNA, and protein products of or derived from the FCP gene, described by Entrez GeneID No. 6280. Analytes of IL6 comprise RNA, DNA, and protein products of or derived from the IL6 gene, described by Entrez GeneID No. 3569. Analytes of IL8 comprise RNA, DNA, and protein products of or derived from the IL8 gene, described by Entrez GeneID No. 3567. Analytes of IL12 comprise RNA, DNA, and protein products of or derived from the IL12 gene, described by Entrez GeneID No. 3593. Analytes of IL17 comprise RNA, DNA, and protein products of or derived from the IL17A gene described by Entrez GeneID No. 3605. Analytes of CD4 comprise RNA, DNA, and protein products of or derived from the CD4 gene, described by Entrez GeneID No. 920. Analytes of CD8 comprise RNA, DNA, and protein products of or derived from the CD8 gene, described by Entrez GeneID No. 925. Analytes of HLA-DR comprise RNA, DNA, and protein products of or derived from the HLA-DR gene family (including, e.g., HLA-DRA, HLA-DRB1, HLA-DRB3, HLA-DRB4, and HLA-DRB5), described, e.g., by Entrez GeneID Nos. 3122, 3123, 3125, 3126, and 3127. Analytes of Foxp3 comprise RNA, DNA, and protein products of or derived from the Foxp3 gene, described by Entrez GeneID No. 50943.

Samples containing analytes of interest, for example, SMAD7, phospho-SMAD3, HLA-DR, TNFα, CRP, IFN-γ, IL6, IL8, IL12, IL17A, CD4 and/or CD8, obtained from the patient having IBD, can comprise blood, serum, plasma samples, or tissue samples, such as a tissue biopsy. Samples containing FCP can comprise stool samples. Stool samples can be wet stool samples or dry stool samples. Samples can also comprise tissue samples such as, but not limited to, tissue, gastrointestinal, mucosal, submucosal, intestinal, esophageal, ileal, rectal, or lymphatic samples. Levels of analytes of interest in a sample from a patient having IBD can be determined using various assays. For example, in methods of the invention, the level of phospho-SMAD3 and/or another analyte can be determined by immunochemistry, for example, by an enzyme-linked immunosorbent assay (ELISA). Alternatively, the level os phosphor-SMAD3 in a sample, such as a tissue biopsy, can be determined in an assay coupling high-pressure liquid chromatography with mass spectrometry (HPLC-MS).

Methods provided herein comprise methods for treating and managing various forms of IBD. For example, the invention comprises methods for treating and managing IBD, where the IBD is Crohn's Disease (CD) or ulcerative colitis (UC). The contemplated invention also provides methods for treating different types of patients with IBD, including, for example, but not limited to, IBD patients that are steroid-dependent patients with active CD; and steroid-resistant patients with active CD

It will be appreciated that the SMAD7 AON administered to the patient having IBD in methods of the invention described herein, can be administered by various administration routes. In various embodiments, the SMAD7 AON can be administered by one or several routes, including orally, topically, parenterally, e.g., by subcutaneous injection, by inhalation spray, or rectally. The term parenteral as used herein comprises subcutaneous injections, intrapancreatic administration, intravenous, intramuscular, intraperitoneal, intrasternal injection or infusion techniques. In a preferred embodiment, the SMAD7 AON may be administered orally to the patient having IBD

The SMAD7 AON described in Section 7.11 can, for example, be used in the methods of the invention described herein.

7.6 IBD Treatment, Management, and Prevention

7.6.1 Treatment and Management

The methods recited in this section are useful to treat or manage IBD in a patient or subject having IBD, including, e.g., mild, medium, or severe forms of IBD (e.g., mild, medium, or severe forms of CD or UC), e.g., as determined by a clinical activity parameter or a biomarker level. In some embodiments, the methods are useful to prevent IBD, e.g., in a patient at risk of developing IBD, e.g., as determined by the presence of certain risk factors in the patient, which are known in the art (e.g., genetic, environmental, or lifestyle factors). In some embodiments, the methods provided herein are useful to prevent the reoccurrence of IBD in a patient who has previously received an IBD treatment (e.g., an aminosalicylate treatment or a steroid treatment), which is failing, or in a treatment naive patient who is experiencing a chronic disease with few or no clinical symptoms.

In some embodiments, treating or managing IBD comprises reducing one or more clinical symptoms of IBD. In some embodiments, treating or managing IBD comprises reducing a symptom of CD, such as belly pain (including, e.g., cramping, soreness to touch, constant ache), diarrhea (including, e.g., blood in stool), loss of appetite, fever, weight loss, anemia, intestinal inflammation, or an infection (e.g., an abscess), an anal fissure, joint pain, eye problems, a skin rash, or liver disease. In some embodiments, treating or managing IBD comprises reducing one or more symptoms of UC, such as intestinal swelling, intestinal inflammation, sores in the lining of the large intestine (colon), diarrhea, belly pain, or bleeding from the rectum. In some embodiments, treating or managing IBD comprises reducing one or more IBD symptoms during a chronic phase of the disease. In some embodiments, treating or managing IBD comprises reducing one or more IBD symptoms during an acute phase of the disease (e.g., during a disease “flare up”). In some embodiments, treating or managing IBD comprises increasing the time to relapse in an IBD patient who has responded to an IBD treatment such as an anti-SMAD7 therapy (e.g., and anti-SMAD7 AON).

In some embodiments, treating or managing IBD comprises reducing the intensity of a disease flare up. In some embodiments, treating or managing IBD comprises reducing the frequency with which flare ups occur the IBD patient.

In some embodiments, treating of managing IBD comprises improving the quality of life of an IBD patient (e.g., as determined by a patient survey), e.g., by reducing pain in the IBD patient, improving appetite in the IBD patient, or improving the IBD patient's sleep (e.g., length of uninterrupted sleep).

In some embodiments, treating or managing IBD comprises, reducing an IBD biomarker level (e.g., hsCRP, FCP, inflammatory cytokine (e.g., IL6, IL8, IL12, IL17, TNFα, IFNγ), phospho-SMAD3 or SMAD7 mRNA or SMAD7 protein levels).

In some embodiments, treating or managing IBD comprises, increasing an IBD biomarker level (e.g., SMAD3 phosphorylation).

In some embodiments FCP levels in a fecal sample from the patient having IBD are ≤50 μg/g stool, ≤75 μg/g stool, ≤100 μg/g stool, ≤125 μg/g stool, ≤150 μg/g stool, ≤175 μg/g stool, ≤200 μg/g stool, ≤225 μg/g stool, ≤250 μg/g stool, ≤275 μg/g stool, ≤300 μg/g stool, ≤325 μg/g stool, ≤350 μg/g stool, ≤375 μg/g stool, or ≤400 μg/g stool at a timepoint during the first treatment period or at a timepoint during the second treatment period. FCP levels can be determined in an IBD patient's wet stool sample or dry stool sample. In some embodiments, FCP levels in a fecal sample from the patient having IBD are ≤200 μg/g stool at the end of the first treatment period or at the end of the second treatment period. In some embodiments, FCP levels in a fecal sample from a patient aged 2-9 years are reduced to between about 100 μg/g stool and about 200 μg/g stool, between about 110 μg/g stool and about 190 μg/g stool, between about 120 μg/g stool and about 180 μg/g stool, between about 130 μg/g stool and about 170 μg/g stool, or between about 140 μg/g stool and about 160 μg/g stool. In some embodiments, FCP levels in a fecal sample from a patient aged 2-9 years are reduced to about 166 tg/mg stool. In some embodiments, FCP levels in a fecal sample from a patient aged 10-59 years are reduced to between about 10 μg/g stool and about 100 μg/g stool, between about 20 μg/g stool and about 90 μg/g stool, between about 30 jμg/g stool and about 80 μg/g stool, between about 40 μg/g stool and about 70 μg/g stool, or between about 50 μg/g stool and about 60 μg/g stool. In some embodiments, FCP levels in a fecal sample from a patient aged 10-59 years are reduced to about 51 μg/g stool. In some embodiments, FCP levels in a fecal sample from a patient aged ≥60 years are reduced to between about 60 μg/g stool and about 160 μg/g stool, between about 70 μg/g stool and about 150 μg/g stool, between about 80 μg/g stool and about 140 μg/g stool, between about 90 μg/g stool and about 130 μg/g stool, or between about 100 μg/g stool and about 120 μg/g stool. In some embodiments, FCP levels in a fecal sample from a patient aged >60 years are reduced to about 112 μg/g stool.

In some embodiments, FCP levels in fecal samples from the patient having IBD are reduced from baseline by ≥20%, ≥30%, ≥40%, ≥50%, ≥60%, ≥70%, ≥80%, or ≥90% at a timepoint during the first treatment period. In some embodiments, FCP levels in fecal samples from the patient having IBD are reduced from baseline by ≥20%, ≥30%, ≥40%, ≥50%, ≥60%, ≥70%, ≥80%, or ≥90% at a timepoint during the second treatment period.

In some embodiments, treating or managing IBD comprises maintaining an IBD biomarker level in a normal, or near-normal range (e.g., as determined by the respective biomarker levels in a healthy control group). In some embodiments, treating or managing IBD comprises decreasing a clinical activity score, such as an SES-CD, CDAI, UCDAI, PRO-2, abdominal pain, average daily liquid or soft stool frequency score, MMS, PMS, TMS, ES or a histological score. In some embodiments, treating or managing IBD comprises maintaining a clinical activity score, such as a SES-CD, CDAI, UCDAI, PRO-2, abdominal pain, average daily liquid or soft stool frequency score, MMS, PMS, TMS, ES score or a histological score below certain threshold levels (e.g., SES-CD ≤2; CDAI <150; PRO-2<8; abdominal pain ≤1; average daily liquid or soft stool frequency ≤1.5 or ≤3.0, TMS score ≤2; ES=0; PMS score ≤2; MMS score ≤2). In some embodiments, treating or managing IBD comprises decreasing a clinical activity score, such as an SES-CD, CDAI, UCDAI, PRO-2, abdominal pain, average daily liquid or soft stool frequency score, MMS, PMS, TMS, ES score or a histological score, by a certain number of points (e.g., a 25% or 50% reduction of SES-CD scores from baseline; reduction of CDAI score by >100 from baseline; reduction of PRO-2 from baseline by >8 points; reduction of abdominal pain >1 from baseline and/or reduction of average daily liquid or soft stool frequency >1 from baseline; decrease of TMS score from baseline ≥30% and ≥3 points; decrease of ES from baseline ≥I; decrease of PMS score from baseline ≥25% and t 2 points; decrease of MMS score from baseline ≥25% and ≥2 points). In some embodiments, treating or managing IBD comprises maintaining a clinical activity score in a normal range or a near-normal range (e.g., as determined by the respective biomarker levels in a healthy control group).

In some embodiments, treating or managing IBD comprises decreasing a clinical activity score, such as a TMS, PMS, MMS score, or an RBS, endoscopic subscore. In some embodiments, treating or managing IBD comprises maintaining a clinical activity score, such as a TMS, PMS, MMS score, or an RBS, endoscopic subscore below certain threshold levels (e.g., an RBS subscore ≤1). In some embodiments, treating or managing IBD comprises decreasing a clinical activity score, such as a TMS, PMS, MMS score, or an RBS, endoscopic subscore, by a percentage or certain number of points from baseline (e.g., a >30% and ≥3 points reduction of TMS scores from baseline; a >25% and ≥2 points reduction of PMS scores from baseline; a >25% and ≥2 points reduction of MMS scores from baseline; a >1 point reduction in RBS subscore). In some embodiments, treating or managing IBD comprises maintaining a clinical activity score in a normal range or a near-normal range (e.g., as determined by the respective score levels in a healthy control group).

In some embodiments, treating or managing IBD comprises a reduction of about 4 points in SES-CD score compared to baseline at a timepoint in or around week 4 of the first treatment period. In some embodiments, the patient having IBD shows a response to the SMAD7 AON if the SES-CD score is reduced by 4 points relative to baseline.

In some embodiments, treating or managing IBD comprises an SES-CD score at a timepoint during the first treatment period or during the second treatment period of ≤80%, ≤75%, ≤70%, ≤65%, 60%, ≤55%, ≤50%, ≤45%, ≤40%, ≤35%, ≤30%, ≤25%, or ≤20% compared to the SES-CD score at baseline (e.g., at a timepoint during week 0 of the first treatment period). In some embodiments, the SES-CD score at a timepoint during the first treatment period is ≤50% compared to the patient's SES-CD score at baseline. In some embodiments, the SES-CD score at a timepoint in or around week 4 of the first treatment period is ≤75% or ≤50% compared to baseline. In some embodiments, the SES-CD score at a timepoint in or around week 12 of the first treatment period is ≤75% or ≤50% compared to baseline.

In some embodiments, the SES-CD score is ≤5, ≤4, ≤3, ≤2, or ≤I at a timepoint during the first treatment period or the second treatment period (e.g., a timepoint during the last week of the first or the second treatment period). In some embodiments, the SES-CD score is ≤2 at a timepoint during first treatment period or the second treatment period (e.g., during the last week of the first or the second treatment period). In some embodiments, the SES-CD score is ≤2 at a timepoint in or around week 12 of the first treatment period.

In some embodiments, the patient has a SES-CD score of ≥7 at the beginning of the first treatment period (e.g., at a timepoint during week 0).

In some embodiments, the patient has a total SES-CD score of >6 or the ileum segmental SES-CD score of >4 at the beginning of the first treatment period (e.g., at a timepoint during week 0).

In some embodiments, treating or managing IBD comprises reducing an average daily liquid or soft stool frequency score by ≥20%, ≥30%, ≥40%, ≥50%, ≥60%, ≥70%, ≥80%, or ≥90% from baseline in the patient having IBD at a timepoint during the first, second and/or third treatment periods, e.g., at a timepoint during week 2, week 4, week 8, or week 12 of the first treatment period, during week 4, week 8, week 12, week 16, week 20, or week 24 of the second treatment period, or during week 24, week 52, week 104, week 156, or week 208 of the third treatment period.

In some embodiments, treating or managing IBD comprises reducing average daily abdominal pain from baseline by ≥20%, ≥30%, ≥40%, ≥50%, ≥60%, ≥70%, ≥80%, or ≥90% in the patient having IBD at a timepoint during the first, second and/or third treatment periods, e.g., at a timepoint during week 2, week 4, week 8, or week 12 of the first treatment period, during week 4, week 8, week 12, week 16, week 20, or week 24 of the second treatment period, or during week 24, week 52, week 104, week 156, or week 208 of the third treatment period.

In some embodiments, treating or managing IBD comprises achieving a PRO-2 score of <14, <12, <10, <8, <6, <4, or <2 in the patient having IBD at a timepoint during the first treatment period. In some embodiments, the PRO-2 score at a timepoint during the second treatment period is <14, <12, <10, <8, <6, <4, or <2.

In some embodiments, the PRO-2 score at a timepoint during the third treatment period is <14, <12, <10, <8, <6, <4, or <2.

In some embodiments, the PRO-2 score is <8 at a timepoint during the first, second and/or third treatment periods (e.g., at a timepoint during week 2, week 4, week 8, or week 12 of the first treatment period, at a timepoint during week 4, week 8, week 12, week 16, week 20 or week 24 of the second treatment period, or at a timepoint during week 24, week 52, week 104, week 156, or week 208 of the third treatment period).

In some embodiments, the PRO-2 score is ≥2, ≥3, ≥4, ≥5, ≥6, ≥7, ≥8, ≥9, ≥10, ≥12, or ≥14 points decreased from baseline at a timepoint during the first, second and/or third treatment periods.

In some embodiments, the PRO-2 is ≥8 points decreased from baseline at a timepoint during or at a timepoint during week 2, week 4, week 8 or week 12 of the first treatment period, during week 4, week 8, week 12, week 16, week 20, or week 24 of the second treatment period, or during week 24, week 52, week 104, week 156, or week 208 of the third treatment period.

In some embodiments, the CDAI score decreases ≥20 points, ≥30 points, ≥40 points, ≥50 points, ≥60 points, ≥70 points, ≥80 points, ≥90 points, ≥100 points, ≥110 points, ≥120 points, ≥130 points, ≥140 points, or ≥150 points from baseline during the first treatment period, during the second treatment period or during the first and second treatment period.

In some embodiments, the CDAI score decreases ≥20 points, ≥30 points, ≥40 points, ≥50 points, ≥60 points, ≥70 points, ≥80 points, ≥90 points, ≥100 points, ≥110 points, ≥120 points, ≥130 points, ≥140 points, or ≥150 points from baseline during the third treatment period, during the second and the third treatment periods, during the first and third treatment periods, or during all three treatment periods.

In some embodiments, the CDAI score is decreased ≥100 points from baseline at a timepoint during the first treatment period, during the second treatment period, or during the first and second treatment periods. In some embodiments, the CDAI score is ≥100 points decreased from baseline at a timepoint during week 2, week 4, week 8 or week 12 of the first treatment period and at a timepoint during week 4, week 8, week 12, week 16, week 20, or week 24 of the second treatment period.

In some embodiments, the CDAI score is decreased ≥100 points from baseline at a timepoint during the third treatment period, during the second and the third treatment periods, during the first and third treatment periods, or during all three treatment periods. In some embodiments, the CDAI score is ≥100 points decreased from baseline at a timepoint during week 24, week 52, week 104, week 156, or week 208 of the third treatment period.

In some embodiments, the CDAI score is <200, <190, <180, <170, <160, <150, <140, <130, <120, <110, or <100 at a timepoint during the first, second or third treatment period (e.g., at a timepoint during the last week of the first, second or third treatment period). In some embodiments, the CDAI score is <150 at a timepoint during the first, second or third treatment period.

In some embodiments, the CDAI score is <150 at a timepoint in or around week 2, week 4, week 8, or week 12 of the first treatment period, at a timepoint in or around week 4, week 8, week 12, week 16, week 20, or week 24 of the second treatment period, or at a timepoint during week 24, week 52, week 104, week 156, or week 208 of the third treatment period. In some embodiments, the CDAI score is <150 at a timepoint in or around week 4 of the first treatment period.

In some embodiments, the CDAI score is <150 at a timepoint in or around week 8 of the first treatment period. In some embodiments, the CDAI score is <150 at a timepoint in or around week 12 of the first treatment period. In some embodiments, the CDAI score is <150 at a timepoint in or around week 24 of the second treatment period. In some embodiments, the CDAI score is <150 at a timepoint in or around week 52 of the second treatment period. In some embodiments, the CDAI score is <150 at a timepoint in or around week 52 of the third treatment period. In some embodiments, the CDAI score is <150 at a timepoint in or around week 208 of the third treatment period.

In some embodiments, treating or managing IBD comprises a reduction of about 3 points in a TMS score compared to baseline at a timepoint in or around week 4 of the first treatment period. In some embodiments, the patient having IBD shows a response to the SMAD7 AON if the TMS score is reduced by 3 points relative to baseline.

In some embodiments, treating or managing IBD comprises a TMS score at a timepoint during the first treatment period or during the second treatment period of ≤80%, ≤75%, ≤70%, ≤65%, ≤60%, ≤55%, ≤50%, ≤45%, ≤40%, ≤35%, ≤30%, ≤25%, or ≤20% compared to the TMS score at baseline (e.g., at a timepoint during week 0 of the first treatment period). In some embodiments, the TMS score at a timepoint during the first treatment period is ≤70% compared to the patient's TMS score at baseline. In some embodiments, the TMS score at a timepoint in or around week 4 of the first treatment period is ≤70% compared to baseline. In some embodiments, the TMS score at a timepoint in or around week 8 of the first treatment period is ≤70% compared to baseline. In some embodiments, the TMS score at a timepoint during the second treatment period is ≤70% compared to the patient's TMS score at baseline.

In some embodiments, the TMS score is ≤9, ≤8, ≤7, ≤6, ≤5, ≤4, ≤3, ≤2, or ≤1 at a timepoint during the first treatment period or the second treatment period (e.g., a timepoint during the last week of the first or the second treatment period). In some embodiments, the TMS score is ≤9 at a timepoint during first treatment period or the second treatment period (e.g., during the last week of the first or the second treatment period). In some embodiments, the TMS score is ≤2 at a time point in or around week 8 of the first treatment period.

In some embodiments, the patient has a TMS score of ≥4 at the beginning of the first treatment period (e.g., at a timepoint during week 0). In some embodiments, the patient has a TMS score of ≥6 at the beginning of the first treatment period (e.g., at a timepoint during week 0). In some embodiments, the patient has a TMS score of ≥9 at the beginning of the first treatment period (e.g., at a timepoint during week 0).

In some embodiments, treating or managing IBD comprises a reduction of about 2 points in a PMS score compared to baseline at a timepoint in or around week 4 of the first treatment period. In some embodiments, the patient having IBD shows a response to the SMAD7 AON if the PMS score is reduced by 2 points relative to baseline.

In some embodiments, treating or managing IBD comprises a PMS score at a timepoint during the first treatment period or during the second treatment period of ≤80%, ≤75%, ≤70%, ≤65%, ≤60%, ≤55%, ≤50%, ≤45%, ≤40%, ≤35%, ≤30%, ≤25%, or ≤20% compared to the PMS score at baseline (e.g., at a timepoint during week 0 of the first treatment period). In some embodiments, the PMS score at a timepoint during the first treatment period is ≤75% compared to the patient's PMS score at baseline. In some embodiments, the PMS score at a timepoint in or around week 4 of the first treatment period is ≤75% compared to baseline. In some embodiments, the PMS score at a timepoint in or around week 8 of the first treatment period is ≤75% compared to baseline. In some embodiments, the PMS score at a timepoint during the second treatment period is ≤75% compared to the patient's PMS score at baseline.

In some embodiments, the PMS score is ≤7, ≤6, ≤5, ≤4, ≤3, ≤2, or ≤1 at a timepoint during the first treatment period or the second treatment period (e.g., a timepoint during the last week of the first or the second treatment period). In some embodiments, the PMS score is ≤7 at a timepoint during first treatment period or the second treatment period (e.g., during the last week of the first or the second treatment period). In some embodiments, the PMS score is ≤2 at a time point in or around week 8 of the first treatment period.

In some embodiments, the patient has a PMS score of ≥4 at the beginning of the first treatment period (e.g., at a timepoint during week 0). In some embodiments, the patient has a PMS score of ≥6 at the beginning of the first treatment period (e.g., at a timepoint during week 0).

In some embodiments, treating or managing IBD comprises a reduction of about 2 points in an MMS score compared to baseline at a timepoint in or around week 4 of the first treatment period. In some embodiments, the patient having IBD shows a response to the SMAD7 AON if the MMS score is reduced by 2 points relative to baseline.

In some embodiments, treating or managing IBD comprises an MMS score at a timepoint during the first treatment period or during the second treatment period of ≤80%, ≤75%, ≤70%, ≤65%, ≤60%, ≤55%, ≤50%, ≤45%, ≤40%, ≤35%, ≤30%, ≤25%, or ≤20% compared to the MMS score at baseline (e.g., at a timepoint during week 0 of the first treatment period). In some embodiments, the MMS score at a timepoint during the first treatment period is ≤75% compared to the patient's MMS score at baseline. In some embodiments, the MMS score at a timepoint in or around week 4 of the first treatment period is ≤75% compared to baseline. In some embodiments, the MMS score at a timepoint in or around week 8 of the first treatment period is ≤75% compared to baseline. In some embodiments, the MMS score at a timepoint during the second treatment period is ≤75% compared to the patient's MMS score at baseline.

In some embodiments, the MMS score is ≤7, ≤6, ≤5, ≤4, ≤3, ≤2, or ≤1 at a timepoint during the first treatment period or the second treatment period (e.g., a timepoint during the last week of the first or the second treatment period). In some embodiments, the MMS score is ≤7 at a timepoint during first treatment period or the second treatment period (e.g., during the last week of the first or the second treatment period). In some embodiments, the MMS score is ≤2 at a time point in or around week 8 of the first treatment period.

In some embodiments, the patient has a MMS score of ≥4 at the beginning of the first treatment period (e.g., at a timepoint during week 0). In some embodiments, the patient has a MMS score of ≥6 at the beginning of the first treatment period (e.g., at a timepoint during week 0).

In some embodiments, treating or managing IBD comprises a reduction of about 1 point in an RBS subscore compared to baseline at a timepoint in or around week 4 of the first treatment period. In some embodiments, the patient having IBD shows a response to the SMAD7 AON if the RBS subscore is reduced by 1 point relative to baseline.

In some embodiments, treating or managing IBD comprises an RBS subscore at a timepoint during the first treatment period or during the second treatment period of 0 or 1 point. In some embodiments, the RBS subscore at a timepoint during the first treatment period is 0 or 1. In some embodiments, the RBS subscore at a timepoint in or around week 4 of the first treatment period is 0 or 1. In some embodiments, the RBS subscore at a timepoint in or around week 8 of the first treatment period is 0 or 1. In some embodiments, the RBS subscore at a timepoint during the second treatment period is 0 or 1.

In some embodiments, the patient has an RBS subscore of ≥1 at the beginning of the first treatment period (e.g., at a timepoint during week 0). In some embodiments, the patient has an RBS subscore of ≥2 at the beginning of the first treatment period (e.g., at a timepoint during week 0).

In some embodiments, treating or managing IBD comprises a reduction of about 1 point in an endoscopic subscore compared to baseline at a timepoint in or around week 4 of the first treatment period. In some embodiments, the patient having IBD shows a response to the SMAD7 AON if the endoscopic subscore is reduced by 1 point relative to baseline.

In some embodiments, treating or managing IBD comprises an endoscopic subscore at a timepoint during the first treatment period or during the second treatment period of 0 or 1 point. In some embodiments, the endoscopic subscore at a timepoint during the first treatment period is 0 or 1. In some embodiments, the endoscopic subscore at a timepoint in or around week 4 of the first treatment period is 0 or 1. In some embodiments, the endoscopic subscore at a timepoint in or around week 8 of the first treatment period is 0 or 1. In some embodiments, the endoscopic subscore at a timepoint during the second treatment period is 0 or 1. In some embodiments, an endoscopic subscore of 1 does not include friability.

In some embodiments, the patient has an endoscopic subscore of ≥1 at the beginning of the first treatment period (e.g., at a timepoint during week 0). In some embodiments, the patient has an endoscopic subscore of ≥2 at the beginning of the first treatment period (e.g., at a timepoint during week 0).

In some embodiments, treating or managing IBD comprises a reduction of at least 3 points and at least 30% in a TMS score compared to baseline, with an accompanying reduction in an RBS score of at least 1 point or absolute RBS of 0 or 1.

In some embodiments, treating or managing IBD comprises a reduction of at least 2 points and at least 25% in an MMS score compared to baseline, with an accompanying reduction in an RBS score of at least 1 point or absolute RBS of 0 or 1.

In some embodiments, treating or managing IBD comprises a reduction of at least 2 points and at least 25% in a PMS score compared to baseline, with an accompanying reduction in an RBS score of at least 1 point or absolute RBS of 0 or 1.

In some embodiments, the patient having IBD shows a response to the SMAD7 AON administration at a timepoint during the first treatment period (e.g., during week 2, week 4, week 8, or week 12) or second treatment period (e.g., during week 4, week 8, week 12, week 16, week 20, or week 24), or at a timepoint following the second treatment period (e.g., 1 week, 2 weeks, 4 weeks, 3 months, 6 months, 9 months, 12 months, 18 months, 2 years, 3 years, 4 years or 5 years after the second treatment period). In some embodiments, the response to the SMAD7 AON administration comprises a reduction of the severity or the frequency of recurrence of one or more clinical symptoms of IBD. In some embodiments, the response to the SMAD7 AON administration comprises a reduction of at least 50% in the SES-CD score compared to baseline (e.g., a timepoint during week 0 of first treatment period). In some embodiments, the response to the SMAD7 AON administration comprises a decrease from baseline of ≥100 points in CDAI score. In some embodiments, the response to the SMAD7 AON administration comprises a decrease from baseline of ≥8 point in PRO-2 score. In some embodiments, the response to the SMAD7 AON administration comprises a decrease from baseline of ≥1 point in average daily liquid or soft stool frequency scores. In some embodiments, the response to the SMAD7 AON administration comprises a decrease from baseline of ≥1.0 point in abdominal pain score. In some embodiments, the response to the SMAD7 AON administration comprises a decrease from baseline of ≥1 point in average daily liquid or soft stool frequency scores and a decrease from baseline of ≥1 point in abdominal pain score. In some embodiments, the response to the SMAD7 AON administration comprises a decrease of TMS from baseline ≥30% and ≥3 points. In some embodiments, the response to the SMAD7 AON administration comprises a decrease of ES from baseline ≥1. In some embodiments, the response to the SMAD7 AON administration comprises a decrease of PMS score from baseline ≥25% and ≥2 points. In some embodiments, the response to the SMAD7 AON administration comprises a decrease of MMS score from baseline ≥25% and ≥2 points.

In some embodiments, the patient having IBD shows a response to the SMAD7 AON administration at a timepoint during the third treatment period (e.g., during week 24, week 52, week 104, or week 208) or at a timepoint following the third treatment period (e.g., 1 week, 2 weeks, 4 weeks, 3 months, 6 months, 9 months, 12 months, 18 months, 2 years, 3 years, 4 years or 5 years after the third treatment period).

In some embodiments, the patient having IBD shows a time to loss of response (i.e., reoccurrence of a clinical IBD symptom) following the first, second or third treatment period of at least 2 weeks, at least 4 weeks, at least 6 weeks, at least 8 weeks, at least 2 months, at least 3 months, at least 6 months, at least 9 months, at least 12 months, at least 18 months, at least 24 months, at least 30 months, at least 36 months, at least 42 months, at least 48 months, at least 54 months or at least 60 months. In some embodiments, time to loss of response is defined, e.g., a determination at 2 consecutive timepoints of a CDAI score ≥150 and an increase of CDAI score ≥50 from the CDAI score at the time point when the patient first showed a response to the SMAD7 AON.

In some embodiments, the patient having IBD shows remission at a timepoint during the first treatment period (e.g., during week 2, week 4, week 8, or week 12) or second treatment period (e.g., during week 4, week 8, week 12, week 16, week 20, or week 24), or at a timepoint following the second treatment period (e.g., 1 week, 2 weeks, 4 weeks, 3 months, 6 months, 9 months, 12 months, 18 months, 2 years, 3 years, 4 years or 5 years after the second treatment period). In some embodiments, remission comprises a reduction of the severity or the frequency of recurrence of one or more clinical symptoms of IBD. In some embodiments, remission comprises a SES-CD score ≤2. In some embodiments, remission comprises a CDAI score ≤150. In some embodiments, remission comprises a PRO-2 score ≤8. In some embodiments, remission comprises mucosal healing, as indicated, e.g., by the absence of an intestinal mucosal ulceration. In some embodiments, remission comprises an abdominal pain score ≤1. In some embodiments, remission comprises an average daily liquid or soft stool frequency score of ≤1.5. In some embodiments, remission comprises an MMS, a PMS, or a TMS score ≤2. In some embodiments, remission comprises an ES=0.

In some embodiments, the patient having IBD shows remission at a timepoint during the third treatment period (e.g., during week 24, week 52, week 104, or week 208) or at a timepoint following the third treatment period (e.g., 1 week, 2 weeks, 4 weeks, 3 months, 6 months, 9 months, 12 months, 18 months, 2 years, 3 years, 4 years or 5 years after the second treatment period).

In some embodiments, the patient having IBD does not experience a fibrotic event (e.g., scarring) during the first treatment period. In some embodiments, the patient having IBD does not experience a fibrotic even during the second treatment period. In some embodiments, the patient having ID does not experience a fibrotic event for a period of at least 1 week, at least 2 weeks, at least 3 weeks, at least 4 weeks, at least 6 weeks, at least 2 months, at least 3 months, at least 4 months, at least 5 months, at least 6 months, at least 9 months, at least 12 months, at least 18 months, at least 24 months, at least 30 months, at least 36 months, at least 42 months, at least 48 months, at least 54 months, or at least 60 months after the end of the second treatment period.

In some embodiments, the patient having IBD does not experience a fibrotic even during the third treatment period. In some embodiments, the patient having ID does not experience a fibrotic event for a period of at least 1 week, at least 2 weeks, at least 3 weeks, at least 4 weeks, at least 6 weeks, at least 2 months, at least 3 months, at least 4 months, at least 5 months, at least 6 months, at least 9 months, at least 12 months, at least 18 months, at least 24 months, at least 30 months, at least 36 months, at least 42 months, at least 48 months, at least 54 months, or at least 60 months after the end of the third treatment period.

7.6.2 Prevention

In some embodiments, preventing IBD or preventing the reoccurrence of IBD comprises preventing the occurrence or reoccurrence of one or more clinical symptoms of IBD, either partially or completely. In some embodiments, preventing IBD or preventing the reoccurrence of IBD comprises preventing the occurrence or reoccurrence of a symptom of CD, such as belly pain (including, e.g., cramping, soreness to touch, constant ache), diarrhea (including, e.g., blood in stool), loss of appetite, fever, weight loss, anemia, intestinal inflammation, or an infection (e.g., an abscess), an anal fissure, joint pain, eye problems, a skin rash, or liver disease. In some embodiments, preventing IBD or preventing the reoccurrence of IBD comprises preventing the occurrence or the reoccurrence of one or more symptoms of UC, such as intestinal swelling, intestinal inflammation, sores in the lining of the large intestine (colon), diarrhea, belly pain, or bleeding from the rectum. In some embodiments, preventing IBD or preventing the reoccurrence of IBD comprises preventing one or more IBD symptoms during a chronic phase of the disease. In some embodiments, preventing IBD or preventing the reoccurrence of IBD comprises reducing one or more IBD symptoms during an acute phase of the disease (e.g., during a disease “flare up”). In some embodiments, preventing the reoccurrence of IBD comprises increasing the time to relapse in an IBD patient who has responded to an IBD treatment such as an anti-SMAD7 therapy (e.g., and anti-SMAD7 AON).

In some embodiments, preventing IBD or preventing the reoccurrence of IBD comprises preventing the occurrence or reoccurrence of a disease flare up or of a disease flare up of a certain intensity. In some embodiments, preventing IBD or preventing the reoccurrence of IBD comprises preventing the occurrence or reoccurrence of flare ups at a certain frequency (e.g., at a frequency observed in the patient having IBD directly prior to the administration of an IBD treatment regiment or at a (e.g., median, average or mean) frequency observed in a control group of untreated IBD patients).

In some embodiments, preventing IBD or preventing the reoccurrence of IBD comprises preventing a (further) deterioration in the quality of life of an IBD patient (e.g., as determined by a patient survey), e.g., by preventing the increase in pain in the IBD patient, preventing (further) loss of appetite in the IBD patient, or preventing a worsening of sleeplessness in the IBD patient.

In some embodiments, preventing IBD or preventing the reoccurrence of IBD comprises, comprises preventing the increase of an IBD biomarker level (e.g., CRP (e.g., measured as hsCRP), FCP, inflammatory cytokine (e.g., IL6, IL8, IL12, or IL17), CD4, CD8, phosphor-SMAD3, HLA-DR or SMAD7 mRNA or SMAD7 protein levels), e.g., an increase over previous IBD biomarker levels observed in the patient or an increase relative to normal or near-normal IBD biomarker levels (e.g., as determined by the respective biomarker levels in a healthy control group). In some embodiments, preventing IBD or preventing the reoccurrence of IBD comprises preventing the (further) increase of a clinical activity score, such as a SES-CD, CDAI, UCDAI, PRO-2, abdominal pain, average daily liquid or soft stool frequency score, MMS, PMS, TMS, ES or a histological score. In some embodiments, preventing IBD or preventing the reoccurrence of IBD comprises preventing a clinical activity score, such as an SES-CD, CDAI, UCDAI, PRO-2, abdominal pain, average daily liquid or soft stool frequency score, MMS, PMS, TMS, ES or a histological score, from increasing above a certain threshold levels (e.g., SES-CD=2; CDAI=150; PRO-2=8; abdominal pain=1.0; average daily liquid or soft stool frequency=1.5 or 3.0, MMS=2, PMS=2, TMS=2, ES=0 or 1). In some embodiments, preventing IBD or preventing the reoccurrence of IBD comprises preventing a clinical activity score, such as an SES-CD, CDAI, UCDAI, PRO-2, abdominal pain, average daily liquid or soft stool frequency score, MMS, PMS, TMS, ES or a histological score, from increasing by a certain number of points (e.g., a doubling of SES-CD scores; an increase in CDAI ≥50 or ≥100; an increase of PRO-2≥8; an increase in abdominal pain by ≥1 point and/or an increase in average daily liquid or soft stool frequency by ≥1 point; an increase in MMS, TMS, or PMS ≥2; an increase in ES ≥1). In some embodiments, preventing IBD or preventing the reoccurrence of IBD comprises preventing a clinical activity score from exceeding a normal range or a near-normal range (e.g., as determined by the respective biomarker levels in a healthy control group).

In some embodiments, preventing IBD or preventing the reoccurrence of IBD comprises preventing the (further) increase of a clinical activity score, such as a TMS, PMS, MMS score, or an RBS, endoscopic subscore. In some embodiments, preventing IBD or preventing the reoccurrence of IBD comprises preventing a clinical activity score, such as a TMS, PMS, MMS score, or an RBS, endoscopic subscore from increasing above a certain threshold levels (e.g., an RBS subscore=1). In some embodiments, preventing IBD or preventing the reoccurrence of IBD comprises preventing a clinical activity score, such as a TMS, PMS, MMS score, or an RBS, endoscopic subscore, from increasing by a percentage or certain number of points from baseline (e.g., a >30% and ≥3 points increase of TMS scores from baseline; a ≥25% and ≥2 points increase of PMS scores from baseline; a ≥25% and ≥2 points increase of MMS scores from baseline; a ≥1 point increase in RBS subscore). In some embodiments, treating or managing IBD comprises maintaining a clinical activity score in a normal range or a near-normal range (e.g., as determined by the respective score levels in a healthy control group).

7.7. Adjustment of Treatment Regimens

In some embodiments, the methods provided herein further comprise adjusting the method of treating or managing IBD at a time when the patient having IBD shows a response to the SMAD7 AON or experiences remission. See, e.g., Section 7.1.1.3 and Section 7.1.1.6.

For example, if the patient having IBD shows a response to the SMAD7 AON prior to the end of the first treatment period, the first treatment period can be ended or shortened (e.g., by any number of days, weeks, or months). In this case, the second treatment period can start early and the second treatment period can comprise administration of the SMAD7 AON at the second dose or a lower dose than the second dose (e.g., 20%, 30%, 40%, or 50% lower). In some embodiments, the dosing schedule during the second treatment period can be altered if the patient having IBD responds to the SMAD7 AON during the first treatment period. For example, an alternating dosing schedule can be altered to have longer periods without treatment.

In some embodiments, if the patient having IBD does not show a response, does not show remission, or cannot fully taper an addition IBD treatment at the end of the first treatment period, the patient does not enter the second treatment period, but instead repeats the first treatment period. In this case, the patient having IBD can be administered with an increased first dose during the repeat of the first treatment period (e.g., the first dose can be increased by 20 mg/day, or by 10%). In some embodiments, the patient having IBD can repeat the first treatment period at continuously increasing first doses of the SMAD7 AON until the patient having IBD shows a response to the SMAD7 AON, shows remission, fully tapers an additional IBD treatment. If the first dose exceeds the maximum tolerated dose, the treatment is terminated.

In some embodiments, the first dose during the repeat of the first treatment period can be increased by 40 mg/day, 80 mg/day, 120, mg/day, 160 mg/day, 240 mg/day, 320 mg/day, or 50%, 100%, 200%, 400%.

In some embodiments, an IBD patient who does not show a response or remission during the second treatment period, can transition from the second treatment period to the third treatment period. During the third treatment period, the IBD patient can be administered with an increased third dose (e.g., the third dose can be increased by 120 mg/day, from 40 mg/day to about 160 mg/day, or 4-fold, relative to the second dose). In some embodiments, the IBD patient can be administered the third dose using a continuous dosing schedule or an alternating dosing schedule (e.g., 4 weeks of SMAD7 AON treatment alternating with 4 weeks of placebo or no treatment) until the IBD patient shows a response to the SMAD7 AON, or experiences remission. If the third dose exceeds the maximum tolerated dose, the treatment is terminated.

In some embodiments, the monitoring the activity of the SMAD7 AON shows that the patient responds to an initial first dose of the SMAD7 AON during the first treatment period and shows a partial or complete loss of response at a timepoint during the second treatment period. In some such embodiments, the second treatment period is ended and the patient reenters the first treatment period, receiving the SMAD7 AON either at the initial first dose or at a dose higher than the initial first dose.

7.8 Pharmacokinetics (PK) and Pharmacodynamic (PD) Assessments

In some embodiments, a method provided herein for the treatment or prevention of IBD further comprises analyzing PK/PD characteristics of the SMAD7 AON.

In some embodiments, analyzing the PK/PD characteristics comprises analyzing biomarkers in a blood sample (e.g., CRP) or in a stool sample (e.g., FCP) from the patient having IBD.

In some embodiments, analyzing the PK/PD characteristics comprises analyzing biomarkers in an intestinal mucosal sample (e.g., TNFα, microbiome) from the patient having IBD. In some embodiments, analyzing the PK/PD characteristics comprises analyzing biomarkers in mononuclear cells sample (e.g., IL-17A, Foxp3, CCR9) from the patient having IBD.

In some embodiments, analyzing the PK/PD characteristics comprises analyzing an intestinal mucosal biopsy from the patient having IBD. In some embodiments, analyzing the PK/PD characteristics comprises analyzing SMAD7 phosphorylation, e.g., in the intestinal mucosal biopsy from the patient having IBD. In some embodiments, analyzing the PK/PD characteristics comprises analyzing the expression of biomarkers, such as CD4, CD8, or HLA-DR, in the intestinal mucosal biopsy from the patient having IBD.

In some embodiments, analyzing the PK/PD characteristics of the SMAD7 AON comprises monitoring the systematic exposure of the SMAD7 AON in the patient having IBD. In some embodiments, monitoring the systematic exposure of the SMAD7 AON in the patient having IBD comprises analyzing the plasma concentration of the SMAD7 AON at a timepoint during week 4, week 8, or week 12 of the first treatment period. In some embodiments, monitoring the systematic exposure of the SMAD7 AON comprises performing a sparse PK analysis (e.g., an analysis of Area Under the Curve and other pharmacokinetic parameters on the basis of only few patient samples, see, e.g., Example 1) during the first treatment period or the second treatment period. In some embodiments, the sparse PK analysis is performed at week 4, week 8, or week 12 of the first treatment period.

In some embodiments, the sparse PK comprises drawing blood samples from the patient having IBD at two time points, a pre-dose time point and a post-dose time point. In some embodiments, the pre-dose timepoint is at least >23 hours after administration of the previous dose of the SMAD7 AON and the post-dose timepoint is 1 to 6 hours after administration of a dose of interest of the SMAD7 AON. The blood samples can be analyzed for SMAD7 AON content using methods known in the art (e.g., HPLC).

7.9 Patient Population

In some embodiments, an IBD patient to be treated with a method provided herein is a UC patient or a CD patient. In some embodiments, the patient having IBD was diagnosed with CD or UC at least 3 months prior to the initial screening period or the first treatment period. In some embodiments, the patient having IBD was diagnosed with ileitis, or ileocolitis, e.g., as determined by endoscopic, radiographic or another imaging method (e.g., magnetic resonance imaging [MRI], computed tomography [CT] scan), within 2 years prior to the screening period or to the first treatment period. In some embodiments, the patient has IBD involving the distal to mid transverse colon. In some embodiments, the patient has extensive colitis.

In some embodiments, an IBD patient to be treated with a method provided herein has active disease, characterized by a CDAI score ≥220 and ≤450 (range: 0 to 600) or by a SES-CD score ≥7 at the beginning of the screening period or the first treatment period.

In some embodiments, an IBD patient to be treated with a method provided herein has active disease, characterized by a CDAI score ≥220 and ≤450 (range: 0 to 600) and by a SES-CD score ≥4 (if the patient has ileitis only) at the beginning of the screening period or the first treatment period. In some embodiments, an IBD patient to be treated with a method provided herein has active disease, characterized by a CDAI score ≥220 and ≤450 (range: 0 to 600) and by a total SES-CD score ≥6 or ileum segmental SES-CD ≥4 at the beginning of the screening period or the first treatment period. In some embodiments, an IBD patient to be treated with a method provided herein has active disease, characterized by an MMS ≥4 and ≤9 and a Mayo endoscopic subscore ≥2.

In some embodiments, an IBD patient to be treated with a method provided herein failed or experienced intolerance to an IBD treatment other than a SMAD7 AON, e.g., an IBD treatment comprising an aminosalicylate, a budesonide, a systemic corticosteroid, an immunosuppressants (6-mercaptopurine [6-MP], azathioprine [AZA], or methotrexate [MTX]), or biologics (e.g., infliximab, adalimumab, certolizumab, or vedolizumab).

In some embodiments, an IBD patient to be treated with a method provided herein who failed treatment with an aminosalicylate showed a sign or symptom of active disease despite a history of receiving ≥8 weeks of treatment with mesalamine or sulafasalazine ≥3 grams. In some embodiments, a patient having IBD who is intolerant to an aminosalicylate experienced a headache, nausea, vomiting, a hypersensitivity reaction (e.g., rash, eosinophilia, fever or lymphadenopathy), nephrotoxicity, hepatotoxicity, a blood disorder, oligospermia or infertility when receiving the aminosalicylate.

In some embodiments, the failure of an IBD patient to be treated with a method provided herein to respond to budenoside treatment is indicated by a sign or symptom of active IBD in the IBD patient despite a history of receiving ≥8 weeks of treatment with budesonide at doses ≥9 grams. In some embodiments, budenoside intolerance is indicated by the development of Cushing's syndrome, osteopenia/osteoporosis, hyperglycemia, insomnia or an infection in an IBD patient receiving budenoside.

In some embodiments, the failure of an IBD patient to be treated with a method provided herein to respond to treatment with a systemic corticosteroid is indicated by a sign or symptom of active IBD in the IBD patient despite a history of receiving at least one 4-week course regimen with prednisone ≥0.75 mg/kg mg daily orally or equivalent, or 1 week of intravenous corticosteroids; or 2 failed attempts to taper corticosteroids to ≤10 mg of prednisone or equivalent on 2 occasions.

In some embodiments, the failure of an IBD patient to be treated with a method provided herein to respond to treatment with an immunosuppressant is indicated by a sign or symptom of active CD in the IBD patient despite a history of ≥8 weeks of treatment with azathioprine (≥1.5 mg/kg), or 6-mercaptopurine (≥0.75 mg/kg), or methotrexate (≥12.5 mg/week). In some embodiments, intolerance to an immunosuppressant is indicated by nausea, vomiting, abdominal pain, pancreatitis, a liver function test abnormality, lymphopenia or an infection in an IBD patient receiving the systemic immunosuppressant.

In some embodiments of the methods provided herein, the failure of an IBD patient to respond to a treatment with biologics is indicated by (a) an inadequate initial response (primary non-responder) after at least 2 doses of induction therapy to: infliximab (doses of >5 mg/kg); adalimumab (doses of 160 mg followed by 80 mg); certolizumab (doses of 400 mg) at least 2 weeks apart; or vedolizumab (doses of 300 mg) at least 8 weeks apart, or (b) recurrence of signs and symptoms, such as worsening of diarrhea, abdominal pain, rectal bleeding, or initiation or increased use of antidiarrheals (secondary non-responders) after at least 2 doses of maintenance therapy to infliximab (doses of >5 mg/kg), adalimumab (doses of 40 mg), certolizumab (doses of 400 mg); or vedolizumab (doses of 300 mg). In some embodiments, intolerance to biologics is indicated by fever, chills, rash, flush, itching, hypotension, urticaria, myalgia, arthralgias; which were related to the treatment.

In some embodiments, an IBD patient to be treated with a method provided herein has received one or more additional IBD treatments prior to the SMAD7 AON treatment. In some embodiments, an IBD patient to be treated with a method provided herein has received one additional IBD treatment prior to the SMAD7 AON treatment. In some embodiments, an IBD patient to be treated with a method provided herein has received two or more additional IBD treatments prior to the SMAD7 AON treatment. In some embodiments, one of the additional IBD treatment is biologics treatment (e.g., infliximab, adalimumab, certolizumab, or vedolizumab) In some embodiments, one of the additional IBD treatment is corticosteroid treatment (e.g., prednisone, budesonide). In some embodiments, one of the additional IBD treatment is immunosuppressant treatment (e.g., AZA, 6-MP, or MTX). In some embodiments, one of the additional IBD treatment is aminosalicylate treatment (e.g., SSZ, ASA).

In some embodiments, the failure of an IBD patient to be treated with a method provided herein receives an additional IBD treatment other than the SMAD7 AON.

In some embodiments, the additional IBD treatment comprises an oral aminosalicylate. In some embodiments, the additional IBD treatment comprising the oral aminosalicylate was initiated ≥6 weeks prior to the beginning of the first treatment period and the oral aminosalicylate has been administered at a stable dose for ≥2 weeks prior to the beginning of the first treatment period and the dose of the oral aminosalicylate remains stable during the first treatment period and/or the second treatment period. In some embodiments, the patient having IBD has discontinued an aminosalicylate at least 2 weeks prior to the beginning of the first treatment period.

In some embodiments, the dose of the oral aminosalicylate remains stable through week 12. In some embodiments, the dose of the oral aminosalicylate may be changed (i.e., tapered, stopped or increased), as clinically indicated, at the discretion of the doctor after week 12.

In some embodiments, the additional IBD treatment comprises an oral corticosteroid. In some embodiments, the oral corticosteroid was administered at a stable dose for at least about 4 weeks prior to the first treatment period (e.g., prednisone ≤20 mg/day or equivalent, budesonide ≤9 mg/day) and the dose of the oral corticosteroid remains stable until the patient having IBD starts corticosteroid tapering.

In some embodiments, the oral corticosteroid was administered at a stable dose for at least about 3 weeks prior to the first treatment period (prednisone <20 mg/day or equivalent, budesonide <9 mg/day) and the dose remains stable until the subject is eligible to start corticosteroids tapering. In some embodiments, the patient having IBD has discontinued an oral corticosteroid at least 3 weeks prior to the beginning of the first treatment period. In some embodiments, the oral corticosteroid was administered at a stable dose for at least about 3 weeks prior to the first treatment period (prednisone <20 mg/day or equivalent, budesonide <9 mg/day) and the dose remains stable through week 12. In some embodiments, the dose of the oral corticosteroid may be changed (i.e., tapered, stopped or increased), as clinically indicated, at the discretion of the doctor after week 12.

In some embodiments, the additional IBD treatment comprises an immunosuppressant, such as 6-MP, AZA, or MTX. In some embodiments, the addition IBD treatment comprising the immunosuppressant was initiated >12 weeks prior to the first treatment period and the immunosuppressant was administered at a stable dose for ≥8 weeks prior to the first treatment period and continues to be administered at a stable dose during the first treatment period and/or the second treatment period. In some embodiments, the patient having IBD has discontinued an immunosuppressant at least 8 weeks prior to the first treatment period.

In some embodiments, the addition IBD treatment comprising the immunosuppressant was initiated >12 weeks prior to the first treatment period and the immunosuppressant was administered at a stable dose for ≥8 weeks prior to the first treatment period and continues to be administered at a stable dose through week 12. In some embodiments, the dose of the oral corticosteroid may be changed (i.e., tapered, stopped or increased), as clinically indicated, at the discretion of the doctor after week 12.

In some embodiments, oral aminosalicylates (such as sulfasalazine. [SSZ] or 5-aminosalicylic acid [5-ASA] compounds); or immunosuppressants (such as AZA, 6-MP, or MTX) may be initiated or changed before the beginning of the first treatment period, or continued from the previous first and/or second treatment period, provided that the dose remains stable through the first 12 weeks of the third treatment period from Week 0 to Week 12. In some embodiments, after Week 12 of the third treatment, patients may taper doses or completely discontinue any of these background CD medications or may increase doses or add any new CD medications as clinically indicated, except for biologics, at the discretion of the doctor.

In some embodiments, oral corticosteroids (with no dose restriction) may be initiated or changed before the beginning of the first treatment period, or continued from the previous first and/or second treatment period, provided that the dose remains stable through the first 4 weeks of the third treatment period from Week 0 to Week 4. In some embodiments, after Week 4, patients may taper corticosteroids doses as clinically indicated, at the discretion of the doctor.

In some embodiments, an IBD patient to be treated with a method provided herein meets one or more of the following laboratory criteria: white blood cell count >3000/mm³ (>3.0×10⁹/L) and <14,000/mm³ (<14.0×10⁹/L); platelet count >100,000/mm³ (>100×10⁹/L); serum creatinine ≤1.5 mg/dL (≤132.6 μmol/L); AST (SGOT) and ALT (SGPT)≤2× upper limit of normal (ULN); total bilirubin ≤2 mg/dL (≤34 μmol/L) or albumin >lower limit of normal (LLN); hemoglobin >9 g/dL (>5.6 mmol/L), activated partial thromboplastin time (APTT)≤1.5×ULN.

In some embodiments, an IBD patient to be treated with a method provided herein is a male or female patient of at least 18 years of age. In some embodiments, the IBD patient is a male or female patient of between about 18 and about 45 years of age. In some embodiments, the IBD patient is a male or female patient of about 18 and about 75 years of age.

In some embodiments, an IBD patient to be treated with a method provided herein is diagnosed as a CD patient using the Vienna classification scheme, the Montreal classification scheme, or the Paris classification scheme. See, e.g., Gasche, C., et al., A simple classification of Crohn's disease: report of the Working Party for the World Congresses of Gastroenterology, Vienna 1998. Inflamm Bowel Dis. 2000 February; 6(1):8-15; Levine, A., et al. A comparison of budesonide and prednisone for the treatment of active pediatric Crohn disease. J Pediatr Gastroenterol Nutr 2003; 36(2):248-52; Levine, A., et al., Pediatric modification of the Montreal classification for inflammatory bowel disease: the Paris classification. Inflamm Bowel Dis. 2011 June; 17(6):1314-21. doi: 10.1002/ibd.21493. Epub 2010 Nov. 8; Satsangi, J., et al., The Montreal classification of inflammatory bowel disease: controversies, consensus, and implications. Gut. 2006 June; 55(6):749-53.

In some embodiments, an IBD patient to be treated with a method provided herein is treatment naïve at the beginning of the first treatment period. In some embodiments, the patient having IBD has never received an anti-SMAD7 therapy prior to the first treatment period. In some embodiments, the patient having IBD has never received a SMAD7 AON prior to the first treatment period. In some embodiments, the patient having IBD has never received an IBD treatment other than an SMAD7 AON prior to the first treatment period.

In some embodiments, an IBD patient to be treated with a method provided herein has received one or more additional IBD treatments other than a SMAD7 AON prior to the first treatment period (e.g., an aminosalicylate, a corticosteroid, or an immunosuppressant). In some embodiments, the IBD patient has discontinued an additional IBD treatment prior to the first treatment period (e.g., at least 1 week, at least 2 weeks, at least 4 weeks, at last 2 months, at least 3 months, at least 6 months, at least 9 months, at least 12 months, at least 18 months, at least 2 years, at least 3 years, at least 4 years, or at least 5 years prior to the first treatment period). In some embodiments, the IBD patient continues to receive an additional IBD treatment during the first treatment period or during parts of the first treatment period. In some embodiments, the IBD patient continues to receive the additional IBD treatment during the second treatment period or during parts of the second treatment period. In some embodiments, an IBD patient receiving a certain additional IBD treatment is not eligible for a treatment according to the methods provided herein. In some embodiments, the IBD patient is cannot tolerate one or more additional treatment. In some embodiments, an IBD patient to be treated with a method provided herein has failed to respond to or to achieve remission with one or more additional IBD treatments. In some embodiments, the IBD patient can taper one or more additional IBD treatments during a treatment according to the methods provided herein (e.g., during the first or second treatment period). In some embodiments, the IBD patient cannot taper one or more additional IBD treatments.

In some embodiments, an IBD patient to be treated with a method provided herein at the beginning of the first treatment period is not diagnosed with ulcerative colitis (UC), indeterminate colitis, ischemic colitis, microscopic colitis, radiation colitis or diverticular disease-associated colitis. In some embodiments, the patient having IBD at the beginning of the first treatment period is not diagnosed with local manifestations of CD, such as strictures, abscesses, fistula, short bowel syndrome or other disease complications for which surgery might be indicated or could confound the evaluation of efficacy. In some embodiments, the patient having IBD has not received an intestinal resection within 6 months or any intra-abdominal surgery within 3 months prior to the first treatment period. In some embodiments, the patient having IBD has not received an ileostomy or a colostomy or does not have intestinal pathogens the beginning of the first treatment period. In some embodiments, the patient having IBD does not have a history of colorectal cancer or colorectal dysplasia. In some embodiments, the patient having IBD has not used mycophenolic acid, tacrolimus, sirolimus, cyclosporine, thalidomide or apheresis (e.g., Adacolumn®) before the beginning of the first treatment period. In some embodiments, the patient having IBD has not used intravenous (IV) corticosteroids within 2 weeks of the beginning of the first treatment period. In some embodiments, the patient having IBD has not used a topical treatment with 5-aminosalicylic acid (5-ASA) or corticosteroid enemas or suppositories within 2 weeks of the beginning of the first treatment period. In some embodiments, the patient having IBD is not stool positive for any enteric pathogen or C. difficile toxin at the beginning of the first treatment period. In some embodiments, the patient having IBD has not received an antibiotic therapy for the treatment of CD within 3 weeks of the beginning of the first treatment period. In some embodiments, the patient having IBD has not used cholestyramine within 3 weeks of the beginning of the first treatment period. In some embodiments, the patient having IBD has not received a treatment with any. biologic agents, including TNF blockers prior to the beginning of the first treatment period. In some embodiments, the patient having IBD has not been administered with total parenteral nutrition (TPN) within 4 weeks of the beginning of the first treatment period. In some embodiments, the patient having IBD does not have a history of one or more of a clinically significant neurological, renal, hepatic, gastrointestinal, pulmonary, metabolic, cardiovascular, psychiatric, endocrine, or hematological disorder or disease. In some embodiments, the patient having IBD is not pregnant or breastfeeding. In some embodiments, the patient having IBD does not have a history of one or more of the following cardiac conditions within 6 months of the beginning of the first treatment period: myocardial infarction, acute coronary syndrome, unstable angina, new onset atrial fibrillation, new onset atrial flutter, second- or third-degree atrioventricular block, ventricular fibrillation, ventricular tachycardia, heart failure, cardiac surgery, interventional cardiac catheterization (with or without a stent placement), interventional electrophysiology procedure, or presence of implanted defibrillator. In some embodiments, the patient having IBD does not have a known active current or history of recurrent bacterial, viral, fungal, mycobacterial or other infections (including but not limited to tuberculosis and atypical mycobacterial disease and Herpes zoster), human immunodeficiency virus (HIV), or any major episode of infection requiring hospitalization or treatment with intravenous (IV) or oral antibiotics within 4 weeks of the beginning of the first treatment period. In some embodiments, the patient having IBD does not have a history of congenital or acquired immunodeficiency (e.g., common variable immunodeficiency disease). In some embodiments, the patient having IBD does not have a history of malignancy, except for: treated (i.e., cured) basal cell or squamous cell in situ skin carcinomas, treated (i.e., cured) cervical intraepithelial neoplasia or carcinoma in situ of the cervix with no evidence of recurrence within the previous 5 years. In some embodiments, the patient having IBD has not received any investigational drug or device within 3 months of the beginning of the first treatment period. In some embodiments, the patient having IBD has not received a prior treatment with a SMAD7 AON. In some embodiments, the patient having IBD does not have a history of alcohol, drug, or chemical abuse within the 6 months prior to the beginning of the first treatment period. In some embodiments, the patient having IBD does not have a known hypersensitivity to oligonucleotides.

7.10 SMAD7 Antisense Oligonucleotides

Antisense oligonucleotides are short synthetic oligonucleotide sequences complementary to the messenger RNA (mRNA), which encodes for the target protein (e.g., SMAD7). Without being bound by theory, antisense oligonucleotide sequences hybridize to the mRNA producing a double-strand hybrid that can lead to the activation of ubiquitary catalytic enzymes, such as RNase H, which degrades DNA/RNA hybrid strands, thus preventing protein translation. Without being bound by theory, an antisense oligonucleotide described in this section and useful in the methods provided herein can hybridize to its target sequence as RNA or DNA. Thus, even if a DNA sequence is provided as target, the corresponding RNA sequence (including uracil instead of thymine) is included. The antisense oligonucleotide can be either RNA or DNA.

The SMAD7 AON used in the methods provided herein can specifically target SMAD7 from any one mammalian organism. Such mammalian organisms comprise, e.g., without limitation, humans, primates (e.g., monkeys, chimpanzees, orangutans, and gorillas), cats, dogs, rabbits, farm animals (e.g., cows, horses, goats, sheep, pigs), and rodents (e.g., mice, rats, hamsters, and guinea pigs).

The SMAD7 AON can target any one region of SMAD7, including any translated region or any untranslated region. Any 8 or more, 10 or more, 12 or more, 14 or more, 16 or more, 18 or more, 20 or more, 22 or more, 24 or more, 26 or more, 28 or more or 30 or more consecutive nucleotides of SMAD7 can be targeted by the SMAD7 AON s.

In some embodiments, the SMAD7 AON can target a region in human SMAD7. In some embodiments, the SMAD7 AON can target a region of 8 or more, 10 or more, 12 or more, 14 or more, 16 or more, 18 or more, 20 or more, 22 or more, 24 or more, 26 or more, 28 or more or 30 or more consecutive nucleotides of human SMAD7. In some embodiments, the SMAD7 AON can target a region in a human SMAD7 including the nucleic acid sequence of SEQ ID NO: 1, or the corresponding RNA sequence.

SEQ ID NO: 1 (Coding Sequence: CDS (288-1568) of NM_005904.3; Homo sapiens SMAD family member 7 (SMAD7), transcript variant 1, mRNA) (region 108-128 underlined):

ATG TTCAGGACCA AACGATCTGC GCTCGTCCGG CGTCTCTGGA
GGAGCCGTGC GCCCGGCGGC GAGGACGAGG AGGAGGGCGC
AGGGGGAGGT GGAGGAGGAG GCGA [symbol] [symbol] [symbol] [symbol]
GGACA GCCGAGCGCA TGGGGCCGGT GGCGGCGGCC
CGGGCAGGGC TGGATGCTGC CTGGGCAAGG CGGTGCGAGG
TGCCAAAGGT CACCACCATC CCCACCCGCC AGCCGCGGGC
GCCGGCGCGG CCGGGGGCGC CGAGGCGGAT CTGAAGGCGC
TCACGCACTC GGTGCTCAAG AAACTGAAGG AGCGGCAGCT
GGAGCTGCTG CTCCAGGCCG TGGAGTCCCG CGGCGGGACG
CGCACCGCGT GCCTCCTGCT GCCCGGCCGC CTGGACTGCA
GGCTGGGCCC GGGGGCGCCC GCCGGCGCGC AGCCTGCGCA
GCCGCCCTCG TCCTACTCGC TCCCCCTCCT GCTGTGCAAA
GTGTTCAGGT GGCCGGATCT CAGGCATTCC TCGGAAGTCA
AGAGGCTGTG TTGCTGTGAA TCTTACGGGA AGATCAACCC
CGAGCTGGTG TGCTGCAACC CCCATCACCT TAGCCGACTC
TGCGAACTAG AGTCTCCCCC CCCTCCTTAC TCCAGATACC
CGATGGATTT TCTCAAACCA ACTGCAGACT GTCCAGATGC
TGTGCCTTCC TCCGCTGAAA CAGGGGGAAC GAATTATCTG
GCCCCTGGGG GGCTTTCAGA TTCCCAACTT CTTCTGGAGC
CTGGGGATCG GTCACACTGG TGCGTGGTGG CATACTGGGA
GGAGAAGACG AGAGTGGGGA GGCTCTACTG TGTCCAGGAG
CCCTCTCTGG ATATCTTCTA TGATCTACCT CAGGGGAATG
GCTTTTGCCT CGGACAGCTC AATTCGGACA ACAAGAGTCA
GCTGGTGCAG AAGGTGCGGA GCAAAATCGG CTGCGGCATC
CAGCTGACGC GGGAGGTGGA TGGTGTGTGG GTGTACAACC
GCAGCAGTTA CCCCATCTTC ATCAAGTCCG CCACACTGGA
CAACCCGGAC TCCAGGACGC TGTTGGTACA CAAGGTGTTC
CCCGGTTTCT CCATCAAGGC TTTCGACTAC GAGAAGGCGT
ACAGCCTGCA GCGGCCCAAT GACCACGAGT TTATGCAGCA
GCCGTGGACG GGCTTTACCG TGCAGATCAG CTTTGTGAAG
GGCTGGGGCC AGTGCTACAC CCGCCAGTTC ATCAGCAGCT
GCCCGTGCTG GCTAGAGGTC ATCTTCAACA GCCGGTAG

In some embodiments, the SMAD7 AON targets region 108-128 of a human SMAD7. In some embodiments, the human SMAD7 has the nucleic acid sequence of SEQ ID NO: 1, or the corresponding RNA sequence. In some embodiments, the human SMAD7 is a naturally occurring variant of the human SMAD7 having the nucleic acid sequence of SEQ IN NO:1.

In some embodiments, the SMAD7 AON targets nucleotides 403, 233, 294, 295, 296, 298, 299 or 533 of the human SMAD7. In some embodiments, the SMAD7 AON targets nucleotides 403, 233, 294, 295, 296, 298, 299 or 533 the nucleic acid sequence of SEQ ID NO: 1, or the corresponding RNA sequence.

In some embodiments, the SMAD7 AON comprises the nucleotide sequence of SEQ ID NO: 2 (5′-GTCGCCCCTTCTCCCCGCAG-3′).

In some embodiments, the SMAD7 AON comprises the nucleotide sequence of SEQ ID NO: 3 (5′-GTCGCCCCTTCTCCCCGCAGC-3′).

The SMAD7 AON used in the methods provided herein can comprise naturally occurring nucleobases, sugars, and covalent internucleotide (backbone) linkages, as well as non-naturally occurring portions. For example, the SMAD7 AON can comprise a mixed-backbone, e.g., including one or more phosphorothioate linkages. In some embodiments, the SMAD7 AON can have one or more cytosine residues replaced by 5-methylcytosine. In some embodiments the one or more cytosine residues form part of a CpG pair.

In some embodiments, the SMAD7 AON can comprise artificial nucleotides, such as deoxycytidine and/or 5-methyl 2′-deoxycytidine, including, but not limited to, 5-methyl-2′-deoxycytidine 5′-monophosphate and 5-methyl-2′-deoxycytidine 5′-monophosphorothioate.

In some embodiments, the SMAD7 AON comprises the nucleic acid sequence of SEQ ID NO:4 (5′-GTXGCCCCTTCTCCCXGCAG-3)′, wherein X is 5-methyl 2′-deoxycytidine.

In some embodiments, the SMAD7 AON comprises the nucleic acid sequence of SEQ ID NO: 5 (5′-GTXYCCCCTTCTCCCXYCAG-3′), whereby X is a nucleotide comprising a nitrogenous base selected from the group consisting of cytosine, 5-methylcytosine and 2-O-methylcytosine, and wherein Y is a nucleotide comprising a nitrogenous base selected from the group consisting of guanine, 5-methylguanine and 2-O-methylguanine, optionally provided that at least one of the nucleotides X or Y comprises a methylated nitrogenous base. In some embodiments, at least one of the internucleoside linkages of the SMAD7 AON is a phosphorothioate linkage. In some embodiments, all of the internucleoside linkages of the SMAD7 AON are phosphorothioate linkages. In some embodiments, the SMAD7 AON is a SMAD7 AON comprising a nucleotide sequence of SEQ ID NO: 5, wherein all internucleoside linkages are phosphothioate linkages.

In some embodiments, the SMAD7 AON comprises the nucleic acid sequence of SEQ ID NO: 6: (5′-GTXGCCCCTTCTCCCXGCAGC-3′), whereby X is 5-methyl-2′-deoxycytidine. In some embodiments, at least one of the internucleoside linkages of the SMAD7 AON is a phosphorothioate linkage. In some embodiments, all of the internucleoside linkages of the SMAD7 AON are phosphorothioate linkages. In some embodiments, the SMAD7 AON is a SMAD7 AON comprising a nucleotide sequence of SEQ ID NO: 6, wherein all internucleoside linkages are phosphothioate linkages.

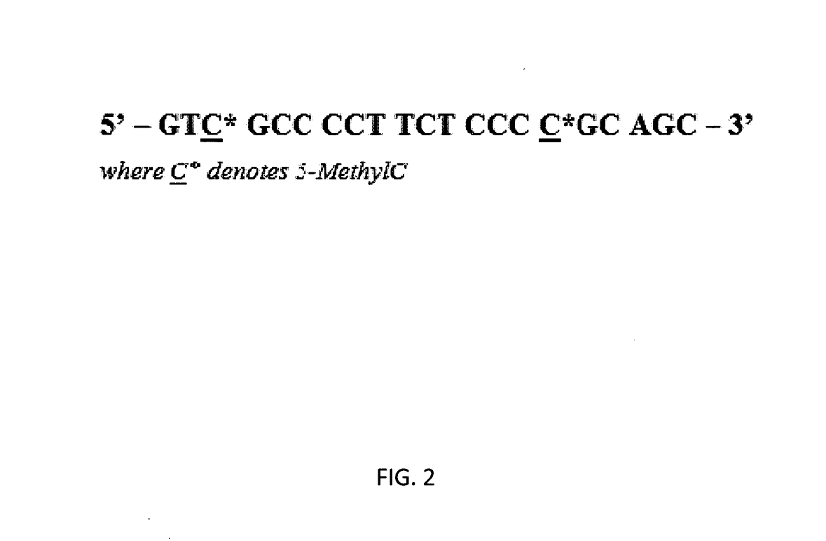

In some embodiments, the SMAD7 AON comprises the AON of FIG. 2.

COMPOUND (I) is a SMAD7 AON with a phosphorothioate backbone. It can be described chemically as the fully neutralized sodium salt of a 3′→5′ linked 2′-deoxyribophosphorothioate oligonucleotide 21-mer in which each of the 20 internucleotide linkages is an O,O linked phosphorothioate. The sequence of heterocyclic bases is shown in FIG. 2 in a standard oligonucleotide structure drawing convention, where T=thymidine, C=2′-deoxycytidine, C*=5-Methyl-2′-deoxycytidine, G=2′-deoxyguanosine, and A=2′-deoxyadenosine, reading left to right from 5′ to 3′.

In some embodiments, the SMAD7 AON has the structure of COMPOUND (I). The following structure of COMPOUND (I) is drawn over four pages:

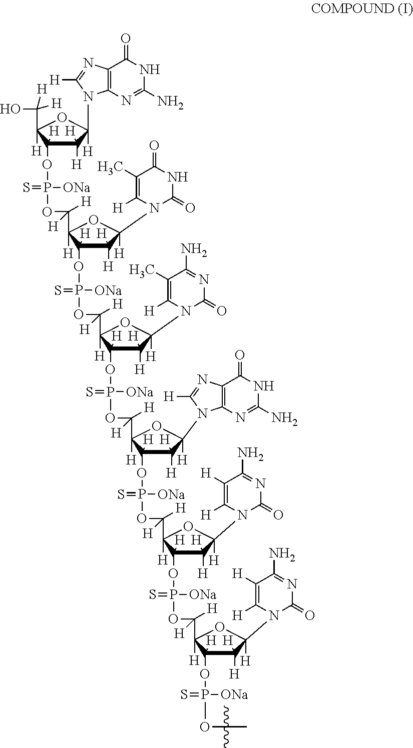

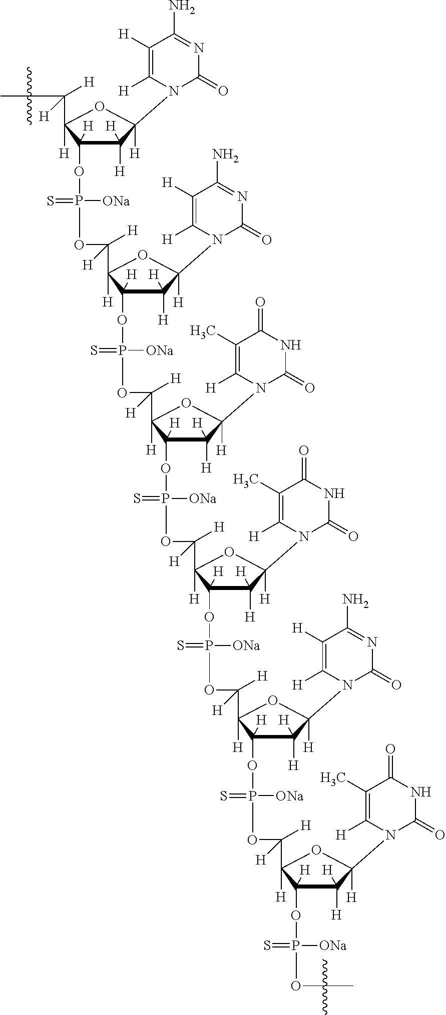

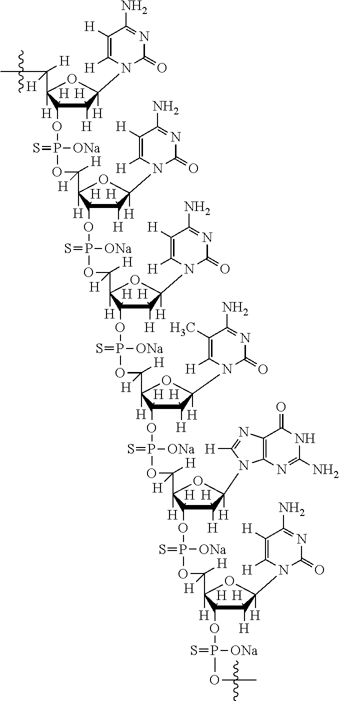

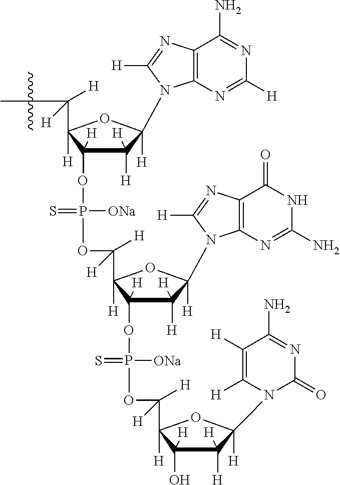

The structure of COMPOUND (I) is presented herein to show the sodium counterion (“Na”). A skilled artisan will understand that COMPOUND (I) may also refer to the anionic form without counterion. A skilled artisan will further understand that an anionic form of COMPOUND (I) can be protonated to form an acidic form of COMPOUND (I). In some embodiments, the phosphorothioate backbone of COMPOUND (I) can be fully or partially protonated to form an acidic form of COMPOUND (I).

In some embodiments, COMPOUND (I) is formulated as a gastro-resistant delayed release pH-dependent tablet designed to deliver the active substance in the distal GI tract (Formulation (I)).

In some embodiments, the SMAD7 AON comprises at least one internucleoside linkage, which is a phosphate linkage, e.g., a monophosphate linkage.

In some embodiments, the SMAD7 AON comprises at least one internucleoside linkage, which is a phosphorothioate linkage. In some embodiments, the SMAD7 AON comprises at least 2, 3, 4, 5, 6, 7, 8, 9, 10, 11, 12, 13, 14, 15, 16, 17, 18, 19, 20, 21, 22, 23, 24, 25, or more phosphorothioate linkages. In some embodiments, at least 5%, 10%, 20%, 25%, 30%, 35%, 40%, 45%, 50%, 55%, 60%, 65%, 70%, 75%, 80%, 85%, 90%, 95%, or 100% of internucleoside linkages in the SMAD7 AON are phosphorothioate linkages. In some embodiments, all internucleoside linkages are phosphorothioate linkages.

In some embodiments, the SMAD7 AON comprises at least one, unnatural nucleoside, e.g., 5-methyl-2′-deoxycytidine-5′-monophosphate and 5-methyl-2′-deoxycytidine-5′-monophosphorothioate. In some embodiments, the SMAD7 AON comprises 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidines. In some embodiments, at least 5%, 10%, 20%, 25%, 30%, 35%, 40%, 45%, 50%, 55%, 60%, 65%, 70%, 75%, 80%, 85%, 90%, 95%, or 100% of nucleotides in the SMAD7 AON comprise deoxycytidine and/or 5-methyl-2′-deoxycytidine. In some embodiments, the SMAD7 AON comprises at least 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidine. In some embodiments, the SMAD7 AON comprises one or more deoxycytidines and no 5-methyl 2′-deoxycytidine. In some embodiments, the SMAD7 AON comprises one or more 5-methyl 2′-deoxycytidine and no deoxycytidine.

In some embodiments, the SMAD7 AON comprises methylphosphonate linkages that are be placed at the 5′- and/or 3′-ends of the SMAD7 antisense oligonucleotides.

In some embodiments, the SMAD7 AON comprises pharmaceutically acceptable salts or solvates. In some embodiments, the solvates are hydrates. In some embodiments, the SMAD7 AON is a sodium salt of the SMAD7 AON including the nuclei acid sequence of COMPOUND (I), that optionally can comprise 1 to 20 O,O-linked phosphorothioate internucleotide linkages. Contemplated salts of SMAD7 AON comprise those that are fully neutralized, e.g., each phosphorothioate linkage is associated with an ion such as Na+. In some embodiments the salts of the SMAD7 AON are only partially neutralized, e.g., less than all phosphorothioate linkages are associated with an ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized).

Exemplary SMAD7 AON s, are be described in U.S. Pat. Nos. 6,159,697, 7,807,818, and 8,648,186 and in International Patent Application Publication WO 2010/054826, each of which is incorporated herein by reference.

In some embodiment, the SMAD7 AON is an isotopically enriched SMAD7 antisense oligonucleotide, e.g., having one or more H replaced with a D.

In some embodiments, the 2′-deoxyribonucleotides in the SMAD7 AON is replaced by corresponding ribonucleotides.

7.11 Pharmaceutical Compositions

The pharmaceutical compositions described in this section can be used in the methods provided herein. In some embodiments, the pharmaceutical composition comprises a SMAD7 AON described and a pharmaceutically acceptable adjuvant and/or excipient. In some embodiments, the pharmaceutical composition is an oral pharmaceutical composition. In some embodiments, the pharmaceutical composition comprises an enteric coating to topically deliver the modified SMAD7 AON to the terminal ileum and/or right colon of an IBD patient. In some embodiments, the pharmaceutical composition is a gastro-resistant granules formulation.

Contemplated SMAD7 AON s comprise oligonucleotides that act against SMAD7 and can be administered orally. Disclosed therapies can, when administered orally to a subject suffering from IBD, deliver an effective amount of an AON to the intestinal system of a patient, e.g. deliver an effective amount of an AON to the terminal ileum and/or right colon of a patient.

In some embodiments of the methods of treating IBD provided herein, the anti-SMAD7 therapy (e.g., a therapy comprising a SMAD7 AON) can be suitable for oral delivery of an AON, e.g., tablets, that comprise an enteric coating, e.g., a gastro-resistant coating, such that the compositions can deliver the antisense compound to, e.g., the terminal ileum and right colon of a patient. For example, such administration can result in a topical effect, substantially topically applying the antisense compound directly to an affected portion of the intestine of a subject. Such administration, can, in some embodiments, substantially avoid unwanted systemic absorption of the antisense compound.

For example, a tablet for oral administration can comprise granules (e.g., is at least partially formed from granules) that comprise a disclosed SMAD7 antisense oligonucleotide and pharmaceutically acceptable excipients. Such a tablet can be coated with an enteric coating. Contemplated tablets can comprise pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations comprise an intra-granular phase that comprises a contemplated SMAD7 AON or a pharmaceutically acceptable salt and a pharmaceutically acceptable filler. For example, COMPOUND (I) and a filler can be blended together, with optionally other excipients, and formed into granules. In some embodiments, the intragranular phase can be formed using wet granulation, e.g. a liquid (e.g., water) is added to the blended antisense compound and filler, and then the combination is dried, milled and/or sieved to produce granules. One of skill in the art would understand that other processes can be used to achieve an intragranular phase.

In some embodiments, contemplated formulations comprise an extra-granular phase, which can comprise one or more pharmaceutically acceptable excipients, and which can be blended with the intragranular phase to form a disclosed formulation.

An anti-SMAD7 therapy formulation can comprise an intragranular phase that comprises a filler. Exemplary fillers comprise, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, an anti-SMAD7 therapy formulation can comprise an intragranular phase and/or an extragranular phase that comprises a binder, which can generally function to hold the ingredients of the pharmaceutical formulation together. Exemplary binders comprise, for example, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others, including combinations thereof.

Contemplated anti-SMAD7 therapy formulations, e.g., that comprise an intragranular phase and/or an extragranular phase, can comprise a disintegrant, such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase can comprise a disintegrant.

In some embodiments, a contemplated anti-SMAD7 therapy formulation comprises an intra-granular phase comprising a disclosed antisense compound and excipients chosen from: mannitol, microcrystalline cellulose, hydroxypropylmethyl cellulose, and sodium starch glycolate, or combinations thereof, and an extra-granular phase comprising one or more of: microcrystalline cellulose, sodium starch glycolate, and magnesium stearate, or mixtures thereof.

In some embodiments, a contemplated anti-SMAD7 therapy formulation can comprise a lubricant, e.g., an extra-granular phase can contain a lubricant. Lubricants comprise but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings can comprise a polymer that disintegrates at different rates according to pH. Enteric coatings can comprise, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In some embodiments, the enteric coating comprises an anionic, cationic, or neutral copolymer based on methacrylic acid, methacrylic/acrylic esters or their derivatives. In some embodiments, the enteric coating comprises an ethylacrylate-methacrylic acid copolymer. Commercially available enteric coatings comprise Opadry® AMB, Acryl-EZE®, Eudragit®. In some embodiments, the enteric coating makes up about 5% to about 10%, about 5% to about 20%, about 8 to about 15%, about 8% to about 18%, about 10% to about 12%, or about 12% to about 16%, of a contemplated tablet by weight.

For example, an anti-SMAD7 therapy in the form of a tablet is provided that comprises or consists essentially of about 0.5% to about 70%, e.g., about 0.5% to about 10%, or about 1% to about 20%, by weight of a SMAD7 antisense oligonucleotide or a pharmaceutically acceptable salt thereof. Such a tablet can comprise for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a contemplated tablet can comprise an intragranular phase that comprises about 30% to about 60%, e.g., about 45% to about 65% by weight, or alternatively, about 5 to about 10% by weight of Compound (I), about 30% to about 50%, or alternatively, about 5% to about 15% by weight mannitol, about 5% to about 15% microcrystalline cellulose, about 0% to about 4%, or about 1% to about 7% hydroxypropylmethyl cellulose, and about 0% to about 4%, e.g., about 2% to about 4% sodium starch glycolate by weight.

Exemplary anti-SMAD7 therapy formulations comprise dosage forms that comprise or consist essentially of about 10 mg to about 500 mg of an SMAD7 antisense oligonucleotide including the nucleic acid sequence of COMPOUND (I), for example, tablets that comprise between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg of COMPOUND (I) are contemplated herein. In some embodiments, the tablets comprise between about 5 mg and about 90 mg, between about 10 mg and about 70 mg, or between about 30 mg and about 50 mg of COMPOUND (I). In some embodiments, the tablets comprise about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg of COMPOUND (I).

In one embodiment, the anti-SMAD7 therapy can be a tablet for oral use comprising: about 0.5% to about 10% by weight of COMPOUND (I) or a pharmaceutically acceptable salt thereof; about 30% to about 50% by weight mannitol; and about 10% to about 30% by weight microcrystalline cellulose.

In an exemplary embodiment of the invention, a pharmaceutically acceptable tablet for oral administration is provided that comprises an intra-granular phase that can comprise about 50% by weight of COMPOUND (I) (or salt thereof), about 11.5% by weight mannitol, about 10% by weight microcrystalline cellulose, about 3% by weight hydroxypropylmethyl cellulose, and about 2.5% by weight sodium starch glycolate; and an extra-granular phase that can comprise about 20% by weight microcrystalline cellulose, about 2.5% by weight sodium starch glycolate, and about 0.5% by weight magnesium stearate. The tablet can also comprise an enteric coating.

In another exemplary embodiment, a pharmaceutically acceptable tablet for oral administration is provided that comprises or consists essentially of: an intra-granular phase that can comprise or consist essentially of about 5% to about 10%, e.g., about 8% by weight of COMPOUND (I), about 40% by weight mannitol, about 8% by weight microcrystalline cellulose, about 5% by weight hydroxypropylmethyl cellulose, and about 2% by weight sodium starch glycolate; and an extra-granular phase that can comprise about 17% by weight microcrystalline cellulose, about 2% by weight sodium starch glycolate, and about 0.4% by weight magnesium stearate.

Contemplated tablets can also comprise an enteric coating, e.g., a disclosed tablet can comprise about 10%, about 11%, about 12%, about 13%, about 14%, about 15%, about 16%, about 17%, or about 18% by weight of an enteric coating, e.g., ethylacrylate-methacrylic acid copolymers (e.g., AcyrlEZE®).

For example, the anti-SMAD7 therapy can be in the form of a pharmaceutically acceptable tablet for oral use comprising an intra-granular phase and extra-granular phase, wherein for example, the intra-granular phase comprises about 5% to about 10%, by weight (for example about 8% by weight) of Compound (I) or a pharmaceutically acceptable salt thereof, about 40% by weight mannitol, about 8% by weight microcrystalline cellulose, about 5% by weight hydroxypropylmethyl cellulose, and about 2% by weight sodium starch glycolate, and, e.g., the extra-granular phase comprises about 17% by weight microcrystalline cellulose, about 2% by weight sodium starch glycolate, and about 0.4% by weight magnesium stearate, where the tablet can further comprise an enteric coating.

Contemplated formulations, e.g., tablets, in some embodiments, when orally administered to the patient can result in minimal plasma concentration of the oligonucleotide in the patient. In another embodiment, contemplated formulations, when orally administered to a patient, topically deliver to the terminal ileum and/or right colon of a patient, e.g., to an affected or diseased intestinal site of a patient.

### 8. EXAMPLES

### Example 1: A Randomized, Double-Blind, Multicenter Study to Explore the Effect of Compound (I) on Endoscopic and Clinical Outcomes in Subjects with Active Crohn's Disease

Study Objectives

The primary objective of the study is to explore the effect of the SMAD7 AON Compound (I) (160 mg QD (QD=once daily)) on endoscopic outcomes, as measured by the SES-CD in subjects with active CD.

The secondary objectives of the study are to evaluate the effect of COMPOUND (I) (40 mg QD and 160 mg QD) on clinical activity, as measured by the CDAI in subjects with active CD and to evaluate the safety and tolerability of COMPOUND (I) (40 mg QD and 160 mg QD) in subjects with active CD.

The exploratory objectives of the study are:
to explore the effect of COMPOUND (I) (40 mg QD and 160 mg QD) on clinical activity, as measured by the PRO-2 in subjects with active CD;
to explore the effect of COMPOUND (I) (40 mg QD and 160 mg QD) on biomarkers of intestinal inflammation and tissue damage, such as but not limited to hsCRP, and FCP in subjects with active CD;
to explore the effects of COMPOUND (I) (160 mg QD) on histological scores in intestinal mucosal biopsies from subjects with active CD; to explore the PD effects of COMPOUND (I) (160 mg QD) on the expression of biomarkers such as, but not limited to CD4, CD8 and HLA-DR, in intestinal mucosal biopsies from subjects with active CD; and
to evaluate the systemic exposure of COMPOUND (I) (160 mg QD) in subjects with active CD.

Drug Identification

COMPOUND (I) is an AS ODN with a phosphorothioate backbone. It may be described chemically as the fully neutralized sodium salt of a 3′-5′ linked 2′-deoxyribophosphorothioate oligonucleotide 21-mer in which each of the 20 internucleotide linkages is an O,O linked phosphorothioate. The sequence of heterocyclic bases is shown in FIG. 2 in a standard oligonucleotide structure drawing convention, where T=thymidine, C=2′-deoxycytidine, C*=5-Methyl-2′-deoxycytidine, G=2′-deoxyguanosine, and A=2′-deoxyadenosine, reading left to right from 5′ to 3′.

Study Design

A schematic diagram illustrating the study design is shown in FIG. 1.

This is a randomized, double-blind, multicenter study to explore the effect of oral COMPOUND (I) on endoscopic and clinical outcomes in subjects with active CD, defined as a CDAI score ≥220 and ≤450 and a SES-CD score ≥7 (or SES-CD >4 if subject has ileitis only).

Approximately 51 subjects will be randomized in a 1:1:1 ratio to receive 1 of 3 treatment regimens in a 12-week Induction Phase:
COMPOUND (I) 160 mg QD for 12 weeks
COMPOUND (I) 160 mg QD for 8 weeks followed by 4 weeks of placebo
COMPOUND (I) 160 mg QD for 4 weeks followed by 8 weeks of placebo

Treatment assignment at baseline will be stratified via an IVRS/IWRS based on previous exposure to TNFα blockers (yes/no) and disease location (disease restricted to the terminal ileum and/or up to the mid transverse colon only, or disease involving at least 1 ulcerated segment distal to mid transverse colon). The number of subjects with previous exposure to TNFα blockers (yes/no) is targeted to be approximately 40%. The number of subjects with disease involving distal to mid transverse colon is targeted to comprise approximately 50% of the study population.

Eligible subjects will enter the Induction Phase at the Baseline Visit (Week 0/Induction Visit 1). Subjects will be assigned randomly to receive IP as described above.

At Induction Week 12, subjects (responders) who achieve clinical remission, defined as a CDAI score <150, or clinical response, defined as a decrease from baseline of ≥100 points in CDAI score, at any of the following Induction Visits (Weeks 4, 8 and/or Week 12) will enter the Observation Phase. The Observation Phase will have a duration of up to 52 weeks. Subjects who are unable to achieve clinical remission or clinical response (nonresponders) at the following Induction Visits (Weeks 4, 8 and Week 12), will be discontinued from the study. Subjects who enter the Observation Phase and were receiving corticosteroids at baseline will start tapering corticosteroids at the end of the Induction Phase (Induction Week 12).

Subjects who enter the Observation Phase will be evaluated by CDAI score every 4 weeks. Subjects will not receive IP during the Observation Phase. Subjects who experience a partial loss of response or are unable to taper corticosteroids during the Observation Phase will enter the Extension Phase. Partial loss of response is defined as 2 consecutive visits with both a CDAI score ≥150 and an increase of CDAI score ≥50 points from the CDAI score at the visit when the subject was first a responder during the Induction Phase. Partial loss of response must be confirmed 2 to 4 weeks post initial identification of partial loss of response. Subjects who do not experience a partial loss of response until Observation Week 52 will have an end-of-study visit.

Subjects who enter the Extension Phase will receive COMPOUND (I) 40 mg QD on a 4-week, alternating dosing schedule (4 weeks of treatment with COMPOUND (I), followed by 4 weeks without COMPOUND (I) treatment) for 24 weeks.

Subjects who complete the Extension Week 24 visit will have two options:
Subjects may proceed to a Long-Term Extension Study (see, e.g., Example 3), if the subjects meet all the inclusion/exclusion criteria of the Long-Term Extension Study.
Subjects can receive a Follow-up Visit if they chose to not enter the Long-Term Extension Study.

Subjects who complete the Extension Phase, as well as subjects who prematurely discontinue from the study, for any reason, will enter the Follow-up Phase, the 4-week period after the last study visit.

The study will consist of 5 phases:
Screening Phase—up to 4 weeks
Induction Phase—12 weeks
Observation Phase—up to 52 weeks
Extension Phase—24 weeks
Follow-up Phase—4 weeks

Study Endpoints

The primary endpoint of this study is the change from baseline in the SES-CD score at Induction Week 12.

The secondary endpoints of the study are:
the proportion of subjects achieving a clinical remission, defined as a CDAI score <150 at Induction Week 4; and
the evaluation of safety and tolerability of COMPOUND (I), assessed by the type, frequency and severity of adverse events, and its relationship to IP, discontinuation due to adverse events, and clinically significant changes in vital signs, ECGs, and/or laboratory findings.

Exploratory endpoints of this study include exploratory efficacy endpoints, exploratory pharmacodynamic (PD)/biomarker endpoints, and exploratory pharmacokinetics (PK) endpoints.

The exploratory efficacy endpoints are:
the proportion of subjects with endoscopic response, defined as a reduction of at least 50% in the SES-CD score, compared to baseline, at Induction Week 12;
the proportion of subjects with endoscopic remission, defined as a SES-CD score ≤2 at Induction Week 12;
the proportion of subjects with mucosal healing, defined as the absence of intestinal mucosal ulcerations at Induction Week 12;
the proportion of subjects who are in clinical remission, defined as a CDAI score <150 at Induction Weeks 2, 8, 12 and Extension Weeks 4, 8, 12, 16, 20, 24;
the proportion of subjects who are in clinical response, defined as a decrease from baseline of ≥100 points in CDAI score at Induction Weeks 2, 4, 8, 12 and Extension Weeks 4, 8, 12, 16, 20, 24;
time to partial loss of response, defined as 2 consecutive visits with both a CDAI score ≥150 and an increase of CDAI score ≥50 points from the CDAI score at the visit when the subject was first a responder during the Induction Phase;
the proportion of subjects who have a PRO-2 score ≤8 at Induction Weeks 2, 4, 8, 12 and Extension Weeks 4, 8, 12, 16, 20, 24;
the proportion of subjects who have a decrease from baseline of ≥8 points in the PRO-2 score at Weeks 2, 4, 8, 12 and Extension Weeks 4, 8, 12, 16, 20, 24;
the proportion of subjects who achieve corticosteroid-free clinical remission at extension Week 24 among subjects receiving oral corticosteroids at baseline;
the change from baseline in the CDAI score at Induction Weeks 2, 4, 8, 12 and Extension Weeks 0, 4, 8, 12, 16, 20, 24;
the change from baseline in the PRO-2 score at Induction Weeks 2, 4, 8, 12 and Extension Weeks 0, 4, 8, 12, 16, 20, 24; the change from baseline in the average daily liquid or soft stool frequency score at Induction Weeks 2, 4, 8, 12 and Extension Weeks 0, 4, 8, 12, 16, 20, 24;
the change from baseline in the average daily abdominal pain score at Induction Weeks 2, 4, 8, 12 and Extension Weeks 0, 4, 8, 12, 16, 20, 24;
the change from baseline in histologic scores from the intestinal mucosa at Induction Week 12; and
the proportion of subjects in clinical response and clinical remission at Observation Week 52.

The exploratory PD/biomarker endpoints are:
the change from baseline in hsCRP at Induction Weeks 4, 8, 12, Observations Week 20, 52 and Extension Weeks 0, 4, 8, 12, 16, 20, 24;
the change from baseline in FCP at Induction Weeks 4, 8, 12, Observation Phase (every 8 weeks for the duration of the Observation Phase), and Extension Weeks 0, 4, 8, 12, 16, 20, 24; and
the change from baseline in PD markers, such as, but not limited to, CD4, CD8 and HLA-DR in intestinal mucosa at Induction Week 12.

The exploratory PK endpoint is the plasma concentration of COMPOUND (I) at Induction Weeks 4, 8 and 12.

Study Population

The study population will consist of female and male subjects 18 years of age and older with active CD. Subjects must have a diagnosis of CD with a duration of at least 3 months prior to screening and a CDAI score ≥220 and ≤450 and SES-CD score ≥7 (or SES-CD >4 if subject has ileitis only) at screening. Subjects must have experienced treatment failure or intolerance to either aminosalicylates, budesonide, systemic corticosteroids, immunosuppressants or TNFα blockers. Enrollment of subjects with prior exposure to TNFα blockers will be limited to approximately 40% of the total subjects enrolled. The number of subjects with disease involving distal to mid transverse colon is targeted to comprise approximately 50% of the study population.

Study Duration

The overall study duration will be up to 97 weeks, with different phases as follows: up to 5 weeks in the Screening Phase, 12 weeks in the Induction Phase, up to 52 weeks in the Observation Phase, 24 Weeks in the Extension Phase, and 4 weeks in the Follow-up Phase.

End of Trial

The End of Trial is defined as either the date of the last visit of the last subject to complete the study, or the date of receipt of the last data point from the last subject that is required for primary, secondary and/or exploratory analysis, as pre-specified in the protocol and/or the SAP, whichever is the later date.

Safety Assessments

Serum and Urine Pregnancy Tests for Females of Childbearing Potential:

A serum pregnancy test with a sensitivity of ≤25 mlU/mL will be required for FCBP at screening. A urine pregnancy test will be performed on all FCBP at the Baseline Visit, at Observation Week 52 if subjects do not experience a partial loss of response by 52 weeks of observation/or Extension Week 24/or the Early Termination Visit, and at the Follow-up Visit. A urine pregnancy test kit will be provided by the central laboratory. Pregnancy tests should be performed if the FCBP has missed a menstrual period or the contraception method has changed.

Vital Signs, Height, and Weight:

Vital signs, including temperature, pulse, and seated blood pressure will be taken during visits. Height will be measured and recorded at screening; weight (to be done in street clothes, no shoes) will also be measured and recorded at different timepoints, including during screening. Body mass index (BMI) will be calculated at screening.

Complete and Limited Physical Examinations:

Complete physical examinations will include evaluation of the skin, nasal cavities, eyes, ears, respiratory, cardiovascular, abdominal, neurological, lymphatic, and musculoskeletal systems. Limited physical examinations will include evaluation of the skin, respiratory, cardiovascular, lymphatic, and musculoskeletal systems. Results of the complete and limited physical examinations will be recorded only in the source documents.

Clinically significant abnormal findings (with the exception of the disease under study [CD]) identified prior to first dose of IP will be recorded on the electronic case report form (eCRF) as medical history; clinically significant findings after the first dose of IP will be recorded as AEs.

Gynecological and urogenital examinations will not be performed unless for cause.

Stool Culture/Microbiology:

Stool culture of enteric pathogens and assessment of Clostridium difficile (C. difficile) toxin will be performed at screening. Subjects who are initially positive for C. difficile may re-screen for the study after they have successfully completed therapy and had 2 months of consecutive negative tests for C. difficile.

Twelve-Lead Electrocardiogram:

The 12-lead ECG will be performed after the subject has been supine for approximately 3 minutes. Sites are to utilize their own local ECG machines for the study and the automated ECG readings will be further interpreted by the Investigator by clinically correlating them with the subject's condition. The Investigator's clinical interpretation will be recorded in the eCRF as: normal; abnormal, not clinically significant; or abnormal, clinically significant. “Abnormal, clinically significant” results should be recorded in the Medical History eCRF if found prior to first dose of IP or in the AE eCRF if found after the first dose of IP.

Clinical Laboratory Evaluations:

A central laboratory will be used for this study. Clinical laboratory evaluations will include:
Hematology: complete blood count (red blood cell [RBC] count, hemoglobin, hematocrit, white blood cell [WBC] count and differential, absolute WBC counts, platelet count)
Coagulation: prothrombin time (PT), activated partial thromboplastin time (APTT)
Serum chemistries: total protein, albumin, calcium, phosphorous, glucose, total cholesterol, triglycerides, uric acid, total bilirubin, alkaline phosphatase, aspartate aminotransferase (AST)/serum glutamic-oxaloacetic transaminase (SGOT), alanine aminotransferase (ALT)/serum glutamic-pyruvic transaminase (SGPT), sodium, potassium, chloride, carbon dioxide, blood urea nitrogen (BUN), creatinine, lactic dehydrogenase (LDH), magnesium, complement activation (Bb, C3a and C5a)
Urinalysis: dipstick urinalysis (specific gravity, pH, glucose, ketones, protein, blood, bilirubin, leukocyte esterase, nitrite, and urobilinogen)
   Microscopic urinalysis (epithelial cells, RBC, WBC, and casts) will be performed only if the dipstick urinalysis is abnormal

Clinical laboratory evaluations are not required to be fasting. However, the site will record whether a clinical laboratory evaluation was fasting or nonfasting on the laboratory requisition form.

Adverse Events

Worsening of a subject's CD, including CD flare, should be considered as worsening of disease under study, and should not be captured as an AE. Worsening or exacerbation of CD, including CD flare, meeting the definition of an SAE should be reported as an SAE.

Efficacy Assessments

Subject Diary for Crohn's Disease Activity

During Screening Visit 1, an electronic subject diary will be given to each subject for CD activity in order to record the following information:
Number of liquid or soft stools per day
Abdominal pain/cramps
General well-being
Fever higher than 37.8° C. during previous week
Taking diphenoxylate/atropine, loperamide, or opiates for diarrhea

The information extracted will be used for calculation of the CDAI and PRO-2, taking into account the data recorded over the last 7 days prior to each study visit.

Crohn's Disease Activity Index

The CDAI is the most commonly used measure in clinical studies evaluating the efficacy of new therapies in CD patients with predominantly inflammatory disease. This index is primarily based on a self-assessment questionnaire completed by the subject. It will assess how CD affects the subject's quality of life and the effect of treatment. The CDAI consists of a questionnaire with responses scored numerically and weighted. Scores (range 0 to 600) are then ranked according to severity of the disease. Mild active disease is defined by a score of ≥150 and ≤219, moderate active disease is defined by a score of ≥220 and ≤450 whereas severe disease is defined as a CDAI score >450. Remission is defined as a CDAI score <150.

The CDAI consists of 8 variables:
Number of liquid or soft stools per day (each day for 7 days)
Abdominal pain/cramps (each day for 7 days)
General well-being (each day for 7 days)
Number of complications:
   Arthritis or arthralgias
   Iritis or uveitis
   Erythema nodosum, pyoderma gangrenosum, or aphthous ulcers
   Anal fissures, fistulae, or abscess
   Other fistula
   Fever higher than 37.8° C. during previous week
Taking loperamide, diphenoxylate, or opiates for diarrhea
Abdominal mass
Hematocrit of less than 0.47 in men and less than 0.42 in women
Percentage of deviation above or below standard weight.

Patient Reported Outcomes

The Patient Reported Outcomes (PROs), defined by patients to quantify symptoms, has been proposed as an important aspect of assessing activity of IBD. PRO-2 for CD consists of 2 items from CDAI: liquid or soft stool frequency and abdominal pain. The total PRO-2 score is calculated similarly to the CDAI, the daily scores of liquid or soft stool frequency and abdominal pain, averaged over 7 days, weighted with the original CDAI multiplication factors. Values corresponding to CDAI scores of 150, 220, and 450 points are 8, 14, and 34 points, and the corresponding values for change from baseline in CDAI scores of 50, 70, and 100 points, are 2, 5, and 8 points for the PRO-2.

Ileocolonoscopy

All subjects are required to have ileocolonoscopies performed during the Screening Phase and Induction Week 12. The screening ileocolonoscopy is required to be performed at least 14 days before the Baseline Visit since it may interfere with the CDAI and PRO-2 assessments.

Intestinal mucosal biopsies will be performed when the ileocolonoscopies are done. Approximately two biopsies will be collected from the involved areas (no ulcer edges).

Conventional histological assessment will also be performed and measured by using a microscopic tissue grading score. During the procedure, biopsies will be collected from the inflamed mucosa (not from ulcers).

Simple Endoscopic Score for CD

The SES-CD is a validated endoscopic index that closely correlates with the Crohn's disease endoscopic index of severity (CDEIS) and is often considered the standard for endoscopic evaluation in subjects with CD. The two score indexes closely correlate; however, SES-CD is considered more suitable for clinical trials due to its simplicity and has been widely adopted for this purpose. Endoscopic response has been defined as a reduction of at least 50% in the SES-CD score from baseline, endoscopic remission defined as a SES-CD ≤2 and mucosal healing defined as the absence of ulceration.

Pharmacodynamic/Biological Markers

High sensitivity C-reactive protein (hsCRP) will be analyzed in blood samples and additional serum biomarkers may be analyzed as well. Fecal calprotectin (FCP) will be assessed in the fecal samples. The expression of biomarkers such as, but not limited to CD4, CD8 and HLA-DR, in intestinal mucosal biopsies will also be analyzed.

Pharmacokinetics

A sparse PK substudy has been incorporated into the study to monitor systematic exposures of COMPOUND (I).

Sparse Pharmacokinetics Blood Draw:

For all subjects enrolled, 2 blood draws will be obtained for sparse PK sampling at Weeks 4, 8, and 12 for a total of 6 blood specimens from a subject. The blood draws will occur at 2 time windows: 1) predose (at least >23 hours after the previous dose); and 2) 1 to 6 hours post-dose.

On all PK visits, subjects must bring their IP to the study center and IP must be administered to subjects at the study center after the collection of the predose PK blood sample.

The last dosing date and time prior to PK blood draw of each visit will be recorded. When the subject is providing a predose blood draw on the day of a PK substudy visit, the subject must be reminded to provide the date and time of his/her last dose from the day before the visit. Actual PK blood sample collection times and associated dosing times (e.g., the dosing times prior to PK sampling times) will be recorded.

Dosing

Subjects will be instructed to take the IP in the morning, 30 minutes before breakfast, with a glass of water.

Study completion for an individual subject is defined as reaching Extension Week 24, or reaching Observation Week 52 if subject does not experience a partial loss of response by 52 weeks of observation and completing the Follow-up Phase. Subjects not meeting this definition will be considered early terminators.

Study Population

Number of Subjects and Sites:

Approximately 48 subjects will be enrolled in this study.

Inclusion Criteria—

Subjects must satisfy the following criteria to be enrolled in the study:
Is a male or female who is >18 years at the time of signing the ICF.
Understand and voluntarily sign an ICF prior to conducting any study related assessments/procedures.
Able to adhere to the study visit schedule and other protocol requirements.
Diagnosis of CD with a duration of at least 3 months prior to screening.
Diagnosis of ileitis, ileocolitis or colitis as determined by endoscopic, radiographic or any other imaging modality (e.g., MRI, CT scan) evaluation performed within 2 years prior to screening. Subjects with colitis restricted to the left colon will not be allowed in the trial.
Active disease, defined as CDAI score ≥220 and ≤450 (range: 0 to 600) at screening.
SES-CD score ≥7 at screening. Subjects with ileitis only will require SES-CD >4.
Must have failed or experienced intolerance to at least one of the following: aminosalicylates, budesonide, systemic corticosteroids or immunosuppressants (e.g., 6-MP, AZA, or MTX) or TNFα blockers (e.g., infliximab, adalimimab or certolizumab).
Subjects receiving oral aminosalicylates may continue their use during the study, provided that treatment was initiated at least 6 weeks prior to the Baseline Visit, and has been given at a stable dose for at least 2 weeks prior to the Baseline Visit. The dose of oral aminosalicylates must remain stable through the duration of the study or early termination from the study. If oral aminosalicylates have been recently discontinued, treatment must have been stopped at least 2 weeks prior to the Baseline Visit.
Subjects receiving oral corticosteroids may continue their use during the Induction Phase, provided that the dose (prednisone ≤20 mg/day or equivalent, budesonide ≤9 mg/day) has been stable for 3 weeks prior to the Baseline Visit. If oral corticosteroids were recently discontinued, discontinuation must have been completed at least 4 weeks prior to screening. Corticosteroid doses should remain stable until the subject is eligible to start corticosteroids tapering.
Subjects receiving immunosuppressants, such as 6-MP, AZA or MTX may continue their use during the study, provided that treatment was initiated ≥12 weeks prior to the Baseline Visit. The dose of immunosuppressants must be at a stable dose for ≥8 weeks prior to the Baseline Visit and must remain stable through the duration of the study or early termination from the study. Subjects who discontinued immunosuppressants should have stopped them at least 8 weeks prior the Baseline Visit.
Must meet the following laboratory criteria:
   White blood cell count ≥3000/mm³ (≥3.0×10⁹/L) and <14,000/mm³ (≤14.0×10⁹/L)
   Platelet count ≥100,000/mm³ (≥100×10⁹/L)
   Serum creatinine ≤1.5 mg/dL (≤132.6 μmol/L)
   AST (SGOT) and ALT (SGPT)≤2× upper limit of normal (ULN)
   Total bilirubin ≤2 mg/dL (≤34 μmol/L) or albumin >lower limit of normal (LLN)
   Hemoglobin ≥9 g/dL (≥5.6 mmol/L)
   Activated partial thromboplastin time (APTT)≤1.5×ULN
Females of childbearing potential (FCBP) must have a negative pregnancy test at Screening and the Baseline Visit.

Exclusion Criteria—

The presence of any of the following will exclude a subject from enrollment:
Diagnosis of Crohn's colitis restricted to the left colon, UC, indeterminate colitis, ischemic colitis, microscopic colitis, radiation colitis or diverticular disease-associated colitis.
Local manifestations of CD such as strictures, abscesses, fistula, short bowel syndrome or other disease complications for which surgery might be indicated or could confound the evaluation of efficacy.
The female subject's chosen form of contraception must be effective by the time the female subject is randomized into the study (for example, hormonal contraception should be initiated at least 28 days before randomization).
Intestinal resection within 6 months or any intra-abdominal surgery within 3 months prior to screening.
Subjects with an ileostomy or a colostomy.
Stool positive for any enteric pathogen or C. difficile toxin at screening.
History of colorectal cancer or colorectal dysplasia.
Prior use of mycophenolic acid, tacrolimus, sirolimus, cyclosporine, thalidomide or apheresis (e.g., Adacolumn) for the treatment of CD. In addition, prior use of any of these treatment modalities for an indication other than CD within 8 weeks of screening is also excluded.
Use of IV corticosteroids within 2 weeks of the Baseline Visit.
Use of topical treatment with 5-ASA or corticosteroid enemas or suppositories within 2 weeks of the Baseline Visit.
Use of antibiotic therapy for the treatment of CD within 3 weeks of screening.
Use of cholestyramine within 3 weeks of screening.
Prior treatment with more than 2 TNFa blockers (e.g., infliximab, adalimumab, or certolizumab) any biologic agents, including TNF blockers.
Prior treatment with any integrin antagonists (e.g., natalizumab or vedolizumab)
Use of TNFα blockers within 12 months of screening.
Administration of total parenteral nutrition (TPN) within 4 weeks of screening.
History of any clinically significant neurological, renal, hepatic, gastrointestinal, pulmonary, metabolic, cardiovascular, psychiatric, endocrine, hematological disorder or disease, or any other medical condition that, in the Investigator's opinion, would prevent the subject from participation in the study.
Any condition, including the presence of laboratory abnormalities, which places the subject at unacceptable risk if he/she was to participate in the study or confounds the ability to interpret data from the study.
Pregnant or breastfeeding.
History of any of the following cardiac conditions within 6 months of screening: myocardial infarction, acute coronary syndrome, unstable angina, new onset atrial fibrillation, new onset atrial flutter, second- or third-degree atrioventricular block, ventricular fibrillation, ventricular tachycardia, heart failure, cardiac surgery, interventional cardiac catheterization (with or without a stent placement), interventional electrophysiology procedure, or presence of implanted defibrillator.
Known active current or history of recurrent bacterial, viral, fungal, mycobacterial or other infections (including but not limited to tuberculosis and atypical mycobacterial disease and Herpes zoster), human immunodeficiency virus (HIV), or any major episode of infection requiring hospitalization or treatment with intravenous (IV) or oral antibiotics within 4 weeks of screening.
History of congenital or acquired immunodeficiency (e.g., common variable immunodeficiency disease).
History of malignancy, except for:
   Treated (i.e., cured) basal cell or squamous cell in situ skin carcinomas
   Treated (i.e., cured) cervical intraepithelial neoplasia or carcinoma in situ of the cervix with no evidence of recurrence within the previous 5 years
Subjects who have received any investigational drug or device within 1 month of screening.
Prior treatment with COMPOUND (I), or participation in a clinical study involving COMPOUND (I).
History of alcohol, drug, or chemical abuse within the 6 months prior to screening.
Known hypersensitivity to oligonucleotides or any ingredient in the IP.

Description of Study Treatments

Description of Investigational Products: COMPOUND (I) will be provided as 40-mg film coated tablets. Placebo will be provided as identically appearing tablets.

Treatment Administration and Schedule: Subjects will receive 1 bottle each visit. Four tablets will be taken by subject once daily in the Induction Phase and 1 tablet will be taken per day in the Extension Phase. Subjects will be instructed to take the IP in the morning, 30 minutes before breakfast, with a glass of water, they will also be instructed to refer to the label for storage instructions. Treatment and administration schedules are described below in Table 2 and Table 3.

TABLE 2
Dosing Schedule for Induction Phase

| | Treatment | | |
|---|---|---|---|
| | Induction | Induction | Induction |
| Treatment Group | Week 0 | Week 4 | Week 8 |
| COMPOUND (1) 160 mg | 160 mg QD | 160 mg QD | 160 mg QD |
| QD for 12 Weeks | | | |
| COMPOUND (1) 160 mg | 160 mg QD | 160 mg QD | Placebo |
| QD for 8 Weeks | | | |
| COMPOUND (1) 160 mg | 160 mg QD | Placebo | Placebo |
| QD for 4 Weeks | | | |

TABLE 3
Dosing Schedule for Extension Phase

| | Treatment | | | | | |
|---|---|---|---|---|---|---|
| | Exten- | Exten- | Exten- | Exten- | Exten- | Exten- |
| | sion | sion | sion | sion | sion | sion |
| Treatment | Week | Week | Week | Week | Week | Week |
| Group | 0 | 4 | 8 | 12 | 16 | 20 |
| COMPOUND (I) | 40 mg | No IP | 40 mg | No IP | 40 mg | No IP |
| 40 mg QD | QD | | QD | | QD | |

Method of Treatment Assignment

Approximately 48 subjects will be randomized in a 1:1:1 ratio to receive 1 of 3 treatment regimens in a 12-week Induction Phase:
COMPOUND (I) 160 mg QD for 12 weeks
COMPOUND (I) 160 mg QD for 8 weeks followed by 4 weeks of placebo
COMPOUND (I) 160 mg QD for 4 weeks followed by 8 weeks of placebo

Treatment assignment at baseline will be stratified via an IVRS/IWRS based on disease location (disease restricted to the terminal ileum and/or up to the mid transverse colon only, or disease involving at least 1 ulcerated segment distal to mid transverse colon). The number of subjects with disease involving distal to mid transverse colon is targeted to comprise approximately 50% of the study population.

Eligible subjects will enter the Induction Phase at the Baseline Visit (Week 0/Induction Visit 1). Subjects will be assigned randomly to receive IP as described above.

At Induction Week 12, subjects (responders) who achieve clinical remission, defined as a CDAI score <150, or clinical response, defined as a decrease from baseline of ≥100 points in CDAI score, at any of the following Induction Visits (Weeks 4, 8 and/or Week 12) will enter the Observation Phase. The Observation Phase will have a duration of up to 52 weeks. Subjects who are unable to achieve clinical remission or clinical response (nonresponders) at the following Induction Visits (Weeks 4, 8 and Week 12), will be discontinued from the study.

Subjects who enter the Observation Phase will be evaluated by CDAI score every 4 weeks. Subjects will not receive IP during the Observation Phase. Subjects who experience a partial loss of response or are unable to taper corticosteroids during the Observation Phase will enter the Extension Phase. Partial loss of response is defined as 2 consecutive visits with both a CDAI score ≥150 and an increase of CDAI score ≥50 points from the CDAI score at the visit when the subject was first a responder during the Induction Phase. Partial loss of response must be confirmed 2 to 4 weeks post initial identification of partial loss of response. Subjects who do not experience a partial loss of response until Observation Week 52 will have an end-of-study visit.

Subjects who enter the Extension Phase will receive COMPOUND (I) 40 mg QD on a 4-week, alternating dosing schedule (4 weeks of treatment with COMPOUND (I), followed by 4 weeks without COMPOUND (I) treatment) for 24 weeks.

Permitted Concomitant Medications and Procedures

The following concomitant medications are permitted during the study:
Oral aminosalicylates (sulfasalazine [SSZ] or 5-ASA compounds) are allowed during the study, provided that treatment was initiated at least 6 weeks prior to the Baseline Visit, and has been given at a stable dose for at least 2 weeks prior to the Baseline Visit. The dose of oral aminosalicylates must remain stable through the duration of the study or early termination from the study. If oral aminosalicylates have been recently discontinued, treatment must have been stopped at least 2 weeks prior to the Baseline Visit.
Oral corticosteroids are allowed during the Induction Phase, provided that the dose (prednisone ≤20 mg/day or equivalent, budesonide ≤9 mg/day) has been stable for 4 weeks prior to the Baseline Visit. If oral corticosteroids were recently discontinued, discontinuation must have been completed at least 4 weeks prior to screening. Corticosteroid doses should remain stable until the subject is eligible to start corticosteroids tapering.
Immunosuppressants, such as AZA, 6-MP or MTX are allowed during the study, provided that treatment was initiated ≥12 weeks prior to the Baseline Visit. The dose of immunosuppressants must be at a stable dose for ≥8 weeks prior to the Baseline Visit and must remain stable through the duration of the study or early termination from the study. Subjects who discontinued immunosuppressants should have stopped them at least 8 weeks prior to the Baseline Visit.
Acetaminophen and low-dose aspirin for cardiovascular prophylaxis are allowed. The dose of concomitant medications noted above may not be increased above the baseline dose during the study. No new CD therapy can be prescribed once the subject has been randomized to the study.

Subjects who enter the Observation Phase and were receiving corticosteroids at baseline will start tapering corticosteroids at the end of the Induction Phase (Induction Week 12). Subjects who are unable to taper corticosteroids during the Observation Phase may start tapering during the Extension Phase at the Investigator's discretion. The tapering schedule is as follows:
For prednisone doses >10 mg (or equivalent) daily dose, each week the daily dose is to be tapered by 5 mg until a dose of 10 mg/day is reached, after that each week the daily dose is to be tapered by 2.5 mg until discontinuation.
For prednisone doses ≤10 mg (or equivalent), each week the daily dose is to be tapered by 2.5 mg until discontinuation.
Subjects receiving budesonide should have their daily dose tapered by 3 mg every weeks.

Prohibited Concomitant Medications and Procedures

The following concomitant medications are prohibited:
Uses of any biologic agents, including TNF blockers through the duration of the study.
Use of mycophenolic acid, tacrolimus, sirolimus, cyclosporine, thalidomide or apheresis (e.g., Adacolumn) through the duration of the study.
Use of topical treatment with 5-ASA or corticosteroid enemas or suppositories within 2 weeks of the Baseline Visit and through the duration of the study.
Use of IV corticosteroids within 2 weeks of the Baseline Visit and through the duration of the study.
Administration of TPN within 4 weeks of screening and through the duration of the study.
Chronic use of nonsteroidal anti-inflammatory drugs (NSAIDs).
Use of antibiotic therapy for the treatment of CD within 3 weeks of screening and through the duration of the study.
Use of cholestyramine within 3 weeks of screening and through the duration of the study.

Required Concomitant Medications and Procedures

There are no required concomitant medications.

Required procedures include ileocolonoscopy and intestinal mucosal biopsy.

Adverse Events

Monitoring, Recording and Reporting of Adverse Events:

An adverse event (AE) is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values, regardless of etiology. Any worsening (i.e., any clinically significant adverse change in the frequency or intensity of a pre-existing condition) should be considered an AE.

Abuse, withdrawal, sensitivity or toxicity to an investigational product should be reported as an AE.

All subjects will be monitored for AEs during the study. Assessments may include monitoring of any or all of the following parameters: the subject's clinical symptoms, laboratory, pathological, radiological or surgical findings, physical examination findings, or findings from other tests and/or procedures.

Evaluation of Adverse Events:

A qualified Investigator will evaluate all adverse events as to:

Seriousness:

A serious adverse event (SAE) is any AE occurring at any dose that:
Results in death;
Is life-threatening (i.e., in the opinion of the Investigator, the subject is at immediate risk of death from the AE);
Requires inpatient hospitalization or prolongation of existing hospitalization (hospitalization is defined as an inpatient admission, regardless of length of stay);
Results in persistent or significant disability/incapacity (a substantial disruption of the subject's ability to conduct normal life functions);
Results in congenital anomaly/birth defect;
Constitutes an important medical event.

Important medical events are defined as those occurrences that may not be immediately life threatening or result in death, hospitalization, or disability, but may jeopardize the subject or require medical or surgical intervention to prevent one of the other outcomes listed above. Medical and scientific judgment should be exercised in deciding whether such an AE should be considered serious.

Events not considered to be SAEs are hospitalizations for:
A standard procedure for protocol therapy administration. However, hospitalization or prolonged hospitalization for a complication of therapy administration will be reported as an SAE.
Routine treatment or monitoring of the studied indication not associated with any deterioration in condition.
The administration of blood or platelet transfusion as routine treatment of studied indication. However, hospitalization or prolonged hospitalization for a complication of such transfusion remains a reportable SAE.
A procedure for protocol/disease-related investigations (e.g., surgery, scans, endoscopy, sampling for laboratory tests, bone marrow sampling). However, hospitalization or prolonged hospitalization for a complication of such procedures remains a reportable SAE.
Hospitalization or prolongation of hospitalization for technical, practical, or social reasons, in absence of an AE.

### Example 2: A Randomized, Double-Blind, Placebo-Controlled, Multicenter Study to Investigate the Efficacy and Safety of COMPOUND (I) for the Treatment of Subjects with Active Crohn's Disease

Study Objectives

The primary objective of the study is to evaluate the efficacy of COMPOUND (I) compared with placebo on clinical activity, as measured by the CDAI in subjects with active CD.

The secondary objectives of the study are:
To evaluate the efficacy of COMPOUND (I) compared with placebo on endoscopic outcomes, as measured by the SES-CD in subjects with active CD;
To evaluate the efficacy of COMPOUND (I) compared with placebo on corticosteroid-free clinical remission in subjects with active CD;
To evaluate the long-term efficacy of COMPOUND (I) compared with placebo on clinical activity and endoscopic outcomes in subjects with active CD;
To evaluate the safety and tolerability of COMPOUND (I) in subjects with active CD.

The exploratory objectives are:
To evaluate additional measures of short-term and long-term efficacy of COMPOUND (I) compared with placebo on clinical activity and endoscopic outcomes in subjects with active CD;
To evaluate the change in biomarkers such as hsCRP and FCP in response to COMPOUND (I), compared with placebo, in subjects with active CD;
To evaluate the change in quality of life and health economic outcomes in response to COMPOUND (I), compared with placebo, in subjects with active CD;
To evaluate the systemic exposure of COMPOUND (I) in subjects with active CD.

Drug Identification

Drug tested is COMPOUND (I) as in Example 1.

Study Design

A schematic diagram illustrating the study design is shown in FIG. 3.

This is a randomized, double-blind, placebo-controlled, multicenter study to evaluate the efficacy and safety of 3 treatment regimens of oral COMPOUND (I) versus placebo in subjects with active CD (defined by a CDAI score ≥220 and <=450 and a total SES-CD >6 at screening, or the ileum segmental SES-CD >4 at screening). Approximately 1064 subjects will be randomized in a 1:1:1:1 ratio (266 subjects per COMPOUND (I) arm [total 798] and 266 subjects in the placebo arm) to receive 1 of 3 double-blind, oral COMPOUND (I) treatment regimens, or identically appearing placebo once daily (QD) for 52 weeks.

Treatment assignment at baseline (Week 0/Visit 2) will be stratified via an Interactive Web Response System (IWRS) based on concomitant use of corticosteroids (yes/no); concomitant use of immunosuppressants (e.g., azathioprine [AZA], 6-mercaptopurine [6-MP], or methotrexate [MIX]) (yes/no), and previous exposure to biologics (e.g., infliximab, adalimumab, certolizumab or vedolizumab) (yes/no). The total number of subjects with previous exposure to biologics is targeted to comprise approximately 35% of the study population.

Subjects will receive double-blind, oral COMPOUND (I) or identically appearing placebo (QD) as follows (see Table 5):
COMPOUND (I) 160 mg QD for 12 weeks; followed by placebo QD for 4 weeks; followed by alternating COMPOUND (I) 160 mg QD for 4 weeks and placebo QD for 4 weeks, until the Week 52 Visit;
COMPOUND (I) 160 mg QD for 12 weeks; followed by placebo QD for 4 weeks; followed by alternating COMPOUND (I) 40 mg QD for 4 weeks and placebo QD for 4 weeks, until the Week 52 Visit;
COMPOUND (I) 160 mg QD for 12 weeks; followed by continuous COMPOUND (I) 40 mg QD, until the Week 52 Visit;
Placebo QD until the Week 52 Visit.

TABLE 5
IP Dispensing Schedule: Double-blind Treatment Period

| Week 0 | Week 12 | Week 16 | Week 20 | Week 24 | Week 28 | Week 32 | Week 36 | Week 40 | Week 44 | Week 48 |
|---|---|---|---|---|---|---|---|---|---|---|
| through | through | through | through | through | through | through | through | through | through | through |
| Week 11 | Week 15 | Week 19 | Week 23 | Week 27 | Week 31 | Week 35 | Week 39 | Week 43 | Week 47 | Week 51 |
| COMP(I) | Placebo | COMP(I) | Placebo | COMP(I) | Placebo | COMP(I) | Placebo | COMP(I) | Placebo | COMP(I) |
| 160 mg | QD | 160 mg QD | QD | 160 mg QD | QD | 160 mg QD | QD | 160 mg QD | QD | 160 mg QD |
| QD | Placebo | COMP(I) | Placebo | COMP(I) | Placebo | COMP(I) | Placebo | COMP(I) | Placebo | COMP(I) |
| | QD | 40 mg QD | QD | 40 mg QD | QD | 40 mg QD | QD | 40 mg QD | QD | 40 mg QD |
| | COMP(I) | COMP(I) | COMP(I) | COMP(I) | COMP(I) | COMP(I) | COMP(I) | COMP(I) | COMP(I) | COMP(I) |
| | 40 mg QD | 40 mg QD | 40 mg QD | 40 mg QD | 40 mg QD | 40 mg QD | 40 mg QD | 40 mg QD | 40 mg QD | 40 mg QD |
| Placebo | Placebo | Placebo | Placebo | Placebo | Placebo | Placebo | Placebo | Placebo | Placebo | Placebo |
| QD | QD | QD | QD | QD | QD | QD | QD | QD | QD | QD |

COMP(I) = COMPOUND (I)

After the Week 12 Visit and thereafter, until the Week 52 Visit, subjects who meet the criteria for “early escape,” will be eligible to enter the Long-term Active-treatment Study (Example 3), or may discontinue from the study. The criteria for “early escape” are defined as a CDAI ≥180 and failure to achieve or maintain a reduction of at least 70 points in the CDAI score, as compared to baseline for 2 consecutive study visits, at least 14 days apart.

Subjects who complete the study described in this Example at the Week 52 Visit will have the option to enter the Example 3 Long-term Active-treatment Study.

Study Endpoints

The primary endpoint of the study is the proportion of subjects achieving clinical remission defined as a CDAI score <150 at Week 4.

The secondary endpoints of the study are:
The proportion of subjects with mucosal healing defined as SES-CD ≤2 at Week 12 and Week 52;
The proportion of subjects with a reduction of at least 50% from baseline in SES-CD at Week 12 and Week 52;
The proportion of subjects achieving clinical remission defined as a CDAI score <150 at Week 12 and Week 52;
The proportion of subjects who achieve corticosteroid-free clinical remission at Week 52 among subjects receiving oral corticosteroids at baseline;
The proportion of subjects with an average daily liquid or soft stool frequency ≤3 and abdominal pain score ≤1 at Week 4, Week 12 and Week 52;
The proportion of subjects with an average daily liquid or soft stool frequency ≤1.5 and abdominal pain score ≤1 point at Week 4, Week 12, and Week 52;
Type, frequency, severity, seriousness, and relationship of AEs to IP;
Number of subjects who discontinue IP due to any AE;
Clinically significant changes in vital signs, ECG and/or laboratory findings.

The Exploratory endpoints of this study include exploratory efficacy endpoints, exploratory pharmacodynamic (PD)/biomarker endpoints, exploratory pharmacokinetics (PK) endpoints, exploratory quality of life endpoints, and exploratory health economics endpoints.

The exploratory efficacy endpoints are:
The proportion of subjects with a reduction of at least 50% in the SES-CD compared with baseline at both Week 12 and Week 52;
The proportion of subjects with mucosal healing defined as SES-CD ≤2 at Week 12 and Week 52;
The proportion of subjects who are in clinical remission, defined as a CDAI score <150 at each time point through Week 52;
The proportion of subjects who have a clinical response defined as a decrease from baseline in CDAI ≥100 points at each time point through Week 52;
The proportion of subjects who have corticosteroid-free clinical remission for at least 12 consecutive weeks among subjects receiving oral corticosteroids at baseline at each time point starting at Week 24;
The proportion of subjects who have corticosteroid-free clinical remission for at least 26 consecutive weeks, among subjects receiving oral corticosteroids at baseline at each time point starting at Week 40;
The change from baseline in the average daily abdominal pain score at each time point through Week 52;
The change from baseline in the average daily liquid or soft stool frequency at each time point through Week 52;
The proportion of subjects with a patient reported outcome (PRO-2) score ≤8 at each time point through Week 52;
The proportion of subjects with a reduction of <8 from baseline in PRO-2 at each time point through Week 52;
The proportion of subjects with an average daily liquid or soft stool frequency ≤3 and abdominal pain score ≤1 at each time point through Week 52;
The proportion of subjects with an average daily liquid or soft stool frequency ≤1.5 and abdominal pain score ≤I at each time point through Week 52;
The change from baseline in the CDAI score at each time point through Week 52;
The change from baseline in the SES-CD at each time point through Week 52;
The change from baseline in the PRO-2 score at each time point through Week 52;
The change from baseline in the Harvey-Bradshaw Index (HBI) score at each time point through Week 52.

The exploratory PD/biomarker endpoints are:
The change from baseline in hsCRP at each time point through Week 52;
The change from baseline in the FCP at each time point through Week 52.

The exploratory PK endpoint is the plasma concentration of COMPOUND (I) at Week 4 and Week 8.

The exploratory quality of life endpoints are:
The change in Short Form 36 Item Health Survey, version 2 (SF-36v2) score compared with baseline at each time point through Week 52;
The change in the Inflammatory Bowel Disease Questionnaire (IBDQ) compared with baseline at each time point through Week 52;
The change in the Work Productivity and Activity Impairment Questionnaire-Crohn's Disease (WPAI-CD) compared with baseline at each time point through Week 52;
The change in the European Quality of Life-5 Dimensions Questionnaire (EQ-5D) compared with baseline at each time point through Week 52.

The exploratory health economics endpoint is the change in the Healthcare Resource Utilization (HRU) compared with baseline at each time point through Week 52.

Study Duration

Subjects will participate for a maximum of 60 weeks in this study: up to 4 weeks in the Screening Period; 52 weeks in the Double-blind Treatment Period; and 4 weeks in the Follow-up Period.

End of Study

The End of Study is defined as either the date of the last visit of the last subject to complete the post-treatment follow-up, or the date of receipt of the last data point from the last subject that is required for primary, secondary and/or exploratory analysis, as prespecified in the protocol, whichever is the later date.

Study Population

Number of Subjects:

Approximately 1064 subjects with active CD will be enrolled in this study. The total number of subjects with previous exposure to biologics is targeted to comprise approximately 35% of the study population.

Inclusion Criteria:

Subjects must satisfy the following criteria to be enrolled in the study:
Subject is a male or female ≥18 years at the time of signing the informed consent form (ICF);
Subject must understand and voluntarily sign an ICF prior to conducting any study-related assessments/procedures;
Subject is willing and able to adhere to the study visit schedule and other protocol requirements;
Subject must have a diagnosis of CD with a duration of at least 3 months prior to the Screening Visit;
Subject must have a diagnosis of ileitis, ileocolitis or colitis, as determined by endoscopic, radiographic or any other imaging modality (e.g., magnetic resonance imaging [MRI], computed tomography [CT] scan);
Subject must have active CD disease, defined as a CDAI score ≥220 and <450 at screening;
Subject must have 7-day average stool frequency >3.5 or abdominal pain >1.5 at screening.
Subject must have a total SES-CD >6 at screening, or the ileum segmental SES-CD >4 at screening;
Subjects must have failed or experienced intolerance to at least one of the following: aminosalicylates; budesonide; systemic corticosteroids; immunosuppressants (e.g., AZA, 6-MP, or MTX); or biologics for the treatment of CD;
Subjects who have at increased risk of colorectal cancer (defined as having an 8-year history of pan-colitis or 12-year history of left-sided colitis) should have undergone a colonoscopy with pan-colonic surveillance biopsies, within 2 years of the Screening Visit. The biopsies must be negative for dysplasia;
Subject must meet the following laboratory criteria
   White blood cell count ≥3000/mm³ (≥3.0×10⁹/L)
   Platelet count ≥100,000/mm³ (≥100×10⁹/L)
   Serum creatinine <1.5 mg/dL (<132.6 μmol/L)
   Aspartate aminotransferase (AST)/serum glutamic oxaloacetic transaminase (SGOT), alanine aminotransferase (ALT)/serum glutamic pyruvic transaminase (SGPT)<2.5× upper limit of normal (ULN)
   Total bilirubin <2 mg/dL (34 μmol/L), unless the subject has a confirmed diagnosis of Gilbert's Disease
   Hemoglobin >8 g/dL (≥4.97 mmol/L)
   Activated partial thromboplastin time (APTT)<1.5×ULN
Females of childbearing potential (FCBP) must have a negative pregnancy test at the Screening and Baseline Visits.
Male subjects (including those who have had a vasectomy) when engaging in sexual activity with females who are able to become pregnant must use barrier contraception while on IP and for at least 28 days after the last dose.

Exclusion Criteria:

The presence of any of the following will exclude a subject from enrollment:
Subject has a diagnosis of ulcerative colitis (UC), indeterminate colitis, ischemic colitis, microscopic colitis, radiation colitis or diverticular disease-associated colitis;
Subject has local manifestations of CD such as strictures, abscesses, short bowel syndrome; or other disease complications for which surgery might be indicated or could confound the evaluation of efficacy;
Subject had any intestinal resection within 6 months or any intra-abdominal surgery within 3 months prior to the Screening Visit;
Subject has an ileostomy or a colostomy;
Subject had prior treatment with mycophenolic acid, tacrolimus, sirolimus, cyclosporine, thalidomide or apheresis (e.g., Adacolumn®) within 8 weeks prior to the Screening Visit;
Use of intravenous (IV) corticosteroids within 2 weeks prior to the Screening Visit;
Use of topical treatment such as 5-aminosalicylic acid (5-ASA) or corticosteroid enemas or suppositories within 2 weeks prior to Screening Visit;
Subject has changed or discontinued the allowed dose of oral aminosalicylates within 2 weeks prior to the Screening Visit;
Use of cholestyramine within 3 weeks prior to Screening Visit;
Subject has changed or discontinued the allowed dose of oral corticosteroids (prednisone <20 mg/day or equivalent, budesonide ≤9 mg/day) within 3 weeks prior to the Screening Visit;
Subject has initiated immunosuppressants (e.g., AZA, 6-MP, or MTX) within 12 weeks prior to the Screening Visit and has changed or discontinued the allowed dose of immunosuppressants within 8 weeks prior to the Screening Visit;
Subject has received topical treatments, such as, 5-aminosalicylic acid (5-ASA) or corticosteroid enemas or suppositories within 2 weeks prior to the Screening Visit;
Subject received cholestyramine within 3 weeks prior to the Screening Visit;
Subject has changed or discontinued antibiotics used for the treatment of CD (e.g., ciprofloxacin, metronidazole) within 2 weeks prior to the Screening Visit;
Subject had prior treatment with more than 3 biologics for the treatment of CD;
Subject had treatment with a biologic within 8 weeks prior to the Screening Visit;
Subject had prior treatment with natalizumab;
Subject has received total parenteral nutrition within 4 weeks prior to the Screening Visit;
Subject has evidence of enteric infection or C. difficile toxin at the Screening Visit;
Subject has a history of any clinically significant neurological, renal, hepatic, gastrointestinal, pulmonary, metabolic, cardiovascular, psychiatric, endocrine, hematological disorder or disease, or any other medical condition that, in the investigator's opinion, would prevent the subject from participating in the study;
Subject has any condition, including the presence of laboratory abnormalities, which places the subject at unacceptable risk if he/she was to participate in the study or confounds the ability to interpret data from the study;
Subject is pregnant or breastfeeding;
Subject has a history of any of the following cardiac conditions within 6 months prior to the Screening Visit and at any time during the Screening Period, up through the first dose of IP: myocardial infarction, acute coronary syndrome, unstable angina, new onset atrial fibrillation, new onset atrial flutter, second- or third-degree atrioventricular block, ventricular fibrillation, ventricular tachycardia, heart failure, cardiac surgery, interventional cardiac catheterization (with or without a stent placement), interventional electrophysiology procedure, or presence of implanted defibrillator;
Subject has a known active current or history of clinically significant recurrent bacterial, viral, fungal, mycobacterial or other infections (including but not limited to tuberculosis and atypical mycobacterial disease and herpes zoster), human immunodeficiency virus (HIV), or any major episode of infection requiring hospitalization or treatment with IV or oral antibiotics within 4 weeks prior to the Screening Visit and at any time during the Screening Period, up through the first dose of IP;
Subject has a history of congenital or acquired immunodeficiency (e.g., common variable immunodeficiency disease);
Subject has a history of colorectal cancer or colorectal dysplasia (with the exception of adenomatous colonic polyps that have been completely resected);
Subject has a history of malignancy, except for:
   Treated (i.e., cured) basal cell or squamous cell in situ skin carcinomas;
   Treated (i.e., cured) cervical intraepithelial neoplasia or carcinoma in situ of the cervix with no evidence of recurrence within the previous 5 years prior to the Screening Visit;
Subject has received any investigational drug or device within 1 month prior to the Screening Visit;
Subject has a history of alcohol, drug, or chemical abuse within the 6 months prior to the Screening Visit;
Subject has a known hypersensitivity to oligonucleotides or any ingredient in the IP;
Subject has received prior treatment with COMPOUND (I), or participated in a clinical study involving COMPOUND (I).
Acetaminophen and low-dose aspirin for cardiovascular prophylaxis are allowed.

Corticosteroid Tapering Procedure

Subjects are eligible to start corticosteroid tapering beginning at the Week 12 Visit, at the discretion of the investigator, according to the following schedule:
For prednisone doses >10 mg (or equivalent), each week the daily dose is to be tapered by 5 mg until a dose of 10 mg/day is reached, after that each week the daily dose is to be tapered by 2.5 mg until discontinuation.
For prednisone doses <10 mg (or equivalent), each week the daily dose is to be tapered by 2.5 mg until discontinuation.
Subjects receiving budesonide should have their daily dose tapered by 3 mg every 3 weeks.

Description of Study Treatments

Description of Investigational Product(s):

COMPOUND (I) will be provided as 40-mg film coated tablets. Placebo will be provided as identically appearing tablets.

Treatment Administration and Schedule:

Subjects will receive COMPOUND (I) (160 mg QD) or placebo during the first 12 weeks of the study, followed by one of 3 dose regimens of COMPOUND (I) (40 mg QD or 160 mg QD) or placebo QD, until the end of the study (Week 52 Visit). All subjects will receive 4 tablets daily during the Double-blind Treatment Period. Matched placebo tablets will also be provided. Subjects will be instructed to take the IP in the morning, 30 minutes before breakfast, with a glass of water. Treatment and administration schedule are described in Table 5.

Subjects will receive double-blind, oral COMPOUND (I) or identically appearing placebo daily (QD) as follows (Table 4):
COMPOUND (I) 160 mg QD for 12 weeks; followed by placebo QD for 4 weeks; followed by alternating COMPOUND (I) 160 mg QD for 4 weeks and placebo QD for 4 weeks, until the Week 52 Visit;
COMPOUND (I) 160 mg QD for 12 weeks; followed by placebo QD for 4 weeks; followed by alternating COMPOUND (I) 40 mg QD for 4 weeks and placebo QD for 4 weeks, until the Week 52 Visit;
COMPOUND (I) 160 mg QD for 12 weeks; followed by continuous COMPOUND (I) 40 mg QD, until the Week 52 Visit;
Placebo QD until the Week 52 Visit.

Concomitant Medications and Procedures

Permitted concomitant medications during the study include:
Oral aminosalicylates (SSZ or 5-ASA compounds), provided that treatment has been given at a stable dose for at least 2 weeks prior to the Screening Visit. The dose of oral aminosalicylates must remain stable through the duration of the study or early termination from the study. If oral aminosalicylates have been recently discontinued, treatment must have been stopped at least 2 weeks prior to the Screening Visit.
Oral corticosteroids, provided that the dose (prednisone ≤20 mg/day or equivalent, budesonide ≤9 mg/day) has been stable for 3 weeks prior to the Screening Visit, and the dose must remain stable until the subject is eligible to start corticosteroids tapering. If oral corticosteroids have been recently discontinued, discontinuation must have been completed at least 3 weeks prior to the Screening Visit.
Immunosuppressants, such as AZA, 6-MP, or MTX, provided that treatment was initiated >12 weeks prior to the Screening Visit, must be at a stable dose for ≥8 weeks prior to the Screening Visit and remain stable for the duration of the study.
Acetaminophen and low-dose aspirin for cardiovascular prophylaxis are allowed.

The following concomitant medications are prohibited during the Double-blind Treatment Period of the study, Baseline Visit (Week 0/Visit 2) through the 52 Visit, or the ET Visit for subjects who discontinue prematurely during the study:
Uses of any biologics are prohibited during the study and must be discontinued at least 8 weeks prior to the Screening Visit.
Use of cholestyramine is prohibited during the study and must be discontinued at least 3 weeks prior to the Screening Visit.
Use of antibiotics for the treatment of CD is prohibited during the study and must be discontinued at least 3 weeks prior to the Screening Visit.
Use of mycophenolic acid, tacrolimus, sirolimus, cyclosporine, thalidomide or apheresis (e.g., Adacolumn®) is prohibited during the study and must be discontinued at least 8 weeks prior to the Screening Visit.
Use of topical treatments with 5-ASA or corticosteroid enemas or suppositories is prohibited during the study and must be discontinued at least 2 weeks prior to the Screening Visit.
Use of IV corticosteroids is prohibited during the study and must be discontinued at least 2 weeks prior to the Screening Visit.
Administration of total parenteral nutrition (TPN) is prohibited and must be discontinued at least 4 weeks prior to the Screening Visit.
Chronic use of nonsteroidal anti-inflammatory drugs (NSAIDs) is prohibited.

There are no required concomitant medications. Required procedures include ileocolonoscopy.

Adverse Events

Monitoring, Recording and Reporting of Adverse Events:

An adverse event (AE) is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values, regardless of etiology. Any worsening (i.e., any clinically significant adverse change in the frequency or intensity of a pre-existing condition) should be considered an AE.

Abuse, withdrawal, sensitivity or toxicity to an investigational product should be reported as an AE.

All subjects will be monitored for AEs during the study. Assessments may include monitoring of any or all of the following parameters: the subject's clinical symptoms, laboratory, pathological, radiological or surgical findings, physical examination findings, or findings from other tests and/or procedures.

Evaluation of Adverse Events:

A qualified Investigator will evaluate all adverse events as to:
Seriousness: A serious adverse event (SAE) is any AE occurring at any dose that:
   Results in death;
   Is life-threatening (i.e., in the opinion of the Investigator, the subject is at immediate risk of death from the AE);
   Requires inpatient hospitalization or prolongation of existing hospitalization (hospitalization is defined as an inpatient admission, regardless of length of stay);
   Results in persistent or significant disability/incapacity (a substantial disruption of the subject's ability to conduct normal life functions);
   Results in congenital anomaly/birth defect;
   Constitutes an important medical event.

Events not considered to be SAEs are hospitalizations for:
A standard procedure for protocol therapy administration. However, hospitalization or prolonged hospitalization for a complication of therapy administration will be reported as an SAE.
Routine treatment or monitoring of the studied indication not associated with any deterioration in condition.
A procedure for protocol/disease-related investigations (e.g., surgery, scans, endoscopy, sampling for laboratory tests, bone marrow sampling). However, hospitalization or prolonged hospitalization for a complication of such procedures remains a reportable SAE.
Hospitalization or prolongation of hospitalization for technical, practical, or social reasons, in absence of an AE.
A procedure that is planned (i.e., planned prior to start of treatment on study); must be documented in the source document and the eCRF. Hospitalization or prolonged hospitalization for a complication remains a reportable SAE.
An elective treatment of or an elective procedure for a pre-existing condition, unrelated to the studied indication that has not worsened from baseline.
Emergency outpatient treatment or observation that does not result in admission, unless fulfilling other seriousness criteria above.

### Example 3: A Long-Term Active Treatment Extension Study of Compound (I) in Subjects with Crohn's Disease

Study Objectives

The primary objective of the study is to evaluate the long-term safety of oral COMPOUND (I) in subjects with CD.

The exploratory objectives of the study are:
To explore the long-term clinical efficacy of COMPOUND (I) over time in subjects with CD who participated in a previous COMPOUND (I) study (see, e.g., Example 2);
To evaluate the long-term benefit of COMPOUND (I) on health-related quality of life (HRQOL) outcome measures in subjects with CD who participated in a previous COMPOUND (I) study (see, e.g., Example 2);
To evaluate the long-term changes in biomarkers such as hsCRP and FCP in response to COMPOUND (I) in subjects with CD who participated in a previous COMPOUND (I) study (see, e.g., Example 2).

Drug Identification

Drug tested is COMPOUND (I), as described in Example 1.

Study Design

A schematic diagram illustrating the study design is shown in FIG. 4.

This is a double-blinded, long-term active treatment study to evaluate the long-term safety, tolerability and exploratory efficacy of COMPOUND (I) in subjects with CD who previously participated in a COMPOUND (I) study (see, e.g., Example 2).

Subjects from the Example 2 Study who completed the study at Week 52, or who met the early escape criteria and were discontinued after Week 12 through Week 52, may be eligible to enter this study. Subjects who discontinued the Example 2 Study are not eligible for this study.

The study will consist of 3 study periods:
Screening Period—up to 4 weeks (i.e., I day to 28 days depending on when long-term active treatment is available for the subject);
Long-term Active Treatment Period—208 Weeks (Week 0 to Week 208);
Follow-up Period—4 weeks (i.e., no IP taken).

Subjects who complete this study through Week 208 will have a 4-week Follow-up Visit. Subjects who prematurely discontinue treatment from this study prior to Week 208 will have an Early Termination (ET) Visit and a 4-week Follow-up Visit. The ET Visit should be scheduled as soon as possible after the last dose of IP. If the ET Visit occurs 28 days after the last dose of IP, then the Follow-up Visit is not required.

Once the subject is eligible and registered, the assigned treatment is based on clinical improvement criteria to determine the subject's course of treatment for the entire 208 weeks in this study.

The clinical improvement criteria are defined as a subject who has a CDAI <180, OR a reduction >70 points in the CDAI score as compared to baseline, in the previously participated COMPOUND (I) study, as compared to baseline for 2 consecutive study visits, at least 14 days apart. The baseline value is the measurement taken at the beginning of the prior COMPOUND (I) study (e.g., Example 2), and is the composite score made up of a 8 factors (Crohn's Disease Activity Index), and then compared to the last measured value (e.g., week 52 for Example 2) of the COMPOUND (I) study.

Previously-Treated COMPOUND (I) Subjects in the Example 2 Study Who Did Meet the Clinical Improvement Criteria at Week 52:

Will continue to receive their same blinded treatment, which will be:
a. PBO QD for 4 weeks/COMPOUND (I) 40 mg QD for 4 weeks, or
b. COMPOUND (I) 40 mg QD, or
c. PBO QD for 4 weeks/COMPOUND (I) 160 mg QD for 4 weeks.

Previously-treated COMPOUND (I) subjects treated in a prior COMPOUND (I) study who meet the clinical improvement criteria at Week 12 will receive blinded treatment with PBO QD for 4 weeks/COMPOUND (I) 160 mg for 4 weeks.

Previously-treated placebo subjects who did meet the clinical improvement criteria at Week 52 in the Example 2 Study or at Week 12 in another prior COMPOUND (I) study will receive blinded treatment with COMPOUND (I) 160 mg QD for 4 weeks/PBO QD for 4 weeks.

Previously-treated COMPOUND (I) or placebo subjects who did not meet the clinical improvement criteria after Week 12 through Week 52 in the Example 2 study, or at Week 12 in another prior COMPOUND (I) study, will receive blinded treatment with:
a. COMPOUND (I) 160 mg QD for 4 weeks/PBO QD for 4 weeks, if the subject previously received COMPOUND (I) treatment, or
b. COMPOUND (I) 160 mg QD for 12 weeks, followed by PBO QD for 4 weeks/COMPOUND (I) 160 mg QD for 4 weeks, if the subject previously received placebo.

Study Endpoints

The primary endpoint of the study is the evaluation of safety and tolerability of COMPOUND (I), assessed by the type, frequency and severity of adverse events, and its relationship to investigational product (IP), discontinuation due to adverse events, and clinically significant changes in electrocardiograms (ECGs), vital signs, and/or laboratory findings.

The Exploratory endpoints of this study include exploratory efficacy endpoints as follows:
The change in the CDAI compared to baseline over time through Week 12;
The change in the HBI compared to baseline over time through Week 208;
The change in the EQ-5D score compared to baseline over time through Week 208;
The change in the EQ-5D score compared to baseline over time through Week 208;
The change in the HRU compared to baseline over time through Week 208;
The change in the hsCRP concentration compared to baseline over time through Week 208;
The change in the FCP concentration compared to baseline over time through Week 52.

Study Duration

Subjects may participate for a maximum of 216 weeks with 3 different study periods: up to 4 weeks in the Screening Period; 208 weeks in the Long-term Active Treatment Period; and 4 weeks in the Follow-up Period.

End of Study

The End of Study is defined as either the date of the last visit of the last subject to complete the post-treatment follow-up, or the date of receipt of the last data point from the last subject that is required for primary and/or exploratory analysis, as prespecified in the protocol, whichever is the later date. There are no secondary objectives or end points in this study.

Study Population

Number of Subjects:

The number of subjects planned to enroll into this long-term active treatment study will be based on the number of eligible subjects who enter from previously conducted COMPOUND (I) studies, such as the Example 2 study, which include subject participation worldwide.

Inclusion Criteria:

Subjects must satisfy the following criteria to be enrolled in the study:
Subject is a male or female >18 years at the time of signing the informed consent form (ICF);
Subject must understand and voluntarily sign an ICF prior to conducting any study-related assessments/procedures;
Subject is willing and able to adhere to the study visit schedule and other protocol requirements;
Subject must have completed through Week 12 from the previous COMPOUND (I) study AND either:
   Completed participation through the last study treatment visit at Week 52 in the Example 2 study, or at Week 12 in another COMPOUND (I) study; OR
   Met the “early escape criteria” and were discontinued after Week 12 in the Example 2 Study.

Exclusion Criteria:

The presence of any of the following will exclude a subject from enrollment:
Subject had experienced a SAE related to the IP while participating in the previous COMPOUND (I) study;
Subject has any continuing serious medical condition, laboratory abnormality, or psychiatric illness that occurred while participating in the previous COMPOUND (I) study;
Subject has or had a flare or worsening of CD that, in the opinion of the Investigator, would not be in the best interest for the subject to participate in this long-term active treatment study;
Subject has initiated biologic agents, such as TNF-α blockers or integrin antagonists;
Subject has been diagnosed with colorectal cancer or colorectal dysplasia while participating in the previous COMPOUND (I) study (with the exception of adenomatous colonic polyps that have been completely resected);
Subject has a newly diagnosed malignancy while participating in the previous COMPOUND (I) study.
Subject is pregnant or breastfeeding;
Subject has been newly diagnosed with substance abuse;
Subject has developed a known hypersensitivity to oligonucleotides, COMPOUND (I) or any ingredient in the IP.

Corticosteroid Tapering Procedure

Subjects are eligible to start corticosteroid tapering beginning at the Week 4 Visit, at the discretion of the investigator, according to the following schedule:
For prednisone doses >10 mg (or equivalent) daily dose, each week the daily dose is to be tapered by 5 mg until a dose of 10 mg/day is reached. Afterwards, each week the daily dose is to be tapered by 2.5 mg until discontinuation.
For prednisone doses <10 mg (or equivalent), each week the daily dose is to be tapered by 2.5 mg until discontinuation.
Subjects receiving budesonide should have their daily dose tapered by 3 mg every 3 weeks.

Description of Study Treatments

Description of Investigational Product(s) [Same as Ex. 2]:

COMPOUND (I) will be provided as 40-mg film coated tablets. Placebo will be provided as identically appearing tablets.

Treatment Administration and Schedule:

Beginning at Week 0 through Week 12, subjects will receive blinded-active COMPOUND (I) (160 mg QD or 40 mg QD) treatment as one of the following numbered treatment groups (see FIG. 4):
1) continuous 160 mg QD for 12 weeks;
2) 160 mg QD for 4 weeks, PBO QD for 4 weeks, 160 mg QD for 4 weeks;
3) PBO QD for 4 weeks, 160 mg QD for 4 weeks, PBO QD for 4 weeks;
4) continuous 40 mg QD for 12 weeks;
5) PBO QD for 4 weeks, 40 mg QD for 4 weeks, PBO QD for 4 weeks.

Beginning at Week 12 through Week 208, subjects from the above-numbered treatment groups will continue to receive blinded-active COMPOUND (I) treatment for 196 weeks as the following:
1) alternating PBO QD for 4 weeks with 160 mg QD for 4 weeks, through Week 208;
2) alternating PBO QD for 4 weeks with 160 mg QD for 4 weeks, through Week 208;
3) alternating 160 mg QD for 4 weeks with PBO QD for 4 weeks, through Week 208;
4) continuous 40 mg QD, through Week 208;
5) alternating 40 mg QD for 4 weeks with PBO QD for 4 weeks, through Week 208.

Subjects will receive 1 blister card when IP is administered. Four tablets are taken once daily during the 208-week Long-term Active Treatment Period. Subjects will be instructed to take the IP in the morning, 30 minutes before breakfast, with a glass of water, and they will also be instructed to refer to the label for storage instructions.

Treatment and administration schedules are described below beginning in Table 8.

TABLE 8
Dosing Schedule for the Treatment Period at Year 1

| | Long-term Active Treatment Period | | | | | | | | | | | | | |
|---|---|---|---|---|---|---|---|---|---|---|---|---|---|---|
| Treatment | (All Treatment is Blinded) | | | | | | | | | | | | | |
| Group from | (Year 1) | | | | | | | | | | | | | |
| Example | Weeks | Weeks | Weeks | Weeks | Weeks | Weeks | Weeks | Weeks | Weeks | Weeks | Weeks | Weeks | Weeks | Weeks |
| 2 Study | 0-3 | 4-7 | 8-11 | 12-15 | 16-19 | 20-23 | 24-27 | 28-31 | 32-35 | 36-39 | 40-43 | 44-47 | 48-51 | 52-55 |
| Placebo | 160 mg | 160 mg | 160 mg | PBO | 160 mg | PBO | 160 mg | PBO | 160 mg | PBO | 160 mg | PBO | 160 mg | PBO |
| (with no | QD | QD | QD | QD | QD | QD | QD | QD | QD | QD | QD | QD | QD | QD |
| improvement) | | | | | | | | | | | | | | |
| COMP(I)40 | 160 mg | PBO | 160 mg | PBO | 160 mg | PBO | 160 mg | PBO | 160 mg | PBO | 160 mg | PBO | 160 mg | PBO |
| mg or 160 mg | QD | QD | QD | QD | QD | QD | QD | QD | QD | QD | QD | QD | QD | QD |
| on/off, or | | | | | | | | | | | | | | |
| 40 mg daily, or | | | | | | | | | | | | | | |
| 160 mg QD | | | | | | | | | | | | | | |
| for 12 weeks | | | | | | | | | | | | | | |
| (with no | | | | | | | | | | | | | | |
| improvement), | | | | | | | | | | | | | | |
| or PBO (with | | | | | | | | | | | | | | |
| improvement) | | | | | | | | | | | | | | |
| COMP(I)160 | PBO | 160 mg | PBO | 160 mg | PBO | 160 mg | PBO | 160 mg | PBO | 160 mg | PBO | 160 mg | PBO | 160 mg |
| mg on/off or | QD | QD | QD | QD | QD | QD | QD | QD | QD | QD | QD | QD | QD | QD |
| 160 mg QD | | | | | | | | | | | | | | |
| for 12 weeks | | | | | | | | | | | | | | |
| (with | | | | | | | | | | | | | | |
| improvement) | | | | | | | | | | | | | | |
| COMP(I) | 40 mg | 40 mg | 40 mg | 40 mg | 40 mg | 40 mg | 40 mg | 40 mg | 40 mg | 40 mg | 40 mg | 40 mg | 40 mg | 40 mg |
| 40 mg daily | QD | QD | QD | QD | QD | QD | QD | QD | QD | QD | QD | QD | QD | QD |
| (with | | | | | | | | | | | | | | |
| improvement) | | | | | | | | | | | | | | |
| COMP(I)40 | PBO | 40 mg | PBO | 40 mg | PBO | 40 mg | PBO | 40 mg | PBO | 40 mg | PBO | 40 mg | PBO | 40 mg |
| mg on/off | QD | QD | QD | QD | QD | QD | QD | QD | QD | QD | QD | QD | QD | QD |
| (with | | | | | | | | | | | | | | |
| improvement) | | | | | | | | | | | | | | |

TABLE 9
Dosing Schedule for the Treatment Period at Year 2

| | Long-term Active Treatment Period | | | | | | | | | | | | |
|---|---|---|---|---|---|---|---|---|---|---|---|---|---|
| Treatment | (All Treatment is Blinded) | | | | | | | | | | | | |
| Group from | (Year 2) | | | | | | | | | | | | |
| Example 2 | Weeks | Weeks | Weeks | Weeks | Weeks | Weeks | Weeks | Weeks | Weeks | Weeks | Weeks | Weeks | Weeks |
| Study | 56-59 | 60-63 | 64-67 | 68-71 | 72-75 | 76-79 | 80-83 | 84-87 | 88-91 | 92-95 | 96-99 | 100-103 | 104-107 |
| Placebo | 160 mg | PBO | 160 mg | PBO | 160 mg | PBO | 160 mg | PBO | 160 mg | PBO | 160 mg | PBO | 160 mg |
| (with no | QD | QD | QD | QD | QD | QD | QD | QD | QD | QD | QD | QD | QD |
| improvement) | | | | | | | | | | | | | |
| COMP(I) 40 | 160 mg | PBO | 160 mg | PBO | 160 mg | PBO | 160 mg | PBO | 160 mg | PBO | 160 mg | PBO | 160 mg |
| mg or 160 mg | QD | QD | QD | QD | QD | QD | QD | QD | QD | QD | QD | QD | QD |
| on/off, or | | | | | | | | | | | | | |
| 40 mg daily, or | | | | | | | | | | | | | |
| 160 mg QD for | | | | | | | | | | | | | |
| 12 weeks (with | | | | | | | | | | | | | |
| no | | | | | | | | | | | | | |
| improvement), | | | | | | | | | | | | | |
| or PBO (with | | | | | | | | | | | | | |
| improvement) | | | | | | | | | | | | | |
| COMP(I) 160 | PBO | 160 mg | PBO | 160 mg | PBO | 160 mg | PBO | 160 mg | PBO | 160 mg | PBO | 160 mg | PBO |
| mg on/off or | QD | QD | QD | QD | QD | QD | QD | QD | QD | QD | QD | QD | QD |
| 160 mg QD for | | | | | | | | | | | | | |
| 12 weeks (with | | | | | | | | | | | | | |
| improvement) | | | | | | | | | | | | | |
| COMP(I) | 40 mg | 40 mg | 40 mg | 40 mg | 40 mg | 40 mg | 40 mg | 40 mg | 40 mg | 40 mg | 40 mg | 40 mg | 40 mg |
| 40 mg daily | QD | QD | QD | QD | QD | QD | QD | QD | QD | QD | QD | QD | QD |
| (with | | | | | | | | | | | | | |
| improvement) | | | | | | | | | | | | | |
| COMP(I) 40 | PBO | 40 mg | PBO | 40 mg | PBO | 40 mg | PBO | 40 mg | PBO | 40 mg | PBO | 40 mg | PBO |
| mg on/off | QD | QD | QD | QD | QD | QD | QD | QD | QD | QD | QD | QD | QD |
| (with | | | | | | | | | | | | | |
| improvement) | | | | | | | | | | | | | |

TABLE 10
Dosing Schedule for the Treatment Period at Year 3

| | Long-term Active Treatment Period | | | | | | | | | | | | |
|---|---|---|---|---|---|---|---|---|---|---|---|---|---|
| Treatment | (All Treatment is Blinded) | | | | | | | | | | | | |
| Group from | (Year 3) | | | | | | | | | | | | |
| Example 2 | Weeks | Weeks | Weeks | Weeks | Weeks | Weeks | Weeks | Weeks | Weeks | Weeks | Weeks | Weeks | Weeks |
| Study | 108-111 | 112-115 | 116-119 | 120-123 | 124-127 | 128-131 | 132-135 | 136-139 | 140-143 | 144-147 | 148-151 | 152-155 | 156-159 |
| Placebo | PBO | 160 mg | PBO | 160 mg | PBO | 160 mg | PBO | 160 mg | PBO | 160 mg | PBO | 160 mg | PBO |
| (with no | QD | QD | QD | QD | QD | QD | QD | QD | QD | QD | QD | QD | QD |
| improvement) | | | | | | | | | | | | | |
| COMP(I) 40 | PBO | 160 mg | PBO | 160 mg | PBO | 160 mg | PBO | 160 mg | PBO | 160 mg | PBO | 160 mg | PBO |
| mg or 160 mg | QD | QD | QD | QD | QD | QD | QD | QD | QD | QD | QD | QD | QD |
| on/off, or | | | | | | | | | | | | | |
| 40 mg daily, or | | | | | | | | | | | | | |
| 160 mg QD for | | | | | | | | | | | | | |
| 12 weeks (with | | | | | | | | | | | | | |
| no | | | | | | | | | | | | | |
| improvement), | | | | | | | | | | | | | |
| or PBO (with | | | | | | | | | | | | | |
| improvement) | | | | | | | | | | | | | |
| COMP(I) 160 | 160 mg | PBO | 160 mg | PBO | 160 mg | PBO | 160 mg | PBO | 160 mg | PBO | 160 mg | PBO | 160 mg |
| mg on/off or | QD | QD | QD | QD | QD | QD | QD | QD | QD | QD | QD | QD | QD |
| 160 mg QD for | | | | | | | | | | | | | |
| 12 weeks (with | | | | | | | | | | | | | |
| improvement) | | | | | | | | | | | | | |
| COMP(I) 40 | 40 mg | 40 mg | 40 mg | 40 mg | 40 mg | 40 mg | 40 mg | 40 mg | 40 mg | 40 mg | 40 mg | 40 mg | 40 mg |
| mg daily (with | QD | QD | QD | QD | QD | QD | QD | QD | QD | QD | QD | QD | QD |
| improvement) | | | | | | | | | | | | | |
| COMP(I) 40 | 40 mg | PBO | 40 mg | PBO | 40 mg | PBO | 40 mg | PBO | 40 mg | PBO | 40 mg | PBO | 40 mg |
| mg on/off | QD | QD | QD | QD | QD | QD | QD | QD | QD | QD | QD | QD | QD |
| (with | | | | | | | | | | | | | |
| improvement) | | | | | | | | | | | | | |

TABLE 11
Dosing Schedule for the Treatment Period at Year 4

| | Long-term Active Treatment Period | | | | | | | | | | | | |
|---|---|---|---|---|---|---|---|---|---|---|---|---|---|
| Treatment | (All Treatment is Blinded) | | | | | | | | | | | | |
| Group from | (Year 4) | | | | | | | | | | | | |
| Example 2 | Weeks | Weeks | Weeks | Weeks | Weeks | Weeks | Weeks | Weeks | Weeks | Weeks | Weeks | Weeks | Weeks |
| Study | 160-133 | 164-167 | 168-171 | 172-175 | 176-179 | 180-183 | 184-187 | 188-191 | 192-195 | 196-199 | 200-203 | 204-207 | 208-211 |
| Placebo | 160 mg | PBO | 160 mg | PBO | 160 mg | PBO | 160 mg | PBO | 160 mg | PBO | 160 mg | PBO | No IP |
| (with no | QD | QD | QD | QD | QD | QD | QD | QD | QD | QD | QD | QD | |
| improvement) | | | | | | | | | | | | | |
| COMP(I) 40 | 160 mg | PBO | 160 mg | PBO | 160 mg | PBO | 160 mg | PBO | 160 mg | PBO | 160 mg | PBO | No IP |
| mg or 160 mg | QD | QD | QD | QD | QD | QD | QD | QD | QD | QD | QD | QD | |
| on/off, or | | | | | | | | | | | | | |
| 40 mg daily, | | | | | | | | | | | | | |
| or 160 mg QD | | | | | | | | | | | | | |
| for 12 weeks | | | | | | | | | | | | | |
| (with no | | | | | | | | | | | | | |
| improvement), | | | | | | | | | | | | | |
| or PBO (with | | | | | | | | | | | | | |
| improvement) | | | | | | | | | | | | | |
| COMP(I) 160 | PBO | 160 mg | PBO | 160 mg | PBO | 160 mg | PBO | 160 mg | PBO | 160 mg | PBO | 160 mg | No IP |
| mg on/off or | QD | QD | QD | QD | QD | QD | QD | QD | QD | QD | QD | QD | |
| 160 mg QD | | | | | | | | | | | | | |
| for 12 weeks | | | | | | | | | | | | | |
| (with | | | | | | | | | | | | | |
| improvement) | | | | | | | | | | | | | |
| COMP(I) 40 | 40 mg | 40 mg | 40 mg | 40 mg | 40 mg | 40 mg | 40 mg | 40 mg | 40 mg | 40 mg | 40 mg | 40 mg | No IP |
| mg daily (with | QD | QD | QD | QD | QD | QD | QD | QD | QD | QD | QD | QD | |
| improvement) | | | | | | | | | | | | | |
| COMP(I) 40 | PBO | 40 mg | PBO | 40 mg | PBO | 40 mg | PBO | 40 mg | PBO | 40 mg | PBO | 40 mg | No IP |
| mg on/off | QD | QD | QD | QD | QD | QD | QD | QD | QD | QD | QD | QD | |
| (with | | | | | | | | | | | | | |
| improvement) | | | | | | | | | | | | | |

The treatment described in each box is the treatment dispensed at a particular week visit. For example, if the subject was previously on placebo (with no improvement) in the previous COMPOUND (I) study, the subject would receive the IP in this study as:
blinded COMPOUND (I) 160 mg QD for a 4-week supply at the Week 0 Visit;
blinded COMPOUND (I) 160 mg QD for a 4-week supply at the Week 4 Visit;
blinded PBO QD for a 4-week supply at Week 52 Visit;
blinded COMPOUND (I) 160 mg QD for a 4-week supply at Week 104 Visit;
blinded PBO QD for a 4-week supply at Week 156 Visit; and
no IP at the Week 208 Visit.

Method of Treatment Assignment:

Eligible subjects will receive COMPOUND (I) treatment for 4-week intervals during this 208-week Long-term Active Treatment Study as either: 1) four 40-mg tablets; 2) one 40-mg tablet and three placebo tablets; or 3) four placebo tablets.

Concomitant Medications and Procedures

Permitted concomitant medications during the study include:
Oral aminosalicylates (such as sulfasalazine [SSZ] or 5-aminosalicylic acid [5-ASA]compounds); or immunosuppressants (such as AZA, 6-MP, or MTX) may be initiated or changed during screening or continued from a previous COMPOUND (I) study, provided that the dose remains stable through the first 12 weeks of this study from Week 0 to Week 12. After Week 12, subjects may taper doses or completely discontinue any of these background CD medications or may increase doses or add any new CD medications as clinically indicated, except for biologics, at the discretion of the Investigator.
Oral corticosteroids (with no dose restriction) may be initiated or changed during screening, or continued from a previous COMPOUND (I) study, provided that the dose remains stable through the first 4 weeks of this study from Week 0 to Week 4. After Week 4, subjects may taper corticosteroids doses as clinically indicated, at the discretion of the Investigator.
The dose of the CD concomitant medications noted above should not be changed from the stable dose beginning at Enrollment Visit 2 (Week 0) through Week 12. However, corticosteroids may be changed beginning at Week 4. No new CD therapy can be prescribed once the subject has been enrolled at Visit 2/Week 0 into the study, until Week 12.

The following concomitant medications are prohibited during the Screening Period and from the Enrollment Visit (i.e., Week 0/Visit 2) through the last study treatment visit (i.e., Week 208/Visit 54), or the ET Visit for subjects who discontinue prematurely during the study:
Use of any biologic agents, including TNF-α blockers or integrin antagonists. If biologic agents are initiated, the subject must be discontinued from the study.

There are no required concomitant medications and procedures in this study.

Adverse Events

Monitoring, Recording and Reporting of Adverse Events:

An adverse event (AE) is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values, regardless of etiology. Any worsening (i.e., any clinically significant adverse change in the frequency or intensity of a pre-existing condition) should be considered an AE.

Abuse, withdrawal, sensitivity or toxicity to an investigational product should be reported as an AE.

All subjects will be monitored for AEs during the study. Assessments may include monitoring of any or all of the following parameters: the subject's clinical symptoms, laboratory, pathological, radiological or surgical findings, physical examination findings, or findings from other tests and/or procedures.

Evaluation of Adverse Events:

A qualified Investigator will evaluate all adverse events as to:
Seriousness: A serious adverse event (SAE) is any AE occurring at any dose that:
   Results in death;
   Is life-threatening (i.e., in the opinion of the Investigator, the subject is at immediate risk of death from the AE);
   Requires inpatient hospitalization or prolongation of existing hospitalization (hospitalization is defined as an inpatient admission, regardless of length of stay);
   Results in persistent or significant disability/incapacity (a substantial disruption of the subject's ability to conduct normal life functions);
   Results in congenital anomaly/birth defect;
   Constitutes an important medical event.

Important medical events are defined as those occurrences that may not be immediately life threatening or result in death, hospitalization, or disability, but may jeopardize the subject or require medical or surgical intervention to prevent one of the other outcomes listed above. Medical and scientific judgment should be exercised in deciding whether such an AE should be considered serious.

Events not considered to be SAEs are hospitalizations for:
A standard procedure for protocol therapy administration. However, hospitalization or prolonged hospitalization for a complication of therapy administration will be reported as an SAE.
Routine treatment or monitoring of the studied indication not associated with any deterioration in condition.
A procedure for protocol/disease-related investigations (e.g., surgery, scans, endoscopy, sampling for laboratory tests, bone marrow sampling). However, hospitalization or prolonged hospitalization for a complication of such procedures remains a reportable SAE.
Hospitalization or prolongation of hospitalization for technical, practical, or social reasons, in absence of an AE.
A procedure that is planned (i.e., planned prior to start of treatment on study); must be documented in the source document and the eCRF. Hospitalization or prolonged hospitalization for a complication remains a reportable SAE.
An elective treatment of or an elective procedure for a pre-existing condition, unrelated to the studied indication that has not worsened from baseline.
Emergency outpatient treatment or observation that does not result in admission, unless fulfilling other seriousness criteria above.

### Example 4: Open-Label, Multicenter Study to Explore the Pharmacodynamic Effects of COMPOUND (I) in Subjects with Active Crohn's Disease

Study Objectives

The primary objective of the study is to explore the mechanism of action of COMPOUND (I) 160 mg once daily (QD) in subjects with active Crohn's disease (CD).

The secondary objectives are:
To explore the effect of COMPOUND (I) 160 mg QD on inflammatory cytokines, and gene expression in the intestinal mucosa
To evaluate the safety and tolerability of COMPOUND (I) 160 mg QD in subjects with active CD

The exploratory objectives are:
To explore the effect of COMPOUND (I) 160 mg QD on clinical activity in subjects with active CD
To explore the effect of COMPOUND (I) 160 mg QD on endoscopic outcomes in subjects with active CD
To explore the effect of COMPOUND (I) 160 mg QD on the expression of immune biomarkers on circulating mononuclear cells
To explore the association of pharmacodynamic (PD) markers with clinical and endoscopic outcomes in subjects with active CD receiving COMPOUND (I) 160 mg QD To explore the effect of COMPOUND (I) 160 mg QD on biomarkers of intestinal inflammation and tissue damage such as high sensitivity C-reactive protein (hsCRP), regenerating islet-derived 3-alpha (Reg-3c), and fecal calprotectin (FCP), in subjects with active CD

### Study Endpoints

The study endpoints are listed in Table 12.

TABLE 12
Study Endpoints

| Endpoint | Name | Description | Timeframe |
|---|---|---|---|
| Primary | PD | Change from baseline of SMAD7 | Week 12 |
| | | expression in the intestinal mucosa | |
| | | at Week 12, from biopsy samples | |
| | | taken during ileocolonoscopy | |
| Secondary | PD | Change from baseline in the | Week 12 |
| | | messenger RNA (mRNA) | |
| | | expression of inflammatory | |
| | | cytokines such as, but not limited | |
| | | to, interleukin (IL)-10, IL-25, | |
| | | chemokine (C-C motif) ligand 20 | |
| | | (CCL20) and tumor necrosis factor | |
| | | alpha, (TNF-α) in the intestinal | |
| | | mucosa at Week 12, from biopsy | |
| | | samples during ileocolonoscopy | |
| | Safety | The evaluation of safety and | Through |
| | | tolerability of COMPOUND (I), | Week 52 |
| | | assessed by the type, frequency | and 4 |
| | | and severity of adverse events, and | weeks |
| | | its relationship to investigational | postdose |
| | | product (IP), discontinuation due | |
| | | to adverse events, and clinically | |
| | | significant changes in | |
| | | electrocardiograms (ECGs), vital | |
| | | signs, and/or laboratory findings | |
| Exploratory | Efficacy | The proportion of subjects | Weeks 4, |
| | | achieving clinical remission, | 8, 12 |
| | | defined as a Crohn's Disease | |
| | | Activity Index (CDAI) score <150, | |
| | | at Weeks 4, 8, and 12 | |
| | Efficacy | The proportion of subjects | Weeks 4, |
| | | achieving a clinical response, | 8, 12 |
| | | defined as a decrease from baseline | |
| | | of ∃ 100 points in CDAI score, at | |
| | | Weeks 4, 8, and 12 | |
| | Efficacy | Change from baseline in the CDAI | Weeks 4, |
| | | score at Weeks 4, 8, and 12 | 8, 12 |
| | Efficacy | Change from baseline in the | Weeks 4, |
| | | two-item patient reported outcome | 8, 12 |
| | | (PRO-2) score at Weeks 4, 8, and | |
| | | 12 | |
| | Efficacy | Change from baseline in the | Through |
| | | Harvey-Bradshaw Index | Week 52 |
| | | (HBI) score through Week 52 and | and 4 |
| | | 4 weeks postdose | weeks |
| | | | postdose |
| | Efficacy | Change from baseline in the | Week 12 |
| | | Simple Endoscopic Score for | |
| | | Crohn's Disease (SES-CD) at | |
| | | Week 12 | |
| | PD | Change from baseline in | Weeks 4, |
| | | percentages of circulating | 8, 12 |
| | | mononuclear cells expressing | |
| | | markers such as, but not limited to, | |
| | | IL-17A, Foxp3 and CCR9 at | |
| | | Weeks 4, 8, and 12, as measured | |
| | | by flow cytometry | |
| | PD | Change from baseline in high | Weeks 4, |
| | | sensitivity C-reactive protein | 8, 12, 24, |
| | | (hsCRP) at Weeks 4, 8, 12, 24, 52 | 52 and 4 |
| | | and 4 weeks postdose | weeks |
| | | | postdose |
| | PD | Change from baseline in fecal | Weeks 4, |
| | | calprotectin (FCP) at Weeks 4, 8, | 8, 12, 24, |
| | | 12, 24, 52 and 4 weeks postdose | 52 and 4 |
| | | | weeks |
| | | | postdose |
| | PD | Change from baseline in serum | Weeks 4, |
| | | biomarkers such as, but not limited | 8, 12, 24, |
| | | to, regenerating islet-derived | 52 and 4 |
| | | 3-alpha (Reg-3a) | weeks |
| | | and CCL20, at Weeks 4, 8, 12, 24, | postdose |
| | | 52 and 4 weeks postdose | |
| | PD | Change from baseline in | Week 12 |
| | | microbiome from feces and | |
| | | intestinal mucosa at Week 12 | |
| | PD/ | The correlation between | Weeks 4, |
| | Efficacy | biomarkers and endoscopic | 8, 12, 24, |
| | | outcomes through Week 12, and | 52 and 4 |
| | | clinical outcomes at | weeks |
| | | Weeks 4, 8, 12, 52 and 4 weeks | postdose |
| | | postdose | |

### Study Design

A schematic diagram illustrating the study design is shown in FIG. 5.

This is an open-label, multicenter study to explore the effect of oral COMPOUND (I) on PD outcomes for 12 weeks in subjects with active CD, defined as having a Crohn's Disease Activity Index (CDAI) score ≥220 and ≤450 and a Simple Endoscopic Score for Crohn's Disease (SES-CD)≥7 (or SES-CD ≥4 if the subject has ileitis only).

Subjects will be screened to provide 16 enrolled subjects who complete 12 weeks of COMPOUND (I) 160 mg QD treatment as open-label therapy. Subjects who discontinue the study prior to the Week 12 Visit will be replaced.

Eligible subjects will be enrolled and enter the Treatment Period at the Baseline Visit (Week 0/Visit 2), and will be assigned to receive IP as COMPOUND (I) 160 mg QD for 12 weeks.

This study also offers subjects with the option to continue with maintenance treatment at the discretion of the Investigator, beginning with alternating no IP for 4 weeks, followed by COMPOUND (I) 160 mg QD for 4 weeks, up to Week 52 during the Maintenance Period.

The study will consist of 4 periods:
Screening Period—up to 4 weeks
Induction Period—12 weeks (Week 0 to Week 12)
Maintenance Period—40 weeks (Week 12 to Week 52)
Follow-up Period—4 weeks (ie, no IP taken)

Subjects who prematurely discontinue treatment from this study prior to Week 52 will have an Early Termination Visit and also enter the 4-week Follow-up Period.

The number of subjects with previous exposure to TNF-α blockers is targeted to be approximately 40% (i.e., approximately 6 subjects).

### Study Duration

The overall study duration will be up to 60 weeks with 4 different periods: up to 4 weeks in the Screening Period; 12 weeks in the Induction Period; 40 weeks in the Maintenance Period; and 4 weeks in the Follow-up Period.

### Study Population

Number of Subjects:

Approximately 16 subjects with active CD who complete 12 weeks of treatment will be enrolled from Europe.

Inclusion Criteria:

Subjects must satisfy the following criteria to be screened and enrolled in the study:
Subject is a male or female >18 years of age at the time of signing the informed consent form (ICF).
Subject must understand and voluntarily sign an ICF prior to any study-related assessments/procedures being conducted.
Subject is willing and able to adhere to the study visit schedule and other protocol requirements.
Subject must have a diagnosis of CD with a duration of at least 3 months prior to Screening Visit 1.
Subject must have a diagnosis of ileitis, ileocolitis or colitis, as determined by endoscopic, radiographic or any other imaging modality (e.g., magnetic resonance imaging [MRI], or computed tomography [CT] scan).
Subject must have active disease, defined as a CDAI score ≥220 and ≤450 at screening.
Subject must have a SES-CD ≥7 at screening; subjects with ileitis only must have a SES-CD ≥4 at screening.
Subject must have failed or experienced intolerance to at least one of the following: aminosalicylates; budesonide; systemic corticosteroids; immunosuppressants (eg, azathioprine [AZA], 6-mercaptopurine [6-MP], or methotrexate [MTX]); or TNF-α blockers (eg, infliximab or adalimumab).
Subject must meet the following laboratory criteria:
(One laboratory test repeat is allowed during the Screening Period after consultation with the medical monitor.)
White blood cell (WBC) count ≥3000/mm3 (≥3.0×10⁹/L)
Platelet count ≥100,000/mm3 (≥100×10⁹/L)
Serum creatinine ≤1.5 mg/dL (≤132.6 μmol/L)
Aspartate aminotransferase (AST)/serum glutamic oxaloacetic transaminase (SGOT), alanine aminotransferase (ALT)/serum glutamic pyruvic transaminase (SGPT)≤2.5× upper limit of normal (ULN)
Total bilirubin ≤2 mg/dL (34 μmol/L), unless the subject has a confirmed diagnosis of Gilbert's disease
Hemoglobin ≥8 g/dL (≥4.98 mmol/L)
Activated partial thromboplastin time (APTT)≤1.5×ULN

Exclusion Criteria:

The presence of any of the following will exclude a subject from screening and enrollment:
Subject has CD involvement of the upper gastrointestinal tract.
Subject has a diagnosis of UC, indeterminate colitis, ischemic colitis, microscopic colitis, radiation colitis or diverticular disease-associated colitis.
Subject has local manifestations of CD such as strictures, abscesses, short bowel syndrome or other disease complications for which surgery might be indicated or could confound the evaluation of efficacy.
Subject had an intestinal resection within 6 months or any intra-abdominal surgery within 3 months prior to Screening Visit 1.
Subject has an ileostomy or a colostomy.
Subject had prior treatment with mycophenolic acid, tacrolimus, sirolimus, cyclosporine, thalidomide or apheresis (e.g., Adacolumn®) within 8 weeks prior to Screening Visit 1.
Subject has received intravenous (IV) corticosteroids within 2 weeks prior to Screening Visit 1.
Subject has initiated, discontinued or changed the dose of oral aminosalicylates within 2 weeks prior to Screening Visit 1.
Subject has initiated, changed or discontinued the allowed dose of oral corticosteroids (prednisone ≤20 mg/day or equivalent, budesonide ≤9 mg/day) within 3 weeks prior to Screening Visit 1.
Subject has initiated immunosuppressants (e.g., AZA, 6-MP, or MTX) within 12 weeks prior to Screening Visit 1.
Subject has discontinued or changed the allowed dose of immunosuppressants (eg, AZA, 6-MP, or MTX) within 8 weeks prior to Screening Visit 1.
Subject has received topical GI treatments, such as, 5-aminosalicylic acid (5-ASA) or corticosteroid enemas or suppositories within 2 weeks prior to Screening Visit 1.
Subject has received cholestyramine within 3 weeks prior to Screening Visit 1.
Subject has received antibiotics for the treatment of CD within 3 weeks prior to Screening Visit 1.
Subject had prior treatment with more than 2 TNF-α blockers (eg, infliximab or adalimumab).
Subject had treatment with a TNF-α blocker within 8 weeks prior to the Screening Visit 1.
Subject had prior treatment with any integrin antagonists (eg, natalizumab or vedolizumab).
Subject has received-total parenteral nutrition (TPN) within 4 weeks prior to the Screening Visit 1.
Subject is stool positive for any enteric pathogen or Clostridium difficile (C difficile) toxin at Screening Visit 1.
Subject has a history of any clinically significant neurological, renal, hepatic, gastrointestinal, pulmonary, metabolic, cardiovascular, psychiatric, endocrine, hematological disorder or disease, or any other medical condition that, in the Investigator's opinion, would prevent the subject from participating in the study.
Subject has any condition, including the presence of laboratory abnormalities, which would place the subject at unacceptable risk if he/she were to participate in the study or would confound the ability to interpret data from the study.
Subject is pregnant or breastfeeding.
Subject has a history of any of the following cardiac conditions within 6 months prior to Screening Visit 1 and at any time during the Screening Period, up through the first dose of IP: myocardial infarction, acute coronary syndrome, unstable angina, new onset atrial fibrillation, new onset atrial flutter, second- or third-degree atrioventricular block, ventricular fibrillation, ventricular tachycardia, heart failure, cardiac surgery, interventional cardiac catheterization (with or without a stent placement), interventional electrophysiology procedure, or presence of implanted defibrillator.
Subject has a known active current or history of recurrent bacterial, viral, fungal, mycobacterial or other infections (including but not limited to tuberculosis and atypical mycobacterial disease and herpes zoster), human immunodeficiency virus (HIV), or any major episode of infection requiring hospitalization or treatment with IV or oral antibiotics within 4 weeks prior to Screening Visit 1 and at any time during the Screening Period, up through the first dose of IP.
Subject has a history of congenital or acquired immunodeficiency (eg, common variable immunodeficiency disease).
Subject has a history of colorectal cancer or colorectal dysplasia.
Subject has a history of malignancy, except for:
   Treated (i.e., cured) basal cell or squamous cell in situ skin carcinomas
   Treated (i.e., cured) cervical intraepithelial neoplasia or carcinoma in situ of the cervix with no evidence of recurrence within the previous 5 years
Subject has received any investigational drug or device within 1 month prior to Screening Visit 1.
Subject has a history of alcohol, drug, or chemical abuse within the 6 months prior to Screening Visit 1.
Subject has a known hypersensitivity to oligonucleotides or any ingredient in the IP.
Subject has received prior treatment with COMPOUND (I), or participation in a clinical study involving COMPOUND (I).

Biomarkers

High sensitivity C-reactive protein will be analyzed in blood samples. Fecal calprotectin will be assessed in the fecal samples. The schedule and frequency of these collections are presented, e.g., in FIG. 5 (indicated by “B” in open circles).

Description of Study Treatments

Description of Investigational Product(s):

COMPOUND (I) will be provided as 40-mg film coated tablets.

Treatment Administration and Schedule:

Subjects will receive 1 bottle when IP is administered. Four tablets will be taken by subjects QD during the 12-week Induction Period and QD on the months when IP is taken during 40-week Maintenance Period. That is, subjects will be taking the IP as four 40-mg tablets for 160 mg QD dosing. All IP will be provided as open-label treatment during the entire study. Subjects will be instructed to take the IP in the morning, 30 minutes before breakfast, with a glass of water, and they will also be instructed to refer to the label for storage instructions. Treatment and administration schedules are described below in Table 13.

TABLE 13
Dosing Schedule for the Treatment Periods Treatment Group.

| | Induction Period | | | Maintenance Period | | | | | | |
|---|---|---|---|---|---|---|---|---|---|---|
| | Weeks | Weeks | Weeks | Weeks | Weeks | Weeks | Weeks | Weeks | Weeks | Weeks |
| | 0-3 | 4-7 | 8-11 | 12-15 | 16-23 | 24-31 | 32-40 | 40-47 | 48-51 | 52-55 |
| COMP(I) | 160 mg | 160 mg | 160 mg | No IP | 160 mg | No IP | 160 mg | No IP | 160 mg | No IP |
| 160 mg QD | QD | QD | QD | | QD | | QD | | QD | |

Concomitant Medication Procedures

The following concomitant medications are permitted during the study:
Oral aminosalicylates (sulfasalazine [SSZ] or 5-ASA compounds) are allowed during the study, provided that the dose has been stable for at least 2 weeks prior to Screening Visit 1.
   The dose of oral aminosalicylates must remain stable through Week 12, and should remain stable through the Early Termination Visit if early termination occurs prior to Week 12.
   If oral aminosalicylates have been recently discontinued, treatment must have been stopped at least 2 weeks prior to Screening Visit 1.
   The dose of oral aminosalicylates can be changed (ie, tapered, stopped or increased), as clinically indicated, at the discretion of the Investigator after Week 12.
Oral corticosteroids are allowed during the Induction Period, provided that the dose (prednisone ≤20 mg/day or equivalent, budesonide ≤9 mg/day) has been stable for 3 weeks prior to Screening Visit 1.
   The dose of oral corticosteroids must remain stable through Week 12, and should remain stable through the Early Termination Visit if early termination occurs prior to Week 12.
   If oral corticosteroids were recently discontinued, treatment must have been stopped at least 3 weeks prior to Screening Visit 1.
The dose of oral corticosteroids can be changed (ie, tapered, stopped or increased), as clinically indicated, at the discretion of the Investigator after Week 12. Immunosuppressants, such as AZA, 6-MP or MTX are allowed during the study, provided that treatment was initiated >12 weeks prior to Screening Visit 1.
   The dose of immunosuppressants must be at a stable dose for ≥8 weeks prior to Screening Visit 1 and must remain stable through Week 12, and should remain stable through the Early Termination Visit if early termination occurs prior to Week 12.
   If immunosuppressants were recently discontinued, treatment must have been stopped at least 8 weeks prior to Screening Visit 1.
The dose of immunosuppressants can be changed (ie, tapered, stopped or increased), as clinically indicated, at the discretion of the Investigator after Week 12.
Note: The dose of concomitant medications noted above may not be changed from the baseline dose through Week 12 during the study. No new CD therapy can be prescribed once the subject has been enrolled in the study through Week 12.

The following concomitant medications are prohibited:
Uses of any biologic agents, including TNF-α blockers within 8 weeks prior to Screening Visit 1 and through the entire duration of the study.
Use of mycophenolic acid, tacrolimus, sirolimus, cyclosporine, thalidomide or apheresis (eg, Adacolumn) within 8 weeks prior to Screening Visit 1 and through Week 12 of the study.
Use of topical treatment with 5-ASA or corticosteroid enemas or suppositories within 2 weeks prior to Screening Visit 1 and through Week 12 of the study.
Use of IV corticosteroids within 2 weeks prior to Screening Visit 1 and through Week 12 of the study.
Administration of TPN within 4 weeks prior to Screening Visit 1 and through Week 12 of the study.
Chronic use of nonsteroidal antiinflammatory drugs (NSAIDs) through the entire duration of the study. However, low-dose aspirin for cardiovascular prophylaxis is allowed.
Use of antibiotic therapy for the treatment of CD within 3 weeks prior to Screening Visit 1 and through Week 12 of the study.
Use of cholestyramine within 3 weeks prior to Screening Visit 1 and through Week 12 of the study.

### Adverse Events

Adverse Events are defined and reported as described in Example 3.

### Example 5: Open-Label, Multicenter Study to Explore the Efficacy and Safety of COMPOUND (I) in Subjects with Active Ulcerative Colitis

### Study Objectives

The primary objective of the study is to explore the effect of COMPOUND (I) on clinical activity, as measured by the MMS in subjects with active UC.

The secondary objectives are:
To explore the effect of COMPOUND (I) on endoscopic outcome, as measured by the Mayo endoscopic subscore, in subjects with active UC
To evaluate the safety and tolerability of COMPOUND (I) in subjects with active UC

The exploratory objectives are:
To evaluate the change in biomarkers such as hsCRP and FCP in response to COMPOUND (I) in subjects with active UC
To explore the effects of COMPOUND (I) on histological scores in intestinal mucosal biopsies from subjects with active UC
To explore the PD effects of COMPOUND (I) on gene expression in the intestinal mucosal in subjects with active UC
To explore the association of the PD parameters with the efficacy of COMPOUND (I) in subjects with active UC
To measure the systemic exposure of COMPOUND (I) in subjects with active UC

### Study Endpoints

The study endpoints are listed in Table 14.

TABLE 14
Study Endpoints

| Endpoint | Name | Description | Timeframe |
|---|---|---|---|
| Primary | Efficacy | The proportion of subjects | Week 8 |
| | | achieving clinical | |
| | | remission in the MMS, | |
| | | defined as a MMS of ≤ 2, | |
| | | with no individual | |
| | | subscore > 1 | |
| Secondary | Efficacy | The proportion of subjects | Week 8 |
| | | achieving a MMS of ≤ 2, | |
| | | with rectal bleeding | |
| | | subscore of 0 and stool | |
| | | frequency subscore and | |
| | | Mayo endoscopic | |
| | | subscore ≤ 1 | |
| | Efficacy | The proportion of subjects | Week 8 |
| | | achieving a Mayo | |
| | | endoscopic subscore ≤ 1 | |
| | Efficacy | The proportion of subjects | Week 8 |
| | | achieving a Mayo | |
| | | endoscopic subscore by | |
| | | individual segment (rectum, | |
| | | sigmoid, descending colon, | |
| | | transverse colon, ascending | |
| | | colon/cecum) ≤ 1 | |
| | Efficacy | The proportion of subjects | Week 8 |
| | | achieving clinical response | |
| | | in the MMS, defined as a | |
| | | decreasefrom baseline of at | |
| | | least 2 points and at least | |
| | | 25%, along with a reduction | |
| | | in the RBS of at least 1 | |
| | | point or an absolute | |
| | | RBS ≤ 1 | |
| | Efficacy | The proportion of subjects | Week 8 |
| | | achieving endoscopic | |
| | | response, defined as a | |
| | | decrease from baseline of at | |
| | | least 1 point in the Mayo | |
| | | endoscopic subscore | |
| | Efficacy | The proportion of subjects | Week 8 |
| | | achieving clinical remission | |
| | | in the TMS, defined as a | |
| | | TMS of ≤ 2, with no | |
| | | individual subscore > 1 | |
| | Efficacy | The proportion of subjects | Week 8 |
| | | achieving clinical response | |
| | | in the TMS, defined as a | |
| | | decrease from baseline in | |
| | | the TMS of at least 3 points | |
| | | and at least 30%, along with | |
| | | a reduction in the RBS of at | |
| | | least 1 point or an absolute | |
| | | RBS of ≤ 1 | |
| | Safety | The evaluation of safety and | Through |
| | | tolerability of COMPOUND | Week 52 |
| | | (1), assessed by the type, | |
| | | frequency and severity of | |
| | | adverse events, and its | |
| | | relationship to IP, | |
| | | discontinuation due to | |
| | | adverse events, and | |
| | | clinically significant | |
| | | changes in vital signs, | |
| | | ECGs, and/or laboratory | |
| | | findings | |
| Exploratory | Efficacy | The proportion of subjects | Week 8 |
| | | achieving endoscopic | Week 32 |
| | | remission, defined as a | |
| | | Mayo endoscopic subscore | |
| | | of 0 | |
| | Efficacy | The proportion of subjects | Week 8 |
| | | achieving endoscopic | Week 32 |
| | | remission by segment | |
| | | (rectum, sigmoid, | |
| | | descending colon, | |
| | | transverse colon, ascending | |
| | | colon/cecum), defined as a | |
| | | Mayo endoscopic subscore | |
| | | of 0 | |
| | Efficacy | The proportion of subjects | Week 8 |
| | | achieving clinical remission | |
| | | in the PMS, defined as a | |
| | | PMS of ≤ 2, with no | |
| | | individual subscore > 1 | |
| | Efficacy | The proportion of subjects | Week 8 |
| | | achieving clinical response | |
| | | in the PMS, defined as a | |
| | | decrease from baseline in | |
| | | the PMS at least 2 points | |
| | | and at least 25%, along with | |
| | | a reduction in the RBS of at | |
| | | least 1 point or an absolute | |
| | | RBS of ≤ 1 point | |
| | Efficacy | The proportion of subjects | Week 52 |
| | | who achieve corticosteroid- | |
| | | free clinical remission in | |
| | | the PMS, among subjects | |
| | | receiving oral | |
| | | corticosteroids at baseline | |
| | Efficacy | The proportion of subjects | Week 52 |
| | | who are corticosteroid free, | |
| | | among subjects receiving | |
| | | oral corticosteroid at | |
| | | baseline | |
| | Efficacy | The proportion of subjects | Week 8 |
| | | achieving a RBS ≤ 1 | |
| | Efficacy | The proportion of subjects | Through |
| | | who are in clinical | Week 52 |
| | | remission per the PMS at | |
| | | each time point | |
| | Efficacy | The proportion of subjects | Through |
| | | who are in clinical response | Week 52 |
| | | per the PMS at each time | |
| | | point | |
| | Efficacy | The change from baseline | Week 8 |
| | | in MMS | Week 32 |
| | Efficacy | The change from baseline | Week 8 |
| | | in TMS | Week 32 |
| | Efficacy | The change from baseline in | Week 8 |
| | | Mayo endoscopic subscore | Week 32 |
| | Efficacy | The proportion of subjects | Week 32 |
| | | achieving a MMS of ≤ 2, | |
| | | with RBS of 0 and stool | |
| | | frequency and endoscopy | |
| | | subscore ≤ 1 | |
| | Efficacy | The proportion of subjects | Week 32 |
| | | achieving clinical response | |
| | | in the MMS | |
| Exploratory | Efficacy | The proportion of subjects | Week 32 |
| | | achieving clinical remission | |
| | | in the TMS | |
| | Efficacy | The proportion of subjects | Week 32 |
| | | achieving clinical response | |
| | | in the TMS | |
| | Efficacy | The change from baseline | Through |
| | | in PMS at each time point | Week 52 |
| | Efficacy | The change from baseline | Week 8 |
| | | in histologic scores from | Week 32 |
| | | the intestinal mucosal | |
| | Biomarkers | The change from baseline | Through |
| | | in hsCRP at each time point | Week 52 |
| | Biomarkers | The change from baseline | Through |
| | | in FCP at each time point | Week 52 |
| | Pharmacodynamic | The change from baseline | Week 8 |
| | | in gene expression of PD | Week 32 |
| | | markers such as, but not | |
| | | limited to, TNF-α, IL-5, | |
| | | IL-13, and IL-17 in | |
| | | intestinal mucosa | |
| | Pharmacokinetics | The plasma concentration | Week 4 |
| | | of COMPOUND (I) | Week 12 |

ECG = electrocardiogram;
FCP = fecal calprotectin;
hsCRP = high sensitivity C-reactive protein;
IL = interleukin;
IP = investigational product;
MMS = modified Mayo score;
PD = pharmacodynamic;
PMS = partial Mayo score;
RBS = rectal bleeding subscore;
TMS = total Mayo score;
TNF-α = tumor necrosis factor α.

Study Design

A schematic diagram illustrating the study design is shown in FIG. 6.

This is an open-label, multicenter study to explore the efficacy and safety of oral COMPOUND (I) in subjects with active UC, defined as a MMS ≥4 and <9 and a Mayo endoscopic subscore ≥2.

Approximately 40 subjects will be enrolled to receive open-label, oral COMPOUND (I) 160 mg for duration of 52 week treatment. Enrollment of subjects with previous exposure to TNF-α blockers will be limited to approximately 15 subjects. The number of subjects with extensive colitis is targeted to comprise approximately 50% of the entire study population.

Eligible subjects will have the Baseline Visit (Week 0/Visit 2) and receive the following treatments:
Induction Phase—COMPOUND (I) 160 mg once daily (QD) for 8 weeks;
Extension Phase—COMPOUND (I) 160 mg on alternating dosing schedule for an additional 44 weeks. The alternating dosing schedule comprises six 4-week periods of COMPOUND (I) 160 mg QD treatments that are alternating with five 4-week periods without COMPOUND (I) treatment. See, e.g., Table 15. Subjects who do not achieve at least a 20% decrease in PMS from baseline at Week 12 will be discontinued from the study.

Based on ongoing safety and efficacy assessments performed during the study, the study may continue with the COMPOUND (I) 160 mg QD dose, a QD dose of COMPOUND (I) up to 320 mg may be added, or the study may be terminated.

If the COMPOUND (I) 160 mg QD dose group is discontinued and a new dose group is added, an additional 40 subjects will be enrolled in the new dose group. Subjects enrolled subsequent to the decision to adjust the dose of COMPOUND (I), will receive the following treatments:
Induction Phase—COMPOUND (I), up to 320 mg QD, for 8 weeks;
Extension Phase—COMPOUND (I), up to 320 mg QD on an alternating dosing schedule for an additional 44 weeks. The alternating dosing schedule comprises six 4-week periods of COMPOUND (I) up to 320 mg QD treatments that are alternating with five 4-week periods without COMPOUND (I) treatment. See, e.g., Table 16. Subjects who do not achieve at least a 20% decrease in the PMS from baseline at Week 12 will be discontinued from the study.

Actively enrolled subjects will not be affected by the dose adjustment.

Subjects receiving corticosteroids at baseline will start tapering their corticosteroids at Week 8 (the end of the Induction Phase) if they achieve clinical response, defined as a decrease from baseline of at least 2 points and at least 25%, along with a reduction in the RBS of at least 1 point or an absolute RBS ≤1 in the MMS. The endoscopy subscore assessed by the investigator will be used for the calculation of the Week 8 MMS.

The study will consist of 4 phases:
Screening Phase—up to 4 weeks
Induction Phase—8 weeks
Extension Phase—44 weeks
Observational Follow-up Phase—4 weeks

Subjects who complete the Extension Phase, and those subjects who prematurely discontinue from the study for any reason, will enter the post-treatment Observational Follow-up Phase, the 4-week period after the last dose of IP.

Study Duration

The overall study duration will be up to 60 weeks, with different phases as follows: up to 4 weeks in the Screening Phase; 8 weeks in the Induction Phase; 44 weeks in Extension Phase, and 4 weeks in the post-treatment Observational Follow-up Phase.

Pharmacokinetics

A sparse and intensive PK substudy has been incorporated into the study to monitor systematic exposure of COMPOUND (I).

Sparse PK sampling is mandatory for all subjects who do not participate in the optional intensive PK substudy. Optional intensive PK sampling will be performed in a subset of approximately 5 subjects.

Sparse Pharmacokinetics Blood Draw:

For all subjects who do not participate in the optional intensive PK substudy, 3 blood draws will be obtained for sparse PK sampling at Week 4 and Week 12, for a total of 6 blood specimens from a subject. The blood draws will occur at 3 time windows: 1) predose (at least >23 hours after the previous dose); 2) 1 to 6 hours postdose; and 3) 6 to 12 hours postdose.

Intensive Pharmacokinetics Blood Draw:

Frequent collection of PK blood samples will be obtained on a subset of subjects who consent to the intensive PK blood draw. Samples will be drawn from approximately 5 subjects. The blood draws will occur at the following time points at Week 4: Predose, and 2, 4, 6, 8, and 24 hours postdose.

Study Population

Number of Subjects:

Approximately 40 subjects will be enrolled worldwide. The total number of subjects may increase to up to 80 if a new dose group is explored.

Inclusion Criteria:

Subjects must satisfy the following criteria to be enrolled in the study:
Subject is a male or female >18 years of age at the time of signing the ICF.
Subject is able to understand and voluntarily sign an ICF prior to conducting any study-related assessments/procedures.
Subject is willing and able to adhere to the study visit schedule and other protocol requirements.
Subject must have a diagnosis of UC with a duration of at least 3 months prior to screening.
Subject must have moderate to severe UC, defined as MMS ≥4 to <9 with RBS >1 at screening.
Subject must have a Mayo endoscopic subscore ≥2 at screening.
Subject must have failed or experienced intolerance to at least one of the following: aminosalicylates; budesonide; systemic corticosteroids; immunosuppressants (eg, 6-mercaptopurine [6-MP], or azathioprine [AZA]) or TNF-α blockers (e.g., infliximab, adalimumab, or golimumab).
Subject must meet the following laboratory criteria:
   a. White blood cell count ≥3000/mm³ (≥3.0×10⁹/L)
   b. Platelet count ≥100,000/mm³ (≥100×10⁹/L)
   c. Serum creatinine ≤1.5 mg/dL (≤132.6 μmol/L)
   d. Aspartate transaminase (AST/serum glutamic oxaloacetic transaminase [SGOT]) and alanine transaminase (ALT/serum pyruvic transaminase [SGPT])≤2.5× upper limit of normal (ULN)
   e. Total bilirubin <2 mg/dL (≤34 μmol/L) unless there is a confirmed diagnosis of Gilbert's disease
   f. Hemoglobin ≥9 g/dL (≥5.6 mmol/L)
   g. Activated partial thromboplastin time (APTT)≤1.5×ULN

### Definition of Ulcerative Colitis Treatment Failure and Intolerance

Aminosalicylates
Failure is defined as having signs and symptoms of active disease despite a history of receiving >8 weeks of treatment with mesalamine or sulfasalazine ≥grams.
Intolerance includes, but is not limited to headache, nausea, vomiting, hypersensitivity reactions (rash, eosinophilia, fever, or lymphadenopathy), nephrotoxicity, hepatotoxicity, blood disorders, oligospermia and infertility.

Systemic Corticosteroids
Failure is defined as having signs and symptoms of active disease despite a long history of receiving at least one 4-week course regimen with prednisone >075 mg/kg daily orally or equivalent, or 1 week of intravenous corticosteroids; or 2 failed attempts to taper corticosteroids to <10 mg of prednisone or equivalent on 2 occasions.
Intolerance includes, but is not limited to, Cushing's syndrome, osteopenia/osteoporosis, hyperglycemia, insomnia, and infection.

Budenoside
Failure is defined as having signs and symptoms of active disease despite a history of receiving >8 weeks of treatment with budenoside at doses >9 grams.
Intolerance is similar to intolerance of systemic corticosteroids.

Immunosuppressants
Failure is defined as signs and symptoms of active UC despite a history of >8 weeks of treatment with azathioprine (≥1.5 mg/kg), or 6-mercaptopurine (≥0.75 mg/kg).
Intolerance includes, but is not limited to nausea, vomiting, abdominal pain, pancreatitis, liver function test abnormalities, lymphopenia, and infection.

TNF-α Blockers
Failure is defined as having an inadequate initial response (primary nonresponder) after an approved labeled dose regimen used for induction therapy or recurrence of disease activity despite scheduled maintenance therapy.
Intolerance includes fever, chills, rash, flush, itching, hypotension, urticaria, myalgia, and arthralgia; which were related to the treatment.

Exclusion Criteria:

The presence of any of the following will exclude a subject from enrollment.
Subject has a diagnosis of CD, indeterminate colitis, ischemic colitis, microscopic colitis, radiation colitis or diverticular disease-associated colitis.
Subject has ulcerative colitis restricted to distal 15 cm or less (eg, ulcerative proctitis).
Subject had surgery as a treatment for UC or who, in the opinion of the Investigator, is likely to require surgery for UC during the study.
Subject has clinical signs suggestive of fulminant colitis or toxic megacolon.
Subject is stool positive for any enteric pathogen or Clostridium difficile (C. difficile) toxin at screening.
Subject has history of colorectal cancer or colorectal dysplasia.
Prior treatment with more than 2 TNF-α blockers (eg, infliximab, adalimumab, or golimumab).
Prior treatment with any integrin antagonists (eg, natalizumab or vedolizumab).
Use of TNF-α blockers within 8 weeks of the screening.
Subject had prior treatment with mycophenolic acid, tacrolimus, sirolimus, cyclosporine, thalidomide or apheresis (eg, Adacolumn®) for the treatment of UC. In addition, prior use of any of these treatment modalities for an indication other than UC within 8 weeks of screening is also excluded.
Subject has received intravenous (IV) corticosteroids within 2 weeks of screening.
Subject has received topical treatment with 5-ASA or corticosteroid enemas or suppositories within 2 weeks of screening.
Subject has received total parenteral nutrition (TPN) within 4 weeks of screening.
Subject has a history of any clinically significant neurological, renal, hepatic, gastrointestinal, pulmonary, metabolic, cardiovascular, psychiatric, endocrine, hematological disorder or disease, or any other medical condition that, in the investigator's opinion, would prevent the subject from participation in the study.
Subject has any condition, including the presence of laboratory abnormalities, which places the subject at unacceptable risk if he/she was to participate in the study or confounds the ability to interpret data from the study.
Subject is pregnant or breastfeeding.
Subject has a history of any of the following cardiac conditions within 6 months of screening: myocardial infarction, acute coronary syndrome, unstable angina, new onset atrial fibrillation, new onset atrial flutter, second- or third-degree atrioventricular block, ventricular fibrillation, ventricular tachycardia, heart failure, cardiac surgery, interventional cardiac catheterization (with or without a stent placement), interventional electrophysiology procedure, or presence of implanted defibrillator.
Subject has a known active current or history of recurrent bacterial, viral, fungal, mycobacterial or other infections (including but not limited to tuberculosis and atypical mycobacterial disease and Herpes zoster), human immunodeficiency virus (HIV), or any major episode of infection requiring hospitalization or treatment with IV or oral antibiotics within 4 weeks of screening.
Subject has a history of congenital or acquired immunodeficiency (eg, common variable immunodeficiency disease).
Subject has a history of malignancy, except for:

Treated (i.e., cured) basal cell or squamous cell in situ skin carcinomas

Treated (i.e., cured) cervical intraepithelial neoplasia or carcinoma in situ of the cervix with no evidence of recurrence within the previous 5 years
Subject has received investigational drug or device within 1 month of screening.
Subject has a history of alcohol, drug, or chemical abuse within the 6 months prior to screening.
Subject has a known hypersensitivity to oligonucleotides or any ingredient in the IP.
Subject has prior treatment with COMPOUND (I) or participated in a clinical study involving COMPOUND (I).

Description of Investigational Product

COMPOUND (I) is 21 nucleotides (21-mer) in length. COMPOUND (I) is an AS ODN (21-mer) with a phosphorothioate backbone. COMPOUND (I) will be provided as 40-mg film coated tablets.

Treatment Administration Schedule

Subjects will receive 1 bottle at each visit. Four tablets will be taken by the subject once daily for the COMPOUND (I) 160 mg QD dose group. Up to eight tablets may be taken by the subject once daily for the new dose group. Subjects will be instructed to take the IP in the morning, 30 minutes before breakfast, with a glass of water. Subjects will also be instructed to refer to the label for storage instructions. The IP treatment and administration schedules are described below in Table 15 and Table 16.

TABLE 15
Dosing Schedule for COMPOUND (I) 160 mg Dose Group

| | Treatment | | | | | | | | | | | | | |
|---|---|---|---|---|---|---|---|---|---|---|---|---|---|---|
| Treatment | WKs | WKs | WK | WKs | WKs | WKs | WKs | Wks | WKs | WKs | WKs | WK s | WK s | WK s |
| Group | 0-3 | 4-7 | 8 | 12-15 | 16-19 | 20-23 | 24-27 | 28-31 | 32-35 | 36-39 | 40-43 | 44-47 | 48-51 | 52-55 |
| COMP(I) | 160 | 160 | 160 | No | 160 | No | 160 | No | 160 | No | 160 | No | 160 | No |
| 160 mg | mg | mg | mg | IP | mg | IP | mg | IP | mg | IP | mg | IP | mg | IP |
| QD | QD | QD | QD | | QD | | QD | | QD | | QD | | QD | |

IP = investigational product; QD = once daily; WK = week.

TABLE 16
Dosing Schedule for a New Dose Group up to 320 mg QD

| | Treatment | | | | | | | | | | | | | |
|---|---|---|---|---|---|---|---|---|---|---|---|---|---|---|
| Treatment | WKs | WKs | WKs | WKs | WKs | WKs | WKs | WKs | WKs | WKs | WKs | WKs | WKs | WKs |
| Group | 0-3 | 4-7 | 8-11 | 12-15 | 16-19 | 20-23 | 24-27 | 28-31 | 32-35 | 36-39 | 40-43 | 44-47 | 48-51 | 52-55 |
| COMP(I) | QD | QD | QD | No | QD | No | QD | No | QD | No | QD | No | QD | No |
| QD dose | dose | dose | dose | IP | dose | IP | dose | IP | dose | IP | dose | IP | dose | IP |
| up to | up to | up to | up to | | up to | | up to | | up to | | up to | | up to | |
| 320 mg | 320 | 320 | 320 | | 320 | | 320 | | 320 | | 320 | | 320 | |
| | mg | mg | mg | | mg | | mg | | mg | | mg | | mg | |

IP = investigational product; QD = once daily; WK = week.

### Concomitant Medications

The following concomitant medications are permitted during the study:
Oral aminosalicylates (sulfasalazine [SSZ] or 5-ASA compounds) are allowed during the study, provided that treatment was initiated at least 6 weeks prior to screening, and has been given at a stable dose for at least 2 weeks prior to screening. The dose of oral aminosalicylates must remain stable through the duration of the study or early termination from the study. If oral aminosalicylates have been recently discontinued, treatment must have been stopped at least 2 weeks prior to screening.
Oral corticosteroids are allowed during the Induction Phase, provided that the dose (prednisone ≤20 mg/day or equivalent, budesonide ≤9 mg/day) has been stable for 3 weeks prior to screening. If oral corticosteroids were recently discontinued, discontinuation must have been completed at least 3 weeks prior to screening. Corticosteroid doses should remain stable until the subject is eligible to start corticosteroids tapering at week 8. The tapering schedule is as follows:
   For prednisone doses >10 mg (or equivalent) daily dose, each week the daily dose is to be tapered by 5 mg until a dose of 10 mg/day is reached, after that each week the daily dose is to be tapered by 2.5 mg until discontinuation.
   For prednisone doses <10 mg (or equivalent), each week the daily dose is to be tapered by 2.5 mg until discontinuation.
   Subjects receiving budesonide should have their daily dose tapered by 3 mg every 3 weeks.
Immunosuppressants, such as AZA, 6-MP or MTX are allowed during the study, provided that treatment was initiated >12 weeks prior to screening. The dose of immunosuppressants must be at a stable dose for ≥8 weeks prior to screening and must remain stable through the duration of the study or early termination from the study. Subjects who discontinued immunosuppressants should have stopped them at least 8 weeks prior to screening.
Acetaminophen and low-dose aspirin for cardiovascular prophylaxis are allowed.
   Note: The dose of concomitant medications noted above may not be increased above the baseline dose during the study. No new UC therapy can be prescribed once the subject has been enrolled into the study.

The following concomitant medications are prohibited:
Use of any biologic agents, including TNF-α blockers within 8 weeks prior to screening and through the duration of the study.
Use of mycophenolic acid, tacrolimus, sirolimus, cyclosporine, thalidomide or apheresis (e.g., Adacolumn) within 8 weeks prior to screening and through the duration of the study.
Use of topical treatment, such as 5-ASA or corticosteroid enemas or suppositories are prohibited during the study and must be discontinued 2 weeks prior to screening.
Use of IV corticosteroids are prohibited during the study and must be discontinued 2 weeks prior to screening.
Administration of TPN within 4 weeks of screening and through the duration of the study.
Chronic use of nonsteroidal anti-inflammatory drugs NSAIDs.

### Adverse Events

Adverse Events are defined and reported as described in Example 3.

### Stopping Criteria

### Individual Subject and Overall Study Stopping Criteria

Individual Subject Stopping Criteria:

The following conditions are considered sufficient reasons for stopping administration of IP to subject(s):
Subjects with a PT (prothrombine time) or an APTT (activated partial thromboplastine time) >2×ULN (upper limit of normal), which is confirmed on repeat testing;
Subjects with clinically significant hemodynamic alterations or signs of systemic inflammatory response associated with elevated complement activation products.

Study Stopping Criteria:

The following conditions are considered sufficient reasons for stopping the entire study:
When 3 or more subjects have been withdrawn from the study for abnormalities in coagulation laboratory parameters or complement activation factors;
Sponsor (safety and clinical) determinations of lack of appropriate benefit/risk balance in patients with ulcerative colitis.

TABLE 4
SEQUENCE LISTING

| ID | SEQUENCE |
|---|---|
| SEQ ID NO: 1 | ATG TTCAGGACCA AACGATCTGC GCTCGTCCGG CGTCTCTGGA |
| | GGAGCCGTGC GCCCGGCGGC GAGGACGAGG AGGAGGGCGC |
| | AGGGGGAGGT GGAGGAGGAG GCGA [symbol] [symbol] [symbol] |
| | [symbol] GACA GCCGAGCGCA TGGGGCCGGT GGCGGCGGCC |
| | CGGGCAGGGC TGGATGCTGC CTGGGCAAGG CGGTGCGAGG |
| | TGCCAAAGGT CACCACCATC CCCACCCGCC AGCCGCGGGC |
| | GCCGGCGCGG CCGGGGGCGC CGAGGCGGAT CTGAAGGCGC |
| | TCACGCACTC GGTGCTCAAG AAACTGAAGG AGCGGCAGCT |
| | GGAGCTGCTG CTCCAGGCCG TGGAGTCCCG CGGCGGGACG |
| | CGCACCGCGT GCCTCCTGCT GCCCGGCCGC CTGGACTGCA |
| | GGCTGGGCCC GGGGGCGCCC GCCGGCGCGC AGCCTGCGCA |
| | GCCGCCCTCG TCCTACTCGC TCCCCCTCCT GCTGTGCAAA |
| | GTGTTCAGGT GGCCGGATCT CAGGCATTCC TCGGAAGTCA |
| | AGAGGCTGTG TTGCTGTGAA TCTTACGGGA AGATCAACCC |
| | CGAGCTGGTG TGCTGCAACC CCCATCACCT TAGCCGACTC |
| | TGCGAACTAG AGTCTCCCCC CCCTCCTTAC TCCAGATACC |
| | CGATGGATTT TCTCAAACCA ACTGCAGACT GTCCAGATGC |
| | TGTGCCTTCC TCCGCTGAAA CAGGGGGAAC GAATTATCTG |
| | GCCCCTGGGG GGCTTTCAGA TTCCCAACTT CTTCTGGAGC |
| | CTGGGGATCG GTCACACTGG TGCGTGGTGG-CATACTGGGA |
| | GGAGAAGACG AGAGTGGGGA GGCTCTACTG TGTCCAGGAG |
| | CCCTCTCTGG ATATCTTCTA TGATCTACCT CAGGGGAATG |
| | GCTTTTGCCT CGGACAGCTC AATTCGGACA ACAAGAGTCA |
| | GCTGGTGCAG AAGGTGCGGA GCAAAATCGG CTGCGGCATC |
| | CAGCTGACGC GGGAGGTGGA TGGTGTGTGG GTGTACAACC |
| | GCAGCAGTTA CCCCATCTTC ATCAAGTCCG CCACACTGGA |
| | CAACCCGGAC TCCAGGACGC TGTTGGTACA CAAGGTGTTC |
| | CCCGGTTTCT CCATCAAGGC TTTCGACTAC GAGAAGGCGT |
| | ACAGCCTGCA GCGGCCCAAT GACCACGAGT TTATGCAGCA |
| | GCCGTGGACG GGCTTTACCG TGCAGATCAG CTTTGTGAAG |
| | GGCTGGGGCC AGTGCTACAC CCGCCAGTTC ATCAGCAGCT |
| | GCCCGTGCTG GCTAGAGGTC ATCTTCAACA GCCGGTAG |
| SEQ ID NO: 2 | 5′-GTCGCCCCTTCTCCCCGCAG-3′ |
| SEQ ID NO: 3 | 5′-GTCGCCCCTTCTCCCCGCAGC-3′ |
| SEQ ID NO: 4 | 5′-GTXGCCCCTTCTCCCXGCAG-3′, wherein X is 5-methyl 2′- |
| | deoxycytidine |
| SEQ ID NO: 5 | 5′-GTXYCCCCTTCTCCCXYCAG-3′, whereby X is a nucleotide |
| | comprising a nitrogenous base selected from the group consisting of |
| | cytosine, 5-methylcytosine and 2-O-methylcytosine, and wherein Y is a |
| | nucleotide comprising a nitrogenous base selected from the group consisting |
| | of guanine, 5-methylguanine and 2-O-methylguanine, optionally provided |
| | that at least one of the nucleotides X or Y comprises a methylated |
| | nitrogenous base. |
| | In some embodiments, at least one of the internucleoside linkages of |
| | the SMAD7 AON is a phosphorothioate linkage. |
| | In some embodiments, all of the internucleoside linkages of the |
| | SMAD7 AON are phosphorothioate linkages. |
| SEQ ID NO: 6 | 5′-GTXGCCCCTTCTCCCXGCAGC-3′, whereby X is a nucleotide |
| | comprising 5-methyl-2′-deoxycytidine. |
| | In some embodiments, at least one of the internucleoside linkages of |
| | the SMAD7 AON is a phosphorothioate linkage. |
| | In some embodiments, all of the internucleoside linkages of the |
| | SMAD7 AON are phosphorothioate linkages. |

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1. A method for treating or managing inflammatory bowel disease (IBD) in a patient having IBD, wherein the method comprises (a) administering to the patient a SMAD7 antisense-oligonucleotide (SMAD7 AON) during a first treatment period at a first dose; and (b) administering to the patient the SMAD7 antisense-oligonucleotide during a second treatment period at a second dose.

2. The method of claim 1, further comprising an observation period after the first treatment period and before the second treatment period, wherein no SMAD7 AON is administered to the patient during the observation period.

3. The method of claim 1, wherein the first dose of the SMAD7 antisense-oligonucleotide is about 40 mg/day or about 160 mg/day.

4. The method of claim 1, wherein the first treatment period is about 4 weeks, about 8 weeks, or about 12 weeks.

5. The method of claim 1, wherein the second dose of the SMAD7 antisense-oligonucleotide is about 40 mg/day or about 160 mg/day.

6. The method of claim 1, wherein the second dose is a lower dose than the first dose.

7. The method of claim 1, wherein the second treatment period is between about 1 week and about 100 weeks, between about 5 weeks and about 95 weeks, between about 10 weeks and about 90 weeks, between about 15 weeks and about 85 weeks, between about 20 weeks and about 80 weeks, between about 25 weeks and about 75 weeks, between about 30 weeks and about 70 weeks, between about 35 weeks and about 65 weeks, between about 40 weeks and about 60 weeks, between about 40 weeks and about 55 weeks, between about 45 weeks and about 55 weeks, or between about 50 weeks and about 55 weeks.

8. The method of claim 7, wherein the second treatment period is about 24 weeks.

9. The method of claim 1, wherein the second treatment period is at least about 1 week, at least about 2 weeks, at least about 4 weeks, at least about 6 weeks, at least about 8 weeks, at least about 10 weeks, at least about 3 months, at least about 6 months, at least about 9 months, at least about 12 months, at least about 18 months, at least about 24 months, at least about 30 months, at least about 3 years, at least about 4 years, at least about 5 years, at least about 6 years, at least about 7 years, at least about 8 years, at least about 9 years, or at least about 10 years.

10. The method of claim 1, wherein during the first treatment period and/or during the second treatment period the SMAD7 antisense-oligonucleotide is administered on an alternating dosing schedule.

11. The method of claim 1, wherein during the second treatment period the SMAD7 antisense-oligonucleotide is administered on an alternating dosing schedule.

12. The method of claim 11, wherein the alternating dosing schedule comprises a) administering the SMAD7 antisense-oligonucleotide at the second dose for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; b) administering a placebo or no SMAD7 antisense-oligonucleotide for about 1 week, about 2 weeks, about 3 weeks, about 4 weeks, about 5 weeks, about 6 weeks, about 8 weeks, about 9 weeks, about 10 weeks, about 11 weeks, about 12 weeks, about 13 weeks, about 14 weeks, about 15 weeks, or about 16 weeks; and repeating a) and optionally b) one or more times.

13. The method of claim 12, wherein a) and optionally b) is repeated at least 2 times, at least 4 times, at least 6 times, at least 8 times, at least 10 times, at least 12 times, at least 14 times, at least 16 times, at least 18 times, at least 20 times, at least 25 times, at least 50 times, at least 100 times, at least 150 times, at least 200 times, or at least 250 times.

14. The method of claim 1, wherein the SMAD7 antisense-oligonucleotide is administered once a day in the morning, at least 30 min before breakfast with water.

15. The method of claim 1, wherein if the patient received an IBD treatment before the first treatment period, then tapering off the IBD treatment at the end of the first treatment period.

16. The method of claim 15, wherein the IBD treatment is selected from the group consisting of a corticosteroid, an aminosalicylate, a budesonide, an immunosuppressant.

17. The method of claim 1, further comprising analyzing the clinical response in the patient at one or more time-points during the first treatment period and/or the second treatment period.

18. The method of claim 1, wherein, if the patient does not show a clinical response at the end of the first treatment period, then terminating the treatment or increasing the first dose and repeating the first treatment period.

19. The method of claim 18, wherein the treatment is terminated if the first dose exceeds the maximum tolerated dose.

20. The method of claim 1, wherein clinical response in the patient is analyzed using the Simple Endoscopic Score for Crohn's Disease (SES-CD), the Crohn's Disease Activity Index (CDAI), a two-item patient reported outcome (PRO-2) test, an intestinal mucosal biopsy.

21. The method of claim 20, wherein the patient shows a clinical response if the patient's CDAI score decreases ≥100 points from baseline during the first treatment period.

22. The method of claim 20, wherein the patient shows a clinical response if the patient's CDAI score is <150 at the end of the first treatment period.

23. The method of claim 20, wherein the patient shows a clinical response if the patient's SES-CD score at the end of the first treatment period is <50% compared to the patient's SES-CD score at the beginning of the first treatment period.

24. The method of claim 20, wherein the patient shows a clinical response if the patient's SES-CD score is ≤2 at the end of the first treatment period.

25. The method of claim 20, wherein the patient shows a clinical response if intestinal mucosal ulcerations are absent in the patient at the end of the first treatment period.

26. The method of claim 1, wherein the patient does not experience a fibrotic event during the first treatment period.

27. The method of claim 26, wherein the patient does not experience a fibrotic event during the first treatment period and the second treatment period.

28. The method of claim 1, further comprising analyzing SMAD7 antisense oligonucleotide levels in a patient sample.

29. The method of claim 28, wherein the patient sample is a serum sample or an intestinal mucosal biopsy sample.

30. The method of claim 1, wherein the patient was diagnosed with ileitis, or ileocilitis.

31. The method of claim 1, wherein the IBD is Crohn's Disease (CD) or ulcerative colitis (UC).

32. The method of claim 1, wherein the patient having IBD has a CDAI score ≥220 and ≤450 and a SES-CD score ≥7 at the beginning of the first treatment period.

33. The method of claim 1, wherein the patient having IBD has experienced treatment failure with or intolerance to an aminosalicylate, a budesonide, a systemic corticosteroid or an immunosuppressant.

34. The method of claim 1, wherein the IBD patient's disease is restricted to the terminal ileum and/or the mid transverse colon.

35. The method of claim 1, wherein the SMAD7 antisense-oligonucleotide is administered orally to the patient having IBD.

36. The method of claim 1, wherein the SMAD7 antisense oligonucleotide targets region 108-128 of human SMAD7 (SEQ ID NO:1).

37. The method of claim 1, wherein the SMAD7 antisense oligonucleotide targets nucleotides 403, 233, 294, 295, 296, 298, 299 or 533 of human SMAD7 (SEQ ID NO: 1).

38. The method of claim 1, wherein the SMAD7 antisense oligonucleotide comprises the nucleotide sequence of SEQ ID NO:2 (5′-GTCGCCCCTTCTCCCCGCAG-3′).

39. The method of claim 1, wherein the SMAD7 AON is COMPOUND (I).

40. (canceled)
